(19) United States
(12) Patent Application Publication
(10) Pub. No.: US 2022/0265791 A1
(43) Pub. Date: Aug. 25, 2022

(54) TARGET PEPTIDES FOR CANCER THERAPY AND DIAGNOSTICS

(71) Applicants: University of Virginia Patent Foundation, Charlottesville, VA (US); The University of Birmingham, Birmingham (GB); The General Hospital Corporation, Boston, MA (US)

(72) Inventors: Donald F. Hunt, Charlottesville, VA (US); Jeffrey Shabanowitz, Charlottesville, VA (US); Keira Mahoney, Charlottesville, VA (US); Jennifer G. Abelin, Boston, MA (US); Mohammad Ovais Azizzanjani, Charlottesville, VA (US); Paisley Trantham Myers, Irmo, SC (US); Stacy Alyse Malaker, Menlo Park, CA (US); Andrew Norris, Oilville, VA (US); Jennifer Hitchcock, Charlottesville, VA (US); Xi Peng, Charlottesville, VA (US); Negin Ghafourian, Charlottesville, VA (US); Mark Cobbold, Winchester, MA (US); Sarah Penny, Birmingham (GB); Nico Buettner, Birmingham (GB); James M. Heather, Boston, MA (US)

(73) Assignees: University of Virginia Patent Foundation, Charlottesville (US); The University of Birmingham, Birmingham (GB); The General Hospital Corporation, Boston, MA (US)

(21) Appl. No.: 17/629,311

(22) PCT Filed: Jul. 21, 2020
(86) PCT No.: PCT/US2020/042897

§ 371 Date: Jan. 21, 2022

Related U.S. Application Data
(60) Provisional application No. 62/876,700, filed on Jul. 21, 2019.

(51) Int. Cl. A61K 39/00; A61K 38/04; A61K 38/17; A61P 35/00; A61K 47/64

(52) U.S. Cl. CPC A61K 39/0011; A61K 38/04; A61K 2039/572; A61P 35/00; A61K 47/6425; A61K 38/1774

(57) ABSTRACT

Provided are compositions that include one or more synthetic target peptides, wherein each synthetic target peptide is about or at least 8-50 amino acids long; and has an amino acid sequence as set forth in Table 2 and/or Table 3. Also provided are in vitro populations of dendritic cells that include the disclosed compositions, in vitro population of CD8+ T cells capable of being activated upon being brought into contact with the disclosed populations of dendritic cells, antibodies or antibody-like molecules that specifically binds to a complex of an MHC class I molecule and a peptide having an amino acid sequence as set forth in Table 2 and/or Table 3, methods for treating and/or preventing cancers by administering a therapeutically effective dose of a composition that includes at least one target peptide having an amino acid sequence as set forth in Table 2 and/or Table 3.

### CROSS-REFERENCE TO RELATED APPLICATION

This application claims the benefit of U.S. Provisional Application Ser. No. 62/876,700, filed Jul. 21, 2019, the disclosure of which is incorporated herein by reference in its entirety.

### GRANT STATEMENT

This invention was made with government support under Grant Nos. AI 033993 and GM 037537 awarded by National Institutes of Health. The Government has certain rights in the invention.

### REFERENCE TO SEQUENCE LISTING

The Sequence Listing associated with the instant disclosure has been electronically submitted to the United States Patent and Trademark Office as a 416 kilobyte ASCII text file created on Jul. 21, 2020 and entitled “3062_64_PCT_ST25.txt”. The Sequence Listing submitted via EFS-Web is hereby incorporated by reference in its entirety.

### TECHNICAL FIELD

The presently disclosed subject matter relates to diagnostics and therapeutics. In particular, it relates to immunotherapies and diagnostics in the context of proliferative diseases such as cancer.

### BACKGROUND

Cells in the mammalian body communicate their health status to the immune system by degrading cellular proteins and presenting fragments of each on the cell surface. The major pathway involves the proteosome, a multi-enzyme particle that converts the linear protein chain into a mixture dominated by 9-12 amino acid peptides. These are then transported into the endoplasmic reticulum via transport associated proteins (TAP). There, one or more chaperone proteins load them onto class I MHC molecules, 47 kDa glycoproteins coded by genes in the major histocompatibility complex. A third protein, beta-microglobulin (12 kDa), stabilizes the resulting complex and the trimer is then transported to the cell surface. Appropriately educated, cytotoxic T-lymphocytes (CTL; CD8^{m} T cells) bind to the class I MHC molecules on the cell surface, sample the peptides being presented and lyse those cells that express new peptides, as a result of viral, bacterial or parasitic infection, tissue transplantation or cellular transformation. Evidence that the immune system plays an active role in the surveillance of tumors includes observations that: (a) immunosuppressed transplant recipients display higher incidences of non-viral cancers than appropriate control populations, (b) cancer patients can exhibit spontaneous adaptive and innate immune responses to their tumor, (c) the presence of tumor infiltrating lymphocytes can be a good indicator of survival, and (d) many healthy blood donors have central memory T cells that respond to and kill cells that present tumor specific class I and class II phosphopeptide antigens.

Identification of cellular antigens is an important goal because these peptides become potential candidates for vaccines and other cancer treatments such as adoptive T cell therapy (ACT). Unfortunately, sequence analysis of antigenic peptides is a daunting task. Each cell expresses several hundred thousand copies of up to six different class I MHC molecules, and more than a hundred different class I MHC molecules exist in the population at large. However, more than eighty percent of the human population has one of five common MHC alleles. These are termed HLA A*0201, A*0101, A*0301, B*0702, and B*4402. Cells synthesize more than ten thousand different proteins each day and it is expected that one or more fragments from most of these will appear on the cell surface in association with an MHC molecule. Mass spectrometry has been used to estimate the number of different peptides presented by a given type of class I MHC molecule, and the total is estimated to be between 6,000 and 10,000. Since each cell can present up to 6 different class I MHC molecules, 36,000 to 60,000 different peptides can be displayed on the cell surface at any one time.

CTLs lyse infected or diseased cells that display as few as 5-50 copies of a particular peptide antigen. On 10⁸ cells, this copy number corresponds to 1-10 fmols of an individual peptide. Diseased cells continue to display the usual number of self peptides along with a small number of additional peptide antigens characteristic of the disease state. The analytical challenge is to be able to identify these antigens in a mixture containing as many as 10,000 self peptides and then sequence them at the low attomole-low femtomole level.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to compositions comprising at least or about 1, 2, 3, 4, 5, 6, 7, 8, 9, 10 or more target peptides each of which are about or at least 8, 9, 10, 11, 12, 13, 14, 15, or more amino acids long wherein the target peptides comprise for example, amino acid sequences as set forth in Table 2; and wherein the composition has the ability to stimulate a T cell mediated immune response to at least one of the target synthetic peptides.

In some embodiments, at least one serine, threonine, or tyrosine residue in any of the peptides is phosphorylated and/or replaced with a homo-serine. In some embodiments, the composition comprises a non-hydrolyzable phosphate.

In some embodiments, the composition comprises at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, f-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP and TPS.

In some embodiments, the composition comprises an adjuvant selected from the group consisting of montanide ISA-51 (Seppic Inc., Fairfield, N.J., United States of America), QS-21 (Aquila Biopharmaceuticals, Inc., Framingham, Mass., United States of America), tetanus helper peptides (such as but not limited to QYIKANSKFIGITEL (SEQ ID NO: 1472) and/or AQYIKANSKFIGITEL (SEQ ID NO: 1473), GM-CSF, cyclophosphamide, bacillus Calmette-Guerin (BCG), Corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

In some embodiments, the presently disclosed subject matter relates to methods of treating or preventing cancer comprising administering to a patient in need thereof a dose of the aforementioned compositions.

In some embodiments, the presently disclosed subject matter relates to methods of making a cancer vaccine comprising combining one or more of the aforementioned peptides with an adjuvant and a pharmaceutically acceptable carrier.

In some embodiments, the presently disclosed subject matter relates to a kit comprising at least one target peptide composition comprising at least one target peptide and a cytokine and/or an adjuvant. In some embodiments, the kit comprises at least 2, 3, 4 or 5 or more compositions. In some embodiments, the cytokine is selected from the group consisting of transforming growth factors (TGFs) such as TGF-alpha and TGF-beta; insulin-like growth factor-I and -II; erythropoietin (EPO); osteoinductive factors; interferons such as interferon-alpha-beta, and -gamma; colony stimulating factors (CSFs) such as macrophage-CSF (M-CSF); granulocyte-macrophage-CSF (GM-CSF); and granulocyte-CSF (G-CSF). In some embodiments, the cytokine is selected from the group consisting of nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha-beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand, or FLT-3, angiostatin, thrombospondin, endostatin, tumor necrosis factor, and LT.

In some embodiments, the kit comprises at least one additional peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, f-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS.

In some embodiments, the kit comprises at least one target peptide that comprises an amino acid as set forth in Table 2.

More particularly, in some embodiments the presently disclosed subject matter provides compositions comprising, consisting essentially of, or consisting of at least or about 1, 2, 3, 4, 5, 6, 7, 8, 9, 10, or more synthetic target peptides, wherein each synthetic target peptide is about or at least 8-50 amino acids long; and comprises, consists essentially of, or consists of an amino acid sequence as set forth in Table 2, and optionally wherein said composition stimulates a T cell-mediated immune response to at least one of the synthetic target peptides. In some embodiments, at least one of the synthetic target peptides comprises a substitution of a serine residue with a homo-serine residue. In some embodiments, at least one of the synthetic target peptides is a phosphopeptide that comprises a phosphate group, optionally a non-hydrolyzable phosphate group. In some embodiments, the composition is immunologically suitable for at least 60%, 70%, 75%, 90%, 85%, 90%, 95%, of patients of a given cancer. In some embodiments, the composition comprises, consists essentially of, or consists of at least 5, 10, 15, or more different target peptides selected from the group consisting of the peptides listed in Table 2 and/or Table 3.

In some embodiments, at least one of the synthetic target peptides is capable of binding to an MHC class I molecule, optionally an MHC class I molecule selected from the group consisting of an HLA A*0101 allele, an HLA-A*0201 allele, an HLA B*2705 allele, an HLA A*0301 allele, an HLA B*0702 allele, and an HLA B*4402 allele.

In some embodiments, the composition is capable of increasing the 5-year survival rate of a cancer patient treated with the composition by at least 20 percent relative to average 5-year survival rates that could have been expected without treatment with the composition. In some embodiments, the composition is capable of increasing the survival rate of a cancer patient treated with the composition by at least 20 percent relative to a survival rate that could have been expected without treatment with the composition. In some embodiments, the composition is capable of increasing the treatment response rate of a cancer patient treated with the composition by at least 20 percent relative to a treatment rate that could have been expected without treatment with the composition. In some embodiments, the composition is capable of increasing the overall median survival of a cancer patient treated with the composition by at least two months relative to an overall median survival that could have been expected without treatment with the composition.

In some embodiments, the composition further comprises at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, R-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

In some embodiments, the composition further comprises an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

In some embodiments, the presently disclosed subject matter also provides in vitro populations of dendritic cells comprising, consisting essentially of, or consisting of the composition of any one of claims 1-14 or a composition comprising at least one target peptide comprising an amino acid sequence as set forth in Table 2.

In some embodiments, the presently disclosed subject matter also provides in vitro populations of CD8⁺ T cells capable of being activated upon being brought into contact with a population of dendritic cells, wherein the dendritic cells comprise, consist essentially of, or consist of a composition as disclosed herein or a composition comprising, consisting essentially of, or consisting of at least one target peptide comprising an amino acid sequence as set forth in Table 2 and/or Table 3.

In some embodiments, the presently disclosed subject matter also provides antibodies and antibody-like molecules that specifically bind to a complex of an MHC class I molecule and a peptide comprising, consisting essentially of, or consisting of an amino acid sequence as set forth in Table 2 and/or Table 3. In some embodiments, the antibody or antibody-like molecule is a member of the immunoglobulin superfamily. In some embodiments, the antibody or antibody-like molecule comprises, consists essentially of, or consists of a binding member selected from the group consisting an Fab, Fab′, F(ab′)₂, Fv, and a single-chain antibody. In some embodiments, the antibody or antibody-like molecule of the presently disclosed subject matter is conjugated to a therapeutic agent, which in some embodiments is optionally selected from the group consisting of an alkylating agent, an antimetabolite, a mitotic inhibitor, a taxoid, a vinca alkaloid, and an antibiotic. In some embodiments, the antibody or antibody-like molecule is a T cell receptor, optionally conjugated to a CD3 agonist.

In some embodiments, the presently disclosed subject matter also provides in vitro populations of T cells transfected with a nucleic acid encoding a T cell receptor of as set forth herein.

In some embodiments, the presently disclosed subject matter also provides methods for treating and/or preventing cancer. In some embodiments, the presently disclosed methods comprise, consist essentially of, or consist of administering to a subject in need thereof a therapeutically effective dose of a composition as described herein or a composition comprising, consisting essentially of, or consisting of at least one target peptide comprising, consisting essentially of, or consisting of an amino acid sequence as set forth in Table 2 and/or Table 3. In some embodiments, the cancer is selected from the group consisting of breast cancer, colorectal cancer, esophageal cancer, intrahepatic cholangiocarcinoma cancer, optionally bile ductcancer, kidney cancer, leukemia and/or lymphoma, melanoma, head and neck cancer, ovarian cancer, pancreatic cancer, a cancer associated with phosphatase inhibitor dysregulation, a cancer associated with partially-transformed pheripheral blood mononuclear cells (PBMCs), a cancer of the tonsils, lung cancer, cervical cancer, or any combination thereof.

In some embodiments, the cancer is breast cancer, and the at least one target peptide comprises, consists essentially of, or consists of an amino acid sequence that is selected from the group consisting of SEQ ID NOs: 33, 39, 53, 54, 57, 58, 80, 109, 124, 126, 127, 134, 144, 148, 157, 160, 164, 189, 201, 204, 208, 212, 219, 233, 240, 253, 267, 286, 287, 290, 297-299, 304, 372, 373, 383, 388, 389, 424, 439, 440, 450, 461, 473, 478, 479, 494, 496, 503, 504, 506, 515, 516, 523, 564, 567, 573, 575, 576, 578-580, 589, 664, 732, 739, 768, 777, 784, 824, 835, 836, 862, 864-866, 871, 884, 886, 890, 894, 896, 897, 924, 927, 929, 980, 986, 987, 1021, 1057, 1086-1088, 1098, 1122, 1123, 1128, 1130, 1134, 1180, 1188, 1190, 1213, 1221, 1226, 1230, 1231, 1252, 1269, 1273, 1274, 1278, 1285, 1286, 1292, 1309, 1312, 1315, 1352, 1360, 1378, 1385, 1393, 1418, 1420, 1421, 1423, 1432, 1437, 1438, 1447, 1448, and 1469, and any combination thereof.

In some embodiments, the cancer is colorectal cancer, and the at least one target peptide comprises, consists essentially of, or consists of an amino acid sequence that is selected from the group consisting of SEQ ID NOs: 8, 10, 15, 21, 25-27, 51, 57, 58, 72, 73, 85, 106, 116, 117, 142, 156, 190, 201, 233, 263, 268, 270, 286, 299, 394, 395, 402, 412, 450, 472, 483, 484, 489, 530, 551, 565, 567, 574, 575, 582, 583, 584, 629, 664, 680, 681, 724, 741, 768, 776, 823, 835, 836, 851, 866, 868, 910, 919, 923, 938, 941, 952, 991, 1062, 1109, 1143, 1144, 1146, 1148, 1164, 1176, 1186, 1188, 1189, 1192, 1195, 1198, 1209, 1237, 1238, 1239, 1277, 1287, 1288-1290, 1301, 1305, 1317, 1319, 1333, 1347, 1387, 1393, 1399, 1409, 1414, 1419, 1420-1424, 1442, 1452, and 1453, and any combination thereof.

In some embodiments, the cancer is esophageal cancer, and the at least one target peptide comprises, consists essentially of, or consists of an amino acid sequence that is selected from the group consisting of SEQ ID NOs: 145, 305, 416, 490, 742, 765, 952, and 1121, and any combination thereof.

In some embodiments, the cancer is intrahepatic cholangiocarcinoma, optionally a bile duct cancer, and the at least one target peptide comprises, consists essentially of, or consists of an amino acid sequence that is selected from the group consisting of SEQ ID NOs: 157, 158, 184, 217, 232, 250, 278, 316, 416, 431, 469, 471, 486, 488, 498, 532-536, 587-589, 654, 788, 946, 979, 985, 1044, 1052-1054, 1063, 1065, 1094, 1099, 1121, 1133, 1203, 1231, 1282, 1411, 1420, 1469, and 1471, and any combination thereof.

In some embodiments, the cancer is kidney cancer, and the at least one target peptide comprises, consists essentially of, or consists of an amino acid sequence that is selected from the group consisting of SEQ ID NOs: 472, 480, 521, 528-530, 726, 823, 836, 1337, and 1386, and any combination thereof.

In some embodiments, the cancer is leukemia and/or lymphoma, and the at least one target peptide comprises, consists essentially of, or consists of an amino acid sequence that is selected from the group consisting of SEQ ID NOs: 1-5, 7-9, 11, 13, 14, 17-20, 22, 23, 31, 38, 41-50, 52, 54-63, 67, 69, 70, 75, 79, 84, 88, 90, 91, 97, 104, 110, 118, 119, 121, 123, 128, 130, 132, 139, 142, 146-149, 152, 159, 161-165, 168, 170-173, 175, 183, 186, 190, 192, 195, 196, 203-206, 208, 210, 215, 222, 231, 232, 234, 237-239, 244, 245, 250, 251, 254, 257, 264, 265, 268, 269, 271, 273, 276, 278, 281-283, 288, 292, 295, 300-302, 305, 308, 310, 317, 319, 322, 325, 328, 333, 336, 340, 343, 347-350, 353, 355, 368-371, 373, 376, 377, 379-382, 384, 387, 391, 393, 396, 400, 405, 410, 413, 416, 419, 420, 427, 433, 434, 443, 444, 446, 447, 453, 454, 456, 462, 463, 465-468, 470, 472, 476, 477, 480-483, 485, 492, 495, 497, 501, 513, 514, 517, 519, 521, 522, 525, 528-530, 533, 534, 537, 548, 549, 552, 553, 558, 563, 564, 568-570, 581, 582, 585, 586, 589, 590, 592, 593, 595-599, 602-604, 611, 614, 623-625, 627, 628, 630, 631, 633, 636, 638, 639, 644, 645, 648-651, 663, 665, 667, 669, 670, 684, 693, 695, 699, 700, 717, 727, 729, 730, 731, 734-736, 739, 740, 744-753, 756, 758, 759, 762, 763, 766, 768, 769, 771, 773, 776, 777, 780-783, 785-787, 789-795, 797, 799-804, 807-817, 819, 821-827, 830, 832, 837, 838, 840, 843, 848, 851-853, 869, 870, 872-875, 878, 879, 885, 889, 894, 898, 901-903, 908, 909, 913-915, 918, 919, 921, 923, 924, 930, 931, 935, 937, 939, 940, 942, 944, 945, 961, 968, 971, 983, 994, 997, 1006, 1010, 1012, 1014, 1021, 1034, 1036, 1037, 1042, 1047, 1052, 1056, 1058, 1065, 1066, 1070, 1072, 1075, 1078, 1093, 1101, 1104, 1105, 1110-1112, 1118, 1119, 1124, 1125, 1132, 1133, 1135, 1145, 1151, 1157, 1158-1161, 1164-1166, 1168-1175, 1177, 1181, 1182, 1185, 1187, 1191, 1193-1196, 1199-1201, 1206-1209, 1211, 1212, 1214-1218, 1228, 1229, 1245, 1249, 1253, 1276, 1280, 1284, 1293, 1296, 1297, 1305, 1318, 1320-1322, 1324, 1326-1332, 1334, 1337, 1343-1345, 1348, 1350, 1351, 1353, 1354, 1362, 1364, 1369, 1370, 1375, 1377, 1378, 1380, 1384, 1386, 1391, 1393-1400, 1403, 1405, 1406, 1410, 1412, 1417, 1426, 1428, 1434-1436, 1440, 1446, 1450, 1451, 1464, 1466, and 1468, and any combination thereof.

In some embodiments, the cancer is melanoma, and the at least one target peptide comprises, consists essentially of, or consists of an amino acid sequence that is selected from the group consisting of SEQ ID NOs: 12, 21, 28, 34, 54, 65, 99, 156, 175, 205, 207, 238, 239, 268, 329, 337, 346, 350, 371, 373, 379, 380, 423, 448, 467, 487, 512, 530, 571, 577, 655, 688, 690, 700, 712, 728, 738, 741, 743, 746, 768, 772, 778, 779, 782, 785, 788-790, 795, 815, 820, 823, 910, 919, 933, 936, 949, 950, 981, 989, 1085, 1110, 1124, 1126, 1153, 1155, 1199, 1202, 1213, 1220, 1277, 1339, 1387, 1393, and 1448, and any combination thereof.

In some embodiments, the cancer is head and neck cancer, and the at last one target peptide comprises, consists essentially of, or consists of an amino acid sequence that is selected from the group consisting of SEQ ID NOs: 6, 58, 64, 65, 68, 73, 76, 77, 80, 82, 86, 124, 129, 148-150, 157, 164, 166, 175, 178, 179, 184, 185, 187, 194, 202, 213, 220, 221, 225, 226, 228-230, 232, 248, 268, 284-286, 289, 291, 294, 296, 318, 325, 371, 388, 392, 398, 399, 405-409, 411, 414-416, 429, 432, 438, 441, 442, 460, 499, 500, 502, 505, 507-511, 540-543, 566, 572, 585, 589, 591, 601, 609, 612, 620, 621, 622, 630, 637, 640, 641, 668, 683, 704, 725, 737, 815, 824, 839, 841, 845-848, 853, 854, 863, 866, 867, 875, 880-883, 887, 891, 893, 895, 899, 900, 905-907, 910-912, 916-918, 920-922, 924, 926, 928, 930, 932, 934, 943, 944, 948, 951, 953-960, 962-966, 969, 970, 972-978, 982, 984, 985, 988, 991, 995, 996, 998, 1001, 1003-1005, 1007, 1009, 1011, 1013, 1015-1020, 1022, 1023, 1026-1029, 1031, 1033, 1035, 1038, 1039, 1043, 1046, 1049-1051, 1055, 1059, 1060, 1063, 1064, 1068, 1081, 1082, 1095, 1099, 1116, 1137, 1144, 1146, 1151, 1184, 1204, 1205, 1209, 1213, 1219, 1221, 1234, 1246, 1256, 1257, 1262, 1271, 1293, 1300, 1307, 1315, 1316, 1325, 1340, 1389, 1407, 1408, 1411, 1413, 1420, 1429, 1430, 1431, 1455, 1456, 1461-1463, 1467, and 1469, and any combination thereof.

In some embodiments, the cancer is ovarian cancer, and the target peptide comprises, consists essentially of, or consists of an amino acid sequence that is selected from the group consisting of SEQ ID NOs: 29, 32, 40, 139, 193, 224, 261, 464, 666, 685, 686, 689-691, 700, 702, 708, 1040, 1052, 1069, 1139, 1149, 1260, 1265, 1346, 1371, 1379, 1387, and 1388, and any combination thereof.

In some embodiments, the cancer is pancreatic cancer, and the target peptide comprises, consists essentially of, or consists of an amino acid sequence that is selected from the group consisting of SEQ ID NOs: 65, 66, 216, 311-314, 417, 420, 435, 436, 653, 1076, 1077, 1120, 1131, 1261, and 1433, and any combination thereof.

In some embodiments, the cancer is a cancer associated with phosphatase inhibitor dysregulation, and the target peptide comprises, consists essentially of, or consists of an amino acid sequence that is selected from the group consisting of SEQ ID NOs: 30, 31, 35, 37, 74, 87, 111-115, 122, 135, 138-142, 149, 151, 161, 162, 174, 175, 182, 197, 224, 258, 259, 262, 266, 277, 278, 280, 282, 293, 295, 308, 309, 327, 330, 331, 334, 335, 341-348, 351, 352, 356-367, 385, 397, 403, 410, 421, 422, 427, 451, 474, 518, 539, 550, 553, 554, 558, 559, 600, 656-661, 668, 672-679, 682, 687, 692-694, 696, 698, 699, 703, 705-707, 710, 711, 713-715, 720-722, 824, 825, 831, 844-847, 861, 885, 942, 971, 1080, 1090, 1113, 1129, 1138, 1140-1142, 1150, 1152, 1154, 1156, 1158, 1215-1217, 1240-1244, 1275, 1283, 1299, 1310, 1313, 1314, 1342, 1355, 1358, 1361, 1363, 1366, 1372-1374, 1380-1383, 1386, 1390, 1401, 1404, 1415, 1416, 1434, 1439, 1441, 1443, 1444, 1457-1460, and 1469, and any combination thereof.

In some embodiments, the cancer is a cancer associated with partially-transformed pheripheral blood mononuclear cells (PBMCs), and the target peptide comprises, consists essentially of, or consists of an amino acid sequence that is selected from the group consisting of SEQ ID NOs: 8, 23, 31, 36, 74, 83, 92-98, 101, 104, 113, 114, 126, 136, 142, 149, 151, 161, 162, 173-175, 190, 191, 222, 223, 226, 227, 235, 236, 246, 247, 250, 272, 274, 278, 295, 333, 338, 339, 343, 410, 416, 425, 430, 437, 452, 455, 457-459, 474, 490, 519, 520, 526, 527, 533, 534, 537-539, 544, 545, 553-557, 560-562, 594, 602, 607, 608, 654, 699, 718, 815, 824, 825, 828, 829, 844-847, 885, 942, 947, 971, 999, 1011, 1012, 1032, 1074, 1095, 1097, 1115, 1142, 1158, 1159, 1197, 1210, 1217, 1223-1225, 1227, 1229, 1235, 1272, 1279, 1286, 1297, 1298, 1308, 1319, 1334, 1339, 1340, 1341, 1355, 1356, 1361, 1365, 1366, 1368, 1380, 1381, 1383, 1402, 1404, 1434, 1435, 1445, 1449, 1459, 1460, and 1469, and any combination thereof.

In some embodiments, the cancer is a cancer of the tonsils, and the target peptide comprises, consists essentially of, or consists of SEQ ID NO: 768.

In some embodiments, the cancer is lung cancer, and the target peptide comprises, consists essentially of, or consists of an amino acid sequence that is selected from the group consisting of SEQ ID NOs: 119, 251, 256, 428, 470, 549, and 952, and any combination thereof.

In some embodiments, the cancer is cervical cancer, and the target peptide comprises, consists essentially of, or consists of an amino acid sequence that is selected from the group consisting of SEQ ID NOs: 108, 255, 779, 1067, and 1323, and any combination thereof.

In some embodiments, the presently disclosed subject matter also provides methods for treating and/or preventing cancers. In some embodiments, the presently disclosed methods comprise, consist essentially of, or consist of administering to a subject in need thereof a therapeutically effective dose of a composition as described herein or a composition comprising, consisting essentially of, or consisting of at least one target peptide as set forth in Table 2 and/or Table 3 in combination with a pharmaceutically acceptable carrier.

In some embodiments, the presently disclosed subject matter also provides methods for treating and/or preventing cancer comprising, consisting essentially of, or consisting of administering to a subject in need thereof a therapeutically effective dose of the CD8⁺ T cells as described herein in combination with a pharmaceutically acceptable carrier.

In some embodiments, the presently disclosed subject matter also provides methods for treating and/or preventing cancer comprising, consisting essentially of, or consisting of administering to a subject in need thereof an in vitro population of dendritic cells as set forth herein in combination with a pharmaceutically acceptable carrier.

In some embodiments, the presently disclosed subject matter also provides methods for treating and/or preventing cancer comprising, consisting essentially of, or consisting of administering to a subject in need thereof the population of CD8⁺ T cells as set forth herein in combination with a pharmaceutically acceptable carrier.

In some embodiments, the presently disclosed subject matter also provides methods for preparing cancer vaccines. In some embodiments, the presently disclosed methods comprise, consist essentially of, or consist of combining a composition as described herein with an the adjuvant, optionally an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

In some embodiments, the presently disclosed subject matter also provides methods for screening target peptides for inclusion in an immunotherapy composition as described herein and/or for use in the method of using a composition as described herein, comprising, consisting essentially of, or consisting of (a) administering the target peptide to a human; (b) determining whether the target peptide is capable of inducing a target peptide-specific memory T cell response in the human; and (c) selecting the target peptide for inclusion in an immunotherapy composition if the target peptide elicits a memory T cell response in the human.

In some embodiments, the presently disclosed subject matter also provides methods for determining a prognosis of a cancer patient. In some embodiments, the presently disclosed methods comprise, consist essentially of, or consist of (a) administering to the patient a target peptide comprising, consisting essentially of, or consisting of an amino acid sequence as set forth in Table 2 and/or Table 2, wherein the target peptide is associated with the patient's cancer; (b) determining whether the target peptide is capable of inducing a target peptide-specific memory T cell response in the patient; and (c) determining that the patient has a better prognosis if the patient mounts a memory T cell response to the target peptide than if the patient did not mount a memory T cell response to the target peptide.

In some embodiments, the presently disclosed subject matter also provides kits comprising, consisting essentially of, or consisting of at least one target peptide composition comprising, consisting essentially of, or consisting of at least one target peptide comprising, consisting essentially of, or consisting of an amino acid sequence as set forth in Table 2 and/or Table 3 and a cytokine and/or an adjuvant. In some embodiments, a kit of the presently disclosed subject matter comprises, consists essentially of, or consists of at least 2, 3, 4, 5, 6, 7, 8, 9, 10, or greater than 10 target peptide compositions as described herein. In some embodiments, the at least one target peptide composition is a compositions as described herein. In some embodiments, the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF). In some embodiments, the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT). In some embodiments, the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT). In some embodiments, a kit of the presently disclosed subject matter further comprises at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, f-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS. In some embodiments, the at least one target peptide comprises, consists essentially of, or consists of an amino acid sequence as set forth in Table 2 and/or Table 3.

In some embodiments, a composition of the presently disclosed subject matter comprises, consists essentially of, or consists of at least one peptide capable of binding to an MHC class I molecule, optionally an MHC class I molecule selected from the group consisting of an HLA A*0101 allele, an HLA-A*0201 allele, an HLA B*2705 allele, an HLA A*0301 allele, an HLA B*0702 allele, and an HLA B*4402 allele.

In some embodiments, the presently disclosed subject matter also provides compositions comprising (a) at least one synthetic target peptide, wherein the at least one synthetic target peptide (i) is from 8 to 50 amino acids long; and (ii) comprises an amino acid sequence selected from the group consisting of SEQ ID NO: 150, wherein the serine at the fourth position is phosphorylated or replaced with a mimetic of phosphoserine; SEQ ID NO: 195, wherein the tyrosine at the sixth position is phosphorylated or replaced with a mimetic of phosphotyrosine; SEQ ID NO: 196, wherein the tyrosine at the sixth position is phosphorylated or replaced with a mimetic of phosphotyrosine; SEQ ID NO: 234, wherein the serine at the fourth position is phosphorylated or replaced with a mimetic of phosphoserine; SEQ ID NO: 257, wherein the serine at the seventh position is phosphorylated or replaced with a mimetic of phosphoserine; SEQ ID NO: 293, wherein the tyrosine at the fourth position is phosphorylated or replaced with a mimetic of phosphotyrosine; SEQ ID NO: 331, wherein the serine at the fourth position is phosphorylated or replaced with a mimetic of phosphoserine; SEQ ID NO: 369, wherein the serine at the fourth position is phosphorylated or replaced with a mimetic of phosphoserine; SEQ ID NO: 381, wherein the serine at the eighth position is phosphorylated or replaced with a mimetic of phosphoserine; SEQ ID NO: 408, wherein the serine at the fourth position is phosphorylated or replaced with a mimetic of phosphoserine; SEQ ID NO: 409, wherein the serine at the fourth position is phosphorylated or replaced with a mimetic of phosphoserine; SEQ ID NO: 432, wherein the serine at the fourth position is phosphorylated or replaced with a mimetic of phosphoserine, and/or wherein the methionine at the third position, the methionine at the eighth position, or both are oxidized, or any combination thereof, SEQ ID NO: 481, wherein the serine at the fourth position is phosphorylated or replaced with a mimetic of phosphoserine; SEQ ID NO: 601, wherein the serine at the fourth position is phosphorylated or replaced with a mimetic of phosphoserine; SEQ ID NO: 726, wherein the serine at the fifth position is phosphorylated or replaced with a mimetic of phosphoserine, and/or wherein the methionine at the seventh position is oxidized, or a combination thereof, SEQ ID NO: 729, wherein the serine at the fifth position is phosphorylated or replaced with a mimetic of phosphoserine; SEQ ID NO: 752, wherein the serine at the eighth position is phosphorylated or replaced with a mimetic of phosphoserine; SEQ ID NO: 792, wherein the serine at the fourth position is phosphorylated or replaced with a mimetic of phosphoserine; SEQ ID NO: 912, wherein the serine at the fourth position is phosphorylated or replaced with a mimetic of phosphoserine; SEQ ID NO: 973, wherein the serine at the fourth position is phosphorylated or replaced with a mimetic of phosphoserine, and/or wherein the methionine at the sixth position is oxidized, or a combination thereof; SEQ ID NO: 1128, wherein the serine at the fourth position is phosphorylated or replaced with a mimetic of phosphoserine; and SEQ ID NO: 1269, wherein one or more of the serines at the fourth, fifth, and/or seventh positions is phosphorylated and/or replaced with a mimetic of phosphoserine, or any combination thereof, and (b) an adjuvant.

In some embodiments, the presently disclosed subject matter also provides compositions comprising, consisting essentially of, or consisting of (a) at least one synthetic target peptide, wherein the at least one synthetic target peptide (i) is from 8 to 50 amino acids long; and (ii) comprises an amino acid sequence selected from the group consisting of SEQ ID NOs: 25-27, 33, 34, 38, 39, 41, 42, 65, 66, 77, 82, 112, 131, 140, 141, 174, 190, 193, 201, 227, 242, 246, 247, 286, 294, 301, 336, 338, 348, 355-362, 364-367, 375, 384, 390, 393, 398, 399, 414, 417, 418, 422, 429, 431, 433, 436, 449, 454, 474, 479, 483, 502, 508, 514, 517, 518, 520, 521, 524-526, 528, 530, 531, 533, 538-540, 542, 543, 545, 552-555, 559, 569, 573, 592, 596-599, 604, 607, 619, 658-661, 668, 671, 672, 705, 707, 722-724, 727, 740, 765, 771, 773, 787, 797, 824, 825, 828, 831, 836, 851, 852, 854, 867, 884, 892, 894-896, 907-910, 915, 922, 929, 932, 940, 944, 950, 962, 973, 987, 998, 1019, 1020, 1022, 1045, 1050, 1054, 1066, 1096, 1101, 1103, 1108, 1117, 1130, 1140, 1142, 1144, 1153, 1154, 1158, 1160, 1224, 1234-1236, 1258, 1260, 1277, 1298, 1311, 1314, 1315, 1321, 1322, 1336, 1340, 1372, 1378, 1380, 1382, 1383, 13861419, 1458, and 1460, wherein at least one methionine is oxidized; and (b) an adjuvant.

In some embodiments, the presently disclosed subject matter also provides compositions comprising, consisting essentially of, or consisting of (a) at least one synthetic target peptide, wherein the at least one synthetic target peptide (i) is from 8 to 50 amino acids long; and (ii) comprises an amino acid sequence selected from the group consisting of SEQ ID NO: 100, wherein the serine at the second position, the serine at the third position, or both are phosphorylated or replaced with a mimetic of phosphoserine; SEQ ID NO: 102, wherein the serine at the second position, the serine at the third position, or both are phosphorylated or replaced with a mimetic of phosphoserine; SEQ ID NO: 103, wherein the serine at the second position, the serine at the third position, or both are phosphorylated or replaced with a mimetic of phosphoserine; SEQ ID NO: 117, wherein the tryotophan at the seventh position is oxidized; SEQ ID NO: 125, wherein the serine at the ninth position is phosphorylated or replaced with a mimetic of phosphoserine and the threonine at the seventh position is unmodified; SEQ ID NO: 207, wherein the serine at the fourth position is phosphorylated or replaced with a mimetic of phosphoserine and the serine at the ninth position is unmodified; SEQ ID NO: 209, wherein the serine at the fourth position is phosphorylated or replaced with a mimetic of phosphoserine, and optionally wherein the serine at the third position is also phosphorylated or replaced with a mimetic of phosphoserine; SEQ ID NO: 233, wherein the serine at the fifth position is phosphorylated or replaced with a mimetic of phosphoserine, and optionally wherein the threonine at the fourth position is also phosphorylated or replaced with a mimetic of phosphothreonine; SEQ ID NO: 245, wherein the threonine at the fifth position is phosphorylated or replaced with a mimetic of phosphothreonine, and optionally wherein the serine at the fourth position is also phosphorylated or replaced with a mimetic of phosphoserine; SEQ ID NO: 287, wherein the serine at the fourth position is phosphorylated or replaced with a mimetic of phosphoserine and the serine at the second position is phosphorylated or replaced with a mimetic of phosphoserine and/or the threonine at the third position is phosphorylated or replaced with a mimetic of phosphothreonine; or the serine at the second position is phosphorylated or replaced with a mimetic of phosphoserine and the threonine at the third position is phosphorylated or replaced with a mimetic of phosphothreonine; SEQ ID NO: 304, wherein the serine at the eighth position is phosphorylated or replaced with a mimetic of phosphoserine, and optionally wherein the serine at the fourth position is also phosphorylated or replaced with a mimetic of phosphoserine; SEQ ID NO: 309, wherein the serine at the eighth position is phosphorylated or replaced with a mimetic of phosphoserine, and optionally wherein the threonine at the fourth position is also phosphorylated or replaced with a mimetic of phosphothreonine; SEQ ID NO: 312 or 314, wherein the serine at the eighth position is phosphorylated or replaced with a mimetic of phosphoserine, and optionally wherein the serine at the fourth position is also phosphorylated or replaced with a mimetic of phosphoserine; SEQ ID NO: 323, wherein the serine at the sixth position is phosphorylated or replaced with a mimetic of phosphoserine, the threonine at the seventh position is phosphorylated or replaced with a mimetic of phosphothreonine, the serine at the twelfth position is phosphorylated or replaced with a mimetic of phosphoserine, or a combination thereof, provided that if the serine at the sixth position is phosphorylated or replaced with a mimetic of phosphoserine, at least one of the threonine at the seventh position and the serine at the twelfth position is also modified; SEQ ID NO: 405, wherein the serine at the fourth position is phosphorylated or replaced with a mimetic of phosphoserine, and optionally wherein the serine at the third position is also phosphorylated or replaced with a mimetic of phosphoserine; SEQ ID NO: 419, wherein the serine at the second position is phosphorylated or replaced with a mimetic of phosphoserine, the serine at the third position is phosphorylated or replaced with a mimetic of phosphoserine, the serine at the fourth position is phosphorylated or replaced with a mimetic of phosphoserine, or any combination thereof, provided that if the serine at the second position is phosphorylated or replaced with a mimetic of phosphoserine, at least one of the serines at the third and fourth positions is also modified; SEQ ID NO: 439, wherein the serine at the sixth position is phosphorylated or replaced with a mimetic of phosphoserine, and optionally wherein the serine at the fourth position is also phosphorylated or replaced with a mimetic of phosphoserine; SEQ ID NO: 445, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 447, wherein the serine at the fifth position is phosphorylated or replaced with a mimetic of phosphoserine and/or the serine in the sixth position is phosphorylated or replaced with a mimetic of phosphoserine, and optionally wherein the serine at the fourth position is also phosphorylated or replaced with a mimetic of phosphoserine; SEQ ID NO: 457, wherein the serine at the third position is phosphorylated or replaced with a mimetic of phosphoserine, the threonine at the fifth position is phosphorylated or replaced with a mimetic of phosphothreonine, the threonine at the sixth position is phosphorylated or replaced with a mimetic of phosphothreonine, or any combination thereof, provided that if the threonine at the sixth position is phosphorylated or replaced with a mimetic of phosphothreonine, at least one of the amino acids at the third and fifth positions is also modified; SEQ ID NO: 476, wherein the serine at the fourth position is phosphorylated or replaced with a mimetic of phosphoserine, the serine in the fifth position is phosphorylated or replaced with a mimetic of phosphoserine, SEQ ID NO: 447, wherein the serine at the sixth position is phosphorylated or replaced with a mimetic of phosphoserine, or any combination thereof, provided that if the serine at the fourth is phosphorylated or replaced with a mimetic of phosphoserine, at least one of the serines at the fifth and sixth positions is also modified; SEQ ID NO: 484, wherein the serine at the sixth position is phosphorylated or replaced with a mimetic of phosphoserine, and optionally wherein the threonine at the fourth position is also phosphorylated or replaced with a mimetic of phosphothreonine; SEQ ID NO: 536, wherein the serine at the third position is phosphorylated or replaced with a mimetic of phosphoserine, and optionally wherein the serine at the sixth position is also phosphorylated or replaced with a mimetic of phosphoserine; SEQ ID NO: 562, wherein the serine at the seventh position is phosphorylated or replaced with a mimetic of phosphoserine, and optionally wherein the threonine at the sixth position is also phosphorylated or replaced with a mimetic of phosphothreonine; SEQ ID NO: 574, wherein at least one of the serines at the sixth and seventh positions are phosphorylated or replaced with a mimetic of phosphoserine, and further wherein the tryptophan at the second position is oxidized; SEQ ID NO: 584, wherein the glutamine at position 1 is modified to a pyroglutamic acid, and optionally wherein one or more of the threonine at the fourth position and the serines at the seventh position is phosphorylated or replaced with a mimetic of phosphoserine or phosphothreonine; SEQ ID NO: 614, wherein the serine at the fourth position is phosphorylated or replaced with a mimetic of phosphoserine, and optionally wherein the serine at the third position is also phosphorylated or replaced with a mimetic of phosphoserine; SEQ ID NO: 642, wherein the threonine at the third position is phosphorylated or replaced with a mimetic of phosphothreonine and the serine at the fourth position is phosphorylated or replaced with a mimetic of phosphoserine; SEQ ID NO: 663, wherein the threonine at the sixth position is phosphorylated or replaced with a mimetic of phosphothreonine, and optionally wherein the serine at the fifth position is also phosphorylated or replaced with a mimetic of phosphoserine; SEQ ID NO: 718, wherein the serine at the fourth position is phosphorylated or replaced with a mimetic of phosphoserine, and optionally wherein the serine at the third position is also phosphorylated or replaced with a mimetic of phosphoserine; SEQ ID NO: 733, wherein the serine at the fourth position is phosphorylated or replaced with a mimetic of phosphoserine, and optionally wherein the threonine at the fifth position is also phosphorylated or replaced with a mimetic of phosphothreonine; SEQ ID NO: 745, wherein the serine at the fifth position is phosphorylated or replaced with a mimetic of phosphoserine, the serine at the sixth position is phosphorylated or replaced with a mimetic of phosphoserine, the serine at the seventh position is phosphorylated or replaced with a mimetic of phosphoserine, or a combination thereof, provided that if the serine at the fifth position is phosphorylated or replaced with a mimetic of phosphoserine, at least one of the serine at the sixth position and the serine at the seventh position is also modified; SEQ ID NO: 762, wherein the serine at the fourth position is phosphorylated or replaced with a mimetic of phosphoserine, the serine at the fifth position is phosphorylated or replaced with a mimetic of phosphoserine, the serine at the sixth position is phosphorylated or replaced with a mimetic of phosphoserine, or a combination thereof, provided that if the serine at the fourth position is phosphorylated or replaced with a mimetic of phosphoserine, at least one of the serine at the fifth position and the serine at the sixth position is also modified; SEQ ID NO: 767, wherein the serine at the fifth position is phosphorylated or replaced with a mimetic of phosphoserine, and optionally wherein the threonine at the fourth position is also phosphorylated or replaced with a mimetic of phosphothreonine; SEQ ID NO: 768, wherein the serine at the seventh position is phosphorylated or replaced with a mimetic of phosphoserine, and optionally wherein the threonine at the sixth position is also phosphorylated or replaced with a mimetic of phosphothreonine; SEQ ID NO: 774, wherein the serine at the sixth position is phosphorylated or replaced with a mimetic of phosphoserine, and optionally wherein the serine at the fifth position is also phosphorylated or replaced with a mimetic of phosphoserine; SEQ ID NO: 775, wherein the threonine at the seventh position is phosphorylated or replaced with a mimetic of phosphothreonine, and optionally wherein the serine at the sixth position is also phosphorylated or replaced with a mimetic of phosphoserine; SEQ ID NO: 776, wherein the serine at the sixth position is phosphorylated or replaced with a mimetic of phosphoserine, and optionally wherein the serine at the fourth position is also phosphorylated or replaced with a mimetic of phosphoserine; SEQ ID NO: 778, wherein the serine at the seventh position is phosphorylated or replaced with a mimetic of phosphoserine, and optionally wherein the serine at the sixth position is also phosphorylated or replaced with a mimetic of phosphoserine; SEQ ID NO: 779, wherein the serine at the seventh position is phosphorylated or replaced with a mimetic of phosphoserine, and optionally wherein the serine at the sixth position is also phosphorylated or replaced with a mimetic of phosphoserine; SEQ ID NO: 782, wherein the serine at the sixth position is phosphorylated or replaced with a mimetic of phosphoserine, and optionally wherein the serine at the fourth position is also phosphorylated or replaced with a mimetic of phosphoserine; SEQ ID NO: 784, wherein the threonine at the fifth position is phosphorylated or replaced with a mimetic of phosphothreonine and/or the serine at the sixth position is phosphorylated or replaced with a mimetic of phosphoserine, and optionally wherein the serine at the fourth position is also phosphorylated or replaced with a mimetic of phosphoserine; SEQ ID NO: 787, wherein the serine at the fourth position is phosphorylated or replaced with a mimetic of phosphoserine, and optionally wherein the serine at the third position is also phosphorylated or replaced with a mimetic of phosphoserine, and further optionally wherein the methionine at the ninth position is oxidized; SEQ ID NO: 788, wherein the serines at both the third and fourth positions are both independently phosphorylated or replaced with a mimetic of phosphoserine; SEQ ID NO: 789, wherein the serines at both the third and fourth positions are both independently phosphorylated or replaced with a mimetic of phosphoserine; SEQ ID NO: 790, wherein the serine at the fourth position is phosphorylated or replaced with a mimetic of phosphoserine, and optionally wherein the serine at the third position is also phosphorylated or replaced with a mimetic of phosphoserine; SEQ ID NO: 791, wherein the serine at the fourth position is phosphorylated or replaced with a mimetic of phosphoserine, and optionally wherein the serine at the third position is also phosphorylated or replaced with a mimetic of phosphoserine; SEQ ID NO: 802, wherein the threonine at the fourth position is phosphorylated or replaced with a mimetic of phosphothreonine, and optionally wherein the serine at the third position is also phosphorylated or replaced with a mimetic of phosphoserine; SEQ ID NO: 803, wherein the serine at the third position is phosphorylated or replaced with a mimetic of phosphoserine, and optionally wherein the threonine at the fourth position is also phosphorylated or replaced with a mimetic of phosphothreonine; SEQ ID NO: 804, wherein the serine at the third position is phosphorylated or replaced with a mimetic of phosphoserine, and optionally wherein the threonine at the fourth position is also phosphorylated or replaced with a mimetic of phosphothreonine; SEQ ID NO: 806, wherein the threonine at the third position is phosphorylated or replaced with a mimetic of phosphothreonine and the serine at the fourth position is also phosphorylated or replaced with a mimetic of phosphoserine; SEQ ID NO: 855, wherein the threonine at the fifth position is phosphorylated or replaced with a mimetic of phosphothreonine, and optionally wherein the serine at the fourth position is also phosphorylated or replaced with a mimetic of phosphoserine; SEQ ID NO: 923, wherein the serines at both the third and fourth positions are both independently phosphorylated or replaced with a mimetic of phosphoserine; SEQ ID NO: 924, wherein the serine at the fourth position is phosphorylated or replaced with a mimetic of phosphoserine, and optionally wherein the serine at the third position is also phosphorylated or replaced with a mimetic of phosphoserine; SEQ ID NO: 969, wherein the serine at the tenth position is phosphorylated or replaced with a mimetic of phosphoserine, and optionally wherein the serine at the fourth position is also phosphorylated or replaced with a mimetic of phosphoserine; SEQ ID NO: 976, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1009, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine; SEQ ID NO: 1011, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine; SEQ ID NO: 1012, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine; SEQ ID NO: 1061, wherein the serines at both the third and fourth positions are both independently phosphorylated or replaced with a mimetic of phosphoserine; SEQ ID NO: 1076, wherein one but not both of the serines at the fourth and sixth positions is phosphorylated or replaced with a mimetic of phosphoserine; SEQ ID NO: 1079, wherein the serine at the sixth position is phosphorylated or replaced with a mimetic of phosphoserine, and optionally wherein the serine at the fifth position is also phosphorylated or replaced with a mimetic of phosphoserine; SEQ ID NO: 1084, wherein the serines at both the ninth and tenth positions are both independently phosphorylated or replaced with a mimetic of phosphoserine; SEQ ID NO: 1111, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1145, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1157, wherein the serine at the fourth position is phosphorylated or replaced with a mimetic of phosphoserine, and optionally wherein the serine at the third position is also phosphorylated or replaced with a mimetic of phosphoserine; SEQ ID NO: 1163, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1165, wherein the serine at one or both of the fifth and sixth positions are independently phosphorylated or replaced with a mimetic of phosphoserine, and optionally wherein the serine at the ninth position is also phosphorylated or replaced with a mimetic of phosphoserine; SEQ ID NO: 1197, wherein the threonine at the sixth position is phosphorylated or replaced with a mimetic of phosphothreonine, and optionally wherein the serine at the fourth position is also phosphorylated or replaced with a mimetic of phosphoserine; SEQ ID NO: 1202, wherein the threonine at the fourth position is phosphorylated or replaced with a mimetic of phosphothreonine, and optionally wherein the serine at the seventh position is also phosphorylated or replaced with a mimetic of phosphoserine; SEQ ID NO: 1216, wherein the serine at the eighth position is phosphorylated or replaced with a mimetic of phosphoserine, and optionally wherein the serine at the fourth position is also phosphorylated or replaced with a mimetic of phosphoserine; SEQ ID NO: 1220, wherein one, two, or all three of the serines at the fourth, fifth, and sixth positions are phosphorylated or replaced with a mimetic of phosphoserine, provided that if the serine at the fourth position is phosphorylated or replaced with a mimetic of phosphoserine, the serine at the fifth position and/or the serine at the sixth position is also phosphorylated or replaced with a mimetic of phosphoserine; SEQ ID NO: 1221 or 1222, wherein one, two, three, or all four of the serines at the third, fourth, fifth, and eighth positions are phosphorylated or replaced with a mimetic of phosphoserine, provided that if the serine at the eighth position is phosphorylated or replaced with a mimetic of phosphoserine, at least one of the serines at the third, fourth, and fifth positions are also phosphorylated or replaced with a mimetic of phosphoserine, and further provided that if the serines at the third and eighth positions are phosphorylated or replaced with a mimetic of phosphoserine, at least one of the serines at the fourth and fifth positions are also phosphorylated or replaced with a mimetic of phosphoserine; SEQ ID NO: 1243, wherein the serine at the twelfth position is phosphorylated or replaced with a mimetic of phosphoserine, and optionally wherein the serine at the second position is also phosphorylated or replaced with a mimetic of phosphoserine; SEQ ID NO: 1251, wherein the serine at the ninth position is phosphorylated or replaced with a mimetic of phosphoserine, and optionally wherein the serine at the fourth position is also phosphorylated or replaced with a mimetic of phosphoserine; SEQ ID NO: 1256, wherein the threonine at the fifth position is phosphorylated or replaced with a mimetic of phosphothreonine, and optionally wherein the serine at the fourth position is also phosphorylated or replaced with a mimetic of phosphoserine; SEQ ID NO: 1279, wherein the threonine at the sixth position is phosphorylated or replaced with a mimetic of phosphothreonine, and optionally wherein the serine at the fourth position is also phosphorylated or replaced with a mimetic of phosphoserine; SEQ ID NO: 1281, wherein the serines at both the third and fourth positions are both independently phosphorylated or replaced with a mimetic of phosphoserine; SEQ ID NO: 1287 or 1288, wherein one, two, three, or all four of the serines at the fifth, sixth, seventh, and eighth positions are phosphorylated or replaced with a mimetic of phosphoserine, provided that if the serine at the fifth position or at the eighth position is phosphorylated or replaced with a mimetic of phosphoserine, at least one of the serines at the sixth and seventh positions is also phosphorylated or replaced with a mimetic of phosphoserine; SEQ ID NO: 1289 or 1290, wherein one, two, three, or all four of the serines at the seventh, eighth, ninth, and tenth positions are phosphorylated or replaced with a mimetic of phosphoserine, provided that if the serine at the seventh position or at the tenth position is phosphorylated or replaced with a mimetic of phosphoserine, at least one of the serines at the eighth and ninth positions is also phosphorylated or replaced with a mimetic of phosphoserine; SEQ ID NO: 1303, wherein the threonine at the first position is phosphorylated or replaced with a mimetic of phosphothreonine, and optionally wherein the serine at the third position is also phosphorylated or replaced with a mimetic of phosphoserine; SEQ ID NO: 1310, wherein one or both of the threonines at the fifth and sixth positions are phosphorylated or replaced with a mimetic of phosphothreonine, and optionally wherein the serine at fourth position is also phosphorylated or replaced with a mimetic of phosphoserine; SEQ ID NO: 1325, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1329 or 1330, wherein the threonine at the seventh position is phosphorylated or replaced with a mimetic of phosphothreonine, and optionally wherein the threonine at the fourth position is also phosphorylated or replaced with a mimetic of phosphothreonine; SEQ ID NO: 1340 or 1341, wherein the threonine at the fifth position is phosphorylated or replaced with a mimetic of phosphothreonine, and optionally wherein the serine at the fourth position is phosphorylated or replaced with a mimetic of phosphoserine, the methionine at the third position is oxidized, or both; SEQ ID NO: 1359, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1368, wherein the tryptophan at the ninth position is oxidized, and optionally wherein the serine at the fourth position is phosphorylated or replaced with a mimetic of phosphoserine; SEQ ID NO: 1369 or 1370, wherein the serine at the ninth position is phosphorylated or replaced with a mimetic of phosphoserine, and optionally wherein the serine at the fourth position is also phosphorylated or replaced with a mimetic of phosphoserine; SEQ ID NO: 1379 or 1380, wherein the serine at the fourth position is phosphorylated or replaced with a mimetic of phosphoserine, and optionally wherein the serine at the fifth position is also phosphorylated or replaced with a mimetic of phosphoserine, and further optionally wherein the methionine at the ninth position is oxidized; SEQ ID NO: 1392, wherein one, two, or all three of the serines at the seventh, eighth, and ninth positions are phosphorylated or replaced with a mimetic of phosphoserine, provided that if the serine at the seventh position is phosphorylated or replaced with a mimetic of phosphoserine, the serine at the eighth position and/or the serine at the ninth position is also phosphorylated or replaced with a mimetic of phosphoserine; SEQ ID NO: 1412, wherein the serine at the fifth position is phosphorylated or replaced with a mimetic of phosphoserine, and optionally wherein the serine at the fourth position is also phosphorylated or replaced with a mimetic of phosphoserine; SEQ ID NO: 1421, 1422, 1423, or 1424, wherein the serine at the eighth position is phosphorylated or replaced with a mimetic of phosphoserine, and optionally wherein the threonine at the seventh position is phosphorylated or replaced with a mimetic of phosphothreonine; SEQ ID NO: 1429, wherein the serine at the fourth position is phosphorylated or replaced with a mimetic of phosphoserine, and optionally wherein the serine at the third position is also phosphorylated or replaced with a mimetic of phosphoserine; SEQ ID NO: 1430 or 1431, wherein the serine at the fourth position is phosphorylated or replaced with a mimetic of phosphoserine, and optionally wherein the serine at the third position is also phosphorylated or replaced with a mimetic of phosphoserine; SEQ ID NO: 1440, wherein the serine at the eighth position is phosphorylated or replaced with a mimetic of phosphoserine, and optionally wherein the serine at the fourth position is also phosphorylated or replaced with a mimetic of phosphoserine; SEQ ID NO: 1448, wherein the serine at the eighth position is phosphorylated or replaced with a mimetic of phosphoserine, and optionally wherein the serine at the seventh position is also phosphorylated or replaced with a mimetic of phosphoserine; and SEQ ID NO: 1467, wherein the serine at the fourth position is phosphorylated or replaced with a mimetic of phosphoserine, and optionally wherein the serine at the third position is also phosphorylated or replaced with a mimetic of phosphoserine; and (b) an adjuvant. In some embodiments, the at least one synthetic target peptide comprises a substitution of a serine residue with a homo-serine residue. In some embodiments, the at least one synthetic target peptide is a phosphopeptide that comprises a non-hydrolyzable phosphate group. In some embodiments, the at least one synthetic target peptide is capable of binding to an MHC class I molecule of the HLA-A*0201 allele. In some embodiments, the at least one synthetic target peptide is capable of binding to an MHC A1, A2, A3, A24, A68, B15, B40, B44, B53, B7, C3, C4, C5, C7, C12, and/or E allele.

In some embodiments, a composition of the presently disclosed subject matter further comprises at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, j-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

In some embodiments, the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, incomplete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

These and other aspects and embodiments which will be apparent to those of skill in the art upon reading the specification provide the art with immunological tools and agents useful for diagnosing, prognosing, monitoring, and/or treating human cancers.

### DETAILED DESCRIPTION

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the phrase “amino acid sequence as set forth in Table 2” refers to any amino acid sequence that is disclosed in Table 2. In some embodiments, the phrase refers to the full length sequence of any amino acid sequence that is disclosed in Table 2, such that an “amino acid sequence as set forth in Table 2” refers to the full length sequence of any of the sequences disclosed in Table 2. By way of example and not limitation, in some embodiments an “amino acid sequence as set forth in Table 2” refers to the full length amino acid sequence of any peptide disclosed in Table 2 and not to a subsequence of a peptide disclosed in Table 2.

There are twenty naturally occurring amino acids, which are referred to in Tables 2 and 3 by one letter codes as set forth in Table 1.

TABLE 1
Amino Acid Abbreviations

| Amino Acid | 3 Letter Code | 1 Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The presently disclosed subject matter relates in some embodiments to post-translationally-modified immunogenic therapeutic target peptides, e.g., phosphopeptides and/or O-GlcNAc peptides and/or peptides comprising other post-translational modifications, for use in immunotherapy and diagnostic methods of using the target peptides, as well as methods of selecting the same to make compositions for immunotherapy, e.g., in vaccines and/or in compositions useful in adaptive cell transfer.

### I. Target Peptides

In some embodiments, the target peptides of the presently disclosed subject matter are post-translationally-modified by being provided with a phosphate group (referred to herein as “phosphopeptides”) and/or an O-linked beta-N-acetylglucosamine (“O-GlcNAc”) moiety (referred to herein as “O-GlcNAc peptides”) and/or another moiety. In some embodiments, the peptides of the presently disclosed subject matter comprise, consist essentially or, or consist of an amino acid sequence as set forth in Tables 2 and 3.

TABLE 2
Exemplary Peptides of the Presently
Disclosed Subject Matter

| SEQ | | |
|---|---|---|
| ID | | |
| NO:. | SEQUENCE* | TUMORS** |
| 1. | AAAsPLHmL | L |
| 2. | AADGtPKHSF | L |
| 3. | AADsPSQNL | L |
| 4. | AADsPSQNLT | L |
| 5. | AADtPPLETL | L; PT |
| 6. | AAsDTERDGLA | N |
| 7. | AAsPGAPQm | L |
| 8. | ADsGEGDFLA | C; L; PT |
| | EGGGVR | |
| 9. | AEAPLPsPRL | L |
| 10. | AEAPPSKsP | C |
| 11. | AEFPSSGsNSVL | L |
| 12. | AEGsPPPKTY | M |
| 13. | AEIsPGSLP | L |
| 14. | AEIsPGSLPVTA | L |
| 15. | AEKsYQNSP | C |
| 16. | AELsPKNLL | H |
| 17. | AELsPSMAP | U |
| 18. | AELsPTTLSP | L |
| 19. | AELsPVEQKL | L |
| 20. | AEMPTQMsP | L |
| 21. | AEPtPEKEKRF | C; M |
| 22. | AERtPELVEL | L |
| 23. | AEsPERVLL | H; L; PT |
| 24. | AFsPVRSV | H |
| 25. | AImRsPQMV | C |
| 26. | AImRsPQmV | C |
| 27. | AIMRsPQmV | C |
| 28. | ALAAPsPPR | M |
| 29. | ALDsPPPPTL | O |
| 30. | ALDsQVPKV | PI |
| 31. | ALGsRESLATL | L; PI; PT |
| 32. | ALLDIIRsL | O |
| 33. | ALmGsPQLVAA | B |
| 34. | ALRSsPImRK | M |
| 35. | ALSsLIHAL | PI |
| 36. | ALStPVVEK | PT |
| 37. | ALTsELANA | PI |
| 38. | AmAAsPHAV | L |
| 39. | AmDsPLUKY | B |
| 40. | AMGAGHFsV | O |
| 41. | AmLGSKsPDP | L |
| | YRL | |
| 42. | APAGGsPRmL | L |
| 43. | APAsPFRQL | L |
| 44. | APAsPFRQLL | L |
| 45. | APAsPLRPL | L |
| 46. | APAsPNHAGVL | L |
| 47. | APAsPRLL | L |
| 48. | APAsPTHPGL | L |
| 49. | APAsPTHPGLm | L |
| 50. | APDsPSKQL | L |
| 51. | APGPGFSSRsL | C |
| 52. | APKsPSQDVKA | L |
| 53. | APLARASsL | B |
| 54. | APRAPSAsPLAL | B; L; |
| | | M |
| 55. | APRtPPGVTF | L |
| 56. | APSRQIsL | L |
| 57. | APSVRSLsL | B; C; |
| | | L |
| 58. | APSVRsLSL | B; C; |
| | | L; N |
| 59. | APVsPLKF | L |
| 60. | APVsPRPGL | L |
| 61. | APVsPSSQKL | U |
| 62. | APYRGQLAsPSSQ | L |
| 63. | AQDsPTHL | L |
| 64. | ARAsPRLHFL | N |
| 65. | ARFsGFYSm | M; N; |
| | | PC |
| 66. | ARFSGFYsm | PC |
| 67. | ARFsPKVSL | H; L |
| 68. | ARGsLRRLL | N |
| 69. | ARIsRSISL | L |
| 70. | ARIsRSIsL | L |
| 71. | AR(ts)PINLGL | H |
| 72. | ARVsPSTSY | C |
| 73. | ASE(s)P(ss) | C; H; |
| | LIFY | N |
| 74. | AsGVAVSDGVI | PFPT |
| | K*** | |
| 75. | ASLsPSVSK | L |
| 76. | ASLsRPLNY | N |
| 77. | ASmsPGHPTHL | N |
| 78. | ASsPPDRIDIF | H |
| 79. | ASSsPVTLR | L |
| 80. | ASSsQIIHI | B; N |
| 81. | ATIPRPFsV | H |
| 82. | ATmKRmLsL | N |
| 83. | AVILPPLsPY | H; PT |
| | FK | |
| 84. | AyAQPQTTTP | L |
| | LPAVSG | |
| 85. | AYGGLTsPGLSY | C |
| 86. | DEGPGHHHKP | N |
| | GLGEGtP | |
| 87. | DERLRINs | PI |
| 88. | DETERAYsF | L |
| 89. | DGRRtFPRI | H |
| 90. | DLRQAHsL | L |
| 91. | DPLSVsPARW | L |
| 92. | DRKsPRVL | PT |
| 93. | DRKsPSVSL | PT |
| 94. | DRLGsRPSL | PT |
| 95. | DRQRsPIAL | PT |
| 96. | DRSSPP(tt)PL | PT |
| 97. | DsFESIESY | L; PT |
| 98. | DSLARILsF | PT |
| 99. | DssEEK | M |
| 100. | DssEEKF | H |
| 101. | DSsEEKF | H; PT |
| 102. | DssEEKFL | H |
| 103. | DssEEKFLR | H |
| 104. | DSsEEKFLR | H; L; PT |
| 105. | DSVsPSESL | H |
| 106. | DTDsAIGSFRY | C |
| 107. | DTIsPTLGF | H |
| 108. | DVYSGtPTKV | V |
| 109. | DYSPYFKtI | B |
| 110. | EASsVTREL | L |
| 111. | EEAPQtPVAF | PI |
| 112. | EEFsPRQAQmF | PI |
| 113. | EEIGtPRKF | PI; PT |
| 114. | EELsPLALGRF | PI; PT |
| 115. | EEQsFLQKF | PI |
| 116. | EERsPSWISA | C |
| 117. | EERsPSwISA | C |
| 118. | EHVPSSSsI | L |
| 119. | EILNRsPRNR | L; U |
| 120. | ELLPRRNsL | H |
| 121. | EPRNSLPAsPAHQL | L |
| 122. | ERLKIRGsL | PI |
| 123. | ERVDSLVsL | L |
| 124. | FAFPGS(t)N(s)L | B; N |
| 125. | FAFPGSTNsL | H |
| 126. | FASPTsPPVL | B; H; PT |
| 127. | FATIRTAsL | B; H |
| 128. | FAVsPIPGRGGVL | L |
| 129. | FAYGSGNsL | N |
| 130. | FAYsPGGAHGM | L |
| 131. | FAYsPGGAHGmL | H |
| 132. | FAYsPGGAHGML | H; L |
| 133. | FGINsPQAL | H |
| 134. | FGLARAFsL | B |
| 135. | FGRKPsL | PI |
| 136. | FGYDsPHDL | PT |
| 137. | FKLSGLsF | H |
| 138. | FLDsAYFRL | PI |
| 139. | FLLsPSDQEM | L; O; PI |
| 140. | FLLsPSDQEm | PI |
| 141. | FLmsDRSLHL | PI |
| 142. | FLsRSIPSL | C; L; PI; PT |
| 143. | FPAsPSVSL | H |
| 144. | FPLARQFsL | B |
| 145. | FPLsPLRKY | E |
| 146. | FPLsPTKLSQY | H; L |
| 147. | FPRAsPRAL | L |
| 148. | FQYSKSPsL | B; L; N |
| 149. | FRFPsGAEL | H; L; N; PI; 13 |
| T | | |
| 150. | FRRsPTEDF | N |
| 151. | FRYsGRTQA | PI; PT |
| 152. | FsFAGFPSA | L |
| 153. | FsFKKSF | H |
| 154. | FSFKKsFKL | H |
| 155. | FsFKKSFKLS | H |
| 156. | F(sss)HEGFSY | C; M |
| 157. | FSVAsPLTL | B; H; I; N |
| 158. | FSVsPASTL | I |
| 159. | FSYsPRLPL | L |
| 160. | FTDVNsILRY | B |
| 161. | FVsEGDGGRL | L; PI; PT |
| 162. | FVTtPTAEL | L; PI; PT |
| 163. | GARTPSPsL | L |
| 164. | GATTTAPsL | B; H; L; N |
| 165. | GAVsPVGEL | L |
| 166. | GDEGPGHHHKPGLGE | N |
| | GtP | |
| 167. | GEAsPLSSL | H |
| 168. | GEFGGFGsV | L |
| 169. | GEIsPTQIL | H |
| 170. | GEKsPPYGVP | L |
| 171. | GELPsPGKV | L |
| 172. | GELPTsPLHLL | L |
| 173. | GEMsPQRFFF | L; PT |
| 174. | GEmsPQRFFF | PI; PT |
| 175. | GENsGIGKLF | H; L; M; N; |
| | | PI; PT |
| 176. | GEPHsSPEL | H |
| 177. | GEQsPNVSL | H |
| 178. | GERsPPRIL | N |
| 179. | GETsLMRTL | N |
| 180. | GETsPHTFQL | H |
| 181. | GETsPRTKITW | H |
| 182. | GETsYIRVY | PI |
| 183. | GGDsPVRL | L |
| 184. | GGLTsPGLSY | I; N |
| 185. | GGPHFsPEHKEL | N |
| 186. | GHGsPFPSL | L |
| 187. | GHHHKPGLGEGtP | N |
| 188. | GHSKtILcM | H |
| 189. | GIFPGtPLKK | B |
| 190. | GImsPLAKK | C; H; L; PT |
| 191. | GKGGSYSQAASSDsAQ | PT |
| | G | |
| 192. | GLAPNtPGKA | L |
| 193. | GLAPtPPSm | O |
| 194. | GLAsPTAITPV | N |
| 195. | GLLARtPPAA | L |
| 196. | GLLNKtPPTA | L |
| 197. | GLSsLSIHL | H; PI |
| 198. | GLTsPGLSY | H |
| 199. | GLTsPGLSYS | H |
| 200. | GLTsPGLSYSL | H |
| 201. | GmLsPGKSIEV | B; C |
| 202. | GPGHHHKPGLGEGtP | N |
| 203. | GPLSRVKsL | H; L |
| 204. | GPLVRQIsL | B; L |
| 205. | GPRAPsPTKPL | L; M |
| 206. | GPRPGsPSALL | L |
| 207. | GPRsASLLSL | M |
| 208. | GPRsPPVTL | B; L |
| 209. | GP(ss)PWTQL | H |
| 210. | GSDsPRSSL | L |
| 211. | G5DVsLTAcKV^{@@} | H |
| 212. | GsGPEIFTF | B |
| 213. | GSKsPISQL | N |
| 214. | GsPHYFSPFRP | H |
| 215. | GsPIKVTL | L |
| 216. | GsPIKVTLA | PC |
| 217. | GTFPKALsI | I |
| 218. | GTIsPTSSL | H |
| 219. | GtPLSQAIIHQY | B |
| 220. | GTVtPALKL | N |
| 221. | GTYVPSsPTRLAY | N |
| 222. | GVIsPQELLK | H; L; PT |
| 223. | GVIsPQELLKK | H; PT |
| 224. | GVIsPRFDVQL | O; PI |
| 225. | GYVQRNLsLVRG | N |
| 226. | HAVsPIAKY | N; PT |
| 227. | HEFmsDTNL | PT |
| 228. | HERGsLASL | N |
| 229. | HHHKPGLGEGtP | N |
| 230. | FIHKPGLGEGtP | N |
| 231. | HIPsPAKKV | L |
| 232. | HKPGLGEGtP | I; L; N |
| 233. | HLY(ts)LPSL | B; C |
| 234. | HPAsPAHPLL | L |
| 235. | HPFHAtPNTY | PT |
| 236. | HPLtPLITY | PT |
| 237. | HPRPTsQDL | L |
| 238. | HPRsPNVL | L; M |
| 239. | HPYsPLPGL | L; M |
| 240. | HQGKFLQtF | B |
| 241. | HRFsINGHFY | H |
| 242. | HRNsmKVFL | H |
| 243. | HRVsVILKL | H |
| 244. | HRYPTsIASLAF | L |
| 245. | HRY(st)PHAF | H; L |
| 246. | HTAsPTGmMK | PT |
| 247. | HTAsPTGMmK | PT |
| 248. | HTFsPSPKL | N |
| 249. | HTIsPLDL | H |
| 250. | HTIsPSFQL | I; L; PT |
| 251. | HVSLItPTKR | H; L; U |
| 252. | HYSsLVRVL | H |
| 253. | HYSsRLGSAIF | B; H |
| 254. | IADDRQsL | L |
| 255. | IAKsPHSTV | V |
| 256. | IAQDHRSsL | U |
| 257. | IARLPSsTL | L |
| 258. | IGKMRYVsV | PI |
| 259. | IIHsLETKL | PI |
| 260. | IIQsPSSTGLLK | H |
| 261. | ILDISEHtL | O |
| 262. | ILGPPPPsFHL | PI |
| 263. | ILYPRPKsL | C |
| 264. | IPAPPSsPL | L |
| 265. | IPHQRSsL | L |
| 266. | IPKsKFLAL | PI |
| 267. | IPLSKIKtL | B |
| 268. | IPRPLsLIGSTL | C; H; L; M; N |
| 269. | IPRsPFKVKVL | L |
| 270. | IPRTPLsPSPM | C |
| 271. | IPSsPQKVAL | L |
| 272. | IPTsPTSKY | PT |
| 273. | IPVSKPLsL | L |
| 274. | IPVsPHIY | PT |
| 275. | IPYAPsGEIPK | H |
| 276. | IRAsLTKHF | L |
| 277. | IRFGRKPs | PI |
| 278. | IRFGRKPsL | I; L; PI; PT |
| 279. | IRGsKIRFL | H |
| 280. | IRKERPIsL | PI |
| 281. | IRNsQTRKI | L |
| 282. | IRS sYIRVL | L; PI |
| 283. | IRYSGHsL | L |
| 284. | ISIDsPQKL | N |
| 285. | ISNsHPLSL | N |
| 286. | ISS smHSLY | B; C; H; N |
| 287. | I(sts)PSVAL | B |
| 288. | ISVsPLATSAL | L |
| 289. | ISVSRSTsF | N |
| 290. | ITDLPDHLLsY | B |
| 291. | ITItPPDRY | N |
| 292. | ITTsPITVRK | L |
| 293. | ITTtINPRF | PI |
| 294. | ITYmsPAKL | N |
| 295. | IVDsPEKL | L; PI; PT |
| 296. | IVSsLRLAY | N |
| 297. | IYRSQsPHYF | B |
| 298. | IYYKsMPNL | B |
| 299. | IYYQsPLSL | B; C |
| 300. | KADsLEVQQM | L |
| 301. | KADsLEVQQm | L |
| 302. | KADtVSKTEL | L |
| 303. | KAFsPVRS | H |
| 304. | KAFsPVRsV | B; H |
| 305. | kAFsPVRSV^{#} | E; H; L |
| 306. | kAFsPVRSV^{#} | H |
| 307. | kAFsPVRSV^{#} | H |
| 308. | KAFsPVRSVR | L; PI |
| 309. | KAFsPVRsVR | PI |
| 310. | KAFsPVRSVRK | L |
| 311. | KAPsPPPLL | PC |
| 312. | KAPsRQIsL | PC |
| 313. | KAPsRQISL | PC |
| 314. | KAPSRQIsL | PC |
| 315. | KAVsLFLcY^{@} | H |
| 316. | KAVsLFLcY^{@@} | H; I |
| 317. | KEDsDEVHL | L |
| 318. | KEKTIHLtL | N |
| 319. | KELsPAGSI | L |
| 320. | KEQsPEPHL | H |
| 321. | KEStLHLVL | H |
| 322. | KEtPDKVEL | H; L |
| 323. | KEVDPSTGELQsL | H |
| 324. | KEVDP(st)GELQSL | H |
| 325. | KFLsPAQYLY | L; N |
| 326. | KFsPVRSV | H |
| 327. | KGFsGTFQL | PI |
| 328. | KIDsPTKV | L |
| 329. | KIDsPTKVK | M |
| 330. | KIHtLELKL | PI |
| 331. | KIIsAELQKA | PI |
| 332. | KIIsIFSG | H |
| 333. | KIIsIFSGTEK | H; L; PT |
| 334. | KIKsFVKVY | PI |
| 335. | KIKsLEEIYL | PI |
| 336. | KImsPRKAL | L |
| 337. | KIMsPRKAL | M |
| 338. | KIm(ss)PLSK | PT |
| 339. | KIM(ss)PLSK | PT |
| 340. | KIRPHIAtL | L |
| 341. | KI(ss)LEIKL | PI |
| 342. | KLAsPSEVVQQV | PI |
| 343. | KLFHGsLEEL | L; PI; PT |
| 344. | KLHsLIGLGI | PI |
| 345. | KLLDFGsLSNL | PI |
| 346. | KLLDFGsLSNLQV | M; PI |
| 347. | KLLsYIQRL | L; PI |
| 348. | KLmsPKADVKL | L; PI |
| 349. | KLPsGSKKV | L |
| 350. | KLRsPKSEL | L; M |
| 351. | KLwtLVSEQTRV^{&} | PI |
| 352. | KLYsISSQV | PI |
| 353. | KLYTyIQSR | L |
| 354. | KLYTyIQSRF | H |
| 355. | KmAsLARKV | L |
| 356. | KmDsFLDMQL | PI |
| 357. | KMDsFLDmQL | PI |
| 358. | KmDsFLDmQL | PI; PT |
| 359. | KmLsCAGADRL | PI |
| 360. | KmLscAGADRL^{@} | PI |
| 361. | KmLscAGADRL^{@@} | PI |
| 362. | KmLtPRIEL | PI |
| 363. | KMIAPRIEL | PI |
| 364. | Km(ss)YAFFV | PI |
| 365. | KmVsMKPPGF | PI |
| 366. | KmVsmKPPGF | PI |
| 367. | KMVsmKPPGF | PI |
| 368. | KPAsPARRLDL | L |
| 369. | KPAsPTPVI | L |
| 370. | KPAVSRsRSSSL | L |
| 371. | KPFKLSGLsF | H; L; M; N |
| 372. | KPGLGEGtP | B |
| 373. | KPLIRSQsL | B; L; M |
| 374. | KPMtPKVVTL | H |
| 375. | KPmtPKVVTL | H |
| 376. | KPPGtPPPSAL | L |
| 377. | KPPsPGTVL | H; L |
| 378. | KPPsPGTVLAL | H |
| 379. | KPRRFsRSL | H; L; M |
| 380. | KPSsLRRVTI | L; M |
| 381. | KPVGVAAsL | L |
| 382. | KPVsPLLL | L |
| 383. | KQKsLTNLSF | B; H |
| 384. | KQPsFSAKKm | L |
| 385. | KQYsGKFF | PI |
| 386. | KRAsALLNL | H |
| 387. | KRAsRIYNT | L |
| 388. | KRFsFKKsFKL | B; H; N |
| 389. | KRFsLDFNL | B |
| 390. | KRIsIFLSm | H |
| 391. | KRIsIFLSM | H; L |
| 392. | KRIsISTSGGSF | N |
| 393. | KRIsRmRLV | L |
| 394. | KRLsVELTSSL | C |
| 395. | KRLsVELTSSLF | C; H |
| 396. | KRLsVERIYQK | L |
| 397. | KRLtHVYDL | PI |
| 398. | KRmsNELENY | N |
| 399. | KRmsVTEGGIKY | N |
| 400. | KRNsIKKIV | L |
| 401. | KRNsRLGFL | H |
| 402. | KRNtFVGTPF | C |
| 403. | KRRtGALVL | PI |
| 404. | KRsPIFF | H |
| 405. | KR(ss)ISQLL | L; N |
| 406. | KRSsVHGVSF | N |
| 407. | KRTsKYFSL | N |
| 408. | KRVsISEGDDKIEY | N |
| 409. | KRYsAEVRL | N |
| 410. | KRYsRSLTI | H; L; PI; PT |
| 411. | KSDGsFIGY | N |
| 412. | KSDsPAIQL | C |
| 413. | KSDsPSTSSI | L |
| 414. | KSKsmDLGI | N |
| 415. | KSLsPSGLKI | N |
| 416. | KSLsPSLLGY | E; H; I; L; N; |
| | PT | |
| 417. | KSPTsPLNm | PC |
| 418. | KSsIIIRm | H |
| 419. | K(sss)LDKQL | L |
| 420. | KSSsLDKQL | L; PC |
| 421. | KSYsFIARMKA | PI |
| 422. | KSYsFIARmKA | PI |
| 423. | KSYsRSRsR | M |
| 424. | KTDGsFIGY | B |
| 425. | KTEsPRTSGVL | PT |
| 426. | KTFsIGKIAK | H |
| 427. | KTIsLTDFL | H; L; PI |
| 428. | KTKsIAEEL | U |
| 429. | KTKsmFFFL | N |
| 430. | KTLsLVKEL | H; PT |
| 431. | KTmsGTFLL | H; I |
| 432. | KTmsPSQmI | N |
| 433. | KTPsHTRmL | L |
| 434. | KTPsLTRRI | L |
| 435. | KTPTsPLKM | PC |
| 436. | KTPTsPLKm | PC |
| 437. | KTQsLPVTEK | PT |
| 438. | KTRsLSVEI | N |
| 439. | KTRsLsVEIVY | B |
| 440. | KTRsLSVEIVY | B |
| 441. | KTVsEPNLKL | N |
| 442. | KTVsPSPAF | N |
| 443. | KVDsPTVTTTL | L |
| 444. | KVIPVTRsL | L |
| 445. | KVL(ss)LVTL | H |
| 446. | KVY(sss)EFL | H; L |
| 447. | KVY(ss)SEFL | L |
| 448. | KVYtPSISK | M |
| 449. | KYELsVIm | H |
| 450. | KYPDVAsPTL | B; C |
| 451. | LADsPLKL | PI |
| 452. | LALTRSSsL | PT |
| 453. | LDEAGQRStM | L |
| 454. | LDEAGQRStm | L |
| 455. | LEAPPsPSL | PT |
| 456. | LEItPPSSEKL | L |
| 457. | LESP(tt)PLL | H; PT |
| 458. | LESPTtPLL | H; PT |
| 459. | LEsPTTPLL | PT |
| 460. | LIDNsFNRY | N |
| 461. | LIMPRPNsV | B |
| 462. | LLARtPPAA | L |
| 463. | LLNKtPPTA | L |
| 464. | LMHsFILKA | O |
| 465. | LPAFKRKtL | L |
| 466. | LPAsPAGRL | L |
| 467. | LPAsPAHQL | L; M |
| 468. | LPAsPRARLSA | L |
| 469. | LPAsPSVSL | H; I |
| 470. | LPAsPVARR | H; L; U |
| 471. | LPDPGsPRL | I |
| 472. | LPEsPRLTL | C; K; L |
| 473. | LPKARPMsL | B |
| 474. | LPKARPmsL | PI; PT |
| 475. | LPLsPKETV | H |
| 476. | LPL(sss)HLNVY | L |
| 477. | LPNsIASRF | L |
| 478. | LPRMIsHSEL | B |
| 479. | LPRmIsHSEL | B |
| 480. | LPRPLsPTKL | K; L |
| 481. | LPRsPPLKVL | L |
| 482. | LPRsPRLGH | L |
| 483. | LPRSSsmAAGL | C; L |
| 484. | LPR(t)P(s)ASSL | C |
| 485. | LPRtPSYSI | L |
| 486. | LPSESVSsL | I |
| 487. | LPsPRGQRVI | M |
| 488. | LPsPTATSQL | H; I |
| 489. | LPSSGRSsL | C |
| 490. | LPTsPLAmEY | E; PT |
| 491. | LPTsPLAmEY | H |
| 492. | LPVsPGHRKT | L |
| 493. | LPYPVsPKQKY | H |
| 494. | LQHSFsFAGF | B |
| 495. | LQIsPPLHQHL | L |
| 496. | LQIsPVSSY | B; H |
| 497. | LQIsPVSSYA | L |
| 498. | LQLPsPTAT | I |
| 499. | LSAsFRSLY | N |
| 500. | LSAsPLTSL | N |
| 501. | LSDDGKAsL | L |
| 502. | LSDsPSmGRY | N |
| 503. | LSEIKFNsY | B |
| 504. | LSKsEHSLF | B |
| 505. | LSSsPPATHF | N |
| 506. | LTDPSsPTIS | B |
| 507. | LTHsLVLHY | N |
| 508. | LTKsPLAQm | N |
| 509. | LTLsPKLQL | N |
| 510. | LTSsRLLKL | N |
| 511. | LTYRRRLsY | N |
| 512. | LVAsPRLEK | M |
| 513. | LVDsVAKTM | L |
| 514. | LVDsVAKTm | L |
| 515. | LVVsPGQQTL | B |
| 516. | LYTyIQSRF | B; H |
| 517. | mLAEsPSVPRL | L |
| 518. | mLPsILNQL | PI |
| 519. | MPGsPTKTVY | L; PT |
| 520. | mPGsPTKTVY | PT |
| 521. | mPHsPTLRV | K; L |
| 522. | MPHsPTLRV | L |
| 523. | MPKFRMPsL | B |
| 524. | mPLsPDPSHTTL | H |
| 525. | mPmRsPSKL | L |
| 526. | mPNsPAPHF | PT |
| 527. | MPNsPAPHF | PT |
| 528. | mPREPsATRL | K; L |
| 529. | MPREPsATRL | K; L |
| 530. | mPRQPsATRL | C; K; L; M |
| 531. | mPsPATLSHSL | H |
| 532. | MPsPATLSHSL | I |
| 533. | mPsPGGRITL | H; I; L; PT |
| 534. | MPsPGGRITL | I; L; PT |
| 535. | MPSPVsPKL | I |
| 536. | MPsPVSPKL | I |
| 537. | MPVP(tt)PEF | L; PT |
| 538. | mPVP(tt)PEF | PT |
| 539. | mRLsRELQL | PI; PT |
| 540. | mTDtYRLKY | N |
| 541. | MTKSsPLKI | N |
| 542. | mTKSsPLKI | N |
| 543. | mTKssPLKI | N |
| 544. | NAEsGRGQVM | PT |
| 545. | NAEsGRGQVm | PT |
| 546. | NAIsLPTI | H |
| 547. | NEFHsPIGL | H |
| 548. | NGIIRSQsF | L |
| 549. | NIAsPGTVHKR | L; U |
| 550. | NIPsFIVRL | PI |
| 551. | NLIsPVRNGAV | C |
| 552. | NMDsPGPmL | L |
| 553. | NmDsPGPML | L; PI; PT |
| 554. | NmDsPGPmL | PI; PT |
| 555. | NP(ss)PEFFm | H |
| 556. | NRFsPKASL | PT |
| 557. | NRLsKGLQI | PT |
| 558. | NRMsRRIVL | L; PI |
| 559. | NRmsRRIVL | PI |
| 560. | NRRKsALAL | PT |
| 561. | NRRsPPPSL | PT |
| 562. | NSDLP(ts)PL | PT |
| 563. | NSLsPRSSL | H; L |
| 564. | NSVsPSESL | B; H; L |
| 565. | NYQLsPTKL | C |
| 566. | PEVsPRPAL | N |
| 567. | PFKVsPLTF | B; C |
| 568. | PIFNRIsV | L |
| 569. | PIFPmARsI | L |
| 570. | PLVSSSDsPPRPQPAF | L |
| 571. | PRsPPRAL | M |
| 572. | PSPPsPLEKTPL | N |
| 573. | P(ts)PLAmEY | B |
| 574. | PwIPPSsPTTF | C |
| 575. | PYDPALGsPSRLF | B; C; H |
| 576. | QAFLRSVsM | B |
| 577. | QEKsPKQAL | M |
| 578. | QKKIsTNL | B |
| 579. | QLDRIsVYY | B |
| 580. | QLSLRTVsL | B |
| 581. | QPRNSLPAsPAHQL | L |
| 582. | QPRsPVPSAF | C; L |
| 583. | QPRTPHsPPL | C |
| 584. | qPRTPHsPPL | C |
| 585. | QPRTPsPLVL | L; N |
| 586. | QPSsPRVNGL | L |
| 587. | QPStPDPFL | I |
| 588. | QSLLsPLVL | I |
| 589. | QTIsPLSTY | B; H; I; L; N |
| 590. | QTPsPRLAL | L |
| 591. | QTSIQsPSSY | N |
| 592. | QVDPKKRIsm | L |
| 593. | RAAsTARHL | L |
| 594. | RAAtPLPSL | PT |
| 595. | RADsPGRLV | L |
| 596. | RADsPVHm | L |
| 597. | RADsPVHmE | L |
| 598. | RADsPVHmEQ | L |
| 599. | RADsPVHmEQQ | L |
| 600. | RAEsDFVKF | PI |
| 601. | RAEsGPDLRY | N |
| 602. | RAEsPGPGSRL | L; PT |
| 603. | RAEsPTPGM | L |
| 604. | RAEsPTPGm | L |
| 605. | RAGsFSRFY | H |
| 606. | RAHsLARQM | H |
| 607. | RAHtPTPGIYm | H; PT |
| 608. | RAHtPTPGIYM | PT |
| 609. | RAIsPREKI | N |
| 610. | RAKRIsQLF | H |
| 611. | RALsPRVAA | L |
| 612. | RALsSSVIREL | N |
| 613. | RALLPSPVM | H |
| 614. | RA(ss)DIV(s)L | H; L |
| 615. | RASsPFRRV | H |
| 616. | RAsVFVKL | H |
| 617. | RA(ts)LPSL | H |
| 618. | RATsNVFAM | H |
| 619. | RATsNVFAm | H |
| 620. | RATsPLVSL | N |
| 621. | RATsRcLQL^{@@} | N |
| 622. | RAVsPFAKI | N |
| 623. | REAPsPLMI | L |
| 624. | REAsIELPSM | L |
| 625. | REAsPLSSNKLIL | L |
| 626. | REEsPLRIKM | H |
| 627. | REGsFRVTTA | L |
| 628. | REGsGRFSLP | L |
| 629. | REIsSSPTS | C |
| 630. | REKsPGRML | L; N |
| 631. | RELsGTIKEIL | L |
| 632. | RENsFGSPL | H |
| 633. | RENsFGSPLEF | L |
| 634. | REPsPALGPNL | H |
| 635. | REPsPLPELAL | H |
| 636. | REPsPVRYDNL | H; L |
| 637. | RERsPGRLF | N |
| 638. | RERWsFIRA | L |
| 639. | REsPIPIEI | L |
| 640. | REsPRPLQL | N |
| 641. | RESsLGFQL | H; N |
| 642. | RE(ts)PNRIGL | H |
| 643. | RETsPNRIGL | H |
| 644. | REVEsLPAV | L |
| 645. | REVsPEPIV | L |
| 646. | REWsPTPSL | H |
| 647. | REWsPTPSSL | H |
| 648. | REYGsPLKA | L |
| 649. | RFsFKKSF | H; L |
| 650. | RGDsRPRLV | L |
| 651. | RGIsPIVF | L |
| 652. | RGsFEVTL | H |
| 653. | RHPKRSVsL | PC |
| 654. | RIDIsPSTL | I; PT |
| 655. | RIHGsPLQK | M |
| 656. | RILsATTSGIFL | PI |
| 657. | RILsGVVTKM | PI |
| 658. | RILsGVVTKm | PI |
| 659. | RILsGVVTKmKM | PI |
| 660. | RILsGVVTKmKm | PI |
| 661. | RILsKEYNm | PI |
| 662. | RIPsVQINF | H |
| 663. | RIRP(st)PSQL | L |
| 664. | RIStPLTGV | B; C |
| 665. | RIVsPKNSDLK | L |
| 666. | RIYsMSLRL | O |
| 667. | RKAsLRQFL | H; L |
| 668. | RKNsFVmEY | PI; N |
| 669. | RKPsAEmNRI | L |
| 670. | RKPsLAKAL | L |
| 671. | RKSsIIIRm | H |
| 672. | RLAsLmNLGm | PI |
| 673. | RLAsSVLRc^{%} | PI |
| 674. | RLAsSVLRc^{%%} | PI |
| 675. | RLAs5VLRc^{@} | PI |
| 676. | RLAs5VLRc^{@} | PI |
| 677. | RLAs5VLRC^{@@} | PI |
| 678. | RLAsSVLRc^{@@@} | PI |
| 679. | RLAsSVLRcG | PI |
| 680. | RLAsYLSGC | C |
| 681. | RLAsYLSGc^{@} | C |
| 682. | RLDsIVGPQL | PI |
| 683. | RLDsPLSNRY | N |
| 684. | RLDsYVRS | L |
| 685. | RLDsYVRsL | O |
| 686. | RLEsLSYQL | O |
| 687. | RLFsHPREPAL | PI |
| 688. | RLFsKELRc^{%%} | M |
| 689. | RLFsKELRc^{@} | O |
| 690. | RLFsKELRc^{@@} | H; M; O |
| 691. | RLGsFHELL | O |
| 692. | RLIsQIVSS | PI |
| 693. | RLIsQIVSSI | L; PI |
| 694. | RLIsQIVSSITA | PI |
| 695. | RLKsIEERQLLK | H; L |
| 696. | RLKsIIQEV | PI |
| 697. | RLLDPSsPLAL | H |
| 698. | RLLsAAEN | PI |
| 699. | RLLsAAENFL | L; PI; PT |
| 700. | RLLsPLSsA | L; M; O |
| 701. | RLLsPPLRPR | H |
| 702. | RLLsVEGSTL | O |
| 703. | RLLsVNIRV | PI |
| 704. | RLLsWSDNW | N |
| 705. | RLmsGKVKV | PI |
| 706. | RLMsGKVKV | PI |
| 707. | RLmtPKPVSI | PI |
| 708. | RLMtPTLSFL | O |
| 709. | RLPSPtSPF | H |
| 710. | RLPtRLPEI | PI |
| 711. | RLQtQVFKL | PI |
| 712. | RLRSsLVFK | M |
| 713. | RLRsSVPGV | PI |
| 714. | RLRSsVPGV | PI |
| 715. | RLRssVPGV | PI |
| 716. | RLsFLVSY | H |
| 717. | RLsPVPVPR | L |
| 718. | RL(ss)VSVTY | PT |
| 719. | RLYKsEPEL | H |
| 720. | RmIsKLEAQV | PI |
| 721. | RMIsKLEAQV | PI |
| 722. | RmLsLRDQRL | PI |
| 723. | RmsLLSVV | H |
| 724. | RmYsPIIYQA | C |
| 725. | RNKsYSFIA | N |
| 726. | RPAKsLmSI | K |
| 727. | RPAKsmDSL | L |
| 728. | RPARPsRKGL | M |
| 729. | RPARsVPSI | L |
| 730. | RPAsPALLL | L |
| 731. | RPAsPLMHI | L |
| 732. | RPAsRFEVL | B |
| 733. | RPA(st)GGLSL | H |
| 734. | RPAtPHLL | L |
| 735. | RPDsRLLEL | L |
| 736. | RPEsPAGPF | L |
| 737. | RPHLSGRKLsL | N |
| 738. | RPHtPTPGI | M |
| 739. | RPHtPTPGIYM | B; H; L |
| 740. | RPHtPTPGIYm | H; L |
| 741. | RPIsVIGGVSL | C; H; M |
| 742. | RPIsVIGGVSLY | E; H |
| 743. | RPKLFIHSLsF | M |
| 744. | RPKPSSsPVI | L |
| 745. | RPKP(sss)PVIF | H; L |
| 746. | RPKsDIVLL | L; M |
| 747. | RPKsPGGIQP | L |
| 748. | RPKSSsPIRL | L |
| 749. | RPKtPNRASP | L |
| 750. | RPKtPPPAP | L |
| 751. | RPLKPLsPL | H; L |
| 752. | RPLPPPSsL | L |
| 753. | RPLsATRKTL | L |
| 754. | RPLsGSGISAF | H |
| 755. | RPLsHYSSF | H |
| 756. | RPLsKQLSA | L |
| 757. | RPLsLIGSTL | H |
| 758. | RPLsPGALEL | L |
| 759. | RPLsPGALQL | L |
| 760. | RPLsPILHI | H |
| 761. | RPLsPTAFSL | H |
| 762. | RPL(sss)HEA | L |
| 763. | RPLtPRTPA | L |
| 764. | RPLTsPESL | H |
| 765. | RPmsESPHm | E |
| 766. | RPPtPTLSL | L |
| 767. | RPP(ts)PGVFGAL | H |
| 768. | RPQRA(ts)NVF | B; C; H; |
| | | L; M; T |
| 769. | RPQtPKEEA | L |
| 770. | RPRDTRRIsL | H |
| 771. | RPRGPsPLVTm | L |
| 772. | RPRHsLNSL | M |
| 773. | RPRPGtGLGRVm | L |
| 774. | RPRP(ss)VL | H |
| 775. | RPRSG(st)GSSL | H |
| 776. | RPR(s)I(s)VEEF | C; L |
| 777. | RPRSLsSPTV | B; L |
| 778. | RPRSL(ss)PTV | M |
| 779. | RPRSL(ss)PTVTL | H; M; V |
| 780. | RPRsPAGQVAA | L |
| 781. | RPRsPGSNSKVP | L |
| 782. | RPR(s)P(s)PIS | H; L; M |
| 783. | RPRsPSSYDL | L |
| 784. | RPR(sts)QSIVSL | B |
| 785. | RPRTNtPKQL | L; M |
| 786. | RPsLGGRTPL | L |
| 787. | RP(ss)APDLm | L |
| 788. | RP(ss)GFYEL | H; I; M |
| 789. | RP(ss)LPDL | H; L; M |
| 790. | RP(ss)PALYF | H; L; M |
| 791. | RP(ss)PIPLL | L |
| 792. | RPSsPPPFL | L |
| 793. | RPSsPRAGAPHAL | L |
| 794. | RPSsPRVEDL | L |
| 795. | RPSsPSTSw^{&} | L; M |
| 796. | RPSsRAVLY | H |
| 797. | RPSsRVALmVL | L |
| 798. | RPSsVLIEQL | H |
| 799. | RPStPGLSV | L |
| 800. | RPStPHTITL | L |
| 801. | RPStPSRLAL | L |
| 802. | RP(st)PTIDVL | L |
| 803. | RP(st)PTINV | L |
| 804. | RP(st)PTINVL | L |
| 805. | RPTsISWDGL | H |
| 806. | RP(ts)PIQIM | H |
| 807. | RPTsRLNRLP | L |
| 808. | RPVDPRRRsL | L |
| 809. | RPVsPAGPP | L |
| 810. | RPVsPAPGA | L |
| 811. | RPVsPGKDITA | L |
| 812. | RPVsPHSDF | L |
| 813. | RPVsPPQKA | L |
| 814. | RPVsPSAYm | L |
| 815. | RPVsPSSLL | H; L; M; |
| | | N; PT |
| 816. | RPVtPITNF | L |
| 817. | RPVtPPRTA | L |
| 818. | RPWsPPPTGSL | H |
| 819. | RPYPsPGAVL | L |
| 820. | RPYsPPFF | M |
| 821. | RPYsPSEYAL | L |
| 822. | RPYsPSQYAL | H; L |
| 823. | RPYtNKVITL | C; H; K; L; M |
| 824. | RQAsIELPSm | B; H; L; N; |
| | | PI; PT |
| 825. | RQAsIELPSmAV | L; PI; PT |
| 826. | RQAsIELPSMAVA | L |
| 827. | RQAsPPRRL | L |
| 828. | RQFmRRTsL | PT |
| 829. | RQFMRRTsL | PT |
| 830. | RQI(st)SGEL | L |
| 831. | RQmsGAQIKI | PI |
| 832. | RQMsRFKEA | L |
| 833. | RQPsEEEII | H |
| 834. | RQPsEEEIIKL | H |
| 835. | RQPsIELPSM | B; C |
| 836. | RQPsIELPSm | B; C; K |
| 837. | RQPsLAKRV | L |
| 838. | RQPsLKRSL | L |
| 839. | RQSsFEPEF | N |
| 840. | RQYsVTDAL | L |
| 841. | RRAsLSYSF | N |
| 842. | RRFsDFLGL | H |
| 843. | RRFsFSGNTL | H; L |
| 844. | RRFsGLLN | PI; PT |
| 845. | RRFsGLLNC | N; PI; PT |
| 846. | RRFsGLLNc | N; PI; PT |
| 847. | RRFsGLLNc | N; PI; PT |
| 848. | RRFsLSPSL | H; L; N |
| 849. | RRFsLTTLRNF | H |
| 850. | RRFsLTTLRNY | H |
| 851. | RRFsPPRRm | C; H; L |
| 852. | RRFsPPRRmL | L |
| 853. | RRFsSSDFSDL | L; N |
| 854. | RRFsSYSQm | N |
| 855. | RRF(st)EYEL | H |
| 856. | RRFsVSTLRNL | H |
| 857. | RRFsVSTLRNLGL | H |
| 858. | RRFsVSTLRNLGLG | H |
| 859. | RRFsVSTLRNLGLGK | H |
| 860. | RRFsVTLRL | H |
| 861. | RRFtEIYEF | PI |
| 862. | RRGsFEVTLL | B |
| 863. | RRGsFPLAA | N |
| 864. | RRGsGPEIF | B |
| 865. | RRGsGPEIFT | B |
| 866. | RRGsGPEIFTF | B; C; N |
| 867. | RRGsLLGSm | N |
| 868. | RRGsLTLTI | C |
| 869. | RRGsNVALM | L |
| 870. | RRGsPVRQL | L |
| 871. | RRGsYPFIDF | B |
| 872. | RRHsLENKV | L |
| 873. | RRIDIsPSTFRK | L |
| 874. | RRIDIsPSTL | L |
| 875. | RRIsIGSLF | H; L; N |
| 876. | RRIsLTKRL | H |
| 877. | RRIsVFKYV | H |
| 878. | RRIsVTSKV | L |
| 879. | RRKsDDVHL | L |
| 880. | RRKsLVLKF | N |
| 881. | RRLsAARLL | N |
| 882. | RRLsFQAEY | N |
| 883. | RRLsFSTRL | N |
| 884. | RRLsGELISm | B |
| 885. | RRLsLFLVL | H; L; PI; PT |
| 886. | RRLsLPGLL | B |
| 887. | RRLsLSRSL | N |
| 888. | RRLsRKL | H |
| 889. | RRLsSQFEN | L |
| 890. | RRLsVEIYDKF | B |
| 891. | RRIALHSVF | N |
| 892. | RRmsFSGIFR | H |
| 893. | RRMsFSGIFR | H; N |
| 894. | RRmsLLSVV | B; H; L |
| 895. | RRmsVAEQVDY | N |
| 896. | RRmsVGDRAG | B |
| 897. | RRNsFIGTPY | B |
| 898. | RRNsISLREL | L |
| 899. | RRNsKIFLDL | N |
| 900. | RRNsLLHGY | N |
| 901. | RRPKtLRL | L |
| 902. | RRPsHEGYL | L |
| 903. | RRPsKPRLI | L |
| 904. | RRPsLQGNTL | H |
| 905. | RRPsQNAISF | N |
| 906. | RRPsQNAISFF | N |
| 907. | RRPsQPYmF | N |
| 908. | RRPsRPHmF | L |
| 909. | RRPsRPHmFP | L |
| 910. | RRPsYTLGm | C; M; N |
| 911. | RRQsFAVLR | N |
| 912. | RRQsVSRLL | N |
| 913. | RRREDsYHV | L |
| 914. | RRRsAPPEL | L |
| 915. | RRRsAVHmL | L |
| 916. | RRRsRVFDL | N |
| 917. | RRSsDIISL | N |
| 918. | RRSsIPITV | H; L; N |
| 919. | RR(ss)IQSTF | C; H; L; M |
| 920. | RRSsISSWL | N |
| 921. | RRSsLLSLM | H; L; N |
| 922. | RRSsLLSLm | H; N |
| 923. | RR(ss)QSWSL | C; H; L |
| 924. | RR(ss)YLLAI | B; H; L; N |
| 925. | RRVsIGVQL | H |
| 926. | RRVsPLNL | N |
| 927. | RRVsPLNLSSVTP | B |
| 928. | RRVsSNGIFDL | N |
| 929. | RRYsASTVDVIEm | B |
| 930. | RRYsDLTTL | H; L; N |
| 931. | RRYsDPPTY | L |
| 932. | RRYsLPLKSIYm | N |
| 933. | RSAsLAKL | M |
| 934. | RSAsLAKLGY | N |
| 935. | RSAsPSSQGW | L |
| 936. | RSAsPTVPR | M |
| 937. | RSAsQERSL | L |
| 938. | RSAsVGAEEY | C |
| 939. | RSD(ss)QPML | L |
| 940. | RSD(ss)QPmL | L |
| 941. | RSEsTENQSY | C |
| 942. | RSFsGLIKR | L; PI; PT |
| 943. | RSFsPKSPLEL | N |
| 944. | RSFsPTmKV | L; N |
| 945. | RSFsVEREL | L |
| 946. | RSFtPLSI | I |
| 947. | RSFtPLSILK | PT |
| 948. | RSHsLHYLF | N |
| 949. | RSHsPLRSK | M |
| 950. | RSHsPmSNR | M |
| 951. | RSHsPPLKL | N |
| 952. | RSHsSPASL | C; E; U |
| 953. | RSIsASDLTF | N |
| 954. | RSIsNEGLTL | N |
| 955. | RSIsSLLRF | N |
| 956. | RSIsTPTcL | H; N |
| 957. | RSIsTPTcL | H; N |
| 958. | RSIsTPTCL | N |
| 959. | RSIsTPTcL | N |
| 960. | RSKsATLLY | N |
| 961. | RSKsLTNLV | L |
| 962. | RSKsSImYF | N |
| 963. | RSKtPPKSY | N |
| 964. | RSLGsVQAPSY | N |
| 965. | RSLsASPAL | N |
| 966. | RSLsERLLQL | N |
| 967. | RSLsFSDEM | H |
| 968. | RSLsPFRRHSW | L |
| 969. | RSLsPFRRHsW | N |
| 970. | RSLsPGGAALGY | N |
| 971. | RSLsPILPGR | L; PI; PT |
| 972. | RSLsPLIKF | N |
| 973. | RSLsPmSGL | N |
| 974. | RSLsPSSNSAF | N |
| 975. | RSLsRVRVL | N |
| 976. | RSL(ss)GESL | N |
| 977. | RSLsSYRGKY | N |
| 978. | RSLsTTNVF | N |
| 979. | RSLsVGSEF | H; I |
| 980. | RSLsVPVDL | B; H |
| 981. | RSLTHLsL | M |
| 982. | RSLtHPPTI | N |
| 983 | RSMsGGHGL | L |
| 984. | RSNsLVSTF | N |
| 985. | RSNsPLPSI | I; N |
| 986. | RsPEPDPYLSY | B |
| 987. | RSPsFNmQL | B |
| 988. | RSPsKPTLAY | N |
| 989. | RSPsPKTSL | M |
| 990. | RSPsPSFRWPF | H |
| 991. | RSPsPTLSYY | C; N |
| 992. | RsPTKSSLDY | H |
| 993. | RsPTKSSLDYR | H |
| 994. | RSRPALsPL | L |
| 995. | RSRsDNALHL | N |
| 996. | RSRsPLGFY | N |
| 997. | RSRsPPPVS | L |
| 998. | RSRsRDRmY | N |
| 999. | RSRsVPVSF | PT |
| 1000. | RSRsYsPRRY | H |
| 1001. | RSRsYTPEY | N |
| 1002. | RsSFLQVF | H |
| 1003. | RSSPRTIsF | N |
| 1004. | RSSQFGsLEF | N |
| 1005. | RSSsAPLGL | N |
| 1006. | RSSsFKDFAK | L |
| 1007. | RSSsFSDTL | N |
| 1008. | RSSsFVLPKL | H |
| 1009. | R(sss)FVLPKL | H; N |
| 1010. | RSSsLQRRV | L |
| 1011. | R(sss)LSDFSW | N; PT |
| 1012. | R(sss)PFLSK | H; L; PT |
| 1013. | RSSsPLQL | N |
| 1014. | RSSsPPILTK | L |
| 1015. | RSSsPVTEL | N |
| 1016. | RSStPLPTI | N |
| 1017. | RSTsLSLKY | N |
| 1018. | RSVsGFLHF | N |
| 1019. | RSVsLDSQm | N |
| 1020. | RSVsLDSQmGY | N |
| 1021. | RSVsPTFL | B; H; L |
| 1022. | RSVsPTTEm | N |
| 1023. | RSVsPVQDL | N |
| 1024. | RSWsPPPEV | H |
| 1025. | RSWsPPPEVSR | H |
| 1026. | RSYsDPPLKF | N |
| 1027. | RSYsPERSKSY | N |
| 1028. | RSYsPERSKSYSF | N |
| 1029. | RSYsRLETL | N |
| 1030. | RTAsLSNQEcQLY | H |
| 1031. | RTAsLVSGL | N |
| 1032. | RTAsPPALPK | PT |
| 1033. | RTAtADDKKLQF | N |
| 1034. | RTDsIGEKL | L |
| 1035. | RTDsIGEKLGRY | H; N |
| 1037. | RTDsRGVNL | L |
| 1038. | RTFsDESNVL | N |
| 1039. | RTFsESSVW | N |
| 1040. | RTFsPTY | O |
| 1041. | RTFsPTYGLLR | H |
| 1042. | RTFsYIKNK | L |
| 1043. | RTGsPALGL | N |
| 1044. | RTIsAQDTLAY | I |
| 1045. | RTIsNPEVVmK | H |
| 1046. | RTIsPPTLGTL | N |
| 1047. | RTIsQSSSL | H; L |
| 1048. | R(t)I(s)VILFL | H |
| 1049. | RTLHsPPLQL | N |
| 1050. | RTLsmDKGF | N |
| 1051. | RTLsPSSGY | N |
| 1052. | RTLsVESLI | H; I; L; O |
| 1053. | RTMsPIQVL | H; I |
| 1054. | RTmsPIQVL | H; I |
| 1055. | RTPsISFFIH | N |
| 1056. | RTPsPARPAL | L |
| 1057. | RTPsPKSLPSYL | B; H |
| 1058. | RTPsQIIRK | L |
| 1059. | RTPsSSSTLAY | N |
| 1060. | RTRsLPITI | N |
| 1061. | RT(ss)FALNL | H |
| 1062. | RTSsQRSTLTY | C |
| 1063. | RTVsPAHVL | I; N |
| 1064. | RTVsPELIL | N |
| 1065. | RTYsLGSAL | H; I; L |
| 1066. | RVAsPKLVm | L |
| 1067. | RVAsPSRKV | V |
| 1068. | RVAsWAVSF | N |
| 1069. | RVDsLEFSL | O |
| 1036. | RTDsREQKL | L |
| 1070. | RVDsPVTV | L |
| 1071. | RVDsTTcLF | H |
| 1072. | RVKsWADNL | L |
| 1073. | RVKVDGPRSPsY | H |
| 1074. | RVMssPSAMK | PT |
| 1075. | RVPsINQKI | L |
| 1076. | RVPsKSLDL | PC |
| 1077. | RVPsKsLDL | PC |
| 1078. | RVPsPTPAPK | L |
| 1079. | RVRR(ss)FLNAK | H |
| 1080. | RVSsLTLHL | PI |
| 1081. | RVSsPLASF | N |
| 1082. | RVVPsPLQF | N |
| 1083. | RVVsPGIDL | H |
| 1084. | RVWEDRP(ss)A | H |
| 1085. | RVYTyIQSRF | M |
| 1086. | RYLGGsMDLSTF | B |
| 1087. | RYPtSIASL | B |
| 1088. | RYRsPEPDPYLSY | B |
| 1089. | SAAsPVVSSM | H |
| 1090. | SAEsKTIEF | PI |
| 1091. | SAGGsAEALLSDLH | H |
| 1092. | SAGGsAEALLSDLHAF | H |
| 1093. | SAIsPKSSL | H; L |
| 1094. | SAIsPTPEI | I |
| 1095. | SAKsPLPSY | H; N; PT |
| 1096. | SAmsPTHHL | H |
| 1097. | SAQGSDVsLTA | PT |
| 1098. | SAYGGLTsPGLS | B |
| 1099. | SAYGGLTsPGLSY | I; N |
| 1100. | SAYGGLTsPGLSYSL | H |
| 1101. | sDDEKmPDLE | L |
| 1102. | SDMPRAHsF | H |
| 1103. | SDmPRAHsF | H |
| 1104. | SDSAQGSESHsL | L |
| 1105. | SDsPPRPQPAF | L |
| 1106. | SDsPPRPQPAFKYQ | H |
| 1107. | SDYAVHPMsPVGRTS | H |
| 1108. | SDYAVHPmsPVGRTS | H |
| 1109. | SEAsPSREA | C |
| 1110. | SEAsPSREAI | H; L; M |
| 1111. | SED(ss)RGAF | L |
| 1112. | SEFTGFSGMsF | L |
| 1113. | SEGsLDRLY | PI |
| 1114. | SELsPGRSV | H |
| 1115. | SELLPSESL | H; PT |
| 1116. | SERIMQLsL | N |
| 1117. | SESKsmPVL | H |
| 1118. | SEVsPSGVGF | H; L |
| 1119. | SEYQWITsP | L |
| 1120. | SFDsGIAGL | PC |
| 1121. | SFDsGSVRL | E; H; I |
| 1122. | SFLPRTLsL | B |
| 1123. | sGPEIFTF | B |
| 1124. | SIDsPEKL | L; M |
| 1125. | sIELPSM | L |
| 1126. | SIGsPVKVGK | M |
| 1127. | sIISPDFSF | H |
| 1128. | SIIsPNFSF | B; H |
| 1129. | SILsRTPSV | PI |
| 1130. | SImsFHIDL | B |
| 1131. | SIPsGYLEL | PC |
| 1132. | SIRYSGHsL | L |
| 1133. | SISsIDREL | I; L |
| 1134. | SISsVSNTF | B |
| 1135. | SISVQVNSIKFDsE | L |
| 1136. | SITItPPDRYDSL | H |
| 1137. | SKSPSLSPSPP | N |
| | sPLEKTPL | |
| 1138. | SLAsLLAKV | PI |
| 1139. | SLDsLDLRV | O |
| 1140. | SLDsPGPEKm | PI |
| 1141. | SLDsPGPEKMAL | PI |
| 1142. | SLDsPGPEKmAL | PI; PT |
| 1143. | SLDsQQDSMKY | C |
| 1144. | SLDsQQDSmKY | C; N |
| 1145. | SLD(ss)NSGF | L |
| 1146. | sLEEPKQANGGAY | C; N |
| 1147. | SLGPIRsL | H |
| 1148. | SLHsLGSVSL | C |
| 1149. | SLIDGyYRL | O |
| 1150. | SLLsELQHA | PI |
| 1151. | SLLsLQTEL | L; N |
| 1152. | SLLsVSHAL | PI |
| 1153. | SLmsPGRRK | M |
| 1154. | SLm(t)I(s)HPGL | PI |
| 1155. | SLNSsPVSK | M |
| 1156. | SLSsERYYL | PI |
| 1157. | SL(ss)PTVTL | L |
| 1158. | SmKsPLYLVSR | L; PI; PT |
| 1159. | SMKsPLYLVSR | L; PT |
| 1160. | SmTRsPPRV | H; L |
| 1161. | SPAsPLKEL | L |
| 1162. | SPDHSDHtL | H |
| 1163. | SPD(ss)QSSL | H |
| 1164. | SPFLSKRsL | C; H; L |
| 1165. | SPFQSsPLSL | L |
| 1166. | SPFQSSPLsL | L |
| 1167. | SPFSSRSPsL | H |
| 1168. | SPGsPLHSL | L |
| 1169. | SPGsPLVSm | L |
| 1170. | SPHsPFYQL | L |
| 1171. | SPHtPSTHF | L |
| 1172. | sPHYFSPFRPY | L |
| 1173. | SPIAPRsPAKL | L |
| 1174. | sPIKVTL | L |
| 1175. | SPKPPTRsP | L |
| 1176. | SPKSGsPKSSSL | C |
| 1177. | SPKsPGLKAM | L |
| 1178. | SPLsKIGIEL | H |
| 1179. | SPLsPTETF | H |
| 1180. | SPLTKSIsL | B; H |
| 1181. | SPPDsPGRTL | L |
| 1182. | SPPNIAPKPL | L |
| 1183. | SPPsPARWSL | H |
| 1184. | SPPsPLEKTPL | N |
| 1185. | SPRDsPAVSL | L |
| 1186. | SPRGSGsSTSL | C |
| 1187. | SPRLPRsPRL | L |
| 1188. | SPRPPNsPSI | B; C |
| 1189. | SPRPPNsPSISI | C |
| 1190. | SPRRsLGLAL | B |
| 1191. | SPRRsRsISL | L |
| 1192. | SPRsESGGL | C |
| 1193. | SPRsPGPLPGARGL | L |
| 1194. | sPRsPGRSL | L |
| 1195. | SPRsPISPEL | C; L |
| 1196. | SPRsPQLSDF | L |
| 1197. | SPR(s)P(t)PSY | PT |
| 1198. | SPRsPVPTTL | C |
| 1199. | SPRtPPQRF | L; M |
| 1200. | SPRtPSNTP | L |
| 1201. | SPRTPtPFKHAL | L |
| 1202. | SPR(t)PV(s)PVKF | M |
| 1203. | SPsFGDPQL | I |
| 1204. | SPSKSPSLSPSPPsPLEK | N |
| | TPL | |
| 1205. | SPSLSPSPPsPLEKTPL | N |
| 1206. | SPSsPRVRL | L |
| 1207. | SPTsPFSSL | L |
| 1208. | SPVNKVRRVsF | L |
| 1209. | SPVsPMKEL | C; L; N |
| 1210. | SQAASSDSAQGSDVsL | PT |
| | TA | |
| 1211. | SQDsPRKL | L |
| 1212. | SQILRTPsL | H; L |
| 1213. | SRHsGPFFTF | B; M; N |
| 1214. | SRIPLVRsF | L |
| 1215. | SRKsFVFEL | L; PI |
| 1216. | SRLsLRRsL | L; PI |
| 1217. | SRLsLRRSL | L; PI; PT |
| 1218. | SRNQsPQRL | L |
| 1219. | SRPsMsPTPL | N |
| 1220. | SRP(sss)RSY | M |
| 1221. | SR(ss)SVLsL | B; N |
| 1222. | SR(sss)VLSL | H |
| 1223. | SRYsGVNQSM | PT |
| 1224. | SRYsGVNQSm | PT |
| 1225. | SSAVDtLRS | PT |
| 1226. | SSDPAsQLSY | B |
| 1227. | SSDSAQGSDVsLTA | PT |
| 1228. | SSDsPPRPQPAF | L |
| 1229. | SSDsPTNHF | L; PT |
| 1230. | SSGRsPSKAVAAR | B |
| 1231. | SSIPSTLsL | B; H; I |
| 1232. | SSIsPVRL | H |
| 1233. | SsLPRYLGL | H |
| 1234. | SSmKsPLYL | N |
| 1235. | SSmsPLPQm | H; PT |
| 1236. | SsPEFFm | H |
| 1237. | SSPRsPTTTL | C |
| 1238. | SSSGsPHLY | C |
| 1239. | SSSSSGsPHLY | C |
| 1240. | SsVPGVRLL | H; PI |
| 1241. | SsVPGVRLLQ | PI |
| 1242. | SsVPGVRLLQD | PI |
| 1243. | SSVPGVRLLQDsVD | PI |
| 1244. | SsVPGVRLLQDSVD | PI |
| 1245. | SSVsPAVSK | L |
| 1246. | SSYPRPLtY | N |
| 1247. | STDsGLGLGcY | H |
| 1248. | STDsGLGLGcY | H |
| 1249. | STDsPRLL | L |
| 1250. | STFsTNYRSL | H |
| 1251. | STFsTNYRsL | H |
| 1252. | STIAILNsV | B |
| 1253. | STIsLVTGETER | H; L |
| 1254. | STIsPSGAFG | H |
| 1255. | STIsPSGAFGLF | H |
| 1256. | STK(st)ELLL | N |
| 1257. | STKsTELLL | N |
| 1258. | STLLAsPmLK | H |
| 1259. | STLLAsPMLK | H |
| 1260. | STmsLNIITV | O |
| 1261. | STPsGYLEL | PC |
| 1262. | STsSGRLLY | N |
| 1263. | SVAsPLTL | H |
| 1264. | SVFRHFGsFQK | H |
| 1265. | SVGsDDELGPIR | O |
| 1266. | sVINVFVGR | H |
| 1267. | SVIsDDSVL | H |
| 1268. | SVIsQERLSY | H |
| 1269. | SVI(ss)E(s)GNTY | B |
| 1270. | SVKsPEVQLL | H |
| 1271. | SVLPRALsL | N |
| 1272. | SVLsYTSVR | PT |
| 1273. | SVLVRQIsL | B |
| 1274. | SVMQsPLVGV | B |
| 1275. | SVPGVRLLQDsVD | PI |
| 1276. | SVQsDQGYISR | L |
| 1277. | SVRRsVLmK | C; H; M |
| 1278. | SVRsLSLSL | B |
| 1279. | SVR(s)P(t)PYK | PT |
| 1280. | SVSRsPVPEK | L |
| 1281. | SV(ss)LEVHF | H |
| 1282. | SVSsLEVHF | I |
| 1283. | SVSsSSYR | PI |
| 1284. | SVTsPIKMK | L |
| 1285. | sYMGHFDLL | B |
| 1286. | SYPsPVPTSF | B; PT |
| 1287. | SYSF(ssss)IGH | C |
| 1288. | SYSFSSSsIGH | C |
| 1289. | SYSYSF(ssss)IGH | C |
| 1290. | SYSYSFSSSsIGH | C |
| 1291. | SYYsLPRSF | H |
| 1292. | SYYsPSIGF | B |
| 1293. | SYYsPSIGFSY | L; N |
| 1294. | TAIsPPLSV | H |
| 1295. | TAPLVPPLsPQY | H |
| 1296. | TASPVAVsL | L |
| 1297. | TATsPLTSY | L; PT |
| 1298. | TEAsPESmL | H; PT |
| 1299. | TEDsNLRLF | PI |
| 1300. | TELPKRLsL | N |
| 1301. | TEPLPEKTQEsL | C |
| 1302. | TESsPGSRQIQLW | H |
| 1303. | tHSLLLLL | H |
| 1304. | THsLLLLL | H |
| 1305. | TIRsPTTVL | C; L |
| 1306. | TItPPDRYDSL | H |
| 1307. | TKSsPLKI | N |
| 1308. | TLAsPSVFK | PT |
| 1309. | TLDsLDFARY | B |
| 1310. | TLE(stt)VGTSV | PI |
| 1311. | TLLAsPmLK | H |
| 1312. | TLLsPS SIKV | B |
| 1313. | TLSsIRHMI | PI |
| 1314. | TLSsIRHmI | PI |
| 1315. | TmAsPGKDNY | B; N |
| 1316. | TMFLRETsL | N |
| 1317. | TPAPSRTAsF | C |
| 1318. | TPAsPNREL | L |
| 1319. | TPAtPTSQF | C; PT |
| 1320. | TPHtPKSLL | L |
| 1321. | TPIsPGRASGm | H; L |
| 1322. | TPIsPGRASGmTTL | L |
| 1323. | TPIsPLKTGV | V |
| 1324. | TPKsPGASNF | L |
| 1325. | TPP(ss)EKLVSVM | N |
| 1326. | TPQPSRPVsPA | L |
| 1327. | TPQPSRPVsPAG | L |
| 1328. | TPRPAsPGPSL | L |
| 1329. | TPRTPRtPQL | L |
| 1330. | TPRtPRtPQL | L |
| 1331. | TPsPARPAL | L |
| 1332. | TPSsFDTHF | L |
| 1333. | TPSsREGTL | C |
| 1334. | TPVsPGSTF | L; PT |
| 1335. | TPVsPRLHV | H |
| 1336. | tPVSPTASm | H |
| 1337. | TPVsPVKF | K; L |
| 1338. | TRDsLLIHL | H |
| 1339. | TRLsPLEL | M; PT |
| 1340. | TRm(st)VSEL | N; PT |
| 1341. | TRM(st)VSEL | PT |
| 1342. | TRSsAVRLR | PI |
| 1343. | TRSsPVRKL | L |
| 1344. | TRYPtILQL | L |
| 1345. | TSDsPPHNDI | L |
| 1346. | TSFSVGsDDELGPIR | O |
| 1347. | TSGPGSRISSSsF | C |
| 1348. | TSIsPALAR | L |
| 1349. | TSIsPSRHGAL | H |
| 1350. | TSPsYIDKL | L |
| 1351. | TSVsPAPDK | L |
| 1352. | TTDPLIRWDsY | B; H |
| 1353. | TVDsPPWQL | L |
| 1354. | TVFsPTLPAAR | L |
| 1355. | TVKQKYLsF | PI; PT |
| 1356. | TVNsPAIYK | PT |
| 1357. | TVNsPAIYKF | H |
| 1358. | TVtPVPPPQ | PI |
| 1359. | TVY(ss)EEAELLK | H |
| 1360. | TYEGIFKtL | B |
| 1361. | VADsPAEVAL | PI; PT |
| 1362. | VADsPRDTASL | L |
| 1363. | VADtSIQKL | PI |
| 1364. | VEFPHsPEI | L |
| 1365. | VEKLPDsPAL | H; PT |
| 1366. | VELsPAR | PI; PT |
| 1367. | VELsPARSW | H |
| 1368. | VELsPARSw | PT |
| 1369. | VETsFRKLsF | H; L |
| 1370. | VETSFRKLsF | L |
| 1371. | VGsDDELGPIR | O |
| 1372. | VImsIRTKL | PI |
| 1373. | VIMsIRTKL | PI |
| 1374. | VIsDGGDSEQF | PI |
| 1375. | VLAsPLKTGR | L |
| 1376. | VLDsPASKK | H |
| 1377. | VLEKsPGKLLV | L |
| 1378. | VLFSsPPQm | B; L |
| 1379. | VLF(ss)PPQM | O |
| 1380. | VLmK(s)P(s)PAL | L; PI; PT |
| 1381. | VLMK(s)P(s)PAL | PI; PT |
| 1382. | VLYsPQmAL | PI |
| 1383. | VmDsPVHL | H; PI; PT |
| 1384. | VMDsPVHL | L |
| 1385. | VMFPGNsPSY | B |
| 1386. | VmFRtPLASV | K; L; PI |
| 1387. | VmIGsPKKV | C; H; M; O |
| 1388. | VMQsPLVGV | O |
| 1389. | VMTsLQQEY | N |
| 1390. | VPGVRLLQDsVD | PI |
| 1391. | VPKKPPPsP | L |
| 1392. | VPKSGR(sss)L | H |
| 1393. | VPKsPAFAL | B; C; L; M |
| 1394. | VPRPStPSRL | L |
| 1395. | VPRsPVIKI | L |
| 1396. | VPRtPSRERSSSA | L |
| 1397. | VPRtPVGKF | L |
| 1398. | VPSsPLRKA | L |
| 1399. | VPTsPKGRLL | C; L |
| 1400. | VPVsPGQQL | L |
| 1401. | VRLLQDsVD | PI |
| 1402. | VRQsPGPAL | PT |
| 1403. | VRTPSVQsL | H; L |
| 1404. | VRYsQLLGL | PI; PT |
| 1405. | VSDsPSHIA | L |
| 1406. | VSDsPSHIAT | L |
| 1407. | VSPSKSPSLSPSPPsPLE | N |
| | KTPL | |
| 1408. | VSPSKsPSLSPSPPsPLE | N |
| | KTPL | |
| 1409. | VSsPPPYTAY | C |
| 1410. | VSSSDsPPRPQPAF | L |
| 1411. | VSSsPRELL | I; N |
| 1412. | VTK(ss)PRAL | L |
| 1413. | VTtPNRLIY | N |
| 1414. | VTtPTGYKY | C |
| 1415. | VTTSTRTYsLG | PI |
| 1416. | VVsEVDIAKAD | PI |
| 1417. | VVSsPKLAPK | L |
| 1418. | VYIPMsPGAHHF | B |
| 1419. | VYIPmsPGAHHF | C |
| 1420. | VYLPTHTsL | B; C; H; I; N |
| 1421. | VYLPTH(ts)LL | B; C |
| 1422. | VYLPTH(ts)LLN | C |
| 1423. | VYLPTH(ts)LLNL | B; C |
| 1424. | VYLPTH(ts)LLNLT | C |
| 1425. | WEFGKRDsL | H |
| 1426. | WIGLNSLsF | L |
| 1427. | YAFEGTGsL | H |
| 1428. | yAQPQTTTPLPAVSG | L |
| 1429. | YA(ss)KLLKI | N |
| 1430. | YcIsPSTAAQF | N |
| 1431. | YcIsPSTAAQF | N |
| 1432. | YEFsPVKML | B |
| 1433. | YEGsPIKVT | PC |
| 1434. | YEGsPIKVTL | H; L; PI; PT |
| 1435. | YEsPGKIFL | L; PT |
| 1436. | YGDRTStF | L |
| 1437. | YGITsPISL | B |
| 1438. | YHLsPRAFLHY | Bw |
| 1439. | YIKtELISV | PI |
| 1440. | YLDSGIHsGA | L |
| 1441. | YLPsFFTKL | PI |
| 1442. | YLPTHTsLL | C |
| 1443. | YLRsVGDGETV | PI |
| 1444. | YLVsPITGEKI | PI |
| 1445. | YPHsPGSQY | PT |
| 1446. | YPLQIsPVSSY | L |
| 1447. | YPRLsIPNL | B |
| 1448. | YPSFRR(ss)L | B; M |
| 1449. | YPVsPKQKY | PT |
| 1450. | YRNDSSSsL | L |
| 1451. | YRRsVPTWL | L |
| 1452. | YSDRsSGGSY | C |
| 1453. | YSEsRSSLDY | C |
| 1454. | YsFcGTVEY | H |
| 1455. | YSFsPSKSY | N |
| 1456. | YSFSSSsIGH | N |
| 1457. | YSLDsPGPEK | PI |
| 1458. | YSLDsPGPEKm | PI |
| 1459. | YSLDsPGPEKMAL | PI; PT |
| 1460. | YSLDsPGPEKmAL | PI; PT |
| 1461. | YSLsPRPSY | N |
| 1462. | YSLsPSKSY | N |
| 1463. | YSLsPSKSYKY | N |
| 1464. | YSsLVRVL | H; L |
| 1465. | YSTtPGGTLY | H |
| 1466. | YTDSESSAsL | L |
| 1467. | YT(ss)RDAFGY | N |
| 1468. | YVDAETsL | L |
| 1469. | YVKLTPVsL | B; H; I; N; |
| | | PI; PT |
| 1470. | YVPDsPALL | H |
| 1471. | YVSsPDPQL | I |

*For amino acid sequences in this column, lowercase letters refer to amino acid positions where in some embodiments one or more modifications are present.
With respect to “s”, “t”, and “y”, these modifications are in some embodiments phosphorylations of serine, threonine, and tyrosine, respectively, and/or substitutions of the serine, threonine, and/or tyrosine with mimetics thereof. For amino acids enclosed within parentheses, one or more of the amino acids can be modified, in some embodiments phosphorylated and/or replaced with a mimetic, and all combinations and subcombinations of modification sites of the enclosed amino acids are encompassed within the presently disclosed subject matter.
“m” refers to an amino acid that in some but not all embodiments is oxidized.
“q” refers to an amino acid that in some embodiments is a pyroglutamic acid.
“w” refers to an amino acid that in some embodiments is an oxidized tryptophan. Where a given entry in Table 2 and/or Table 3 includes more than one indicator of a modification, it is understood that all combinations and subcombinations are encompassed by the presently disclosed subject matter.
**Tumor abbreviations are as follows: B: breast. C: colorectal. E: esophageal. I: intrahepatic cholangiocarcinoma (bile duct). K: kidney. L: leukemia/lymphoma. M: melanoma. N: head and neck. O: ovarian. PC: pancreatic. PI: phosphatase inhibitor. PT: Partially Transformed Peripheral Blood Mononuclear Cells (PBMCs). T: tonsil. U: lung. V: cervical.
***this is a peptide that in some embodiments comprises an N-terminal acetylation.
^{#}this peptide has an as-yet undetermined N-terminal modification, which can be a modification to the side chain of the N-terminal lysine and/or a modification of the N-terminal amine of the peptide.
^{@}“c” = homocysteinyl cysteine; ^{@@}“c” = cysteinyl cysteine; ^{@@@}“c” = methyl-cysteine
^{%}“c” cysteine; ^{%%}“c” = trioxidized cysteine
^{&}In some embodiments, the tryptophan can be modified to kynurenine

TABLE 3
Exemplary Peptides of the Presently
Disclosed Subject Matter With
Exemplary Uniprot Accessions and HLA Alleles

| | SEQ | Exemplary Parental | |
|---|---|---|---|
| | ID | Uniprot | HLA |
| Peptide* | NO: | Accession Nos. | Allele |
| AAsDTERDGLA | 6 | Q7L4I2 | |
| AAsPGAPQm | 7 | Q15637 | C5 |
| AEAPLPsPKL | 9 | Q09666 | B40 |
| AEFPSSGsNSVL | 11 | Q4LE39 | B40 |
| AELsPVEQKL | 19 | Q9Y5P8 | B40 |
| AERtPELVEL | 22 | Q9NS56 | B40 |
| ALAAPsPPR | 28 | Q96GP6 | A3 |
| AmDsPLLKY | 39 | P54920 | A1 |
| ARFsGFYSm | 65 | Q9NZV1 | C7 |
| ARFSGFYsm | 66 | Q9NZV1 | C7 |
| ARVsPSTSY | 72 | Q9BTE3 | C7 |
| ASmsPGHPTHL | 77 | O75376 | B15 |
| ASsPPDRIDIF | 78 | Q9NWB6 | A3 |
| ATIPRPFsV | 81 | P00439 | |
| ATmKRmLsL | 82 | Q9Y324 | B15 |
| AYGGLTsPGLSY | 85 | P05787 | A1 |
| DETERAYsF | 88 | O75582 | B44 |
| DGRRtFPRI | 89 | Q99759 | |
| DssEEK | 99 | P02808; | |
| | | P05060 | |
| DVYSGtPTKV | 108 | Q14493 | A68 |
| EPRNSLPAsPAHQL | 121 | Q8TEK3 | |
| FAVsPIPGRGGVL | 128 | P41162 | C3 |
| FPLARQFsL | 144 | P22455 | B53 |
| FRRsPTEDF | 150 | Q8IXT5 | |
| FSYsPRLPL | 159 | Q86WZ6 | C3 |
| FTDVNsILRY | 160 | P07814 | A1 |
| GAVsPVGEL | 165 | Q6WKZ4 | C3 |
| GELPTsPLHLL | 172 | Q969R5 | B40 |
| GGDsPVRL | 183 | Q92628 | C5 |
| GGPHFsPEHKEL | 185 | Q9UQ35 | |
| GIFPGtPLKK | 189 | Q86XN7 | A3 |
| GLLARtPPAA | 195 | Q9HCM7 | |
| GLLNKtPPTA | 196 | Q8WXX7 | |
| GSDVsLTAcKV^{@@} | 211 | P10316; | |
| | | P05534; | |
| | | P01891; | |
| | | P13746; | |
| | | Q09160; | |
| | | P01892; | |
| | | P30447; | |
| | | P16188; | |
| | | P30455; | |
| | | P04439; | |
| | | P30443 | |
| GsGPEIFTF | 212 | P46695 | |
| GSKsPISQL | 213 | Q04726 | B15 |
| GsPIKVTL | 215 | P06748 | B40 |
| GsPIKVTLA | 216 | P06748 | |
| GTVtPALKL | 220 | Q9BY77 | B15 |
| GTYVPSsPTRLAY | 221 | Q9Y4P8 | |
| GYVQRNLsLVRG | 225 | Q9UQ35 | |
| HPAsPAHPLL | 234 | Q8TBE0 | |
| HPYsPLPGL | 239 | P15408 | B7 |
| HTFsPSPKL | 248 | Q86YP4 | B15 |
| IAKsPHSTV | 255 | Q9Y618 | C12 |
| IARLPSsTL | 257 | Q86Y91 | |
| IIHsLETKL | 259 | H0Y7E2; | A2 |
| | | Q9Y6X7; | |
| | | H0Y2S9 | |
| IPAPPSsPL | 264 | Q9H9A5 | B7 |
| IPLSKIKtL | 267 | P13010 | B53 |
| IPRsPFKVKVL | 269 | O75369 | B7 |
| IPRTPLsPSPM | 270 | Q69YQ0 | B7 |
| ISIDsPQKL | 284 | Q12888 | B15 |
| I(sts)PSVAL | 287 | Q6W2J9 | C3 |
| ISVsPLATSAL | 288 | Q03164 | C3 |
| ITItPPDRY | 291 | P31751 | B15 |
| ITTtINPRF | 293 | P26010 | |
| ITYmsPAKL | 294 | Q9NPJ3 | B15 |
| IYYKsMPNL | 298 | O95490 | |
| KAPsPPPLL | 311 | Q8TDM6 | E |
| KAPsRQIsL | 312 | Q15390 | E |
| KAPsRQISL | 313 | Q15390 | E |
| KAPSRQIsL | 314 | Q15390 | E |
| KIDsPTKVK | 329 | Q15468 | A3 |
| KIIsAELQKA | 331 | P29375 | |
| KIIsIFSG | 332 | Q13177 | |
| KIRPHIAtL | 340 | Q14671 | B7 |
| KLwtLVSEQTRV^{&} | 351 | P46776 | A2 |
| KPAsPTPVI | 369 | P35606 | |
| KPGLGEGtP | 372 | P06702 | |
| KPVGVAAsL | 381 | Q14687 | |
| KRFsLDFNL | 389 | Q96MM3; | C7 |
| | | P25490; | |
| | | O15391 | |
| KRmsNELENY | 398 | Q8TAP9 | C7 |
| KRmsVTEGGIKY | 399 | P24928 | C7 |
| KRTsKYFSL | 407 | Q96NE9 | C7 |
| KRVsISEGDDKIEY | 408 | P22087 | |
| KRYsAEVRL | 409 | Q6NV74 | |
| KSDsPSTSSI | 413 | Q14157 | C5 |
| KSKsmDLGI | 414 | Q8TEW0 | B15 |
| KSPTsPLNm | 417 | P28370 | E |
| KSSsLDKQL | 420 | Q6WKZ4 | E |
| KSYsRSRsR | 423 | Q13243 | A3 |
| KTFsIGKIAK | 426 | O75366 | |
| KTmsPSQmI | 432 | Q9ULJ6 | |
| KTPTsPLKM | 435 | O60264 | E |
| KTPTsPLKm | 436 | O60264 | E |
| KTRsLSVEI | 438 | Q9NS56 | B15 |
| KTRsLsVEIVY | 439 | Q9NS56 | |
| KTRsLSVEIVY | 440 | Q9NS56 | |
| KTVsPSPAF | 442 | A8MYE0 | B15 |
| KVDsPTVTTTL | 443 | Q07866 | C5 |
| KVYtPSISK | 448 | Q5VV42 | A3 |
| LADsPLKL | 451 | Q8IV32 | |
| LEItPPSSEKL | 456 | Q5BJF6 | B40 |
| LIDNsFNRY | 460 | Q8IY81 | A1 |
| LPAFKRKtL | 465 | O14936; | B7 |
| | | Q00013 | |
| LPLsPKETV | 475 | Q9Y2F5 | |
| LPRsPPLKVL | 481 | Q96E14 | |
| LPSSGRSsL | 489 | O95817 | B7 |
| LSAsFRSLY | 499 | Q9UPW0 | |
| LSDsPSmGRY | 502 | O60245 | B15 |
| LSSsPPATHF | 505 | P11388 | B15 |
| LTDPSsPTIS | 506 | Q8IX90 | A1 |
| LTLsPKLQL | 509 | Q6P2E9 | B15 |
| LTSsRLLKL | 510 | Q3V6T2 | B15 |
| LVAsPRLEK | 512 | Q8IZW8 | A3 |
| LVVsPGQQTL | 515 | Q96SK2 | |
| NAIsLPTI | 546 | Q9UKI2 | |
| NP(ss)PEFFm | 555 | O75444 | |
| PLVSSSDsPPRPQPAF | 570 | Q9NQC3 | |
| P(ts)PLAmEY | 573 | O75444; | A1 |
| | | Q9Y5Q3 | |
| PwIPPSsPTTF | 574 | Q8IYN6 | A24 |
| QLDRIsVYY | 579 | P07437 | A1 |
| QVDPKKRIsm | 592 | Q14680 | B7 |
| RAEsGPDLRY | 601 | P15924 | |
| RAIsPREKI | 609 | A6H8Y1 | B15 |
| RATsPLVSL | 620 | Q96G74 | B15 |
| RATsRcLQL^{@@} | 621 | Q9HBH9 | |
| RAVsPFAKI | 622 | Q9Y2H2 | B15 |
| REAsPLSSNKLIL | 625 | Q9Y462 | B40 |
| RELsGTIKEIL | 631 | P30050 | B40 |
| REsPIPIEI | 639 | Q5TC82 | B40 |
| RHPKRSVsL | 653 | O60238 | E |
| RIHGsPLQK | 655 | O00566 | A3 |
| RIStPLTGV | 664 | Q08999 | A2 |
| RLFsHPREPAL | 687 | Q8IY67-2 | A2 |
| RLPtRLPEI | 710 | B7Z6U4 | A2 |
| RLRSsLVFK | 712 | Q5T0W9 | A3 |
| RLYKsEPEL | 719 | Q9HAU0 | A2 |
| RPAKsLmSI | 726 | Q4VCS5 | |
| RPARsVPSI | 729 | Q8IY63 | |
| RPAsPLMHI | 731 | Q96FC9 | B7 |
| RPDsRLLEL | 735 | O75638 | B7 |
| RPEsPAGPF | 736 | Q9HCC9 | B7 |
| RPHtPTPGI | 738 | P62995 | B7 |
| RPKLHHSLsF | 743 | P47974 | B7 |
| RPLPPPSsL | 752 | Q96MK2 | |
| RPPtPTLSL | 766 | Q6ZRS2 | B7 |
| RPRHsLNSL | 772 | Q9UGL1 | |
| RPRsPGSNSKVP | 781 | P78347 | B7 |
| RPsLGGRTPL | 786 | [unknown] | B7 |
| RP(ss)APDLm | 787 | P30305 | B7 |
| RPSsPPPFL | 792 | Q4KMQ1 | |
| RPSsPSTSw^{&} | 795 | Q3KQU3 | B7 |
| RP(st)PTIDVL | 802 | Q15910 | B7 |
| RPVsPHSDF | 812 | B0AZV3 | B7 |
| RPYsPSEYAL | 821 | Q14494 | B7 |
| RQPsEEEII | 833 | Q15121 | |
| RQPsEEEIIKL | 834 | Q15121 | |
| RQSsFEPEF | 839 | Q9H9A7 | |
| RRAsLSYSF | 841 | P00973-4 | C7 |
| RRFsSYSQm | 854 | Q13835 | C7 |
| RRGsFEVTLL | 862 | Q8IZQ5 | C7 |
| RRGsGPEIF | 864 | P46695 | |
| RRGsGPEIFT | 865 | P46695 | C7 |
| RRGsLLGSm | 867 | Q9HDC5 | C7 |
| RRGsYPFIDF | 871 | Q9NP56 | C7 |
| RRLsAARLL | 881 | O95613 | C7 |
| RRLsFQAEY | 882 | Q76N32 | C7 |
| RRLsGELISm | 884 | Q9HB15 | |
| RRLsLPGLL | 886 | Q96Q42 | C7 |
| RRLsLSRSL | 887 | Q8IXZ2 | C7 |
| RRLsRKL | 888 | O75167 | |
| RRLsVEIYDKF | 890 | O95069 | |
| RRLtLHSVF | 891 | Q6ZMZ0 | C7 |
| RRmsVAEQVDY | 895 | Q96Q15 | C7 |
| RRmsVGDRAG | 896 | Q9HBL0 | |
| RRNsFIGTPY | 897 | Q7Z2Y5 | C7 |
| RRNsKIFLDL | 899 | Q92995 | C7 |
| RRNsLLHGY | 900 | Q9HA65 | C7 |
| RRPsQPYmF | 907 | P13631 | C7 |
| RRQsVSRLL | 912 | Q9UHV5 | |
| RRSsDIISL | 917 | Q9UJX6 | C7 |
| RRVsPLNL | 926 | Q9UJX2 | C7 |
| RRVsPLNLSSVTP | 927 | Q9UJX2 | |
| RRVsSNGIFDL | 928 | Q6DN12 | C7 |
| RRYsASTVDVIEm | 929 | Q9H2U1 | C7 |
| RRYsLPLKSIYm | 932 | Q92611 | C7 |
| RSAsLAKL | 933 | Q76I76 | |
| RSAsPTVPR | 936 | Q92817 | A3 |
| RSAsVGAEEY | 938 | Q6ISB3 | A1 |
| RSEsTENQSY | 941 | Q96RT1 | A1 |
| RSHsPLRSK | 949 | Q9UKV3 | A3 |
| RSHsPmSNR | 950 | Q13595 | A3 |
| RSKsATLLY | 960 | Q96RT1 | B15 |
| RSKtPPKSY | 963 | Q05519 | B15 |
| RSLGsVQAPSY | 964 | P05783 | |
| RSLsASPAL | 965 | O95544 | B15 |
| RSLsPmSGL | 973 | A2VDJ0 | |
| RSLsPSSNSAF | 974 | Q9P266 | B15 |
| RSLsRVRVL | 975 | P20702 | B15 |
| RSL(ss)GESL | 976 | O15021 | B15 |
| RSLsTTNVF | 978 | Q16531 | B15 |
| RSNsLVSTF | 984 | Q9HB21 | B15 |
| RsPEPDPYLSY | 986 | P49761 | |
| RSPsFNmQL | 987 | Q8IXS8 | C7 |
| RSPsKPTLAY | 988 | Q86XR8 | |
| RSRsPLGFY | 996 | Q8IXT5 | |
| RSRsRDRmY | 998 | Q9NYF8 | |
| RSRsYTPEY | 1001 | Q13595 | A1 |
| RSSPRTIsF | 1003 | O00515 | B15 |
| RSSQFGsLEF | 1004 | Q8NHZ8 | B15 |
| RSSsAPLGL | 1005 | Q9HCM7 | B15 |
| RSSsFSDTL | 1007 | P15924 | B15 |
| RSSsPLQL | 1013 | O15055; | B15 |
| | | P56645 | |
| RSVsGFLHF | 1018 | Q92621 | B15 |
| RSVsLDSQm | 1019 | Q86UU0 | B15 |
| RSVsLDSQmGY | 1020 | Q86UU0 | |
| RSVsPVQDL | 1023 | O00409 | B15 |
| RTAsLVSGL | 1031 | Q9Y2I9 | B15 |
| RTDsRGVNL | 1037 | Q8NDL9 | C5 |
| RTGsPALGL | 1043 | Q9H201 | B15 |
| RTIsNPEVVmK | 1045 | P55196 | |
| RTIsPPTLGTL | 1046 | Q9UQ84 | B15 |
| RTLsPSSGY | 1051 | Q9P206 | |
| RTPsISFHH | 1055 | Q9C0I3 | B15 |
| RTRsLPITI | 1060 | Q8IZ21 | B15 |
| RTSsQRSTLTY | 1062 | Q99569 | A1 |
| RTVsPELIL | 1064 | Q6NZ36 | B15 |
| RVAsPSRKV | 1067 | P51587 | A68 |
| RVPsKSLDL | 1076 | Q8IYS0 | E |
| RVPsKsLDL | 1077 | Q81YS0 | E |
| RVVPsPLQF | 1082 | Q9Y2F5 | |
| RYLGGsMDLSTF | 1086 | O95983 | |
| RYRsPEPDPYLSY | 1088 | P49761 | |
| SAYGGLTsPGLS | 1098 | P05787 | |
| SDsPPRPQPAF | 1105 | Q9NQC3 | |
| SFDsGIAGL | 1120 | O95235 | C4 |
| sGPEIFTF | 1123 | P46695 | |
| SIGsPVKVGK | 1126 | Q96JJ7 | A3 |
| sIISPDFSF | 1127 | Q13115; | |
| | | P28562 | |
| SIIsPNFSF | 1128 | Q13115; | |
| | | P28562 | |
| SIPsGYLEL | 1131 | A0A087WUL8 | E |
| sLEEPKQANGGAY | 1146 | P18827 | A1 |
| SLNSsPVSK | 1155 | Q92879-4 | A3 |
| SPDHSDHtL | 1162 | Q68CZ2 | B7 |
| sPHYFSPFRPY | 1172 | Q13242 | B7 |
| sPIKVTL | 1174 | P06748 | B40 |
| SPKSGsPKSSSL | 1176 | Q5TCZ1 | B7 |
| SPRGSGsSTSL | 1186 | Q9H7P9 | B7 |
| SPRLPRsPRL | 1187 | O15083; | B7 |
| | | Q8IUD2 | |
| SPRsESGGL | 1192 | Q8TB72; | B7 |
| | | Q14671 | |
| SPRsPVPTTL | 1198 | Q63HR2 | B7 |
| SPRtPPQRF | 1199 | Q14CS0 | B7 |
| SRHsGPFFTF | 1213 | P25686 | C7 |
| SSDsPPRPQPAF | 1228 | Q9NQC3 | |
| SSGRsPSKAVAAR | 1230 | P60468 | |
| SSmKsPLYL | 1234 | Q8WYP5 | B15 |
| SsPEFFm | 1236 | O75444 | |
| SSSGsPHLY | 1238 | Q15464 | A1 |
| SSSSSGsPHLY | 1239 | Q15464 | A1 |
| SSYPRPLtY | 1246 | P15407 | |
| STIAILNsV | 1252 | P42695 | A2 |
| STKsTELLL | 1257 | Q6R327 | B15 |
| STPsGYLEL | 1261 | Q86T75; | E |
| | | Q3BBV0; | |
| | | Q6P3W6; | |
| | | Q5TAG4; | |
| | | P0DPF3; | |
| | | B4DH59; | |
| | | A0A087WUL8; | |
| | | Q3BBV2; | |
| | | P0DPF2; | |
| | | Q5TI25 | |
| SVFRHFGsFQK | 1264 | Q14162 | A3 |
| SVI(ss)E(s)GNTY | 1269 | P55040 | |
| SVKsPEVQLL | 1270 | Q99590 | |
| SVLPRALsL | 1271 | Q96MH7 | |
| SVMQsPLVGV | 1274 | Q8NFH5 | A2 |
| SYSFSSSsIGH | 1288 | P15924 | |
| SYSYSFSSSsIGH | 1290 | P15924 | |
| TAPLVPPLsPQY | 1295 | Q6ZW13 | A3 |
| TASPVAVsL | 1296 | Q15154 | C3 |
| TEPLPEKTQEsL | 1301 | P55327 | |
| TIRsPTTVL | 1305 | Q2KHR2 | B7 |
| TLDsLDFARY | 1309 | Q9P260 | A1 |
| TLLsPSSIKV | 1312 | Q15911 | A2 |
| TPAPSRTAsF | 1317 | P53396 | B7 |
| TPIsPLKTGV | 1323 | Q9NQW6 | |
| TPKsPGASNF | 1324 | Q9Y4E8 | B7 |
| TPSsREGTL | 1333 | P21860 | B7 |
| TPVsPRLHV | 1335 | P00748 | |
| tPVSPTASm | 1336 | Q9NR83 | |
| TSDsPPHNDI | 1345 | Q12778 | C5 |
| TSGPGSRISSSsF | 1347 | P05787 | |
| TYEGIFKtL | 1360 | Q8WWM7 | |
| VLDsPASKK | 1376 | Q8N5I9 | A3 |
| VPVsPGQQL | 1400 | Q9H4Z2 | B7 |
| VSsPPPYTAY | 1409 | Q96CS7 | A1 |
| VSSSDsPPRPQPAF | 1410 | Q9NQC3 | |
| VTtPNRLIY | 1413 | Q9Y2R4 | |
| VTtPTGYKY | 1414 | Q96BW1 | A1 |
| VYIPMsPGAHHF | 1418 | Q9UQC2 | |
| VYIPmsPGAHHF | 1419 | Q9UQC2 | A24 |
| YEFsPVKML | 1432 | Q6BEB4 | B40 |
| YEGsPIKVT | 1433 | P06748 | |
| YGITsPISL | 1437 | P51003; | C3 |
| | | Q9BWT3 | |
| YHLsPRAFLHY | 1438 | P50548 | |
| YSDRsSGGSY | 1452 | Q14011 | A1 |
| YSEsRSSLDY | 1453 | P30414 | A1 |
| YSFsPSKSY | 1455 | Q86YS7 | |
| YSFSSSsIGH | 1456 | P15924 | |
| YSLsPRPSY | 1461 | Q9C091 | |
| YTDSESSAsL | 1466 | Q96JK2 | C5 |
| YT(ss)RDAFGY | 1467 | Q6R327 | A1 |
| YVDAETsL | 1468 | Q9Y6M7 | C4 |
| YVPDsPALL | 1470 | Q9UQ88 | A2 |

*For amino acid sequences in this column, lowercase letters refer to amino acid positions where in some embodiments one or more modifications are present.
With respect to “s”, “t”, and “y”, these modifications are in some embodiments phosphorylations of serine, threonine, and tyrosine, respectively. For amino acids enclosed within parentheses, one or both of the amino acids can be modified, in some embodiments phosphorylated, and all combinations and subcombinations of phosphorylations of the enclosed amino acids are encompassed within the presently disclosed subject matter.
“m” refers to an amino acid that in some embodiments is oxidized.
“q” refers to an amino acid that in some embodiments is a pyroglutamic acid.
“w” refers to an amino acid that in some embodiments is an oxidized tryptophan.
**Tumor abbreviations are as follows: B: breast. C: colorectal. E: esophageal. I: intrahepatic cholangiocarcinoma (bile duct). K: kidney. L: leukemia/lymphoma. M: melanoma. N: head and neck. O: ovarian. PC: pancreatic. PI: phosphatase inhibitor. PT: Partially Transformed Peripheral Blood Mononuclear Cells (PBMCs). T: tonsil. U: lung. V: cervical.
^{@@}cysteinyl cysteine
^{&}In some embodiments, the tryptophan can be modified to kynurenine

The target peptides of the presently disclosed subject matter are in some embodiments not the entire proteins from which they are derived. They are in some embodiments from 8 to 50 contiguous amino acid residues of the native human protein. They can in some embodiments contain exactly, about, or at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. The peptides of the presently disclosed subject matter can also in some embodiments have a length that falls in the ranges of 8-10, 9-12, 10-13, 11-14, 12-15, 15-20, 20-25, 25-30, 30-35, 35-40, and 45-50 amino acids. Exactly, about, or at least 1, 2, 3, 4, 5, 6, 7, 8, 9, 10, 11, 12, 13, 14, 15, 16, 17, or more of the amino acid residues within the recited sequence of a target peptide can be phosphorylated and/or contain one or more N-terminal, C-terminal, and/or internal modifications. Exemplary modifications include oxidation of an amino acid (including but not limited to monooxidized, dioxidized, and/or trioxidized methionine, tryptophan, and cysteine), methylation (for example, of cysteine), modification to pyroglutamic acid, N- and/or C-terminal acetylation and/or acylation (for example, modification to include an O-linked beta-N-acetylglucosamine (“O-GlcNAc”) moiety), or any combination thereof. In some embodiments, a cysteine residue is modified to a homocysteinyl cysteine or a cysteinyl cysteine.

In some embodiments, substitutions can be made in the target peptides at residues known to interact with the MHC molecule. Such substitutions can in some embodiments have the effect of increasing the binding affinity of the target peptides for the MHC molecule and can also increase the half-life of the target peptide-MHC complex, the consequence of which is that the analog is in some embodiments a more potent stimulator of an immune response than is the original peptide.

Additionally, the substitutions can in some embodiments have no effect on the immunogenicity of the target peptide per se, but rather can prolong its biological half-life or prevent it from undergoing spontaneous alterations which might otherwise negatively impact on the immunogenicity of the peptide.

The target peptides disclosed herein can in some embodiments have differing levels of immunogenicity, MHC binding and ability to elicit CTL responses against cells displaying a native target peptide, e.g., on the surface of a tumor cell.

The amino acid sequences of the target peptides can in some embodiments be modified such that immunogenicity and/or binding is enhanced. In some embodiments, the modified target peptide binds an MHC class I molecule about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, or more tightly than its native (unmodified) counterpart.

However, given the exquisite sensitivity of the T cell receptor, it cannot be foreseen whether such enhanced binding and/or immunogenicity will render a modified target peptide still capable of inducing an activated CTL that will cross react with the native target peptide being displayed on the surface of a tumor. Indeed, it is disclosed herein that the binding affinity of a target peptide does not predict its functional ability to elicit a T cell response.

Target peptides of the presently disclosed subject matter can in some embodiments be mixed together to form a cocktail. The target peptides can in some embodiments be in an admixture, or they can in some embodiments be linked together in a concatamer as a single molecule. Linkers between individual target peptides can in some embodiments be used; these can, for example, in some embodiments be formed by any 10 to 20 amino acid residues. The linkers can in some embodiments be random sequences, or they can in some embodiments be optimized for degradation by dendritic cells.

In certain specified positions, a native amino acid residue in a native human protein can in some embodiments be altered to enhance the binding to the MHC class I molecule. These can occur in “anchor” positions of the target peptides, often in positions 1, 2, 3, 9, or 10. Valine, alanine, lysine, leucine tyrosine, arginine, phenylalanine, proline, glutamic acid, threonine, serine, aspartic acid, tryptophan, and methionine can also be used in some embodiments as improved anchoring residues. Anchor residues for different HLA molecules are listed below. Anchor residues for HLA molecules are listed in Table 4.

TABLE 4
Anchor Residues for Different HLA Molecules

| | |
|---|---|
| HLA A*0201 | Residue 2 = L, M |
| | Residue 9 or last residue = V |
| HLA A*0301 | Residue 2 = L, M |
| | Residue 9 or last residue = K |
| HLA A*0101 | Residue 2 = T, S |
| | Residue 3 = D, E |
| | Residue 9 or last residue = Y |
| HLA B*2705 | Residue 1 = R |
| | Residue 2 = R |
| | Residue 9 or last residue L, F, K, R, M |
| HLA B*0702 | Residue 2 = P |
| | Residue 9 or last residue = L, M, V, F |
| HLA B*4402 | Residue 2 = E |
| | Residue 9 or last residue = F, Y, W |

In some embodiments, the immunogenicity of a target peptide is measured using transgenic mice expressing human MHC class I genes. For example, “ADD Tg mice” express an interspecies hybrid class I MHC gene, AAD, which contains the alpha-1 and alpha-2 domains of the human HLA-A2.1 gene and the alpha-3 transmembrane and cytoplasmic domains of the mouse H-2Dd gene, under the direction of the human HLA-A2.1 promoter. Immunodetection of the HLA-A2.1 recombinant transgene established that expression was at equivalent levels to endogenous mouse class I molecules. The mouse alpha-3 domain expression enhances the immune response in this system. Compared to unmodified HLA-A2.1, the chimeric HLA-A2.1/H2-Dd MHC Class I molecule mediates efficient positive selection of mouse T cells to provide a more complete T cell repertoire capable of recognizing peptides presented by HLA-A2.1 Class I molecules. The peptide epitopes presented and recognized by mouse T cells in the context of the HLA-A2.1/H2-Dd class I molecule are the same as those presented in HLA-A2.1⁺ humans. This transgenic strain facilitates the modeling of human T cell immune responses to HLA-A2 presented antigens, and identification of those antigens. This transgenic strain is a preclinical model for design and testing of vaccines for infectious diseases or cancer therapy involving optimal stimulation of CD8⁺ cytolytic T cells.

In some embodiments, the immunogenicity of a modified target peptide is determined by the degree of Interferon gamma and/or TNF-alpha production of T cells from ADD Tg mice immunized with the target peptide, e.g., by immunization with target peptide pulsed bone marrow derived dendritic cells.

In some embodiments, the modified target peptides are about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 1000%, 1500%, 2000%, 2500%, 3000%, 4000%, 5000%, or more immunogenic, e.g., in terms of numbers of Interferon gamma and/or TNF-alpha positive (i.e., “activated”) T cells relative to numbers elicited by native target peptides in ADD Tg mice immunized with target peptides pulsed bone marrow derived dendritic cells. In some embodiments, the modified target peptides are able to elicit CD8⁺ T cells which are cross-reactive with the modified and the native target peptide in general and when such modified and native target peptides are complexed with MHC class I molecules in particular. In some embodiments, the CD8⁺ T cells which are cross-reactive with the modified and the native target peptides are able to reduce tumor size by about or at least 10%, 20%, 30%, 40%, 50%, 60%, 70%, 80%, 90%, 95%, 97%, or 99% in a NOD/SCID/IL-2Rγc^{-/-} knock out mouse (which has been provided transgenic T cells specific form an immune competent donor) relative to IL-2 treatment without such cross-reactive CD8⁺ T cells.

The term “capable of inducing a target peptide-specific memory T cell response in a patient” as used herein relates to eliciting a response from memory T cells (also referred to as “antigen-experienced T cell”) which are a subset of infection- and cancer-fighting T cells that have previously encountered and responded to their cognate antigen. Such T cells can recognize foreign invaders, such as bacteria or viruses, as well as cancer cells. Memory T cells have become “experienced” by having encountered antigen during a prior infection, encounter with cancer, or previous vaccination. At a second encounter with the cognate antigen, e.g., by way of an initial inoculation with a target peptide of the invention, memory T cells can reproduce to mount a faster and stronger immune response than the first time the immune system responded to the invader (e.g., through the body's own consciously unperceived recognition of a target peptide being associated with diseased tissue). This behavior can be assayed in T lymphocyte proliferation assays, which can reveal exposure to specific antigens. Memory T cells comprise two subtypes: central memory T cells (T_{CM} cells) and effector memory T cells (T_{EM} cells). Memory cells can be either CD4⁺ or CD8⁺. Memory T cells typically express the cell surface protein CD45RO. Central memory T_{CM} cells generally express L-selectin and CCR7, they secrete IL-2, but not IFNγ or IL-4. Effector memory T_{EM} cells, however, generally do not express L-selectin or CCR7 but produce effector cytokines like IFNγ and IL-4.

A memory T cell response generally results in the proliferation of memory T cell and/or the upregulation or increased secretion of the factors such as CD45RO, L-selectin, CCR7, IL-2, IFNγ, CD45RA, CD27 and/or IL-4. In some embodiments, the target peptides of the presently disclosed subject matter are capable of inducing a T_{CM} cell response associated with L-selectin, CCR7, IL-2 (but not IFNγ or IL-4) expression and/secretion. See e.g., Hamann et al. (1997) J Exp Med 186:1407-1418. In some embodiments, a T_{CM} cell response is associated with an at least or about 1%, 5%, 10%, 15%, 20%, 25%, 30%, 35%, 45%, 50%, 55%, 60%, 65%, 70%, 75%, 80%, 90%, 95%, 97%, 98%, 99%, 100%, 125%, 150%, 175%, 200%, 250%, 300%, 400%, 500%, 600%, 700%, 800%, 900%, 1000%, 1500%, 2000%, or more increase in T cell CD45RO/RA, L-selectin, CCR7, or IL-2 expression and/secretion.

In some embodiments, the target peptides of the presently disclosed subject matter are capable of inducing a CD8⁺ T_{CM} cell response in a patient the first time that patient is provided the composition including the selected target peptides. As such, the target peptides of the presently disclosed subject matter can in some embodiments be referred to as “neo-antigens”. Although target peptides might be considered “self” for being derived from self-tissue, they generally are only found on the surface of cells with a dysregulated metabolism, e.g., aberrant phosphorylation, they are likely never presented to immature T cells in the thymus. As such, these “self” antigens act are neo-antigens because they are nevertheless capable of eliciting an immune response.

In some embodiments, about or at least 1%, 5%, 10%, 15%, 20%, 25%, 30%, 35%, 45%, 50%, 55%, 60%, 65%, 70%, 75%, 80%, 90%, 95%, 97%, 98%, or 99% of T cells activated by particular target peptide in a particular patient sample are T_{CM} cells. In some embodiments, a patient sample is taken exactly, about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days after an initial exposure to a particular target peptide and then assayed for target peptide specific activated T cells and the proportion of T_{CM} cells thereof. In some embodiments, the compositions of the presently disclosed subject matter are able to elicit a CD8⁺ T_{CM} cell response in at least or about 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers. In some embodiments, the compositions of the presently disclosed subject matter are able to elicit a CD8⁺ T_{CM} cell response in a patient about or at least 1%, 5%, 10%, 15%, 20%, 25%, 30%, 35%, 45%, 50%, 55%, 60%, 65%, 70%, 75%, 80%, 90%, 95%, 97%, 98%, 99%, or 100% of patients and/or healthy volunteers specific to all or at least or about 1, 2, 3, 4, 5, 6, 7, 8, 9, or 10 target peptides in the composition. In some embodiments, the aforementioned T cell activation tests are done by ELISpot assay.

### II. Phosphopeptides

The term “phosphopeptides” includes MHC class I and MHC class II specific phosphopeptides. Exemplary MHC class I phosphopeptides of the presently disclosed subject matter are set forth in Table 2, for example.

In some embodiments, the phosphopeptides of the presently disclosed subject matter comprise the sequences of at least one of the MHC class I binding peptides listed in Table 2. Moreover, in some embodiments about or at least 1, 2, 3, 4, 5, 6, 7, 8, 9, 10, 11, 12, 13, 14, 15, or more of the serine, homo-serine, threonine, or tyrosine residues within the recited sequence is phosphorylated. The phosphorylation can in some embodiments be with a natural phosphorylation (—CH₂—O—PO₃H) or with an enzyme non-degradable, modified phosphorylation, such as (—CH₂—CF₂—PO₃H or —CH₂—CH₂—PO₃H). Some phosphopeptides can contain more than one of the peptides listed in Table 2, for example, if they are overlapping, adjacent, or nearby within the native protein from which they are derived.

The chemical structure of a phosphopeptide mimetic appropriate for use in the presently disclosed subject matter can in some embodiments closely approximate the natural phosphorylated residue which is mimicked, and also can in some embodiments be chemically stable (e.g., resistant to dephosphorylation by phosphatase enzymes). This can be achieved with a synthetic molecule in which the phosphorous atom is linked to the amino acid residue, not through oxygen, but through carbon. In some embodiments, a CF₂ group links the amino acid to the phosphorous atom. Mimetics of several amino acids which are phosphorylated in nature can be generated by this approach. Mimetics of phosphoserine, phosphothreonine, and phosphotyrosine can be generated by placing a CF₂ linkage from the appropriate carbon to the phosphate moiety. The mimetic molecule L-2-amino-4 (diethylphosphono)-4,4-difluorobutanoic acid (F2Pab) can in some embodiments substitute for phosphoserine (Otaka et al., Tetrahedron Letters 36: 927-930 (1995)). L-2-amino-4-phosphono-4,4difluoro-3-methylbutanoic acid (F2Pmb) can in some embodiments substitute for phosphothreonine. L-2-amino-4-phosphono (difluoromethyl) phenylalanine (F2Pmp) can in some embodiments substitute for phosphotyrosine (Akamatsu et al. (1997) Bioorg Med Chem 5:157-163; Smyth et al. (1992) Tetrahedron Lett 33:4137-4140). Alternatively, the oxygen bridge of the natural amino acid can in some embodiments be replaced with a methylene group. In some embodiments, serine and threonine residues are substituted with homo-serine and homo-threonine residues, respectively. A phosphorimidic can in some embodiments also include vanadate, pyrophosphate or fluorophosphates.

### III. O-GlcNAc Peptides

The term “O-GlcNAc peptides” includes MHC class I and MHC class II specific O-GlcNAc peptides. Modification of proteins with O-linked P-N-acetylglucosamine (O-GlcNAc) was previously technically difficult to detect. However, it rivals phosphorylation in both abundance and distribution of the protein targets for this modification. Like phosphorylation, O-GlcNAcylation is a reversible modification of nuclear and cytoplasmic proteins and consists of the attachment of a single R-N-acetyl-glucosamine moiety to hydroxyl groups of serine or threonine residues. Modification by O-GlcNAcylation is often competitive with phosphorylation at the same sites or at proximal sites on proteins. Furthermore, crosstalk between O-GlcNAcylation and phosphorylation affects the posttranslational state of hundreds of proteins in response to nutrients and stress and plays an important role in chronic diseases of metabolism, such as diabetes and neurodegeneration.

O-GlcNAc transferase (OGT) catalyzes the addition of the sugar moiety from the donor substrate uridine 5′-diphosphate (UDP)-GlcNAc to proteins. During M phase, OGT localizes to discrete structures, such as centrosomes (metaphase) and the spindle (anaphase), and then moves to the midbody during cytokinesis. OGT, along with O-GlcNAcase (OGA), the enzyme that removes the sugar, dynamically interacts with AURKB and PP1 at the midbody. Together, these proteins form a complex regulating M-phase O-GlcNAcylation, which in turn influences the phosphorylation state, of vimentin. However, the identity of other OGT mitotic substrates is currently not known.

Peptides modified with O-GlcNAc can be difficult to detect by standard mass spectrometric methods. The modification is usually present at sub-stoichiometric amounts, modified and unmodified peptides co-elute during high-performance liquid chromatography (HPLC), and ionization of the modified peptide is suppressed in the presence of unmodified peptides. Consequently, sample enrichment is often required to successfully detect and characterize O-GlcNAcylated peptides. Enrichment can be achieved through chemoenzymatic approaches that biotinylate O-GlcNAc peptides and capture them by avidin chromatography. Alternatively, a chemoenzymatic approach using a photocleavable biotin-alkyne reagent (PCbiotin-alkyne) tag can be used (see Fig. S1A of Wang et al. (2010) Sci Signal 3(104):ra2 (hereinafter “Wang”, incorporated herein by reference). Photocleavage not only allows efficient and quantitative recovery from the affinity column, but also tags the peptide with a charged moiety that facilitates O-GlcNAc site mapping by electron-transfer dissociation (ETD) mass spectrometry. This tagging approach also makes it possible to use conventional collision-activated dissociation mass spectrometry (CAD MS) to screen samples for the presence of 0-GlcNAc-modified peptides by monitoring for two-signature fragment ions characteristic of the tag (see Fig. S1B of Wang).

OGlcNAcylation rivals phosphorylation in both abundance and distribution of the modified proteins and alterations in O-GlcNAcylation disrupt both the chromosomal passenger complex, containing AURKB, INCENP, PP1, Borealin, and Surviven, and the circuits regulating CDK1 activity.

O-GlcNAc is nearly as abundant as phosphate on proteins associated with the spindle and midbody. Many of the O-GlcNAcylation sites identified are identical or proximal to known phosphorylation sites. O-GlcNAcylation and phosphorylation work together to control complicated mitotic processes, such as spindle formation. For example, OGT overexpression altered the abundance of transcripts and proteins encoded by several mitotic genes, changed the localization of NuMA1, and disrupted the chromosomal passenger complex and the CDK1 activation circuit.

An interplay exists between O-GlcNAcylation and phosphorylation for several protein classes, most noticeably transcriptional regulators and cytoskeletal proteins. Many of the 0-GlcNAcylation and phosphorylation sites are located in the regulatory head domains of intermediate filament proteins. Phosphorylation of these sites causes filament disassociation during M phase. For example, vimentin is phosphorylated at multiple sites during M phase and there is an O-GlcNAcylation site that is also a mitotic phosphorylation site (Ser55; Slawson et al. (2005) J Biol Chem 280:32944-32956; Slawson et al. (2008) Mol Biol Cell 19:4130-4140; Wang et al. (2007) Mol Cell Proteomics 6:1365-1379; Molina et al. (2007) Proc Natl Acad Sci USA 104:2199-2204). There are three additional O-GlcNAcylation sites on vimentin at Ser7, Thr33, and Ser34 (see Tables S5 and S6 of Wang), all of which are in the regulatory head domain of the protein. Two of these, Ser7 and Ser34, are also phosphorylation sites (Dephoure et al. (2008) Proc Natl Acad Sci USA 105:10762-10767; Molina et al. (2007) Proc Natl Acad Sci USA 104:2199-2204). Signaling pathways involving cytoskeletal proteins are regulated by reciprocal occupancy on specific sites by phosphate and 0-GlcNAc. In these classes of molecules, areas of multiple phosphorylation are also likely to be targeted for OGlcNAcylation.

OGT overexpression profoundly affects multiple mitotic signaling circuits. Although overexpression of OGT does not interfere with the formation of the midbody complex or localization of AURKB, AURKB activity is altered toward the cytoskeletal protein, vimentin. The reduction in the abundance of AURKB or INCENP dampens kinase activity to a point that retards mitotic progression especially during anaphase and telephase. Furthermore, OGT overexpression reduced phosphorylation of INCENP and borealin, but to what extent this alters the function of the midbody complex is unclear.

Multiple components of the cyclin B-CDK1 activation circuit were disrupted by the overexpression of OGT. The loss of PLK1 inhibitory phosphorylation on MYT1 and the increase in the abundance of MYT1 are likely contributors to the loss in cyclin B-CDK1 activity observed in OGT-overexpressing cells (see FIG. 7 of Wang). However, the reduction in cyclin B-CDK1 activity is likely only partially due to the increase in MYT1 activity, because the mRNA for CDC25C, the key CDK1 dual-specific phosphatase, is substantially reduced. The “on” switch for CDK1 activation, the reduction of MYT1 and the increase in CDC25C activity, is pushed toward “off” by OGT overexpression. Both MYT1 and CDC25C are substrates for PLK1. The protein and transcript abundance of PLK1 is substantially reduced in response to OGT overexpression, but there is little change in the extent of activating phosphorylation of PLK1.

Because O-GlcNAcylation is directly coupled to nutrient uptake and metabolism, the sugar residue is an ideal metabolic sensor for regulating mitotic progression. Whereas, phosphorylation might act as a master switch initiating the mitotic process, O-GlcNAcylation might act as an adjuster of signals to make these processes more responsive to environmental cues. How O-GlcNAcylation exerts control on specific mitotic proteins and how OGlcNAcylation will integrate into well-known signaling pathways represent another layer of cellular regulation.

### IV. Immunosuitablity

In some embodiments, the target peptides of the presently disclosed subject matter are combined into compositions which can be used in vaccine compositions for eliciting anti-tumor immune responses or in adoptive T cell therapy of cancer patients. Table 2 provides target peptides presented on the surface of cancer cells.

Although individuals in the human population display hundreds of different HLA alleles, some are more prevalent than others. For example, 88% of melanoma patients carry at least one of the six HLA alleles: HLA-A*0201 (51%), HLA-A*0101(29%), HLA-A*0301 (21%), HLA-A*4402 (27%), HLA-A*0702 (30%), and HLA-A*2705 (7%).

The presently disclosed subject matter provides in some embodiments target peptides which are immunologically suitable for each of the foregoing HLA alleles and, in particular, HLA-A*0201. “Immunologically suitable” means that a target peptide will bind at least one allele of an MHC class I molecule in a given patient. Compositions of the presently disclosed subject matter are in some embodiments immunologically suitable for a patient when at least one target peptide of the composition will bind at least one allele of an MHC class I molecule in a given patient. Compositions of multiple target peptides presented by each of the most prevalent alleles used in a cocktail, ensures coverage of the human population and to minimize the possibility that the tumor will be able to escape immune surveillance by down-regulating expression of any one class I target peptide.

The compositions of the presently disclosed subject matter can in some embodiments have at least one target peptide specific for HLA-A*0201. The compositions can in some embodiments have at least one phosphopeptide specific from at least the HLA-A*0201 allele. In some embodiments, the compositions can further comprise additional phosphopeptides from other MHC class I alleles.

As such, the compositions of the presently disclosed subject matter containing various combinations of target peptides will in some embodiments be immunologically suitable for between or about 3-88%, 80-89%, 70-79%, 60-69%, 57-59%, 55-57%, 53-55% or 51-53% or 5-90%, 10-80%, 15-75%, 20-70%, 25-65%, 30-60%, 35-55%, or 40-50% of the population of a particular cancer. In some embodiments, the compositions of the presently disclosed subject matter are able to act as vaccine compositions for eliciting anti-tumor immune responses or in adoptive T cell therapy of cancer patients, wherein the compositions are immunologically suitable for about or at least 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 20, 19, 18, 17, 16, 15, 14, 13, 12, 11, 10, 9, 8, 7, 6, 5, 4 or 3 percent of patients having a particular tumor and/or cancer.

### V. Compositions

“Target peptide compositions” as used herein refers to at least one target peptide formulated for example, as a vaccine; or as a preparation for pulsing cells in a manner such that the pulsed cells, e.g., dendritic cells, will display the at least one target peptide in the composition on their surface, e.g., to T cells in the context of adoptive T cell therapy.

The compositions of the presently disclosed subject matter can include in some embodiments about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-65, 65-80, 80-120, 90-150, 100-175, or 175-250 different target peptides.

The compositions of the presently disclosed subject matter generally include MHC class I specific target peptide(s) but in some embodiments can also include one or more target peptides specific for MHC class II or other peptides associated with tumors, e.g., tumor-associated antigen (“TAA”).

The identification of target peptides associated with various cancers are disclosed, for example, in U.S. Pat. Nos. 9,279,011; 9,561,266; 10,640,535; and 10,682,399 and in U.S. Patent Application Publication Nos. 2016/0000893, 2019/0015494, and 2019/0374627, and in PCT International Patent Application Serial No. PCT/US2020/024348, each of which is incorporated by reference in its entirety.

Compositions comprising the presently disclosed target peptide are typically substantially free of other human proteins or peptides. They can be made synthetically or by purification from a biological source. They can be made recombinantly. In some embodiments, they are at least 90%, 92%, 93%, 94%, at least 95%, or at least 99% pure. For administration to a human body, in some embodiments they do not contain other components that might be harmful to a human recipient. The compositions are typically devoid of cells, both human and recombinant producing cells. However, as noted below, in some cases, it can be desirable to load dendritic cells with a target peptide and use those loaded dendritic cells as either an immunotherapy agent themselves, or as a reagent to stimulate a patient's T cells ex vivo. The stimulated T cells can be used as an immunotherapy agent. In some embodiments, it can be desirable to form a complex between a target peptide and an HLA molecule of the appropriate type. Such complexes can in some embodiments be formed in vitro or in vivo. Such complexes are typically tetrameric with respect to an HLA-target peptide complex. Under certain circumstances it can be desirable to add additional proteins or peptides, for example, to make a cocktail having the ability to stimulate an immune response in a number of different HLA type hosts. Alternatively, additional proteins or peptide can provide an interacting function within a single host, such as an adjuvant function or a stabilizing function. As a non-limiting example, other tumor antigens can be used in admixture with the target peptides, such that multiple different immune responses are induced in a single patient.

Administration of target peptides to a mammalian recipient can in some embodiments be accomplished using long target peptides, e.g., longer than 15 residues, or using target peptide loaded dendritic cells. See Melief (2009) J Med Sci 2:43-45. The immediate goal is to induce activation of CD8⁺ T cells. Additional components which can be administered to the same patient, either at the same time or close in time (e.g., within 21 days of each other) include TLR-ligand oligonucleotide CpG and related target peptides that have overlapping sequences of at least 6 amino acid residues. To ensure efficacy, mammalian recipients should express the appropriate human HLA molecules to bind to the target peptides. Transgenic mammals can be used as recipients, for example, if they express appropriate human HLA molecules. If a mammal's own immune system recognizes a similar target peptide then it can be used as model system directly, without introducing a transgene. Useful models and recipients can in some embodiments be at increased risk of developing metastatic cancer. Other useful models and recipients can be predisposed, e.g., genetically or environmentally, to develop cancer.

V.A. Selection of Target Peptides

Disclosed herein is the finding that immune responses can be generated against phosphorylated peptides tested in healthy and diseased individuals. The T cells associated with these immune responses, when expanded in vitro, are able to recognize and kill malignant tissue (both established cells lines and primary tumor samples). Cold-target inhibition studies reveal that these target peptide-specific T cell lines kill primary tumor tissue in a target peptide-specific manner.

When selecting target peptides of the presently disclosed subject matter for inclusion in immunotherapy, e.g., in adaptive cell therapy or in the context of a vaccine, one can preferably pick target peptides that in some embodiments: 1) are associated with a particular cancer/tumor cell type; 2) are associated with a gene/protein involved in cell proliferation; 3) are specific for an HLA allele carried the group of patients to be treated; and/or 4) are capable of inducing a target peptide-specific memory T cell response in the patients to be treated upon a first exposure to a composition including the selected target peptides.

V.B. Target Peptide Vaccines

The antigen target peptides can also in some embodiments be used to vaccinate an individual. The antigen target peptides can be injected alone or in some embodiments can be administered in combination with an adjuvant and a pharmaceutically acceptable carrier. Vaccines are envisioned to prevent or treat certain diseases in general and cancers in particular.

The target peptides compositions of the presently disclosed subject matter can in some embodiments be used as a vaccine for cancer, and more specifically for melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, and cervical cancer. The compositions can in some embodiments include target peptides. The vaccine compositions can in some embodiments include only the target peptides, or peptides disclosed herein, or they can include other cancer antigens that have been identified.

In some embodiments, the cancer is breast cancer, and the target peptide comprises, consists essentially of, or consists of an amino acid sequence that is selected from the group consisting of SEQ ID NOs: 33, 39, 53, 54, 57, 58, 80, 109, 124, 126, 127, 134, 144, 148, 157, 160, 164, 189, 201, 204, 208, 212, 219, 233, 240, 253, 267, 286, 287, 290, 297-299, 304, 372, 373, 383, 388, 389, 424, 439, 440, 450, 461, 473, 478, 479, 494, 496, 503, 504, 506, 515, 516, 523, 564, 567, 573, 575, 576, 578-580, 589, 664, 732, 739, 768, 777, 784, 824, 835, 836, 862, 864-866, 871, 884, 886, 890, 894, 896, 897, 924, 927, 929, 980, 986, 987, 1021, 1057, 1086-1088, 1098, 1122, 1123, 1128, 1130, 1134, 1180, 1188, 1190, 1213, 1221, 1226, 1230, 1231, 1252, 1269, 1273, 1274, 1278, 1285, 1286, 1292, 1309, 1312, 1315, 1352, 1360, 1378, 1385, 1393, 1418, 1420, 1421, 1423, 1432, 1437, 1438, 1447, 1448, and 1469, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 33, 39, 53, 54, 57, 58, 80, 109, 124, 126, 127, 134, 144, 148, 157, 160, 164, 189, 201, 204, 208, 212, 219, 233, 240, 253, 267, 286, 287, 290, 297-299, 304, 372, 373, 383, 388, 389, 424, 439, 440, 450, 461, 473, 478, 479, 494, 496, 503, 504, 506, 515, 516, 523, 564, 567, 573, 575, 576, 578-580, 589, 664, 732, 739, 768, 777, 784, 824, 835, 836, 862, 864-866, 871, 884, 886, 890, 894, 896, 897, 924, 927, 929, 980, 986, 987, 1021, 1057, 1086-1088, 1098, 1122, 1123, 1128, 1130, 1134, 1180, 1188, 1190, 1213, 1221, 1226, 1230, 1231, 1252, 1269, 1273, 1274, 1278, 1285, 1286, 1292, 1309, 1312, 1315, 1352, 1360, 1378, 1385, 1393, 1418, 1420, 1421, 1423, 1432, 1437, 1438, 1447, 1448, and 1469.

In some embodiments, the cancer is colorectal cancer, and the target peptide comprises, consists essentially of, or consists of an amino acid sequence that is selected from the group consisting of SEQ ID NOs: 8, 10, 15, 21, 25-27, 51, 57, 58, 72, 73, 85, 106, 116, 117, 142, 156, 190, 201, 233, 263, 268, 270, 286, 299, 394, 395, 402, 412, 450, 472, 483, 484, 489, 530, 551, 565, 567, 574, 575, 582, 583, 584, 629, 664, 680, 681, 724, 741, 768, 776, 823, 835, 836, 851, 866, 868, 910, 919, 923, 938, 941, 952, 991, 1062, 1109, 1143, 1144, 1146, 1148, 1164, 1176, 1186, 1188, 1189, 1192, 1195, 1198, 1209, 1237, 1238, 1239, 1277, 1287, 1288-1290, 1301, 1305, 1317, 1319, 1333, 1347, 1387, 1393, 1399, 1409, 1414, 1419, 1420-1424, 1442, 1452, and 1453, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 8, 10, 15, 21, 25-27, 51, 57, 58, 72, 73, 85, 106, 116, 117, 142, 156, 190, 201, 233, 263, 268, 270, 286, 299, 394, 395, 402, 412, 450, 472, 483, 484, 489, 530, 551, 565, 567, 574, 575, 582, 583, 584, 629, 664, 680, 681, 724, 741, 768, 776, 823, 835, 836, 851, 866, 868, 910, 919, 923, 938, 941, 952, 991, 1062, 1109, 1143, 1144, 1146, 1148, 1164, 1176, 1186, 1188, 1189, 1192, 1195, 1198, 1209, 1237, 1238, 1239, 1277, 1287, 1288-1290, 1301, 1305, 1317, 1319, 1333, 1347, 1387, 1393, 1399, 1409, 1414, 1419, 1420-1424, 1442, 1452, and 1453.

In some embodiments, the cancer is esophageal cancer, and the target peptide comprises, consists essentially of, or consists of an amino acid sequence that is selected from the group consisting of SEQ ID NOs: 145, 305, 416, 490, 742, 765, 952, and 1121, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 145, 305, 416, 490, 742, 765, 952, and 1121.

In some embodiments, the cancer is intrahepatic cholangiocarcinoma (e.g., bile duct) cancer, and the target peptide comprises, consists essentially of, or consists of an amino acid sequence that is selected from the group consisting of SEQ ID NOs: 157, 158, 184, 217, 232, 250, 278, 316, 416, 431, 469, 471, 486, 488, 498, 532-536, 587-589, 654, 788, 946, 979, 985, 1044, 1052-1054, 1063, 1065, 1094, 1099, 1121, 1133, 1203, 1231, 1282, 1411, 1420, 1469, and 1471, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 157, 158, 184, 217, 232, 250, 278, 316, 416, 431, 469, 471, 486, 488, 498, 532-536, 587-589, 654, 788, 946, 979, 985, 1044, 1052-1054, 1063, 1065, 1094, 1099, 1121, 1133, 1203, 1231, 1282, 1411, 1420, 1469, and 1471.

In some embodiments, the cancer is kidney cancer, and the target peptide comprises, consists essentially of, or consists of an amino acid sequence that is selected from the group consisting of SEQ ID NOs: 472, 480, 521, 528-530, 726, 823, 836, 1337, and 1386, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 472, 480, 521, 528-530, 726, 823, 836, 1337, and 1386.

In some embodiments, the cancer is leukemia and/or lymphoma, and the target peptide comprises, consists essentially of, or consists of an amino acid sequence that is selected from the group consisting of SEQ ID NOs: 1-5, 7-9, 11, 13, 14, 17-20, 22, 23, 31, 38, 41-50, 52, 54-63, 67, 69, 70, 75, 79, 84, 88, 90, 91, 97, 104, 110, 118, 119, 121, 123, 128, 130, 132, 139, 142, 146-149, 152, 159, 161-165, 168, 170-173, 175, 183, 186, 190, 192, 195, 196, 203-206, 208, 210, 215, 222, 231, 232, 234, 237-239, 244, 245, 250, 251, 254, 257, 264, 265, 268, 269, 271, 273, 276, 278, 281-283, 288, 292, 295, 300-302, 305, 308, 310, 317, 319, 322, 325, 328, 333, 336, 340, 343, 347-350, 353, 355, 368-371, 373, 376, 377, 379-382, 384, 387, 391, 393, 396, 400, 405, 410, 413, 416, 419, 420, 427, 433, 434, 443, 444, 446, 447, 453, 454, 456, 462, 463, 465-468, 470, 472, 476, 477, 480-483, 485, 492, 495, 497, 501, 513, 514, 517, 519, 521, 522, 525, 528-530, 533, 534, 537, 548, 549, 552, 553, 558, 563, 564, 568-570, 581, 582, 585, 586, 589, 590, 592, 593, 595-599, 602-604, 611, 614, 623-625, 627, 628, 630, 631, 633, 636, 638, 639, 644, 645, 648-651, 663, 665, 667, 669, 670, 684, 693, 695, 699, 700, 717, 727, 729, 730, 731, 734-736, 739, 740, 744-753, 756, 758, 759, 762, 763, 766, 768, 769, 771, 773, 776, 777, 780-783, 785-787, 789-795, 797, 799-804, 807-817, 819, 821-827, 830, 832, 837, 838, 840, 843, 848, 851-853, 869, 870, 872-875, 878, 879, 885, 889, 894, 898, 901-903, 908, 909, 913-915, 918, 919, 921, 923, 924, 930, 931, 935, 937, 939, 940, 942, 944, 945, 961, 968, 971, 983, 994, 997, 1006, 1010, 1012, 1014, 1021, 1034, 1036, 1037, 1042, 1047, 1052, 1056, 1058, 1065, 1066, 1070, 1072, 1075, 1078, 1093, 1101, 1104, 1105, 1110-1112, 1118, 1119, 1124, 1125, 1132, 1133, 1135, 1145, 1151, 1157, 1158-1161, 1164-1166, 1168-1175, 1177, 1181, 1182, 1185, 1187, 1191, 1193-1196, 1199-1201, 1206-1209, 1211, 1212, 1214-1218, 1228, 1229, 1245, 1249, 1253, 1276, 1280, 1284, 1293, 1296, 1297, 1305, 1318, 1320-1322, 1324, 1326-1332, 1334, 1337, 1343-1345, 1348, 1350, 1351, 1353, 1354, 1362, 1364, 1369, 1370, 1375, 1377, 1378, 1380, 1384, 1386, 1391, 1393-1400, 1403, 1405, 1406, 1410, 1412, 1417, 1426, 1428, 1434-1436, 1440, 1446, 1450, 1451, 1464, 1466, and 1468, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 1-5, 7-9, 11, 13, 14, 17-20, 22, 23, 31, 38, 41-50, 52, 54-63, 67, 69, 70, 75, 79, 84, 88, 90, 91, 97, 104, 110, 118, 119, 121, 123, 128, 130, 132, 139, 142, 146-149, 152, 159, 161-165, 168, 170-173, 175, 183, 186, 190, 192, 195, 196, 203-206, 208, 210, 215, 222, 231, 232, 234, 237-239, 244, 245, 250, 251, 254, 257, 264, 265, 268, 269, 271, 273, 276, 278, 281-283, 288, 292, 295, 300-302, 305, 308, 310, 317, 319, 322, 325, 328, 333, 336, 340, 343, 347-350, 353, 355, 368-371, 373, 376, 377, 379-382, 384, 387, 391, 393, 396, 400, 405, 410, 413, 416, 419, 420, 427, 433, 434, 443, 444, 446, 447, 453, 454, 456, 462, 463, 465-468, 470, 472, 476, 477, 480-483, 485, 492, 495, 497, 501, 513, 514, 517, 519, 521, 522, 525, 528-530, 533, 534, 537, 548, 549, 552, 553, 558, 563, 564, 568-570, 581, 582, 585, 586, 589, 590, 592, 593, 595-599, 602-604, 611, 614, 623-625, 627, 628, 630, 631, 633, 636, 638, 639, 644, 645, 648-651, 663, 665, 667, 669, 670, 684, 693, 695, 699, 700, 717, 727, 729, 730, 731, 734-736, 739, 740, 744-753, 756, 758, 759, 762, 763, 766, 768, 769, 771, 773, 776, 777, 780-783, 785-787, 789-795, 797, 799-804, 807-817, 819, 821-827, 830, 832, 837, 838, 840, 843, 848, 851-853, 869, 870, 872-875, 878, 879, 885, 889, 894, 898, 901-903, 908, 909, 913-915, 918, 919, 921, 923, 924, 930, 931, 935, 937, 939, 940, 942, 944, 945, 961, 968, 971, 983, 994, 997, 1006, 1010, 1012, 1014, 1021, 1034, 1036, 1037, 1042, 1047, 1052, 1056, 1058, 1065, 1066, 1070, 1072, 1075, 1078, 1093, 1101, 1104, 1105, 1110-1112, 1118, 1119, 1124, 1125, 1132, 1133, 1135, 1145, 1151, 1157, 1158-1161, 1164-1166, 1168-1175, 1177, 1181, 1182, 1185, 1187, 1191, 1193-1196, 1199-1201, 1206-1209, 1211, 1212, 1214-1218, 1228, 1229, 1245, 1249, 1253, 1276, 1280, 1284, 1293, 1296, 1297, 1305, 1318, 1320-1322, 1324, 1326-1332, 1334, 1337, 1343-1345, 1348, 1350, 1351, 1353, 1354, 1362, 1364, 1369, 1370, 1375, 1377, 1378, 1380, 1384, 1386, 1391, 1393-1400, 1403, 1405, 1406, 1410, 1412, 1417, 1426, 1428, 1434-1436, 1440, 1446, 1450, 1451, 1464, 1466, and 1468.

In some embodiments, the cancer is melanoma, and the target peptide comprises, consists essentially of, or consists of an amino acid sequence that is selected from the group consisting of SEQ ID NOs: 12, 21, 28, 34, 54, 65, 99, 156, 175, 205, 207, 238, 239, 268, 329, 337, 346, 350, 371, 373, 379, 380, 423, 448, 467, 487, 512, 530, 571, 577, 655, 688, 690, 700, 712, 728, 738, 741, 743, 746, 768, 772, 778, 779, 782, 785, 788-790, 795, 815, 820, 823, 910, 919, 933, 936, 949, 950, 981, 989, 1085, 1110, 1124, 1126, 1153, 1155, 1199, 1202, 1213, 1220, 1277, 1339, 1387, 1393, and 1448, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 12, 21, 28, 34, 54, 65, 99, 156, 175, 205, 207, 238, 239, 268, 329, 337, 346, 350, 371, 373, 379, 380, 423, 448, 467, 487, 512, 530, 571, 577, 655, 688, 690, 700, 712, 728, 738, 741, 743, 746, 768, 772, 778, 779, 782, 785, 788-790, 795, 815, 820, 823, 910, 919, 933, 936, 949, 950, 981, 989, 1085, 1110, 1124, 1126, 1153, 1155, 1199, 1202, 1213, 1220, 1277, 1339, 1387, 1393, and 1448.

In some embodiments, the cancer is head and neck cancer, and the target peptide comprises, consists essentially of, or consists of an amino acid sequence that is selected from the group consisting of SEQ ID NOs: 6, 58, 64, 65, 68, 73, 76, 77, 80, 82, 86, 124, 129, 148-150, 157, 164, 166, 175, 178, 179, 184, 185, 187, 194, 202, 213, 220, 221, 225, 226, 228-230, 232, 248, 268, 284-286, 289, 291, 294, 296, 318, 325, 371, 388, 392, 398, 399, 405-409, 411, 414-416, 429, 432, 438, 441, 442, 460, 499, 500, 502, 505, 507-511, 540-543, 566, 572, 585, 589, 591, 601, 609, 612, 620, 621, 622, 630, 637, 640, 641, 668, 683, 704, 725, 737, 815, 824, 839, 841, 845-848, 853, 854, 863, 866, 867, 875, 880-883, 887, 891, 893, 895, 899, 900, 905-907, 910-912, 916-918, 920-922, 924, 926, 928, 930, 932, 934, 943, 944, 948, 951, 953-960, 962-966, 969, 970, 972-978, 982, 984, 985, 988, 991, 995, 996, 998, 1001, 1003-1005, 1007, 1009, 1011, 1013, 1015-1020, 1022, 1023, 1026-1029, 1031, 1033, 1035, 1038, 1039, 1043, 1046, 1049-1051, 1055, 1059, 1060, 1063, 1064, 1068, 1081, 1082, 1095, 1099, 1116, 1137, 1144, 1146, 1151, 1184, 1204, 1205, 1209, 1213, 1219, 1221, 1234, 1246, 1256, 1257, 1262, 1271, 1293, 1300, 1307, 1315, 1316, 1325, 1340, 1389, 1407, 1408, 1411, 1413, 1420, 1429, 1430, 1431, 1455, 1456, 1461-1463, 1467, and 1469, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 6, 58, 64, 65, 68, 73, 76, 77, 80, 82, 86, 124, 129, 148-150, 157, 164, 166, 175, 178, 179, 184, 185, 187, 194, 202, 213, 220, 221, 225, 226, 228-230, 232, 248, 268, 284-286, 289, 291, 294, 296, 318, 325, 371, 388, 392, 398, 399, 405-409, 411, 414-416, 429, 432, 438, 441, 442, 460, 499, 500, 502, 505, 507-511, 540-543, 566, 572, 585, 589, 591, 601, 609, 612, 620, 621, 622, 630, 637, 640, 641, 668, 683, 704, 725, 737, 815, 824, 839, 841, 845-848, 853, 854, 863, 866, 867, 875, 880-883, 887, 891, 893, 895, 899, 900, 905-907, 910-912, 916-918, 920-922, 924, 926, 928, 930, 932, 934, 943, 944, 948, 951, 953-960, 962-966, 969, 970, 972-978, 982, 984, 985, 988, 991, 995, 996, 998, 1001, 1003-1005, 1007, 1009, 1011, 1013, 1015-1020, 1022, 1023, 1026-1029, 1031, 1033, 1035, 1038, 1039, 1043, 1046, 1049-1051, 1055, 1059, 1060, 1063, 1064, 1068, 1081, 1082, 1095, 1099, 1116, 1137, 1144, 1146, 1151, 1184, 1204, 1205, 1209, 1213, 1219, 1221, 1234, 1246, 1256, 1257, 1262, 1271, 1293, 1300, 1307, 1315, 1316, 1325, 1340, 1389, 1407, 1408, 1411, 1413, 1420, 1429, 1430, 1431, 1455, 1456, 1461-1463, 1467, and 1469.

In some embodiments, the cancer is ovarian cancer, and the target peptide comprises, consists essentially of, or consists of an amino acid sequence that is selected from the group consisting of SEQ ID NOs: 29, 32, 40, 139, 193, 224, 261, 464, 666, 685, 686, 689-691, 700, 702, 708, 1040, 1052, 1069, 1139, 1149, 1260, 1265, 1346, 1371, 1379, 1387, and 1388, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 29, 32, 40, 139, 193, 224, 261, 464, 666, 685, 686, 689-691, 700, 702, 708, 1040, 1052, 1069, 1139, 1149, 1260, 1265, 1346, 1371, 1379, 1387, and 1388.

In some embodiments, the cancer is pancreatic cancer, and the target peptide comprises, consists essentially of, or consists of an amino acid sequence that is selected from the group consisting of SEQ ID NOs: 65, 66, 216, 311-314, 417, 420, 435, 436, 653, 1076, 1077, 1120, 1131, 1261, and 1433, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 65, 66, 216, 311-314, 417, 420, 435, 436, 653, 1076, 1077, 1120, 1131, 1261, and 1433.

In some embodiments, the cancer is a cancer associated with phosphatase inhibitor dysregulation, and the target peptide comprises, consists essentially of, or consists of an amino acid sequence that is selected from the group consisting of SEQ ID NOs: 30, 31, 35, 37, 74, 87, 111-115, 122, 135, 138-142, 149, 151, 161, 162, 174, 175, 182, 197, 224, 258, 259, 262, 266, 277, 278, 280, 282, 293, 295, 308, 309, 327, 330, 331, 334, 335, 341-348, 351, 352, 356-367, 385, 397, 403, 410, 421, 422, 427, 451, 474, 518, 539, 550, 553, 554, 558, 559, 600, 656-661, 668, 672-679, 682, 687, 692-694, 696, 698, 699, 703, 705-707, 710, 711, 713-715, 720-722, 824, 825, 831, 844-847, 861, 885, 942, 971, 1080, 1090, 1113, 1129, 1138, 1140-1142, 1150, 1152, 1154, 1156, 1158, 1215-1217, 1240-1244, 1275, 1283, 1299, 1310, 1313, 1314, 1342, 1355, 1358, 1361, 1363, 1366, 1372-1374, 1380-1383, 1386, 1390, 1401, 1404, 1415, 1416, 1434, 1439, 1441, 1443, 1444, 1457-1460, and 1469, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 30, 31, 35, 37, 74, 87, 111-115, 122, 135, 138-142, 149, 151, 161, 162, 174, 175, 182, 197, 224, 258, 259, 262, 266, 277, 278, 280, 282, 293, 295, 308, 309, 327, 330, 331, 334, 335, 341-348, 351, 352, 356-367, 385, 397, 403, 410, 421, 422, 427, 451, 474, 518, 539, 550, 553, 554, 558, 559, 600, 656-661, 668, 672-679, 682, 687, 692-694, 696, 698, 699, 703, 705-707, 710, 711, 713-715, 720-722, 824, 825, 831, 844-847, 861, 885, 942, 971, 1080, 1090, 1113, 1129, 1138, 1140-1142, 1150, 1152, 1154, 1156, 1158, 1215-1217, 1240-1244, 1275, 1283, 1299, 1310, 1313, 1314, 1342, 1355, 1358, 1361, 1363, 1366, 1372-1374, 1380-1383, 1386, 1390, 1401, 1404, 1415, 1416, 1434, 1439, 1441, 1443, 1444, 1457-1460, and 1469.

In some embodiments, the cancer is a cancer associated with partially-transformed pheripheral blood mononuclear cells (PBMCs), and the target peptide comprises, consists essentially of, or consists of an amino acid sequence that is selected from the group consisting of SEQ ID NOs: 8, 23, 31, 36, 74, 83, 92-98, 101, 104, 113, 114, 126, 136, 142, 149, 151, 161, 162, 173-175, 190, 191, 222, 223, 226, 227, 235, 236, 246, 247, 250, 272, 274, 278, 295, 333, 338, 339, 343, 410, 416, 425, 430, 437, 452, 455, 457-459, 474, 490, 519, 520, 526, 527, 533, 534, 537-539, 544, 545, 553-557, 560-562, 594, 602, 607, 608, 654, 699, 718, 815, 824, 825, 828, 829, 844-847, 885, 942, 947, 971, 999, 1011, 1012, 1032, 1074, 1095, 1097, 1115, 1142, 1158, 1159, 1197, 1210, 1217, 1223-1225, 1227, 1229, 1235, 1272, 1279, 1286, 1297, 1298, 1308, 1319, 1334, 1339, 1340, 1341, 1355, 1356, 1361, 1365, 1366, 1368, 1380, 1381, 1383, 1402, 1404, 1434, 1435, 1445, 1449, 1459, 1460, and 1469, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 8, 23, 31, 36, 74, 83, 92-98, 101, 104, 113, 114, 126, 136, 142, 149, 151, 161, 162, 173-175, 190, 191, 222, 223, 226, 227, 235, 236, 246, 247, 250, 272, 274, 278, 295, 333, 338, 339, 343, 410, 416, 425, 430, 437, 452, 455, 457-459, 474, 490, 519, 520, 526, 527, 533, 534, 537-539, 544, 545, 553-557, 560-562, 594, 602, 607, 608, 654, 699, 718, 815, 824, 825, 828, 829, 844-847, 885, 942, 947, 971, 999, 1011, 1012, 1032, 1074, 1095, 1097, 1115, 1142, 1158, 1159, 1197, 1210, 1217, 1223-1225, 1227, 1229, 1235, 1272, 1279, 1286, 1297, 1298, 1308, 1319, 1334, 1339, 1340, 1341, 1355, 1356, 1361, 1365, 1366, 1368, 1380, 1381, 1383, 1402, 1404, 1434, 1435, 1445, 1449, 1459, 1460, and 1469.

In some embodiments, the cancer is a cancer of the tonsils, and the target peptide comprises, consists essentially of, or consists of SEQ ID NO: 768. As such, in some embodiments a vaccine composition of the presently disclosed subject matter comprises, consists essentially of, or consists of a peptide comprising, consisting essentially of, and/or consisting of the amino acid sequence of SEQ ID NO: 768.

In some embodiments, the cancer is lung cancer, and the target peptide comprises, consists essentially of, or consists of an amino acid sequence that is selected from the group consisting of SEQ ID NOs: 119, 251, 256, 428, 470, 549, and 952, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 119, 251, 256, 428, 470, 549, and 952.

In some embodiments, the cancer is cervical cancer, and the target peptide comprises, consists essentially of, or consists of an amino acid sequence that is selected from the group consisting of SEQ ID NOs: 108, 255, 779, 1067, and 1323, and any combination thereof. As such, in some embodiments a vaccine composition of the presently disclosed subject matter comprises, consists essentially of, or consists of one or more peptides comprising, consisting essentially of, and/or consisting of an amino acid sequence selected from the group consisting of SEQ ID NOs: 108, 255, 779, 1067, and 1323.

The vaccine compositions can in some embodiments be used prophylactically for the purposes of preventing, reducing the risk of, and/or delaying initiation of a cancer in an individual that does not currently have cancer. Alternatively, they can be used to treat an individual that already has cancer, so that recurrence or metastasis is delayed and/or prevented. Prevention relates to a process of prophylaxis in which the individual is immunized prior to the induction or onset of cancer. For example, individuals with a history of poor lifestyle choices and at risk for developing cancer can in some embodiments be immunized prior to the onset of the disease.

Alternatively or in addition, individuals that already have cancer can be immunized with the antigens of the presently disclosed subject matter so as to stimulate an immune response that would be reactive against the cancer. A clinically relevant immune response would be one in which the cancer partially or completely regresses and/or is eliminated from the patient, and it would also include those responses in which the progression of the cancer is blocked without being eliminated. Similarly, prevention need not be total, but can in some embodiments result in a reduced risk, delayed onset, and/or delayed progression or metastasis.

The target peptide vaccines of the presently disclosed subject matter can in some embodiments be given to patients before, after, or during any of the aforementioned stages of cancer. In some embodiments, they are given to patients with malignant cancer.

In some embodiments, the 5-year survival rate of patients treated with the vaccines of the presently disclosed subject matter is increased by a statistically significant amount, e.g., by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more percent, relative to the average 5-year survival rates described above.

In some embodiments, the target peptide vaccine composition of the presently disclosed subject matter will increase survival rates in patients with cancer including but not limited to metastatic cancer by a statistically significant amount of time, e.g., by about or at least, 0.25, 0.5, 0.75, 1.0, 1.25, 1.5, 1.75, 2.0, 2.25, 2.5, 2.75, 3.0, 3.25, 3.5, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.50, 9.75, 10.0, 10.25, 10.5, 10.75, 11.0, 11.25, 11.5, 11.75, or 12 months or more compared to what could have been expected without vaccine treatment at the time of filing of this disclosure.

In some embodiments, the survival rate, e.g., the 1, 2, 3, 4, or 5-year survival rate, of patients treated with the vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, relative to the average 5-year survival rates described above.

The target peptide vaccines of the presently disclosed subject matter are in some embodiments envisioned to illicit a T cell associated immune response, e.g., generating activated CD8⁺ T cells specific for native target peptide/MHC class I expressing cells, specific for at least or about 1, 2, 3, 4, 5, 6, 7, 8, 9, 10, or more of the target peptides in the vaccine in a patient for about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, or 100 days after providing the vaccine to the patient.

In some embodiments, the treatment response rates of patients treated with the target peptide vaccines of the presently disclosed subject matter are increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, 100, 150, 200, 250, 300, 350, 400, 450, 500, or more percent, relative to treatment without the vaccine.

In some embodiments, overall median survival of patients treated with the target peptide vaccines of the presently disclosed subject matter is increased by a statistically significant amount, e.g., by about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine. In some embodiments, the overall median survival of cancer patients treated the target peptide vaccines is envisioned to be about or at least 10.0, 10.25, 10.5, 10.75, 11.0, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24, 25, 26, 27, 28, 29, 30, 31, 32, 33, 34, 35, 36, or more months.

In some embodiments, tumor size of patients treated with the target peptide vaccines of the presently disclosed subject matter is decreased by a statistically significant amount, e.g., by about, or by at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, or more percent, relative to treatment without the vaccine.

In some embodiments, the compositions of the presently disclosed subject matter provide an clinical tumor regression by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide a CTL response specific for the cancer being treated by a statistically significant amount, e.g., in about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of patients treated with a composition of the presently disclosed subject matter.

In some embodiments, the compositions of the presently disclosed subject matter provide an increase in progression free survival in the cancer being treated of about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more percent compared to the progression free survival or patients not treated with the composition.

In some embodiments, progression free survival, CTL response rates, clinical tumor regression rates, tumor size, survival rates (including but not limited to overall survival rates), and/or response rates are determined, assessed, calculated, and/or estimated weekly, monthly, bi-monthly, quarterly, semi-annually, annually, and/or bi-annually over a period of about or at least 1, 2, 3, 4, 5, 6, 7, 8, 9, 10, 11, 12, 13, 14, 15 or more years or about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500, or more weeks.

V.C. Compositions for Priming T Cells

Adoptive cell transfer is the passive transfer of cells, in some embodiments immune-derived cells, into a recipient host with the goal of transferring the immunologic functionality and characteristics into the host. Clinically, this approach has been exploited to transfer either immune-promoting or tolergenic cells (often lymphocytes) to patients to enhance immunity against cancer. The adoptive transfer of autologous tumor infiltrating lymphocytes (TIL) or genetically re-directed peripheral blood mononuclear cells has been used to successfully treat patients with advanced solid tumors, including but not limited to melanoma and ovarian carcinoma, as well as patients with CD19-expressing hematologic malignancies. In some embodiments, adoptive cell transfer (ACT) therapies achieve T cell stimulation ex vivo by activating and expanding autologous tumor-reactive T cell populations to large numbers of cells that are then transferred back to the patient. See e.g., Gattinoni et al. (2006) Nature Rev Immunol 6:383-393.

The target peptides of the presently disclosed subject matter can in some embodiments take the form of antigen peptides formulated in a composition added to autologous dendritic cells and used to stimulate a T helper cell or CTL response in vitro. The in vitro generated T helper cells or CTL can then be infused into a patient with cancer (Yee et al. (2002) Proc Natl Acad Sci USA 99:16168-16173), and specifically a patient with a form of cancer that expresses one or more of antigen target peptides.

Alternatively or in addition, the target peptides of the presently disclosed subject matter can be added to dendritic cells in vitro, with the loaded dendritic cells being subsequently transferred into an individual with cancer in order to stimulate an immune response. Alternatively or in addition, the loaded dendritic cells can be used to stimulate CD8⁺ T cells ex vivo with subsequent reintroduction of the stimulated T cells to the patient. Although a particular target peptide can be identified on a particular cancer cell type, it can be found on other cancer cell types.

The presently disclosed subject matter envisions treating cancer by providing a patient with cells pulsed with a composition of target peptides. The use of dendritic cells (“DCs”) pulsed with target peptide antigens allows for manipulation of the immunogen in two ways: varying the number of cells injected and varying the density of antigen presented on each cell. Exemplary methods for DC-based treatments can be found for example in Mackensen et al. (2000) Int J Cancer 86:385-392.

V.D. Additional Peptides Present in Target Peptide Compositions

The target peptide compositions (or target peptide composition kits) of the presently disclosed subject matter can in some embodiments also include at least one additional peptide derived from tumor-associated antigens. Examples of tumor-associated antigens include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 0-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-i, RCAS1, SDCCAG16, TA-90 (Mac-2 binding protein/cyclophilin C-associated protein), TAAL6, TAG72, TLP, TPS, prostatic acid phosphatase, and the like. Particular examples of additional peptides derived from tumor-associated antigens that can be employed alone or in combination with the compositions of the presently disclosed subject matter those set forth in Table 5 below.

TABLE 5
Exemplary Peptides Derived from Tumor-associated Antigens

| | | | Exemplary |
|---|---|---|---|
| | | SEQ | GENBANK® |
| Polypeptide Name^{a} | Amino Acid Sequence^{b} | ID NO: | Acc. No(s).^{c} |
| CEA₆₁₋₆₉ | HLFGYSWYK | 1474 | NP_001264092.1 |
| | | | XP_005278431.1 |
| CEA₆₀₄₋₆₁₂ | YLSGADLNL | 1475 | XP_005278431.1 |
| FBP/FOLR1₁₉₁₋₁₉₉ | EIWTHSYKV | 1476 | NP_000793.1 |
| gp100₁₇₋₂₅ | ALLAVGATK | 1477 | NP_001186982.1 |
| gp100₄₄₋₅₉ | WNRQLYPEWTEAQRLD | 1478 | NP_008859.1 |
| gp100₈₇₋₉₅ | ALNFPGSQK | 1479 | NP_008859.1 |
| gp100₈₉₋₉₅ | SQNFPGSQK | 1480 | NP_008859.1 |
| gp100₁₅₄₋₁₆₂ | KTWGQYWQV | 1481 | NP_008859.1 |
| gp100₂₀₉₋₂₁₇ | ITDQVPFSV | 1482 | NP_008859.1 |
| gp100₂₀₉₋₂₁₇ | IMDQVPFSV | 1483 | NP_008859.1 |
| gp100₂₈₀₋₂₈₈ | YLEPGPVTA | 1484 | NP_008859.1 |
| gp100₄₇₆₋₄₈₅ | VLYRYGSFSV | 1485 | NP_008859.1 |
| gp100₆₁₄₋₆₂₂ | LIYRRRLMK | 1486 | NP_008859.1 |
| Her2/neu₃₆₉₋₃₇₇ | KIFGSLAFL | 1487 | NP_004439.2 |
| Her2/neu₇₅₄₋₇₆₂ | VLRENTSPK | 1488 | NP_004439.2 |
| MAGE-A1₁₁₄₋₁₂₇ | LLKYRAREPVTKAE | 1489 | NP_004979.3 |
| MAGE-A2,3,6₁₂₁₋₁₃₄ | | | NP_005352.1 |
| | | | NP_005353.1 |
| | | | NP_005354.1 |
| MAGE-A1₉₆₋₁₀₄ | SLFRAVITK | 1490 | NP_004979.3 |
| MAGE-A1₁₆₁₋₁₆₉ | EADPTGHSY | 1491 | NP_004979.3 |
| MAGE-A3₁₆₈₋₁₇₆ | EVDPIGHLY | 1492 | NP_005353.1 |
| MAGE-A3₂₈₁₋₂₉₅ | TSYVKVLHHMVKISG | 1493 | NP_005353.1 |
| MAGE-A10₂₅₄₋₂₆₂ | GLYDGMEHL | 1494 | NP_001011543.2 |
| MART-1/MelanA₂₇₋₃₅ | AAGIGILTV | 1495 | NP_005502.1 |
| MART-1/MelanA₅₁₋₇₃ | RNGYRALMDKSLHVGTQCALTRR | 1496 | NP_005502.1 |
| MART-1/MelanA₉₇₋₁₁₆ | VPNAPPAYEKLsAEQSPPPY | 1497 | NP_005502.1 |
| MART-1/MelanA₉₈₋₁₀₉ | PNAPPAYEKLsA | 1498 | NP_005502.1 |
| MART-1/MelanA₉₉₋₁₁₀ | PNAPPAYEKLsA | 1499 | NP_005502.1 |
| MART-1/MelanA₁₀₀₋₁₀₈ | APPAYEKLs | 1500 | NP_005502.1 |
| MART-1/MelanA₁₀₀₋₁₁₁ | APPAYEKLsAEQ | 1501 | NP_005502.1 |
| MART-1/MelanA₁₀₀₋₁₁₄ | APPAYEKLsAEQSPP | 1502 | NP_005502.1 |
| MART-1/MelanA₁₀₀₋₁₁₅ | APPAYEKLsAEQSPPP | 1503 | NP_005502.1 |
| MART-1/MelanA₁₀₀₋₁₁₆ | APPAYEKLsAEQSPPPY | 1504 | NP_005502.1 |
| MART-1/MelanA₁₀₁₋₁₀₉ | PPAYEKLsA | 1505 | NP_005502.1 |
| MART-1/MelanA₁₀₁₋₁₁₂ | PPAYEKLsAEQS | 1506 | NP_005502.1 |
| MART-1/MelanA₁₀₂₋₁₁₀ | PAYEKLsAE | 1507 | NP_005502.1 |
| MART-1/MelanA₁₀₂₋₁₁₃ | PAYEKLsAEQSP | 1508 | NP_005502.1 |
| MART-1/MelanA₁₀₃₋₁₁₄ | AYEKLsAEQSPP | 1509 | NP_005502.1 |
| MART-1/MelanA₁₀₄₋₁₁₅ | YEKLsAEQSPPP | 1510 | NP_005502.1 |
| NY-ESO-1 | AAQERRVPR | 1511 | AAD05203.1 |
| | | | CAA10193.1 |
| NY-ESO-1 | LLGPGRPYR | 1512 | NP_001913.2 |
| NY-ESO-1₅₃₋₆₂ | ASGPGGGAPR | 1513 | NP_001318.1 |
| p2₈₃₀₋₈₄₄ | AQYIKANSKFIGITEL | 1514 | NP_783831.1 |
| TAG-1,2 | RLSNRLLLR | 1515 | |
| Tyr₅₆₋₇₀ | AQNILLSNAPLGPQFP | 1516 | NP_000363.1 |
| Tyr₁₄₆₋₁₅₆ | SSDYVIPIGTY | 1517 | NP_000363.1 |
| Tyr₂₄₀₋₂₅₁ | SDAEKSDICTDEY | 1518 | NP_000363.1 |
| Tyr₂₄₃₋₂₅₁ | KCDICTDEY | 1519 | NP_000363.1 |
| Tyr₃₆₉₋₃₇₇ | YMDGTMSQV | 1520 | NP_000363.1 |
| Tyr₃₈₈₋₄₀₆ | FLLHHAFVDSIFEQWLQRHRP | 1521 | NP_000363.1 |

^{a}Numbers listed in subscript are the amino acids positions of the listed peptide sequence in the corresponding polypeptide including, but not limited to the amino acid sequences provided in the GENBANK® biosequence database.
^{b}lower case amino acids in this column are optionally phosphorylated.
^{c}GENBANK® biosequence database Accession Numbers listed here are intended to be exemplary only and should not be interpreted to limit the disclosed peptide sequences to only these polypeptides.
Such tumor specific peptides (including the MHC class I phosphopeptides disclosed in Table 2 and/or Table 3) can be added to the target peptide compositions in a manner, number, and/or in an amount as if they were an additional target peptide added to the target peptide compositions as described herein.

V.E. Combination Therapies

In some embodiments, the target peptide compositions (or target peptide composition kits) of the presently disclosed subject matter are administered as a vaccine or in the form of pulsed cells as first, second, third, or fourth line treatment for the cancer. In some embodiments, the compositions of the presently disclosed subject matter are administered to a patient in combination with one or more therapeutic agents, e.g., anti-CA125 (or oregovomab Mab B43.13), anti-idiotype Ab (ACA-125), anti-HER-2 (trastuzumab, pertuzumab), anti-MUC-1 idiotypic Ab (HMFG1), HER-2/neu peptide, NY-ESO-1, anti-Programed Death-1 (“PD1”) (or PD1-antagonists such as BMS-936558), anti-CTLA-4 (or CTLA-4 antagonists), vermurafenib, ipilimumab, dacarbazine, IL-2, IFN-α, IFN-γ, temozolomide, receptor tyrosine kinase inhibitors (e.g., imatinib, gefitinib, erlotinib, sunitinib, tyrphostins, telatinib), sipileucel-T, tumor cells transfected with GM-CSF, a platinum-based agent, a taxane, an alkylating agent, an antimetabolite and/or a vinca alkaloid or combinations thereof. In an embodiment, the cancer is sensitive to or refractory, relapsed or resistant to one or more chemotherapeutic agents, e.g., a platinum-based agent, a taxane, an alkylating agent, an anthracycline (e.g., doxorubicin (e.g., liposomal doxorubicin)), an antimetabolite and/or a vinca alkaloid. In some embodiments, the cancer is refractory, relapsed, or resistant to a platinum-based agent (e.g., carboplatin, cisplatin, oxaliplatin), a taxane (e.g., paclitaxel, docetaxel, larotaxel, cabazitaxel) and/or an anthracycline (e.g., doxorubicin (e.g., liposomal doxorubicin)). In some embodiments, the cancer is refractory, relapsed, or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin). In some embodiments, the cancer is, e.g., lung cancer, and the cancer is refractory, relapsed or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., breast cancer, and the cancer is refractory, relapsed or resistant to a taxane (e.g., paclitaxel, docetaxel, larotaxel, cabazitaxel), a vascular endothelial growth factor (VEGF) pathway inhibitor, an anthracycline (e.g., daunorubicin, doxorubicin (e.g., liposomal doxorubicin), epirubicin, valrubicin, idarubicin), a platinum-based agent (e.g., carboplatin, cisplatin, oxaliplatin), and/or an antimetabolite (e.g., an antifolate (e.g., pemetrexed, floxuridine, raltitrexed) and a pyrimidine analogue (e.g., capecitabine, cytrarabine, gemcitabine, 5FU)). In some embodiments, the cancer is, e.g., gastric cancer, and the cancer is refractory, relapsed or resistant to an antimetabolite (e.g., an antifolate (e.g., pemetrexed, floxuridine, raltitrexed) and a pyrimidine analogue (e.g., capecitabine, cytrarabine, gemcitabine, 5FU)) and/or a platinum-based agent (e.g., carboplatin, cisplatin, oxaliplatin).

In some embodiments, the target peptide compositions (or target peptide composition kits) of the presently disclosed subject matter are associated with agents that inhibit T cell apoptosis or anergy thus potentiating a T cell response (“T cell potentiator”). Such agents include B7RP1 agonists, B7-H3 antagonists, B7-H4 antagonists, HVEM antagonists, HVEM antagonists, GAL9 antagonists or alternatively CD27 agonists, OX40 agonists, CD137 agonists, BTLA agonists, ICOS agonists CD28 agonists, or soluble versions of PDL1, PDL2, CD80, CD96, B7RP1, CD137L, OX40 or CD70. See Pardoll, National Reviews of Cancer, Focus on Tumour Immunology & Immunotherapy, 254, April 2012, Volume 12.

In some embodiments, the T cell potentiator is a PD1 antagonist. Programmed death 1 (PD-1) is a key immune checkpoint receptor expressed by activated T cells, and it mediates immunosuppression. PD-1 functions primarily in peripheral tissues, where T cells can encounter the immunosuppressive PD-1 ligands PD-L1 (B7-H1) and PD-L2 (B7-DC), which are expressed by tumor cells, stromal cells, or both. In some embodiments, the anti-PD-1 monoclonal antibody BMS-936558 (also known as MDX-1106 and ONO-4538) is used. In some embodiments, the T cell potentiator, e.g., PD1 antagonist, is administered as an intravenous infusion at least or about every 1, 1.5, 2, 2.5, 3, 3.5, or 4 weeks of each 4, 5, 6, 7, 8, 9, or 10-week treatment cycle of about for at least 1, 2, 3, 4, 5, 6, 7, 8, 9, 10, 11, 12, 13, 14, 15, 16, 17, 18, 19, 20, or more cycles. Exemplary, non-limiting doses of the PD1 antagonists are envisioned to be exactly, about, or at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, or more mg/kg. See Brahmer et al., N Engl J Med 2012; 366:2455-65.

The exemplary therapeutic agents disclosed herein above are envisioned to be administered at a concentration of, e.g., about 1 to 100 mg/m², about 10 to 80 mg/m², about 40 to 60 mg/m², e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more mg/mm². Alternatively, the exemplary therapeutic agents disclosed herein above are envisioned to be administered at a concentration of, e.g., about or at least 0.001 to 100 mg/kg or 0.1 to 1 mg/kg. In some embodiments, the exemplary therapeutic agents disclosed herein above are envisioned to be administered at a concentration of, e.g., about or at least from 0.01 to 10 mg/kg.

The target peptide compositions (or target peptide composition kits) of the presently disclosed subject matter can in some embodiments also be provided with administration of cytokines such as lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha-beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

The target peptide compositions of the presently disclosed subject matter can in some embodiments be provided with administration of cytokines around the time, (e.g., about or at least 1, 2, 3, or 4 weeks or days before or after) of the initial dose of a target peptide composition.

Exemplary, non-limiting doses of a cytokine would be about or at least 1-100, 10-80, 20-70, 30-60, 40-50, or 1, 2, 3, 4, 5, 6, 7, 8, 9, or 10 Mu/m²/day over about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. The cytokine can in some embodiments be delivered at least or about once every 1, 2, 3, 4, 5, 6, 7, 8, 9, 10, 11, 12, 13, 14, 15, 16, 17, 18, 19, 20, 21, 22, 23, or 24 hours. Cytokine treatment can in some embodiments be provided in at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cycles of at least or about 1, 2, 3, 4, 5, 6, 7, 8, 9, or 10 weeks, wherein each cycle has at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cytokine doses. Cytokine treatment can be on the same schedule as administration of the target peptide compositions or on a different (but in some embodiments overlapping) schedule.

In some embodiments, the cytokine is IL-2 and is dosed in an amount of about or at least 100,000 to 1,000,000; 200,000-900,000; 300,000-800,000; 450,000-750,000; 600,000-800,000; or 700,000-800,000; or 720,000 units (IU)/kg administered, e.g., as a bolus, every 1, 2, 3, 4, 5, 6, 7, 8, 9, 10, 11, 12, 13, 14, 15, 16, 17, 18, 19, or 20 hours for 1, 2, 3, 4, 5, 6, 7, 8, 9, 10, 11, 12, 13, or 14 days, in a cycle, for example.

### VI Types of Proliferative Disease

The compositions of the presently disclosed subject matter are envisioned to useful in the treatment of benign and malignant proliferative diseases. Excessive proliferation of cells and turnover of cellular matrix can contribute significantly to the pathogenesis of several diseases, including but not limited to cancer, atherosclerosis, rheumatoid arthritis, psoriasis, idiopathic pulmonary fibrosis, scleroderma and cirrhosis of the liver, ductal hyperplasia, lobular hyperplasia, papillomas, and others.

In some embodiments, the proliferative disease is cancer, which in some embodiments is selected from the group consisting of breast cancer, colorectal cancer, esophageal cancer, intrahepatic cholangiocarcinoma (bile duct), lung cancer including but not limited to squamous carcinoma of the lung, sarcoma, kidney cancer including but not limited to renal cell carcinoma, pancreatic carcinomas, squamous tumors of the head and neck, leukemia, lymphoma, brain cancer, liver cancer, prostate cancer, ovarian cancer, cervical cancer, melanoma, head and neck cancer, cancers associated with phosphatase inhibitor dysregulation, cancers associated with partially-transformed pheripheral blood mononuclear cells (PBMCs), and cancers of the tonsils. In some embodiments, the compositions of the presently disclosed subject matter are used to treat colorectal cancer, acute myelogenous leukemia (AML), acute lyphocytic leukemia (ALL), chronic lymphocytic lymphoma (CLL), chronic myelogenous leukemia (CML), breast cancer, renal cancer, pancreatic cancer, and/or ovarian cancer.

The target peptide compositions of the presently disclosed subject matter are in some embodiments used to treat cancer. When metastatic, the cancer can be in the lung, bone, liver, and/or brain.

In some embodiments, the cancer is a cancer of the bladder (including accelerated and metastatic bladder cancer), breast (e.g., estrogen receptor positive breast cancer, estrogen receptor negative breast cancer, HER-2 positive breast cancer, HER-2 negative breast cancer, triple negative breast cancer, inflammatory breast cancer), colon (including colorectal cancer), kidney (e.g., renal cell carcinoma), liver, lung (including small cell lung cancer and non-small cell lung cancer (including adenocarcinoma, squamous cell carcinoma, bronchoalveolar carcinoma and large cell carcinoma), genitourinary tract, e.g., ovary (including fallopian, endometrial and peritoneal cancers), cervix, prostate and testes, lymphatic system, rectum, larynx, pancreas (including exocrine pancreatic carcinoma), stomach (e.g., gastroesophageal, upper gastric or lower gastric cancer), gastrointestinal cancer (e.g., anal cancer), gall bladder, thyroid, lymphoma (e.g., Burkitt's, Hodgkin's, or non-Hodgkin's lymphoma), leukemia (e.g., acute myeloid leukemia), Ewing's sarcoma, nasoesophageal cancer, nasopharyngeal cancer, neural and glial cell cancers (e.g., glioblastoma multiforme), and head and neck. Exemplary cancers include but are not limited to melanoma, breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's, or non-Hodgkin's lymphoma), leukemia (e.g., acute myeloid leukemia) and gastrointestinal cancer.

### VII. Administration of Vaccine Compositions

VIIA. Routes of Administration

The target peptide compositions of the presently disclosed subject matter can in some embodiments be administered parenterally, systemically, and/or topically. By way of example and not limitation, composition injection can be performed by intravenous (i.v). injection, sub-cutaneous (s.c). injection, intradermal (i.d). injection, intraperitoneal (i.p). injection, and/or intramuscular (i.m). injection. One or more such routes can be employed. Parenteral administration can be, for example, by bolus injection or by gradual perfusion over time. Alternatively or concurrently, administration can be by the oral route.

In some embodiments, intradermal (i.d). injection is employed. The target peptide compositions of the presently disclosed subject matter are suitable for administration of the peptides by any acceptable route such as oral (enteral), nasal, ophthal, or transdermal. In some embodiments, the administration is subcutaneous and can be administered by an infusion pump.

VII.B. Formulation

Pharmaceutical carriers, diluents, and excipients are generally added to the target peptide compositions or (target peptide compositions kits) that are compatible with the active ingredients and acceptable for pharmaceutical use. Examples of such carriers include, but are not limited to, water, saline solutions, dextrose, and/or glycerol. Combinations of carriers can also be used. The vaccine compositions can further incorporate additional substances to stabilize pH and/or to function as adjuvants, wetting agents, and/or emulsifying agents, which can serve to improve the effectiveness of the vaccine.

The target peptide compositions can include one or more adjuvants such but not limited to montanide ISA-51 (Seppic, Inc.); QS-21 (Aquila Pharmaceuticals, Inc.); Arlacel A; oeleic acid; tetanus helper peptides (e.g., QYIKANSKFIGITEL or AQYIKANSKFIGITEL); GM-CSF; cyclophosamide; bacillus Calmette-Guerin (BCG); corynbacterium parvum; levamisole, azimezone; isoprinisone; dinitrochlorobenezene (DNCB); keyhole limpet hemocyanins (KLH) including Freunds adjuvant (complete and incomplete); mineral gels; aluminum hydroxide (Alum); lysolecithin; pluronic polyols; polyanions; peptides; oil emulsions; nucleic acids (e.g., dsRNA) dinitrophenol; diphtheria toxin (DT); toll-like receptor (TLR, e.g., TLR3, TLR4, TLR7, TLR8 or TLR9) agonists (e.g, endotoxins such as lipopolysaccharide (LPS); monophosphoryl lipid A (MPL); polyinosinic-polycytidylic acid (poly-ICLC/HILTONOL®; Oncovir, Inc., Washington, D.C., United States of America); IMO-2055, glucopyranosyl lipid A (GLA), QS-21—a saponin extracted from the bark of the Quillaja saponaria tree, also known as the soap bark tree or Soapbark; resiquimod (TLR7/8 agonist), CDX-1401—a fusion protein consisting of a fully human monoclonal antibody with specificity for the dendritic cell receptor DEC-205 linked to the NY-ESO-1 tumor antigen; Juvaris' Cationic Lipid-DNA Complex; Vaxfectin; and combinations thereof.

Polyinosinic-Polycytidylic acid (Poly IC) is a double-stranded RNA (dsRNA) that acts as a TLR3 agonist. To increase half-life, it has been stabilized with polylysine and carboxymethylcellulose as poly-ICLC. It has been used to induce interferon in cancer patients, with intravenous doses up to 300 μg/kg. Like poly-IC, poly-ICLC is a TLR3 agonist. TLR3 is expressed in the early endosome of myeloid DC; thus poly ICLC preferentially activates myeloid dendritic cells, thus favoring a Th1 cytotoxic T cell response. Poly ICLC activates natural killer (NK) cells, induces cytolytic potential, and induces IFN-gamma from myeloid DC.

In some embodiments, the adjuvant is provided at about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 micrograms per dose or per kg in each dose. In some embodiments, the adjuvant is provided at least or about 0.1, 0.2, 0.3, 0.40, 0.50, 0.60, 0.70, 0.80, 0.90, 0.100, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, or 9.90 grams per dose or per kg in each dose. In some embodiments, the adjuvant is given at about or at least 10, 15, 20, 25, 50, 75, 100, 125, 150, 175, 150, 175, 200, 225, 250, 275, 300, 325, 350, 375, 400, 425, 450, 500, 525, 550, 575, 600, 625, 675, 700, 725, 750, 775, 800, 900, 1000, 1100, 1200, 1300, 1400, 1500, 1600, 1700, 1800, 1900, or 2000 endotoxin units (“EU”) per dose.

The target peptide compositions of the presently disclosed subject matter can in some embodiments be provided with an administration of cyclophosamide around the time, (e.g., about or at least 1, 2, 3, or 4 weeks or days before or after) the initial dose of a target peptide composition. An exemplary dose of cyclophosamide would in some embodiments be about or at least 100, 200, 300, 400, 500, 600, 700, 800, 900, or 1000 mg/m²/day over about or at least 1, 2, 3, 4, 5, 6, 7, 8, 9, or 10 days.

The compositions of the presently disclosed subject matter can in some embodiments comprise the presently disclosed target peptides in the free form and/or in the form of a pharmaceutically acceptable salt.

As used herein, “a pharmaceutically acceptable salt” refers to a derivative of the disclosed target peptides wherein the target peptide is modified by making acid or base salts of the target peptide. For example, acid salts are prepared from the free base (typically wherein the neutral form of the drug has a neutral —NH₂ group) involving reaction with a suitable acid. Suitable acids for preparing acid salts include both organic acids such as but not limited to acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids such as but not limited to hydrochloric acid, hydrobromic acid, sulfuric acid, nitric acid, phosphoric acid, and the like. Conversely, basic salts of acid moieties which can be present on a target peptide are prepared using a pharmaceutically acceptable base such as sodium hydroxide, potassium hydroxide, ammonium hydroxide, calcium hydroxide, trimmethylamine or the like. By way of example and not limitation, the compositions can in some embodiments comprise the target peptides as salts of acetic acid (acetates), ammonium, or hydrochloric acid (chlorides).

In some embodiments, a composition can include one or more sugars, sugar alcohols, amino acids such a glycine, arginine, glutaminic acid, and others as framework former. The sugars can be mono-, di- or trisaccharide. These sugars can be used alone, as well as in combination with sugar alcohols. Examples of sugars include glucose, mannose, galactose, fructose or sorbose as monosaccharides, sucrose, lactose, maltose or trehalose as disaccharides and raffinose as a trisaccharide. A sugar alcohol can be, for example, mannitose. In some embodiments, the composition comprises sucrose, lactose, maltose, trehalose, mannit and/or sorbit. In some embodiments, the composition comprises mannitol.

Furthermore, in some embodiments the presently disclosed compositions can include physiological well-tolerated excipients (see e.g., the Handbook of Pharmaceutical Excipients, 5^{th} ed. (2006) Rowe et al. (eds)., Pharmaceutical Press, London, United Kingdom), such as antioxidants like ascorbic acid or glutathione, preserving agents such as phenol, m-cresole, methyl- or propylparabene, chlorobutanol, thiomersal or benzalkoniumchloride, stabilizer, framework former such as sucrose, lactose, maltose, trehalose, mannitose, mannitol and/or sorbitol, mannitol and/or lactose and solubilizer such as polyethyleneglycols (PEG), i.e. PEG 3000, 3350, 4000, or 6000, or cyclodextrines, i.e. hydroxypropyle-β-cyclodextrine, sulfobutylethyl-β-cyclodextrine or γ-cyclodextrine, or dextranes or poloxaomers, i.e. poloxaomer 407, poloxamer 188, or TWEEN™20, TWEEN™ 80. In some embodiments, one or more well tolerated excipients can be included, selected from the group consisting of antioxidants, framework formers, and stabilizers.

In some embodiments, the pH for intravenous and intramuscular administration is selected from pH 2 to pH 12, while the pH for subcutaneous administration is selected from pH 2.7 to pH 9.0 as the rate of in vivo dilution is reduced resulting in more potential for irradiation at the injection site. (Strickley (2004) Pharm Res 21:201-230).

VII.C. Dosage

It is understood that a suitable dosage of a target peptide composition vaccine immunogen will depend upon the age, sex, health, and weight of the recipient, the kind of concurrent treatment, if any, the frequency of treatment, and the nature of the effect desired. However, a desired dosage can be tailored to the individual subject, as determined by the researcher or clinician. The total dose employed for any given treatment can typically be determined with respect to a standard reference dose based on the experience of the researcher or clinician, such dose being administered either in a single treatment or in a series of doses, the success of which can depend on the production of a desired immunological result (i.e., successful production of a T helper cell and/or CTL-mediated response to the target peptide immunogen composition, which response gives rise to the prevention and/or treatment desired). Thus, in some embodiments the overall administration schedule can be considered in determining the success of a course of treatment and not whether a single dose, given in isolation, would or would not produce the desired immunologically therapeutic result or effect. As such, a therapeutically effective amount (i.e., that producing the desired T helper cell and/or CTL-mediated response) can in some embodiments depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and/or the sound judgment of the clinician or researcher. Needless to say, the efficacy of administering additional doses and of increasing or decreasing the interval can be re-evaluated on a continuing basis, in view of the recipient's immunocompetence (for example, the level of T helper cell and/or CTL activity with respect to tumor-associated or tumor-specific antigens).

The concentration of the T helper or CTL stimulatory target peptides of the invention in pharmaceutical formulations are subject to wide variation, including anywhere from less than 0.01% by weight to as much as 50% or more. Factors such as volume and viscosity of the resulting composition can also be considered. The solvents, or diluents, used for such compositions can include one or more of water, phosphate buffered saline (PBS), saline itself, and/or other possible carriers and/or excipients. The immunogens of the presently disclosed subject matter can in some embodiments also be contained in artificially created structures such as liposomes, which structures can in some embodiments contain additional molecules, such as proteins or polysaccharides, inserted in the outer membranes of the structures and having the effect of targeting the liposomes to particular areas of the body, or to particular cells within a given organ or tissue. Such targeting molecules can in some embodiments be some type of immunoglobulin. Antibodies can work particularly well for targeting the liposomes to tumor cells.

Single i.d., i.m., s.c., i.p., and i.v. doses of e.g., about 1 to 50 μg, 1 to 100 μg, 1 to 500 μg, 1 to 1000 μg, or about 1 to 50 mg, 1 to 100 mg, 1 to 500 mg, or 1 to 1000 mg of a target peptide composition of the presently disclosed subject matter can in some embodiments be given and in some embodiments can depend from the respective compositions of target peptides with respect to total amount for all target peptides in the composition or alternatively for each individual target peptide in the composition. A single dose of a target peptide vaccine composition of the presently disclosed subject matter can in some embodiments have a target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 μg. Alternatively, a single dose of a target peptide composition of the presently disclosed subject matter can in some embodiments have a total target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 mg. In some embodiments, the target peptides of a composition of the presently disclosed subject matter are present in equal amounts of about 100 micrograms per dose in combination with an adjuvant peptide present in an amount of about 200 micrograms per dose.

In a single dose of the target peptide composition of the presently disclosed subject matter, the amount of each target peptide in the composition is in some embodiments equal or is in some embodiments substantially equal. Alternatively, the ratio of the target peptides present in the least amount relative to the target peptide present in the greatest amount is in some embodiments about or at least 1:1.25, 1:1.5, 1:1.75, 1:2.0, 1:2.25, 1:2.5, 1:2.75, 1:3, 1:4, 1:5, 1:6, 1:7, 1:8, 1:9, 1:10, 1:20, 1:30; 1:40, 1:50, 1:100, 1:200, 1:500, 1:1000, 1:5000; 1:10,000; or 1:100,000. Alternatively, the ratio of the target peptides present in the least amount relative to the target peptide present in the greatest amount is in some embodiments about or at least 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15 to 20; 20 to 25; 1 to 40; 1 to 30; 1 to 20; 1 to 15; 10 to 40; 10 to 30; 10 to 20; 10 to 15; 20 to 40; 20 to 30; or 20 to 25; 1 to 100; 25 to 100; 50 to 100; 75 to 100; 25 to 75, 25 to 50, or 50 to 75; 25 to 40; 25 to 50; 30 to 50; 30 to 40; or 30 to 75.

Single dosages can in some embodiments be given to a patient about or at least 1, 2, 3, 4, or 5 times per day. Single dosages can in some embodiments be given to a patient about or at least 1, 2, 3, 4, 5, 6, 7, 8, 9, 10, 11, 12, 13, 14, 15, 16, 18, 19, 20, 21, 22, 23, 24, 36, 48, 60, or 72 hours subsequent to a previous dose.

Single dosages can in some embodiments be given to a patient about or at least 1, 2, 3, 4, 5, 6, or 7 times per week or every other, third, fourth, or fifth day. Single doses can in some embodiments also be given every week, every other week, or only during 1, 2, or 3 weeks per month. A course of treatment can in some embodiments last about or at least 1, 2, 3, 4, 5, 6, 7, 8, 9, 10, 11, or 12 months.

In some embodiments, single dosages of the compositions of the presently disclosed subject matter are provided to a patient in at least two phases, e.g., during an initial phase and then a subsequent phase. An initial phase can in some embodiments be about or at least 1, 2, 3, 4, 5, or 6 weeks in length. The subsequent phase can in some embodiments last at least or about 1, 2, 3, 4, 5, 6, 7, or 8 times as long as the initial phase. The initial phase can in some embodiments be separated from the subsequent phase by about or at least 1, 2, 3, 4, 5, 6, 7, 8, 9, 10, 11, or 12 weeks or months.

The target peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times greater than during the initial phase. The target peptide composition dosage during the subsequent phase can in some embodiments be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times lower than during the initial phase.

In some embodiments, the initial phase is about three weeks and the second phase is about 9 weeks. In some embodiments, the target peptide compositions would be administered to the patient on or about days 1, 8, 15, 36, 57, and 78.

VII.D. Kits and Storage

In some embodiments, the presently disclosed subject matter provides a kit. In some embodiments the kit comprises (a) a container that contains at least one target peptide composition as described above in solution or in lyophilized form; (b) optionally, a second container containing a diluent or reconstituting solution for the lyophilized formulation; and (c) also optionally, instructions for (i) use of the solution; and/or (ii) reconstitution and/or use of the lyophilized formulation. The kit can in some embodiments further comprise one or more of (iii) a buffer, (iv) a diluent, (v) a filter, (vi) a needle, and/or (v) a syringe. In some embodiments, the container is selected from the group consisting of a bottle, a vial, a syringe, a test tube, and a multi-use container. In some embodiments, the target peptide composition is lyophilized.

The kits can in some embodiments contain exactly, about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, or more target peptide-containing compositions. Each composition in the kit can in some embodiments be administered at the same time or at different times to a subject.

In some embodiments, the kits can comprise a lyophilized formulation of the presently disclosed compositions and/or vaccines in a suitable container and instructions for its reconstitution and/or use. Suitable containers include, for example, bottles, vials (e.g. dual chamber vials), syringes (such as dual chamber syringes), and test tubes. The container can in some embodiments be formed from a variety of materials such as glass or plastic. In some embodiments, the kit and/or container include instructions on or associated with the container that indicate directions for reconstitution and/or use. For example, the label can in some embodiments indicate that the lyophilized formulation is to be reconstituted to target peptide concentrations as described above. The label can in some embodiments further indicate that the formulation is useful or intended for subcutaneous administration. Lyophilized and liquid formulations are in some embodiments stored at −20° C. to −80° C.

The container holding the target peptide composition(s) can in some embodiments be a multi-use vial, which allows for repeat administrations (e.g., from 2-6 administrations) of the reconstituted formulation. The kit can in some embodiments further comprise a second container comprising a suitable diluent such as, but not limited to a sodium bicarbonate solution.

In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0, or 10 mg/mL/target peptide. In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0 or 10 μg/mL/target peptide.

The kit can in some embodiments further comprise other materials desirable from a commercial and user standpoint, including but not limited to other buffers, diluents, filters, needles, syringes, and/or package inserts with instructions for use.

The kits can in some embodiments have a single container that comprises the formulation of the target peptide compositions with or without other components (e.g., other compounds or compositions of these other compounds) or can in some embodiments have a distinct container for each component.

Additionally, the kits can in some embodiments comprise a formulation of the presently disclosed target peptide compositions and/or vaccines packaged for use in combination with the co-administration of a second compound such as but not limited to adjuvants (e.g. imiquimod), a chemotherapeutic agent, a natural product, a hormone or antagonist, an anti-angiogenesis agent or inhibitor, an apoptosis-inducing agent, or a chelator or a composition thereof. The components of the kit can in some embodiments be pre-complexed or each component can in some embodiments be in a separate distinct container prior to administration to a patient. The components of the kit can in some embodiments be provided in one or more liquid solutions. In some embodiments, the liquid solution is an aqueous solution. In some embodiments, the liquid solution is a sterile aqueous solution. The components of the kit can in some embodiments also be provided as solids, which in some embodiments are converted into liquids by addition of suitable solvents, which can in some embodiments be provided in another distinct container.

The container of a therapeutic kit can in some embodiments be a vial, a test tube, a flask, a bottle, a syringe, or any other article suitable to enclose a solid or liquid. In some embodiments, when there is more than one component, the kit can contain a second vial and/or other container, which allows for separate dosing. The kit can in some embodiments also contain another container for a pharmaceutically acceptable liquid. In some embodiments, a therapeutic kit contains an apparatus (e.g., one or more needles, syringes, eye droppers, pipette, etc.) that facilitates administration of the agents of the disclosure that are components of the present kit.

VIII.E. Markers for Efficacy

When administered to a patient, the vaccine compositions of the presently disclosed subject matter are envisioned to have certain physiological effects, including but not limited to the induction of a T cell mediated immune response.

VIII.E.1 Immunohistochemistry, Immunofluorescence, Western Blots, and Flow Cytometry

Validation and testing of antibodies for characterization of cellular and molecular features of lymphoid neogenesis has been performed. Commercially available antibodies for use in immunohistochemistry (IHC), immunofluorescence (IF), flow cytometry (FC), and western blot (WB) can in some embodiments be employed. In some embodiments, such techniques can be employed to analyze patient samples, e.g., formalin-fixed, paraffin-embedded tissue samples, for CD1a, S100, CD83, DC-LAMP, CD3, CD4, CD8, CD20, CD45, CD79a, PNAd, TNFalpha, LIGHT, CCL19, CCL21, CXCL12, TLR4, TLR7, FoxP3, PD-1 and Ki67 expression. In some embodiments, flow cytometry is used to determine CD3, CD4, CD8, CD13, CD14, CD16, CD19, CD45RA, CD45RO, CD56, CD62L, CD27, CD28, CCR7, FoxP3 (intracellular), and MHC-peptide tetramers for I MHC associated (phospho)-peptides. In some embodiments, positive control tissue selected from among normal human peripheral blood lymphocytes (PBL), PBL activated with CD3/CD28 beads (activated PBL), human lymph node tissue from non-cancer patients (LN), and inflamed human tissue from a surgical specimen of Crohn's disease (Crohn's) can be employed.

VII.E.2. ELISpot Assay

In some embodiments, vaccination site infiltrating lymphocytes and lymphocytes from the sentinel immunized nod (SIN) and vaccine site can be evaluated by ELISpot. ELISpot permits the direct counting of T cells reacting to antigen by production of INFγ. Peripheral blood lymphocytes can be evaluated by ELISpot assay for the number of peptide-reactive T cells. Vaccine site infiltrating lymphocytes and SIN lymphocytes can be compared to those in peripheral blood. It is envisioned that positive results of the ELISpot assay correlate with increased patient progression free survival. Progression free survival is in some embodiments defined as the time from start of treatment until death from any cause or date of last follow up.

VII.E.3. Tetramer Assay

Peripheral blood lymphocytes and lymphocytes from the SIN and vaccine site can be evaluated by flow cytometry after incubation with MHC-peptide tetramers for the number of peptide-reactive T cells.

VII.E.4. Proliferation Assay/Cytokine Analysis

Peripheral blood mononuclear cells (PBMC), vaccine-site inflammatory cells, and lymphocytes from the SIN from patients can in some embodiments be evaluated for CD4 T cell reactivity to, e.g., tetanus helper peptide mixture, using a ³H-thymidine uptake assay. Additionally, Th1 (IL-2, IFN-gamma, TNFa), Th2 (IL-4, IL-5, IL-10), Th17 (IL-17, and IL23), and T-reg (TGF-beta) cytokines in media from 48 hours in that proliferation assay can be employed to determine if the microenvironment supports generation of Th1, Th2, Th17, and/or T-reg responses. In some embodiments, two peptides are used as negative controls: a tetanus peptide and the PADRE peptide (AK(X)VAAWTLKAA).

VII.E.5. Evaluation of Tumors

In some embodiments tumor tissue collected prior to treatment or at the time of progression can be evaluated by routine histology and immunohistochemistry. Alternatively or in addition, in vitro evaluations of tumor tissue and tumor infiltrating lymphocytes can be completed.

VII.E.6. Studies of Homing Receptor Expression

Patient samples can in some embodiments be studied for T cell homing receptors induced by vaccination the compositions of the invention. These include, but are not limited to, integrins (including alphaE-beta7, alpha1-beta1, alpha4-beta1), chemokine receptors (including CXCR3), and selectin ligands (including CLA, PSL) on lymphocytes, and their ligands in the vaccine sites and SIN. These can be assayed by immunohistochemistry, flow cytometry or other techniques.

VII.E.7. Studies of Gene and Protein Expression

Differences in gene expression and/or for differences in panels of proteins can in some embodiments be assayed by high-throughput screening assays (e.g. nucleic acid chips, protein arrays, etc.) in the vaccine sites and sentinel immunized nodes.

### VIII. Antibodies Including Antibody-Like Molecules

Antibodies and antibody-like molecules (e.g. T cell receptors) specific for target peptides or target peptide/MHC complexes are, for example, useful, inter alia, for analyzing tissue to determine the pathological nature of tumor margins and/or can be employed in some embodiments as therapeutics. Alternatively, such molecules can in some embodiments be employed as therapeutics targeting cells, e.g., tumor cells, which display target peptides on their surface. In some embodiments, the antibodies and antibody-like molecules bind the target peptides or target peptide-MHC complex specifically and do not substantially cross react with non-phosphorylated native peptides.

As used herein, “antibody” and “antibody peptide(s)” refer to intact antibodies, antibody-like molecules, and binding fragments thereof that compete with intact antibodies for specific binding. Binding fragments are in some embodiments produced by recombinant DNA techniques or in some embodiments by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody in some embodiments substantially inhibits adhesion of a receptor to a counterreceptor when an excess of antibody reduces the quantity of receptor bound to counterreceptor by at least about 20%, 40%, 60%, 80%, 85%, 90%, 91%, 92%, 93%, 94%, 95%, 96%, 97%, 98%, 99%, or greater than 99% as measured, for example, in an in vitro competitive binding assay.

The term “MHC” as used herein refers to the Major Histocompatibility Complex, which is defined as a set of gene loci specifying major histocompatibility antigens. The term “HLA” as used herein refers to Human Leukocyte Antigens, which are defined as the histocompatibility antigens found in humans. As used herein, “HLA” is the human form of “MHC”.

The terms “MHC light chain” and “MHC heavy chain” as used herein refer to portions of MHC molecules. Structurally, class I molecules are heterodimers comprised of two non-covalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “02-microglobulin”, and “p2m” are used interchangeably herein.

The term “epitope” includes any protein determinant capable of specific binding to an immunoglobulin or T cell receptor. Epitopic determinants usually consist of chemically active surface groupings of molecules such as amino acids or sugar side chains and usually have specific three dimensional structural characteristics, as well as specific charge characteristics. An antibody or antibody like molecule is said to “specifically” bind an antigen when the dissociation constant is in some embodiments less than 1 μM, in some embodiments less than 100 nM, and in some embodiments less than 10 nM.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv), as well as “antibody-like molecules” so long as they exhibit the desired biological activity. Antibodies (Abs) and immunoglobulins (Igs) are glycoproteins having the same structural characteristics. The term is also meant to encompass “antibody like molecules” and other members of the inmunoglobulin superfamily, e.g., T cell receptors, MHC molecules, containing e.g., an antigen-binding regions and/or variable regions, e.g., complementary determining regions (CDRs) which specifically bind the target peptides disclosed herein.

In some embodiments, antibodies and antibody-like molecules bind to the target peptides of the presently disclosed subject matter but do not substantially and/or specifically cross react with the same peptide in a modified form. See e.g., U.S. Patent Application Publication No. 2009/0226474, which is incorporated by reference.

The presently disclosed subject matter also includes antibodies that recognize target peptides associated with a tumorigenic or disease state, wherein the peptides are displayed in the context of HLA molecules. These antibodies typically mimic the specificity of a T cell receptor (TCR) but can in some embodiments have higher binding affinity such that the molecules can be employed as therapeutic, diagnostic, and/or research reagents. Methods of producing a T cell receptor mimic of the presently disclosed subject matter include identifying a target peptide of interest, wherein the target peptide of interest comprises an amino acid sequence as set forth in Table 2. Then, an immunogen comprising at least one target peptide/MHC complex is formed. An effective amount of the immunogen is then administered to a host for eliciting an immune response, and serum collected from the host is assayed to determine if desired antibodies that recognize a three-dimensional presentation of the target peptide in the binding groove of the MHC molecule are being produced. The desired antibodies can differentiate the target peptide/MHC complex from the MHC molecule alone, the target peptide alone, and a complex of MHC and irrelevant target peptide. Finally, in some embodiments the desired antibodies are isolated.

The term “antibody” also encompasses soluble T cell receptors (TCR) cytoplasmic domains which are stable at low concentrations and which can recognize MHC-peptide complexes. See e.g., U.S. Patent Application Publication No. 2002/0119149, which is incorporated by reference. Such soluble TCRs might for example be conjugated to immunostimulatory peptides and/or proteins or moieties, such as CD3 agonists (anti-CD3 antibody), for example. The CD3 antigen is present on mature human T cells, thymocytes, and a subset of natural killer cells. It is associated with the TCR and is responsible for the signal transduction of the TCR.

Antibodies specific for the human CD3 antigen are well-known. One such antibody is the murine monoclonal antibody OKT3 which was the first monoclonal antibody approved by the FDA. OKT3 is reported to be a potent T cell mitogen (Van Wauve (1980) J Immunol 124:2708-2718; see also U.S. Pat. No. 4,361,539) and a potent T cell killer (Wong (1990) Transplantation 50:683-389). Other antibodies specific for the CD3 antigen have also been reported. (see PCT International Patent Application Publication No. WO 2004/0106380; U.S. Patent Application Publication No. 2004/0202657; U.S. Pat. Nos. 6,750,325; 6,706,265; GB 2249310A; Clark et al. (1989) Eur J Immunol 19:381-388; U.S. Pat. No. 5,968,509; and U.S. Patent Application Publication No. 2009/0117102). ImmTACs (Immunocore Limited, Milton Park, Abington, Oxon, United Kingdom) are innovative bifunctional proteins that combine high-affinity monoclonal T cell receptor (mTCR) targeting technology with a clinically-validated, highly potent therapeutic mechanism of action (Anti-CD3 scFv).

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. The number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al. (1985) J Mol Biol 186:651-66; Novotny & Haber (1985) Proc Natl Acad Sci USA 82:4592-4596).

An “isolated” antibody is one which has been separated, identified, and/or recovered from a component of the environment in which it was produced. Contaminant components of its production environment are materials which would interfere with diagnostic or therapeutic uses for the antibody, and can include enzymes, hormones, and other proteinaceous or nonproteinaceous solutes. In some embodiments, the antibody is purified as measurable by at least one of the following three different methods: 1) to in some embodiments greater than 50% by weight of antibody as determined by the Lowry method, such as but not limited to in some embodiments greater than 75% by weight, in some embodiments greater than 85% by weight, in some embodiments greater than 95% by weight, in some embodiments greater than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, in some embodiments, silver stain. Isolated antibodies include the antibody in situ within recombinant cells since at least one component of the antibody's natural environment is not present. In some embodiments, however, isolated antibodies are prepared by a method that includes at least one purification step.

The terms “antibody mutant”, “antibody variant”, and “antibody derivative” refer to an amino acid sequence variant of an antibody wherein one or more of the amino acid residues of a reference antibody has been modified (e.g., substituted, deleted, chemically modified, etc.). Such mutants necessarily have less than 100% sequence identity or similarity with the amino acid sequence of either the heavy or light chain variable domain of the reference antibody. The resultant sequence identity or similarity between the modified antibody and the reference antibody is thus in some embodiments at least 80%, in some embodiments at least 85%, in some embodiments at least 90%, in some embodiments at least 95%, in some embodiments at least 97%, and in some embodiments at least 99%.

The term “variable” in the context of variable domain of antibodies, refers to the fact that certain portions of the variable domains differ extensively in sequence among antibodies and are used in the binding and specificity of each particular antibody for its particular antigen(s). However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al. (1987) Sequences of Proteins of Immunological Interest National Institute of Health, Bethesda, Md., United States of America); and (2) an approach based on crystallographic studies of antigen-antibody complexes (Chothia et al. (1989) Nature 342:877-883). The more highly conserved portions of variable domains are called the framework (FR) regions. The variable domains of native heavy and light chains each comprise four FR regions, largely adopting a R-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., 1987, op. cit.). The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L}dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, immunoglobulins can be assigned to different classes. There are at least five (5) major classes of immunoglobulins: IgA, IgD, IgE, IgG and IgM, and several of these can be further divided into subclasses (isotypes), e.g., IgG₁, IgG₂, IgG₃, and IgG₄; IgA₁ and IgA₂. The heavy chains constant domains that correspond to the different classes of immunoglobulins are called alpha (a), delta (A), epsilon (E), gamma (γ), and mu (p), respectively. The subunit structures and three-dimensional configurations of different classes of immunoglobulins are well-known.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, monoclonal antibodies can be advantageous in that they can be synthesized in hybridoma culture, uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter can in some embodiments be made by the hybridoma method first described by Kohler & Milstein (1975) Nature 256:495, or can in some embodiments be made by recombinant methods, e.g., as described in U.S. Pat. No. 4,816,567. The monoclonal antibodies for use with the presently disclosed subject matter can in some embodiments also be isolated from phage antibody libraries using the techniques described in Clackson et al. (1991) Nature 352:624-628 or in Marks et al. (1991) J Mol Biol 222:581-597.

Utilization of the monoclonal antibodies of the presently disclosed subject matter can in some embodiments require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed subject matter can be “humanized”: that is, the antibodies can be engineered such that antigenic portions thereof are removed and like portions of a human antibody are substituted therefor, while the antibodies' affinity for specific peptide/MHC complexes is retained. This engineering can in some embodiments only involve a few amino acids, or can in some embodiments include entire framework regions of the antibody, leaving only the complementarity determining regions of the antibody intact. Several methods for humanizing antibodies are known in the art and are disclosed, for example, in U.S. Pat. No. 6,180,370 to Queen et al.; U.S. Pat. No. 6,054,927 to Brickell; U.S. Pat. No. 5,869,619 to Studnicka; U.S. Pat. No. 5,861,155 to Lin; U.S. Pat. No. 5,712,120 to Rodriquez et al.; and 4,816,567 to Cabilly et al., the entire content of each of which is hereby expressly incorporated herein by reference in its entirety.

Humanized forms of antibodies are chimeric immunoglobulins, immunoglobulin chains, or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. In some embodiments, humanization can be performed following the method of Winter and co-workers (see e.g., Jones et al. (1986) Nature 321:522-525; Riechmann et al. (1988) Nature 332:323-327; Verhoeyen et al. (1988) Science 239:1534-1536) by substituting rodent CDRs or CDR sequences for the corresponding sequences of a human antibody. See also U.S. Pat. No. 5,225,539. In some embodiments, Fᵥ framework residues of a human immunoglobulin are replaced by corresponding non-human residues.

Humanized antibodies can also comprise residues which are found neither in the recipient antibody nor in the imported CDR or framework sequences. In general, a humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally can in some embodiments also comprise at least a portion of an immunoglobulin constant region (Fc), typically that of a human immunoglobulin. See e.g., Jones et al. (1986) Nature 321:522-525; Riechmann et al. (1988) Nature 332:323-327; Presta (1992) Proc Natl Acad Sci USA 89:4285-4289.

Many articles relating to the generation or use of humanized antibodies teach useful examples of protocols that can be utilized with the presently disclosed subject matter, such as but not limited to Sandborn et al. (2001) Gastroenterology 120:1330-1338; Mihara et al. (2001) Clin Immunol 98:319; Yenari et al. (2001) Neurol Res 23:72; Morales et al. (2000) Nucl Med Biol 27:199; Richards et al. (1999) Cancer Res 59:2096; Yenari et al. (1998) Exp Neurol 153:223; and Shinkura et al. (1998) Anticancer Res 18:1217, all of which are expressly incorporated in their entireties by reference. For example, a treatment protocol that can be utilized in such a method includes a single dose, generally administered intravenously, of 10-20 mg of humanized mAb per kg (Sandborn, et al. (2001) Gastroenterology 120:1330-1338). In some embodiments, alternative dosing patterns can be appropriate, such as but not limited to the use of three infusions, administered once every two weeks, of 800 to 1600 mg or even higher amounts of humanized mAb (Richards et al., 1999, op. cit.). However, it is to be understood that the presently disclosed subject matter is not limited to the treatment protocols described above, and other treatment protocols that are known to a person of ordinary skill in the art can be utilized in the methods of the presently disclosed subject matter.

The presently disclosed and claimed subject matter further includes in some embodiments fully human monoclonal antibodies against specific target peptide/MHC complexes. Fully human antibodies essentially relate to antibody molecules in which the entire sequence of both the light chain and the heavy chain, including the CDRs, arise from human genes. Such antibodies are referred to herein as “human antibodies” or “fully human antibodies”. Human monoclonal antibodies can be prepared by the trioma technique; the human B-cell hybridoma technique (see Kozbor et al. (1983) Hybridoma, 2:7), and the EBV hybridoma technique to produce human monoclonal antibodies (see Cole et al. (1985) Proc Natl Acad Sci USA 82:859). Human monoclonal antibodies can in some embodiments be utilized in the practice of the presently disclosed subject matter and can in some embodiments be produced by using human hybridomas (see Cote et al. (1983) Proc Natl Acad Sci US A 80:2026) or by transforming human B-cells with Epstein Barr Virus in vitro (see Cole et al., 1985, op. cit.).

In addition, human antibodies can also be produced using additional techniques, including but not limited to phage display libraries (Hoogenboom et al. (1991) Nucleic Acids Res 19:4133; Marks et al. (1991) J Mol Biol 222:581). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016; and in Marks et al. (1992) J Biol Chem 267:16007; Lonberg et al. (1994) Nature 368:856; Fishwild et al. (1996) Nature Biotechnol 14:845; Neuberger (1996) Nature Biotechnol 14:826; and Lonberg & Huszar (1995) Intl Rev Immunol 13:65.

Human antibodies can in some embodiments additionally be produced using transgenic nonhuman animals which are modified so as to produce fully human antibodies rather than the animal's endogenous antibodies in response to challenge by an antigen. See PCT International Patent Application Publication No. WO 1994/02602). Typically, the endogenous genes encoding the heavy and light immunoglobulin chains in the non-human host are incapacitated, and active loci encoding human heavy and light chain immunoglobulins are inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications.

A non-limiting example of such a nonhuman animal is a mouse, and is termed the XENOMOUSE™ as disclosed in PCT International Patent Application Publication Nos. WO 1996/33735 and WO 1996/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a non-human host, exemplified as a mouse, lacking expression of an endogenous immunoglobulin heavy chain is disclosed in U.S. Pat. No. 5,939,598 to Kucherlapati et al. (incorporated herein by reference). It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

An exemplary method for producing an antibody of interest, such as a human antibody, is disclosed in U.S. Pat. No. 5,916,771 to Hori et al. (incorporated herein by reference). It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

The antigen target peptides are known to be expressed on a variety of cancer cell types. Thus, antibodies and antibody-like molecules can be used where appropriate, in treating, diagnosing, vaccinating, preventing, retarding, and/or attenuating melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

Antibodies generated with specificity for the antigen target peptides can be used to detect the corresponding target peptides in biological samples. The biological sample could come from an individual who is suspected of having cancer and thus detection would serve to diagnose the cancer. Alternatively, the biological sample can in some embodiments come from an individual known to have cancer, and detection of the antigen target peptides would serve as an indicator of disease prognosis, cancer characterization, or treatment efficacy. Appropriate immunoassays are well-known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine. Antigens recognized by T cells, whether helper T lymphocytes or CTL, are not recognized as intact proteins, but rather as small peptides that associate with class I or class II MHC proteins on the surface of cells. During the course of a naturally occurring immune response antigens that are recognized in association with class II MHC molecules on antigen presenting cells are acquired from outside the cell, internalized, and processed into small peptides that associate with the class II MHC molecules. Conversely, the antigens that give rise to proteins that are recognized in association with class I MHC molecules are generally proteins made within the cells, and these antigens are processed and associate with class I MHC molecules. It is now well-known that the peptides that associate with a given class I or class II MHC molecule are characterized as having a common binding motif, and the binding motifs for a large number of different class I and II MHC molecules have been determined. It is also well-known that synthetic peptides can be made which correspond to the sequence of a given antigen and which contain the binding motif for a given class I or II MHC molecule. These peptides can then be added to appropriate antigen presenting cells, and the antigen presenting cells can be used to stimulate a T helper cell or CTL response either in vitro or in vivo. The binding motifs, methods for synthesizing the peptides, and methods for stimulating a T helper cell or CTL response are all well-known and readily available.

Kits can in some embodiments be composed for help in diagnosis, monitoring, and/or prognosis. The kits are to facilitate the detecting and/or measuring of cancer-specific target peptides or proteins. Such kits can in some embodiments contain in a single or divided container, a molecule comprising an antigen-binding region. Such molecules can in some embodiments be antibodies and/or antibody-like molecules. Additional components that can be included in the kit include, for example, solid supports, detection reagents, secondary antibodies, instructions for practicing, vessels for running assays, gels, control samples, and the like. The antibody and/or antibody-like molecules can in some embodiments be directly or indirectly labeled, as an option.

Alternatively or in addition, the antibody or antibody-like molecules specific for target peptides and/or target peptide/MHC complexes can in some embodiments be conjugated to therapeutic agents. Exemplary therapeutic agents include:

Alkylating Agents: Alkylating agents are drugs that directly interact with genomic DNA to prevent cells from proliferating. This category of chemotherapeutic drugs represents agents that affect all phases of the cell cycle, that is, they are not phase-specific. An alkylating agent can in some embodiments include, but is not limited to, a nitrogen mustard, an ethylenimene, a methylmelamine, an alkyl sulfonate, a nitrosourea or a triazines. They include but are not limited to busulfan, chlorambucil, cisplatin, cyclophosphamide (cytoxan), dacarbazine, ifosfamide, mechlorethamine (mustargen), and melphalan.

Antimetabolites: Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to 5-fluorouracil (5-FU), cytarabine (Ara-C), fludarabine, gemcitabine, and methotrexate.

Natural Products: Natural products generally refer to compounds originally isolated from a natural source, and identified as having a pharmacological activity. Such compounds, as well as analogs and derivatives thereof, can in some embodiments be isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include, but are not limited to, compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antibiotics: Certain antibiotics have both antimicrobial and cytotoxic activity. These drugs can also interfere with DNA by chemically inhibiting enzymes and mitosis or altering cellular membranes. These agents are typically not phase-specific so they work in all phases of the cell cycle. Examples of cytotoxic antibiotics include but are not limited to bleomycin, dactinomycin, daunorubicin, doxorubicin (Adriamycin), plicamycin (mithramycin), and idarubicin.

Miscellaneous Agents: Miscellaneous cytotoxic agents that do not fall into the previous categories include but are not limited to platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic, and/or cytocidal agent can be conjugated or otherwise attached to targeting peptides and administered to a targeted organ, tissue, and/or cell type within the scope of the presently disclosed subject matter.

Chemotherapeutic (cytotoxic) agents include but are not limited to 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

The peptides identified and tested thus far in peptide-based vaccine approaches have generally fallen into one of three categories: 1) mutated on individual tumors, and thus not displayed on a broad cross section of tumors from different patients; 2) derived from unmutated tissue-specific proteins, and thus compromised by mechanisms of self-tolerance; and 3) expressed in subsets of cancer cells and normal testes.

Antigens linked to transformation or oncogenic processes are of primary interest for immunotherapeutic development based on the hypothesis that tumor escape through mutation of these proteins can be more difficult without compromising tumor growth or metastatic potential.

The target peptides of the presently disclosed subject matter are unique in that the identified target peptides are modified by intracellular modification. This modification is of particular relevance because it is associated with a variety of cellular control processes, some of which are dysregulated in cancer cells. For example, the source proteins for class I MHC-associated phosphopeptides are often known phosphoproteins, supporting the idea that the phosphopeptides are processed from folded proteins participating in signaling pathways.

Although not wishing to be bound by any particular theory, it is envisioned that the target peptides of the presently disclosed subject matter are unexpectedly superior to known tumor-associated antigen-derived peptides for use in immunotherapy because: 1) they only displayed on the surface of cells in which intracellular phosphorylation is dysregulated, i.e., cancer cells, and not normal thymus cells, and thus they are not are not compromised by self-tolerance (as opposed to TAA which are associated with overexpression or otherwise expressed on non-mutated cells); and/or 2) they identify a cell displaying them on their surface as having dysregulated phosphorylation. Thus, post-translationally-modified phosphopeptides that are differentially displayed on cancer cells and derived from source proteins objectively linked to cellular transformation and metastasis allow for more extensive anti-tumor responses to be elicited following vaccination. Target peptides are, therefore, better immunogens in peptide-based vaccines, as target peptides are derived from proteins involved with cellular growth control, survival, or metastasis and alterations in these proteins as a mechanism of immune escape can interfere with the malignant phenotype of tumors.

As such, the presently disclosed subject matter also relates in some embodiments to methods for identifying target peptides for use in immunotherapy which are displayed on transformed cells but are not substantially expressed on normal tissue in general or in the thymus in particular. In some embodiments, target peptides bind the MHC class I molecule more tightly than their non-phosphorylated native counterparts. Moreover, such target peptides can in some embodiments have additional binding strength by having amino acid substitutions at certain anchor positions. In some embodiments, such modified target peptides can remain cross-reactive with TCRs specific for native target peptide MHC complexes. Additionally, it is envisioned that the target peptides associated with proteins involved in intracellular signaling cascades or cycle regulation are of particular interest for use in immunotherapy. In some cases, the TCR binding can specifically react with the phosphate groups on the target peptide being displayed on an MHC class I molecule.

In some embodiments, the method of screening target peptides for use in immunotherapy, e.g., in adaptive cell therapy or in a vaccine, involves determining whether the candidate target peptides are capable of inducing a memory T cell response. The contemplated screening methods can include providing target peptides, e.g., those disclosed herein or those to be identified in the future, to a healthy volunteer and determining the extent to which a target peptide-specific T cell response is observed. In some embodiments, the extent to which the T cell response is a memory T cell response is also determined. In some embodiments, it is determined the extent to which a T_{CM} response is elicited, e.g., relative to other T cell types. In some embodiments, those target peptides which are capable of inducing a memory T cell response in health and/or diseased patients are selected for inclusion in the therapeutic compositions of the presently disclosed subject matter.

In some embodiments, the presently disclosed subject matter provides methods for inducing a target peptide-specific memory T cell response (e.g., T_{CM}) response in a patient by providing the patient with a composition comprising the target peptides disclosed herein. In some embodiments, the compositions are those disclosed herein and are provided in a dosing regimen disclosed herein.

In some embodiments, the presently disclosed subject matter relates to methods for determining a cancer disease prognosis. These methods involve providing a patient with target peptide compositions and determining the extent to which the patient is able to mount a target peptide specific T cell response. In some embodiments, the target peptide composition contains target peptides selected in the same substantially the same manner that one would select target peptides for inclusion in a therapeutic composition. If a patient is able to mount a significant target peptide-specific T cell response, then the patient is likely to have a better prognosis than a patient with the similar disease and therapeutic regimen that is not able to mount a target peptide-specific T cell response. In some embodiments, the methods involve determining whether the target peptide specific T cell response is a T_{CM} response. In some embodiments, the presence of a target peptide-specific T cell response as a result of the presently disclosed diagnostic methods correlates with an at least or about 5, 10, 15, 20, 25, 30, 35, 40, 45, 50, 55, 60, 65, 70, 75, 80, 85, 90, 95, 100, 125, 150, 175, 200, 250, 300, 400, 500, or more percent increase in progression free survival over standard of care.

### REFERENCES

All references listed in the instant disclosure, including but not limited to all patents, United States and PCT International patent applications and publications thereof, scientific journal articles, and database entries (including but not limited to Uniprot, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altman et al. (1996) Science 274:94-96.
Arentz-Hansen et al. (2000) J Exp Med 191:603-612.
Arozarena et al. (2011) Oncogene 30:4531-4543.
Bachmann et al. (2005) Clin Cancer Res 11:8606-8614.
Baron et al. (2005) J Clin Oncol 23:1993-2003.
Bertoletti et al. (1994) Nature 369:407-410.
Berzofsky et al. (1988) Immunol Rev 106:5-31.
Bullock et al. (2000) J Immunol 164:2354-2361.
Chi (2011) Nature 471:537-538.
Chien et al. (2009) Proc Natl Acad Sci USA 106:1193-1198.
Cobbold et al. (2005) J Exp Med 202:379-386.
Crawford et al. (1999) Oncogene 18:2883-2891.
Demunter et al. (2002) Modern Pathol 15:454-461.
Dephoure et al. (2008) Proc Natl Acad Sci USA 105:10762-10767.
Depontieu et al. (2009) Proc Natl Acad Sci USA 106:12073-12078.
Dudley et al. (2008) J Clin Oncol 26:5233-5239.
DuPage et al. (2012) Nature 482:405-409.
Finn (2003) J Exp Med 198:1623-1626.
Fiol et al. (1988) Arch Biochem Biophys 267:797-802.
Fiol et al. (1990) J Biol Chem 265:6061-6065.
Fischbein et al. (2000) J Immunol 165:7316-7322.
Gale et al. (1994) Ann Intern Med 120:646-652.
Gerdes et al. (1999) Digestion 60:544-548.
Girbal-Neuhauser et al. (1999) J Immunol 162:585-594.
Goldman & DeFrancesco (2009) Nat Biotech 27:129-139.
Hall et al. (2010) Mol Cell Proteomics 9:2853-2863.
Haluska et al. (2006) Clin Cancer Res 12:2301s-2307s.
He et al. (1998) Science 281:1509-1512.
Hirohashi et al. (2009) Cancer Sci 100:798-806.
Ho et al. (2006) J Immunol Methods 310:40-52.
Hoek et al. (2006) Pigment Cell Res 19:290-302.
Hogan et al. (1998) Cancer Res 58:5144-5150.
Homfray et al. (1998) Human Mutation 11:114-120.
Horowitz et al. (1990) Blood 75:555-562.
Hulsken et al. (1994) J Cell Biol 127:2061-2069.
Ilyas et al. (1997) Proc Natl Acad Sci USA 94:10330-10334.
Isakoff et al. (2005) Cancer Res 65:10992-11000.
Jimbow et al. (1975) J Cell Biol 66:663-670.
Jones et al. (2008) Science 321:1801-1806.
Kageshita et al. (2001) Br J Dermatol 145:210-216.
Kantoff et al. (2010) N Engl J Med 363:411-422.
Kielhorn et al. (2003) Intl J Cancer 103:652-656.
Kim et al. (2000) Pathol Intl 50:725-730.
Kimelman & Xu (2006) Oncogene 25:7482-7491.
Klenerman et al. (1994) Nature 369:403-407.
Kolb et al. (1990) Blood 76:2462-2465.
Kolb (2008) Blood 112:4371-4383.
Krengel et al. (2004) J Cutaneous Pathol 31:1-7.
Kroger et al. (2005) Bone Marrow Transplant 35:37-43.
Ley et al. (2008) Nature 456:66-72.
Liu et al. (2002) Cell 108:837-847.
Lucas & Coulie (2008) Sem Immunol 20:301-307.
Maelandsmo et al. (2003) Clin Cancer Res 9:3383-3388.
Mamula et al. (1999) J Biol Chem 274:22321-22327.
Marafioti et al. (2004) Haematologica 89:957-964.
Matsushita et al. (2012) Nature 482:400-404.
Meyer et al. (2009) J Proteome Res 8:3666-3674.
Miyake et al. (2001) Pathol Intl 51:680-685.
Mohammed et al. (2008) Nat Immunol 9:1236-1243.
Molina et al. (2007) Proc Natl Acad Sci USA 104:2199-2204.
Morin et al. (1997) Science 275:1787-1790.
Newberg et al. (1992) J Immunol 149:136-142.
Niedermann et al. (1995) Immunity 2:289-299.
Nunes et al. (2011) Cancer Res 71:5435-5444.
Offringa (2009) Curr Opin Immunol 21:190-199.
Ogasawara et al. (2006) Histopathology 49:612-621.
Ohgaki et al. (2004) Cancer Lett 207:197-203.
Oliva et al. (2006) J Pathol 208:708-713.
Olmeda et al. (2003) Mol Biol Cell 14:2844-2860.
Omholt et al. (2001) Intl J Cancer 92:839-842.
Parsons et al. (2011) Science 331:435-439.
Pavletic et al. (2007) Leukemia 21:2452-2455.
PCT International Patent Application Publication Nos. WO 2011/0149909; WO 2014/036562; WO 2014/039675; WO 2014/093855; WO 2017/027403; WO 2017/192969; each of which is incorporated herein by reference in its entirety.
PCT International Patent Application Serial No. PCT/US2020/024348.
Pecina-Slaus et al. (2007) J Cutaneous Pathol 34:239-246.
Petersen et al. (2009) Proc Natl Acad Sci USA 106:2776-2781.
Pollock & Hayward (2002) Melanoma Res 12:183-186.
Preudhomme et al. (2010) N Engl J Med 363:2511-2521.
Rappsilber et al. (2007) Nature Protocols 2:1896-1906.
Restifo et al. (1993) J Exp Med 177:265-272.
Rimm et al. (1999) Am J Pathol 154:325-329.
Robila et al. (2008) J Immunol 181:7843-7852.
Rosenberg & Dudley (2009) Curr Opin Immunol 21:233-240.
Rosenberg et al. (1986) Science 233:1318-1321.
Rosenberg et al. (2004) Nat Med 10:909-915.
Ruppert et al. (1993) Cell 74:929-937.
Sadot et al. (2002) J Cell Sci 115:2771-2780.
Sanders et al. (1999) Mol Pathol 52:151-157.
Schreiber et al. (2011) Science 331:1565-1570.
Seidensticker & Behrens (2000) Biochim Biophys Acta 1495:168-182.
Sette et al. (1994) J Immunol 153:5586-5592.
Slawson et al. (2005) J Biol Chem 280:32944-32956.
Slawson et al. (2008) Mol Biol. Cell 19:4130-4140.
Slingluff et al. (2000) Cancer Immunol Immunother 48:661-672.
Sun et al. (2005) J Neurooncol 73:131-134.
Takahashi et al. (2002) Oncogene 21:5861-5867.
Takemaru et al. (2008) Handb Exp Pharmacol 186:261-84.
Talpaz et al. (1986) N Engl J Med 314:1065-1069.
Tetsu & McCormick (1999) Nature 398:422-426.
U.S. Patent Application Publication Nos. 2005/0277161; 2013/0259883; 2015/0328297; 2016/0000893; 2019/0015494; 2019/0374627.
U.S. Pat. Nos. 9,279,011; 9,561,266; 10,640,535; 10,682,399.
Utz et al. (1997) J Exp Med 185:843-854.
van Doom et al. (2005) J Clin Oncol 23:3886-3896.
Wang et al. (2007) Mol Cell Proteomics 6:1365-1379.
Wang et al. (2010) Sci Signal 3(104):ra2, including Supplemental Materials.
Worm et al. (2004) Oncogene 23:5215-5226.
Wuttge et al. (1999) Immunol 98:273-279.
Yewdell (2002) Mol Immunol 39:139-146.
Yost et al. (1996) Genes Dev 10:1443-1454.
Zarling et al. (2000) J Exp Med 192:1755-1762.
Zarling et al. (2006) Proc Natl Acad Sci USA 103:14889-14894.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising, consisting essentially of, or consisting of at least or about 1, 2, 3, 4, 5, 6, 7, 8, 9, 10, or more synthetic target peptides, wherein each synthetic target peptide:
(i) is about or at least 8-50 amino acids long; and
(ii) comprises, consists essentially of, or consists of an amino acid sequence as set forth in Table 2,
and further wherein said composition optionally stimulates a T cell-mediated immune response to at least one of the synthetic target peptides.

2. The composition of claim 1, wherein at least one of the synthetic target peptides comprises a substitution of a serine residue with a homo-serine residue.

3. The composition of claim 1, wherein at least one of the synthetic target peptides is a phosphopeptide that comprises a phosphate group, optionally a non-hydrolyzable phosphate group.

4. The composition of claim 1, wherein the composition is immunologically suitable for at least 60%, 70%, 75%, 90%, 85%, 90%, 95%, of patients of a given cancer.

5. The composition of claim 1, wherein the composition comprises, consists essentially of, or consists of at least 5 different target peptides.

6. The composition of claim 1, wherein the composition comprises, consists essentially of, or consists of at least 10 different target peptides.

7. The composition of claim 1, wherein the composition comprises, consists essentially of, or consists of at least 15 different target peptides.

8. The composition of claim 1, wherein at least one of the synthetic target peptides is capable of binding to an MHC class I molecule, optionally an MHC class I molecule selected from the group consisting of an HLA A*0101 allele, an HLA-A*0201 allele, an HLA B*2705 allele, an HLA A*0301 allele, an HLA B*0702 allele, and an HLA B*4402 allele.

9. The composition of claim 1, wherein the composition is capable of increasing the 5-year survival rate of a cancer patient treated with the composition by at least 20 percent relative to average 5-year survival rates that could have been expected without treatment with the composition.

10. The composition of claim 1, wherein the composition is capable of increasing the survival rate of a cancer patient treated with the composition by at least 20 percent relative to a survival rate that could have been expected without treatment with the composition.

11. The composition of claim 1, wherein the composition is capable of increasing the treatment response rate of a cancer patient treated with the composition by at least 20 percent relative to a treatment rate that could have been expected without treatment with the composition.

12. The composition of claim 1, wherein the composition is capable of increasing the overall median survival of a cancer patient treated with the composition by at least two months relative to an overall median survival that could have been expected without treatment with the composition.

13. The composition of claim 1, further comprising at least one peptide derived from MelanA (MART-1), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

14. The composition of claim 1, wherein the composition further comprises an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

15. An in vitro population of dendritic cells comprising, consisting essentially of, or consisting of the composition of claim 1 or a composition comprising at least one target peptide comprising an amino acid sequence as set forth in Table 2.

16. An in vitro population of CD8⁺ T cells capable of being activated upon being brought into contact with a population of dendritic cells, wherein the dendritic cells comprise, consist essentially of, or consist of a composition of claim 1 or a composition comprising, consisting essentially of, or consisting of at least one target peptide comprising an amino acid sequence as set forth in Table 2.

17. An antibody or antibody-like molecule that specifically binds to a complex of an MHC class I molecule and a peptide comprising, consisting essentially of, or consisting of an amino acid sequence as set forth in Table 2.

18. The antibody or antibody-like molecule of claim 17, wherein the antibody or antibody-like molecule is a member of the immunoglobulin superfamily.

19. The antibody or antibody-like molecule of claim 17, wherein the antibody or antibody-like molecule comprises, consists essentially of, or consists of a binding member selected from the group consisting an Fab, Fab′, F(ab′)₂, Fv, and a single-chain antibody.

20. The antibody or antibody-like molecule of claim 17 conjugated to a therapeutic agent selected from the group consisting of an alkylating agent, an antimetabolite, a mitotic inhibitor, a taxoid, a vinca alkaloid, and an antibiotic.

21. The antibody or antibody-like molecule of claim 17, wherein the antibody or antibody-like molecule is a T cell receptor, optionally conjugated to a CD3 agonist.

22. An in vitro population of T cells transfected with a nucleic acid encoding a T cell receptor of claim 21.

23. A method for treating and/or preventing cancer comprising, consisting essentially of, or consisting of administering to a subject in need thereof a therapeutically effective dose of a composition of claim 1 or a composition comprising, consisting essentially of, or consisting of at least one target peptide comprising, consisting essentially of, or consisting of an amino acid sequence as set forth in Table 2.

24. The method of claim 23, wherein the cancer is selected from the group consisting of breast cancer, colorectal cancer, esophageal cancer, intrahepatic cholangiocarcinoma cancer, optionally bile ductcancer, kidney cancer, leukemia and/or lymphoma, melanoma, head and neck cancer, ovarian cancer, pancreatic cancer, a cancer associated with phosphatase inhibitor dysregulation, a cancer associated with partially-transformed pheripheral blood mononuclear cells (PBMCs), a cancer of the tonsils, lung cancer, cervical cancer, or any combination thereof.

25. The method of claim 24, wherein the cancer is breast cancer, and the at least one target peptide comprises, consists essentially of, or consists of an amino acid sequence that is selected from the group consisting of SEQ ID NOs: 33, 39, 53, 54, 57, 58, 80, 109, 124, 126, 127, 134, 144, 148, 157, 160, 164, 189, 201, 204, 208, 212, 219, 233, 240, 253, 267, 286, 287, 290, 297-299, 304, 372, 373, 383, 388, 389, 424, 439, 440, 450, 461, 473, 478, 479, 494, 496, 503, 504, 506, 515, 516, 523, 564, 567, 573, 575, 576, 578-580, 589, 664, 732, 739, 768, 777, 784, 824, 835, 836, 862, 864-866, 871, 884, 886, 890, 894, 896, 897, 924, 927, 929, 980, 986, 987, 1021, 1057, 1086-1088, 1098, 1122, 1123, 1128, 1130, 1134, 1180, 1188, 1190, 1213, 1221, 1226, 1230, 1231, 1252, 1269, 1273, 1274, 1278, 1285, 1286, 1292, 1309, 1312, 1315, 1352, 1360, 1378, 1385, 1393, 1418, 1420, 1421, 1423, 1432, 1437, 1438, 1447, 1448, and 1469, and any combination thereof.

26. The method of claim 24, wherein the cancer is colorectal cancer, and the at least one target peptide comprises, consists essentially of, or consists of an amino acid sequence that is selected from the group consisting of SEQ ID NOs: 8, 10, 15, 21, 25-27, 51, 57, 58, 72, 73, 85, 106, 116, 117, 142, 156, 190, 201, 233, 263, 268, 270, 286, 299, 394, 395, 402, 412, 450, 472, 483, 484, 489, 530, 551, 565, 567, 574, 575, 582, 583, 584, 629, 664, 680, 681, 724, 741, 768, 776, 823, 835, 836, 851, 866, 868, 910, 919, 923, 938, 941, 952, 991, 1062, 1109, 1143, 1144, 1146, 1148, 1164, 1176, 1186, 1188, 1189, 1192, 1195, 1198, 1209, 1237, 1238, 1239, 1277, 1287, 1288-1290, 1301, 1305, 1317, 1319, 1333, 1347, 1387, 1393, 1399, 1409, 1414, 1419, 1420-1424, 1442, 1452, and 1453, and any combination thereof.

27. The method of claim 24, wherein the cancer is esophageal cancer, and the at least one target peptide comprises, consists essentially of, or consists of an amino acid sequence that is selected from the group consisting of SEQ ID NOs: 145, 305, 416, 490, 742, 765, 952, and 1121, and any combination thereof.

28. The method of claim 24, wherein the cancer is intrahepatic cholangiocarcinoma, optionally a bile duct cancer, and the at least one target peptide comprises, consists essentially of, or consists of an amino acid sequence that is selected from the group consisting of SEQ ID NOs: 157, 158, 184, 217, 232, 250, 278, 316, 416, 431, 469, 471, 486, 488, 498, 532-536, 587-589, 654, 788, 946, 979, 985, 1044, 1052-1054, 1063, 1065, 1094, 1099, 1121, 1133, 1203, 1231, 1282, 1411, 1420, 1469, and 1471, and any combination thereof.

29. The method of claim 24, wherein the cancer is kidney cancer, and the at least one target peptide comprises, consists essentially of, or consists of an amino acid sequence that is selected from the group consisting of SEQ ID NOs: 472, 480, 521, 528-530, 726, 823, 836, 1337, and 1386, and any combination thereof.

30. The method of claim 24, wherein the cancer is leukemia and/or lymphoma, and the at least one target peptide comprises, consists essentially of, or consists of an amino acid sequence that is selected from the group consisting of SEQ ID NOs: 1-5, 7-9, 11, 13, 14, 17-20, 22, 23, 31, 38, 41-50, 52, 54-63, 67, 69, 70, 75, 79, 84, 88, 90, 91, 97, 104, 110, 118, 119, 121, 123, 128, 130, 132, 139, 142, 146-149, 152, 159, 161-165, 168, 170-173, 175, 183, 186, 190, 192, 195, 196, 203-206, 208, 210, 215, 222, 231, 232, 234, 237-239, 244, 245, 250, 251, 254, 257, 264, 265, 268, 269, 271, 273, 276, 278, 281-283, 288, 292, 295, 300-302, 305, 308, 310, 317, 319, 322, 325, 328, 333, 336, 340, 343, 347-350, 353, 355, 368-371, 373, 376, 377, 379-382, 384, 387, 391, 393, 396, 400, 405, 410, 413, 416, 419, 420, 427, 433, 434, 443, 444, 446, 447, 453, 454, 456, 462, 463, 465-468, 470, 472, 476, 477, 480-483, 485, 492, 495, 497, 501, 513, 514, 517, 519, 521, 522, 525, 528-530, 533, 534, 537, 548, 549, 552, 553, 558, 563, 564, 568-570, 581, 582, 585, 586, 589, 590, 592, 593, 595-599, 602-604, 611, 614, 623-625, 627, 628, 630, 631, 633, 636, 638, 639, 644, 645, 648-651, 663, 665, 667, 669, 670, 684, 693, 695, 699, 700, 717, 727, 729, 730, 731, 734-736, 739, 740, 744-753, 756, 758, 759, 762, 763, 766, 768, 769, 771, 773, 776, 777, 780-783, 785-787, 789-795, 797, 799-804, 807-817, 819, 821-827, 830, 832, 837, 838, 840, 843, 848, 851-853, 869, 870, 872-875, 878, 879, 885, 889, 894, 898, 901-903, 908, 909, 913-915, 918, 919, 921, 923, 924, 930, 931, 935, 937, 939, 940, 942, 944, 945, 961, 968, 971, 983, 994, 997, 1006, 1010, 1012, 1014, 1021, 1034, 1036, 1037, 1042, 1047, 1052, 1056, 1058, 1065, 1066, 1070, 1072, 1075, 1078, 1093, 1101, 1104, 1105, 1110-1112, 1118, 1119, 1124, 1125, 1132, 1133, 1135, 1145, 1151, 1157, 1158-1161, 1164-1166, 1168-1175, 1177, 1181, 1182, 1185, 1187, 1191, 1193-1196, 1199-1201, 1206-1209, 1211, 1212, 1214-1218, 1228, 1229, 1245, 1249, 1253, 1276, 1280, 1284, 1293, 1296, 1297, 1305, 1318, 1320-1322, 1324, 1326-1332, 1334, 1337, 1343-1345, 1348, 1350, 1351, 1353, 1354, 1362, 1364, 1369, 1370, 1375, 1377, 1378, 1380, 1384, 1386, 1391, 1393-1400, 1403, 1405, 1406, 1410, 1412, 1417, 1426, 1428, 1434-1436, 1440, 1446, 1450, 1451, 1464, 1466, and 1468, and any combination thereof.

31. The method of claim 24, wherein the cancer is melanoma, and the at least one target peptide comprises, consists essentially of, or consists of an amino acid sequence that is selected from the group consisting of SEQ ID NOs: 12, 21, 28, 34, 54, 65, 99, 156, 175, 205, 207, 238, 239, 268, 329, 337, 346, 350, 371, 373, 379, 380, 423, 448, 467, 487, 512, 530, 571, 577, 655, 688, 690, 700, 712, 728, 738, 741, 743, 746, 768, 772, 778, 779, 782, 785, 788-790, 795, 815, 820, 823, 910, 919, 933, 936, 949, 950, 981, 989, 1085, 1110, 1124, 1126, 1153, 1155, 1199, 1202, 1213, 1220, 1277, 1339, 1387, 1393, and 1448, and any combination thereof.

32. The method of claim 24, wherein the cancer is head and neck cancer, and the at last one target peptide comprises, consists essentially of, or consists of an amino acid sequence that is selected from the group consisting of SEQ ID NOs: 6, 58, 64, 65, 68, 73, 76, 77, 80, 82, 86, 124, 129, 148-150, 157, 164, 166, 175, 178, 179, 184, 185, 187, 194, 202, 213, 220, 221, 225, 226, 228-230, 232, 248, 268, 284-286, 289, 291, 294, 296, 318, 325, 371, 388, 392, 398, 399, 405-409, 411, 414-416, 429, 432, 438, 441, 442, 460, 499, 500, 502, 505, 507-511, 540-543, 566, 572, 585, 589, 591, 601, 609, 612, 620, 621, 622, 630, 637, 640, 641, 668, 683, 704, 725, 737, 815, 824, 839, 841, 845-848, 853, 854, 863, 866, 867, 875, 880-883, 887, 891, 893, 895, 899, 900, 905-907, 910-912, 916-918, 920-922, 924, 926, 928, 930, 932, 934, 943, 944, 948, 951, 953-960, 962-966, 969, 970, 972-978, 982, 984, 985, 988, 991, 995, 996, 998, 1001, 1003-1005, 1007, 1009, 1011, 1013, 1015-1020, 1022, 1023, 1026-1029, 1031, 1033, 1035, 1038, 1039, 1043, 1046, 1049-1051, 1055, 1059, 1060, 1063, 1064, 1068, 1081, 1082, 1095, 1099, 1116, 1137, 1144, 1146, 1151, 1184, 1204, 1205, 1209, 1213, 1219, 1221, 1234, 1246, 1256, 1257, 1262, 1271, 1293, 1300, 1307, 1315, 1316, 1325, 1340, 1389, 1407, 1408, 1411, 1413, 1420, 1429, 1430, 1431, 1455, 1456, 1461-1463, 1467, and 1469, and any combination thereof.

33. The method of claim 24, wherein the cancer is ovarian cancer, and the target peptide comprises, consists essentially of, or consists of an amino acid sequence that is selected from the group consisting of SEQ ID NOs: 29, 32, 40, 139, 193, 224, 261, 464, 666, 685, 686, 689-691, 700, 702, 708, 1040, 1052, 1069, 1139, 1149, 1260, 1265, 1346, 1371, 1379, 1387, and 1388, and any combination thereof.

34. The method of claim 24, wherein the cancer is pancreatic cancer, and the target peptide comprises, consists essentially of, or consists of an amino acid sequence that is selected from the group consisting of SEQ ID NOs: 65, 66, 216, 311-314, 417, 420, 435, 436, 653, 1076, 1077, 1120, 1131, 1261, and 1433, and any combination thereof.

35. The method of claim 24, wherein the cancer is a cancer associated with phosphatase inhibitor dysregulation, and the target peptide comprises, consists essentially of, or consists of an amino acid sequence that is selected from the group consisting of SEQ ID NOs: 30, 31, 35, 37, 74, 87, 111-115, 122, 135, 138-142, 149, 151, 161, 162, 174, 175, 182, 197, 224, 258, 259, 262, 266, 277, 278, 280, 282, 293, 295, 308, 309, 327, 330, 331, 334, 335, 341-348, 351, 352, 356-367, 385, 397, 403, 410, 421, 422, 427, 451, 474, 518, 539, 550, 553, 554, 558, 559, 600, 656-661, 668, 672-679, 682, 687, 692-694, 696, 698, 699, 703, 705-707, 710, 711, 713-715, 720-722, 824, 825, 831, 844-847, 861, 885, 942, 971, 1080, 1090, 1113, 1129, 1138, 1140-1142, 1150, 1152, 1154, 1156, 1158, 1215-1217, 1240-1244, 1275, 1283, 1299, 1310, 1313, 1314, 1342, 1355, 1358, 1361, 1363, 1366, 1372-1374, 1380-1383, 1386, 1390, 1401, 1404, 1415, 1416, 1434, 1439, 1441, 1443, 1444, 1457-1460, and 1469, and any combination thereof.

36. The method of claim 24, wherein the cancer is a cancer associated with partially-transformed peripheral blood mononuclear cells (PBMCs), and the target peptide comprises, consists essentially of, or consists of an amino acid sequence that is selected from the group consisting of SEQ ID NOs: 8, 23, 31, 36, 74, 83, 92-98, 101, 104, 113, 114, 126, 136, 142, 149, 151, 161, 162, 173-175, 190, 191, 222, 223, 226, 227, 235, 236, 246, 247, 250, 272, 274, 278, 295, 333, 338, 339, 343, 410, 416, 425, 430, 437, 452, 455, 457-459, 474, 490, 519, 520, 526, 527, 533, 534, 537-539, 544, 545, 553-557, 560-562, 594, 602, 607, 608, 654, 699, 718, 815, 824, 825, 828, 829, 844-847, 885, 942, 947, 971, 999, 1011, 1012, 1032, 1074, 1095, 1097, 1115, 1142, 1158, 1159, 1197, 1210, 1217, 1223-1225, 1227, 1229, 1235, 1272, 1279, 1286, 1297, 1298, 1308, 1319, 1334, 1339, 1340, 1341, 1355, 1356, 1361, 1365, 1366, 1368, 1380, 1381, 1383, 1402, 1404, 1434, 1435, 1445, 1449, 1459, 1460, and 1469, and any combination thereof.

37. The method of claim 24, wherein the cancer is a cancer of the tonsils, and the target peptide comprises, consists essentially of, or consists of SEQ ID NO: 768.

38. The method of claim 24, wherein the cancer is lung cancer, and the target peptide comprises, consists essentially of, or consists of an amino acid sequence that is selected from the group consisting of SEQ ID NOs: 119, 251, 256, 428, 470, 549, and 952, and any combination thereof.

39. The method of claim 24, wherein the cancer is cervical cancer, and the target peptide comprises, consists essentially of, or consists of an amino acid sequence that is selected from the group consisting of SEQ ID NOs: 108, 255, 779, 1067, and 1323, and any combination thereof.

40. A method of treating and/or preventing a cancer comprising, consisting essentially of, or consisting of administering to a subject in need thereof a therapeutically effective dose of a composition of claim 1 or a composition comprising, consisting essentially of, or consisting of at least one target peptide in combination with a pharmaceutically acceptable carrier.

41. A method for treating and/or preventing cancer comprising, consisting essentially of, or consisting of administering to a subject in need thereof a therapeutically effective dose of the CD8⁺ T cells of claim 16 in combination with a pharmaceutically acceptable carrier.

42. A method for treating and/or preventing cancer comprising, consisting essentially of, or consisting of administering to a subject in need thereof an in vitro population of dendritic cells of claim 15 in combination with a pharmaceutically acceptable carrier.

43. A method for treating and/or preventing cancer comprising, consisting essentially of, or consisting of administering to a subject in need thereof the population of CD8⁺ T cells of claim 16 in combination with a pharmaceutically acceptable carrier.

44. A method for making a cancer vaccine comprising, consisting essentially of, or consisting of combining the composition of claim 1 with an the adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

45. A method for screening target peptides for inclusion in an immunotherapy composition of claim 1 or for use in the method of using a composition of claim 1, comprising, consisting essentially of, or consisting of:
(a) administering the target peptide to a human;
(b) determining whether the target peptide is capable of inducing a target peptide-specific memory T cell response in the human; and
(c) selecting the target peptide for inclusion in an immunotherapy composition if the target peptide elicits a memory T cell response in the human.

46. A method for determining a prognosis of a cancer patient, the method comprising, consisting essentially of, or consisting of:
(a) administering to the patient a target peptide comprising, consisting essentially of, or consisting of an amino acid sequence as set forth in Table 2 and/or Table 3, wherein the target peptide is associated with the patient's cancer;
(b) determining whether the target peptide is capable of inducing a target peptide-specific memory T cell response in the patient; and
(c) determining that the patient has a better prognosis if the patient mounts a memory T cell response to the target peptide than if the patient did not mount a memory T cell response to the target peptide.

47. A kit comprising, consisting essentially of, or consisting of at least one target peptide composition comprising, consisting essentially of, or consisting of at least one target peptide comprising, consisting essentially of, or consisting of an amino acid sequence as set forth in Table 2 and/or Table 3 and a cytokine and/or an adjuvant.

48. The kit of claim 47, comprising, consisting essentially of, or consisting of at least 2, 3, 4, or 5 target peptide compositions.

49. The kit of claim 47, wherein the at least one target peptide composition is one of the compositions of claim 1.

50. The kit of claim 47, wherein the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF).

51. The kit of claim 47, wherein the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT).

52. The kit of claim 47, wherein the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT).

53. The kit of claim 46, further comprising at least one peptide derived from MelanA (MART-1), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS.

54. The kit of claim 46, wherein the at least one target peptide comprises, consists essentially of, or consists of an amino acid sequence as set forth in Table 2.

55. The composition of claim 1, comprising, consisting essentially of, or consisting of a peptide capable of binding to an MHC class I molecule, optionally an MHC class I molecule selected from the group consisting of an HLA A*0101 allele, an HLA-A*0201 allele, an HLA B*2705 allele, an HLA A*0301 allele, an HLA B*0702 allele, and an HLA B*4402 allele.

56. A composition comprising:
(a) at least one synthetic target peptide, wherein the at least one synthetic target peptide:
(i) is from 8 to 50 amino acids long; and
(ii) comprises an amino acid sequence selected from the group consisting of:
SEQ ID NO: 150, wherein the serine at the fourth position is phosphorylated or replaced with a mimetic of phosphoserine;
SEQ ID NO: 195, wherein the tyrosine at the sixth position is phosphorylated or replaced with a mimetic of phosphotyrosine;
SEQ ID NO: 196, wherein the tyrosine at the sixth position is phosphorylated or replaced with a mimetic of phosphotyrosine;
SEQ ID NO: 234, wherein the serine at the fourth position is phosphorylated or replaced with a mimetic of phosphoserine;
SEQ ID NO: 257, wherein the serine at the seventh position is phosphorylated or replaced with a mimetic of phosphoserine;
SEQ ID NO: 293, wherein the tyrosine at the fourth position is phosphorylated or replaced with a mimetic of phosphotyrosine;
SEQ ID NO: 331, wherein the serine at the fourth position is phosphorylated or replaced with a mimetic of phosphoserine;
SEQ ID NO: 369, wherein the serine at the fourth position is phosphorylated or replaced with a mimetic of phosphoserine;
SEQ ID NO: 381, wherein the serine at the eighth position is phosphorylated or replaced with a mimetic of phosphoserine;
SEQ ID NO: 408, wherein the serine at the fourth position is phosphorylated or replaced with a mimetic of phosphoserine;
SEQ ID NO: 409, wherein the serine at the fourth position is phosphorylated or replaced with a mimetic of phosphoserine;
SEQ ID NO: 432, wherein the serine at the fourth position is phosphorylated or replaced with a mimetic of phosphoserine, and/or wherein the methionine at the third position, the methionine at the eighth position, or both are oxidized, or any combination thereof;
SEQ ID NO: 481, wherein the serine at the fourth position is phosphorylated or replaced with a mimetic of phosphoserine;
SEQ ID NO: 601, wherein the serine at the fourth position is phosphorylated or replaced with a mimetic of phosphoserine;
SEQ ID NO: 726, wherein the serine at the fifth position is phosphorylated or replaced with a mimetic of phosphoserine, and/or wherein the methionine at the seventh position is oxidized, or a combination thereof;
SEQ ID NO: 729, wherein the serine at the fifth position is phosphorylated or replaced with a mimetic of phosphoserine;
SEQ ID NO: 752, wherein the serine at the eighth position is phosphorylated or replaced with a mimetic of phosphoserine;
SEQ ID NO: 792, wherein the serine at the fourth position is phosphorylated or replaced with a mimetic of phosphoserine;
SEQ ID NO: 912, wherein the serine at the fourth position is phosphorylated or replaced with a mimetic of phosphoserine;
SEQ ID NO: 973, wherein the serine at the fourth position is phosphorylated or replaced with a mimetic of phosphoserine, and/or wherein the methionine at the sixth position is oxidized, or a combination thereof;
SEQ ID NO: 1128, wherein the serine at the fourth position is phosphorylated or replaced with a mimetic of phosphoserine; and
SEQ ID NO: 1269, wherein one or more of the serines at the fourth, fifth, and/or seventh positions is phosphorylated and/or replaced with a mimetic of phosphoserine, or any combination thereof; and
(b) an adjuvant.

57. A composition comprising:
(a) at least one synthetic target peptide, wherein the at least one synthetic target peptide:
(i) is from 8 to 50 amino acids long; and
(ii) comprises an amino acid sequence selected from the group consisting of SEQ ID NOs: 25-27, 33, 34, 38, 39, 41, 42, 65, 66, 77, 82, 112, 131, 140, 141, 174, 190, 193, 201, 227, 242, 246, 247, 286, 294, 301, 336, 338, 348, 355-362, 364-367, 375, 384, 390, 393, 398, 399, 414, 417, 418, 422, 429, 431, 433, 436, 449, 454, 474, 479, 483, 502, 508, 514, 517, 518, 520, 521, 524-526, 528, 530, 531, 533, 538-540, 542, 543, 545, 552-555, 559, 569, 573, 592, 596-599, 604, 607, 619, 658-661, 668, 671, 672, 705, 707, 722-724, 727, 740, 765, 771, 773, 787, 797, 824, 825, 828, 831, 836, 851, 852, 854, 867, 884, 892, 894-896, 907-910, 915, 922, 929, 932, 940, 944, 950, 962, 973, 987, 998, 1019, 1020, 1022, 1045, 1050, 1054, 1066, 1096, 1101, 1103, 1108, 1117, 1130, 1140, 1142, 1144, 1153, 1154, 1158, 1160, 1224, 1234-1236, 1258, 1260, 1277, 1298, 1311, 1314, 1315, 1321, 1322, 1336, 1340, 1372, 1378, 1380, 1382, 1383, 13861419, 1458, and 1460, wherein at least one methionine is oxidized; and
(b) an adjuvant.

58. A composition comprising:
(a) at least one synthetic target peptide, wherein the at least one synthetic target peptide:
(i) is from 8 to 50 amino acids long; and
(ii) comprises an amino acid sequence selected from the group consisting of:
SEQ ID NO: 100, wherein the serine at the second position, the serine at the third position, or both are phosphorylated or replaced with a mimetic of phosphoserine;
SEQ ID NO: 102, wherein the serine at the second position, the serine at the third position, or both are phosphorylated or replaced with a mimetic of phosphoserine;
SEQ ID NO: 103, wherein the serine at the second position, the serine at the third position, or both are phosphorylated or replaced with a mimetic of phosphoserine;
SEQ ID NO: 117, wherein the tryotophan at the seventh position is oxidized;
SEQ ID NO: 125, wherein the serine at the ninth position is phosphorylated or replaced with a mimetic of phosphoserine and the threonine at the seventh position is unmodified;
SEQ ID NO: 207, wherein the serine at the fourth position is phosphorylated or replaced with a mimetic of phosphoserine and the serine at the ninth position is unmodified;
SEQ ID NO: 209, wherein the serine at the fourth position is phosphorylated or replaced with a mimetic of phosphoserine, and optionally wherein the serine at the third position is also phosphorylated or replaced with a mimetic of phosphoserine;
SEQ ID NO: 233, wherein the serine at the fifth position is phosphorylated or replaced with a mimetic of phosphoserine, and optionally wherein the threonine at the fourth position is also phosphorylated or replaced with a mimetic of phosphothreonine;
SEQ ID NO: 245, wherein the threonine at the fifth position is phosphorylated or replaced with a mimetic of phosphothreonine, and optionally wherein the serine at the fourth position is also phosphorylated or replaced with a mimetic of phosphoserine;
SEQ ID NO: 287, wherein:
the serine at the fourth position is phosphorylated or replaced with a mimetic of phosphoserine and the serine at the second position is phosphorylated or replaced with a mimetic of phosphoserine and/or the threonine at the third position is phosphorylated or replaced with a mimetic of phosphothreonine; or
the serine at the second position is phosphorylated or replaced with a mimetic of phosphoserine and the threonine at the third position is phosphorylated or replaced with a mimetic of phosphothreonine;
SEQ ID NO: 304, wherein the serine at the eighth position is phosphorylated or replaced with a mimetic of phosphoserine, and optionally wherein the serine at the fourth position is also phosphorylated or replaced with a mimetic of phosphoserine;
SEQ ID NO: 309, wherein the serine at the eighth position is phosphorylated or replaced with a mimetic of phosphoserine, and optionally wherein the threonine at the fourth position is also phosphorylated or replaced with a mimetic of phosphothreonine;
SEQ ID NO: 312 or 314, wherein the serine at the eighth position is phosphorylated or replaced with a mimetic of phosphoserine, and optionally wherein the serine at the fourth position is also phosphorylated or replaced with a mimetic of phosphoserine;
SEQ ID NO: 323, wherein the serine at the sixth position is phosphorylated or replaced with a mimetic of phosphoserine, the threonine at the seventh position is phosphorylated or replaced with a mimetic of phosphothreonine, the serine at the twelfth position is phosphorylated or replaced with a mimetic of phosphoserine, or a combination thereof, provided that if the serine at the sixth position is phosphorylated or replaced with a mimetic of phosphoserine, at least one of the threonine at the seventh position and the serine at the twelfth position is also modified;
SEQ ID NO: 405, wherein the serine at the fourth position is phosphorylated or replaced with a mimetic of phosphoserine, and optionally wherein the serine at the third position is also phosphorylated or replaced with a mimetic of phosphoserine;
SEQ ID NO: 419, wherein the serine at the second position is phosphorylated or replaced with a mimetic of phosphoserine, the serine at the third position is phosphorylated or replaced with a mimetic of phosphoserine, the serine at the fourth position is phosphorylated or replaced with a mimetic of phosphoserine, or any combination thereof, provided that if the serine at the second position is phosphorylated or replaced with a mimetic of phosphoserine, at least one of the serines at the third and fourth positions is also modified;
SEQ ID NO: 439, wherein the serine at the sixth position is phosphorylated or replaced with a mimetic of phosphoserine, and optionally wherein the serine at the fourth position is also phosphorylated or replaced with a mimetic of phosphoserine;
SEQ ID NO: 445, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 447, wherein the serine at the fifth position is phosphorylated or replaced with a mimetic of phosphoserine and/or the serine in the sixth position is phosphorylated or replaced with a mimetic of phosphoserine, and optionally wherein the serine at the fourth position is also phosphorylated or replaced with a mimetic of phosphoserine;
SEQ ID NO: 457, wherein the serine at the third position is phosphorylated or replaced with a mimetic of phosphoserine, the threonine at the fifth position is phosphorylated or replaced with a mimetic of phosphothreonine, the threonine at the sixth position is phosphorylated or replaced with a mimetic of phosphothreonine, or any combination thereof, provided that if the threonine at the sixth position is phosphorylated or replaced with a mimetic of phosphothreonine, at least one of the amino acids at the third and fifth positions is also modified;
SEQ ID NO: 476, wherein the serine at the fourth position is phosphorylated or replaced with a mimetic of phosphoserine, the serine in the fifth position is phosphorylated or replaced with a mimetic of phosphoserine, SEQ ID NO: 447, wherein the serine at the sixth position is phosphorylated or replaced with a mimetic of phosphoserine, or any combination thereof, provided that if the serine at the fourth is phosphorylated or replaced with a mimetic of phosphoserine, at least one of the serines at the fifth and sixth positions is also modified;
SEQ ID NO: 484, wherein the serine at the sixth position is phosphorylated or replaced with a mimetic of phosphoserine, and optionally wherein the threonine at the fourth position is also phosphorylated or replaced with a mimetic of phosphothreonine;
SEQ ID NO: 536, wherein the serine at the third position is phosphorylated or replaced with a mimetic of phosphoserine, and optionally wherein the serine at the sixth position is also phosphorylated or replaced with a mimetic of phosphoserine;
SEQ ID NO: 562, wherein the serine at the seventh position is phosphorylated or replaced with a mimetic of phosphoserine, and optionally wherein the threonine at the sixth position is also phosphorylated or replaced with a mimetic of phosphothreonine;
SEQ ID NO: 574, wherein at least one of the serines at the sixth and seventh positions are phosphorylated or replaced with a mimetic of phosphoserine, and further wherein the tryptophan at the second position is oxidized;
SEQ ID NO: 584, wherein the glutamine at position 1 is modified to a pyroglutamic acid, and optionally wherein one or more of the threonine at the fourth position and the serines at the seventh position is phosphorylated or replaced with a mimetic of phosphoserine or phosphothreonine;
SEQ ID NO: 614, wherein the serine at the fourth position is phosphorylated or replaced with a mimetic of phosphoserine, and optionally wherein the serine at the third position is also phosphorylated or replaced with a mimetic of phosphoserine;
SEQ ID NO: 642, wherein the threonine at the third position is phosphorylated or replaced with a mimetic of phosphothreonine and the serine at the fourth position is phosphorylated or replaced with a mimetic of phosphoserine;
SEQ ID NO: 663, wherein the threonine at the sixth position is phosphorylated or replaced with a mimetic of phosphothreonine, and optionally wherein the serine at the fifth position is also phosphorylated or replaced with a mimetic of phosphoserine;
SEQ ID NO: 718, wherein the serine at the fourth position is phosphorylated or replaced with a mimetic of phosphoserine, and optionally wherein the serine at the third position is also phosphorylated or replaced with a mimetic of phosphoserine;
SEQ ID NO: 733, wherein the serine at the fourth position is phosphorylated or replaced with a mimetic of phosphoserine, and optionally wherein the threonine at the fifth position is also phosphorylated or replaced with a mimetic of phosphothreonine;
SEQ ID NO: 745, wherein the serine at the fifth position is phosphorylated or replaced with a mimetic of phosphoserine, the serine at the sixth position is phosphorylated or replaced with a mimetic of phosphoserine, the serine at the seventh position is phosphorylated or replaced with a mimetic of phosphoserine, or a combination thereof, provided that if the serine at the fifth position is phosphorylated or replaced with a mimetic of phosphoserine, at least one of the serine at the sixth position and the serine at the seventh position is also modified;
SEQ ID NO: 762, wherein the serine at the fourth position is phosphorylated or replaced with a mimetic of phosphoserine, the serine at the fifth position is phosphorylated or replaced with a mimetic of phosphoserine, the serine at the sixth position is phosphorylated or replaced with a mimetic of phosphoserine, or a combination thereof, provided that if the serine at the fourth position is phosphorylated or replaced with a mimetic of phosphoserine, at least one of the serine at the fifth position and the serine at the sixth position is also modified;
SEQ ID NO: 767, wherein the serine at the fifth position is phosphorylated or replaced with a mimetic of phosphoserine, and optionally wherein the threonine at the fourth position is also phosphorylated or replaced with a mimetic of phosphothreonine;
SEQ ID NO: 768, wherein the serine at the seventh position is phosphorylated or replaced with a mimetic of phosphoserine, and optionally wherein the threonine at the sixth position is also phosphorylated or replaced with a mimetic of phosphothreonine;
SEQ ID NO: 774, wherein the serine at the sixth position is phosphorylated or replaced with a mimetic of phosphoserine, and optionally wherein the serine at the fifth position is also phosphorylated or replaced with a mimetic of phosphoserine;
SEQ ID NO: 775, wherein the threonine at the seventh position is phosphorylated or replaced with a mimetic of phosphothreonine, and optionally wherein the serine at the sixth position is also phosphorylated or replaced with a mimetic of phosphoserine;
SEQ ID NO: 776, wherein the serine at the sixth position is phosphorylated or replaced with a mimetic of phosphoserine, and optionally wherein the serine at the fourth position is also phosphorylated or replaced with a mimetic of phosphoserine;
SEQ ID NO: 778, wherein the serine at the seventh position is phosphorylated or replaced with a mimetic of phosphoserine, and optionally wherein the serine at the sixth position is also phosphorylated or replaced with a mimetic of phosphoserine;
SEQ ID NO: 779, wherein the serine at the seventh position is phosphorylated or replaced with a mimetic of phosphoserine, and optionally wherein the serine at the sixth position is also phosphorylated or replaced with a mimetic of phosphoserine;
SEQ ID NO: 782, wherein the serine at the sixth position is phosphorylated or replaced with a mimetic of phosphoserine, and optionally wherein the serine at the fourth position is also phosphorylated or replaced with a mimetic of phosphoserine;
SEQ ID NO: 784, wherein the threonine at the fifth position is phosphorylated or replaced with a mimetic of phosphothreonine and/or the serine at the sixth position is phosphorylated or replaced with a mimetic of phosphoserine, and optionally wherein the serine at the fourth position is also phosphorylated or replaced with a mimetic of phosphoserine;
SEQ ID NO: 787, wherein the serine at the fourth position is phosphorylated or replaced with a mimetic of phosphoserine, and optionally wherein the serine at the third position is also phosphorylated or replaced with a mimetic of phosphoserine, and further optionally wherein the methionine at the ninth position is oxidized;
SEQ ID NO: 788, wherein the serines at both the third and fourth positions are both independently phosphorylated or replaced with a mimetic of phosphoserine;
SEQ ID NO: 789, wherein the serines at both the third and fourth positions are both independently phosphorylated or replaced with a mimetic of phosphoserine;
SEQ ID NO: 790, wherein the serine at the fourth position is phosphorylated or replaced with a mimetic of phosphoserine, and optionally wherein the serine at the third position is also phosphorylated or replaced with a mimetic of phosphoserine;
SEQ ID NO: 791, wherein the serine at the fourth position is phosphorylated or replaced with a mimetic of phosphoserine, and optionally wherein the serine at the third position is also phosphorylated or replaced with a mimetic of phosphoserine;
SEQ ID NO: 802, wherein the threonine at the fourth position is phosphorylated or replaced with a mimetic of phosphothreonine, and optionally wherein the serine at the third position is also phosphorylated or replaced with a mimetic of phosphoserine;
SEQ ID NO: 803, wherein the serine at the third position is phosphorylated or replaced with a mimetic of phosphoserine, and optionally wherein the threonine at the fourth position is also phosphorylated or replaced with a mimetic of phosphothreonine;
SEQ ID NO: 804, wherein the serine at the third position is phosphorylated or replaced with a mimetic of phosphoserine, and optionally wherein the threonine at the fourth position is also phosphorylated or replaced with a mimetic of phosphothreonine;
SEQ ID NO: 806, wherein the threonine at the third position is phosphorylated or replaced with a mimetic of phosphothreonine and the serine at the fourth position is also phosphorylated or replaced with a mimetic of phosphoserine;
SEQ ID NO: 855, wherein the threonine at the fifth position is phosphorylated or replaced with a mimetic of phosphothreonine, and optionally wherein the serine at the fourth position is also phosphorylated or replaced with a mimetic of phosphoserine;
SEQ ID NO: 923, wherein the serines at both the third and fourth positions are both independently phosphorylated or replaced with a mimetic of phosphoserine;
SEQ ID NO: 924, wherein the serine at the fourth position is phosphorylated or replaced with a mimetic of phosphoserine, and optionally wherein the serine at the third position is also phosphorylated or replaced with a mimetic of phosphoserine;
SEQ ID NO: 969, wherein the serine at the tenth position is phosphorylated or replaced with a mimetic of phosphoserine, and optionally wherein the serine at the fourth position is also phosphorylated or replaced with a mimetic of phosphoserine;
SEQ ID NO: 976, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1009, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine;
SEQ ID NO: 1011, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine;
SEQ ID NO: 1012, wherein one, two, or all three of the serines at the second, third, and fourth positions are phosphorylated or replaced with a mimetic of phosphoserine, provided that if the serine at the second position is phosphorylated or replaced with a mimetic of phosphoserine, the serine at the third position and/or the serine at the fourth position is also phosphorylated or replaced with a mimetic of phosphoserine;
SEQ ID NO: 1061, wherein the serines at both the third and fourth positions are both independently phosphorylated or replaced with a mimetic of phosphoserine;
SEQ ID NO: 1076, wherein one but not both of the serines at the fourth and sixth positions is phosphorylated or replaced with a mimetic of phosphoserine;
SEQ ID NO: 1079, wherein the serine at the sixth position is phosphorylated or replaced with a mimetic of phosphoserine, and optionally wherein the serine at the fifth position is also phosphorylated or replaced with a mimetic of phosphoserine;
SEQ ID NO: 1084, wherein the serines at both the ninth and tenth positions are both independently phosphorylated or replaced with a mimetic of phosphoserine;
SEQ ID NO: 1111, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1145, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1157, wherein the serine at the fourth position is phosphorylated or replaced with a mimetic of phosphoserine, and optionally wherein the serine at the third position is also phosphorylated or replaced with a mimetic of phosphoserine;
SEQ ID NO: 1163, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1165, wherein the serine at one or both of the fifth and sixth positions are independently phosphorylated or replaced with a mimetic of phosphoserine, and optionally wherein the serine at the ninth position is also phosphorylated or replaced with a mimetic of phosphoserine;
SEQ ID NO: 1197, wherein the threonine at the sixth position is phosphorylated or replaced with a mimetic of phosphothreonine, and optionally wherein the serine at the fourth position is also phosphorylated or replaced with a mimetic of phosphoserine;
SEQ ID NO: 1202, wherein the threonine at the fourth position is phosphorylated or replaced with a mimetic of phosphothreonine, and optionally wherein the serine at the seventh position is also phosphorylated or replaced with a mimetic of phosphoserine;
SEQ ID NO: 1216, wherein the serine at the eighth position is phosphorylated or replaced with a mimetic of phosphoserine, and optionally wherein the serine at the fourth position is also phosphorylated or replaced with a mimetic of phosphoserine;
SEQ ID NO: 1220, wherein one, two, or all three of the serines at the fourth, fifth, and sixth positions are phosphorylated or replaced with a mimetic of phosphoserine, provided that if the serine at the fourth position is phosphorylated or replaced with a mimetic of phosphoserine, the serine at the fifth position and/or the serine at the sixth position is also phosphorylated or replaced with a mimetic of phosphoserine;
SEQ ID NO: 1221 or 1222, wherein one, two, three, or all four of the serines at the third, fourth, fifth, and eighth positions are phosphorylated or replaced with a mimetic of phosphoserine, provided that if the serine at the eighth position is phosphorylated or replaced with a mimetic of phosphoserine, at least one of the serines at the third, fourth, and fifth positions are also phosphorylated or replaced with a mimetic of phosphoserine, and further provided that if the serines at the third and eighth positions are phosphorylated or replaced with a mimetic of phosphoserine, at least one of the serines at the fourth and fifth positions are also phosphorylated or replaced with a mimetic of phosphoserine;
SEQ ID NO: 1243, wherein the serine at the twelfth position is phosphorylated or replaced with a mimetic of phosphoserine, and optionally wherein the serine at the second position is also phosphorylated or replaced with a mimetic of phosphoserine;
SEQ ID NO: 1251, wherein the serine at the ninth position is phosphorylated or replaced with a mimetic of phosphoserine, and optionally wherein the serine at the fourth position is also phosphorylated or replaced with a mimetic of phosphoserine;
SEQ ID NO: 1256, wherein the threonine at the fifth position is phosphorylated or replaced with a mimetic of phosphothreonine, and optionally wherein the serine at the fourth position is also phosphorylated or replaced with a mimetic of phosphoserine;
SEQ ID NO: 1279, wherein the threonine at the sixth position is phosphorylated or replaced with a mimetic of phosphothreonine, and optionally wherein the serine at the fourth position is also phosphorylated or replaced with a mimetic of phosphoserine;
SEQ ID NO: 1281, wherein the serines at both the third and fourth positions are both independently phosphorylated or replaced with a mimetic of phosphoserine;
SEQ ID NO: 1287 or 1288, wherein one, two, three, or all four of the serines at the fifth, sixth, seventh, and eighth positions are phosphorylated or replaced with a mimetic of phosphoserine, provided that if the serine at the fifth position or at the eighth position is phosphorylated or replaced with a mimetic of phosphoserine, at least one of the serines at the sixth and seventh positions is also phosphorylated or replaced with a mimetic of phosphoserine;
SEQ ID NO: 1289 or 1290, wherein one, two, three, or all four of the serines at the seventh, eighth, ninth, and tenth positions are phosphorylated or replaced with a mimetic of phosphoserine, provided that if the serine at the seventh position or at the tenth position is phosphorylated or replaced with a mimetic of phosphoserine, at least one of the serines at the eighth and ninth positions is also phosphorylated or replaced with a mimetic of phosphoserine;
SEQ ID NO: 1303, wherein the threonine at the first position is phosphorylated or replaced with a mimetic of phosphothreonine, and optionally wherein the serine at the third position is also phosphorylated or replaced with a mimetic of phosphoserine;
SEQ ID NO: 1310, wherein one or both of the threonines at the fifth and sixth positions are phosphorylated or replaced with a mimetic of phosphothreonine, and optionally wherein the serine at fourth position is also phosphorylated or replaced with a mimetic of phosphoserine;
SEQ ID NO: 1325, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1329 or 1330, wherein the threonine at the seventh position is phosphorylated or replaced with a mimetic of phosphothreonine, and optionally wherein the threonine at the fourth position is also phosphorylated or replaced with a mimetic of phosphothreonine;
SEQ ID NO: 1340 or 1341, wherein the threonine at the fifth position is phosphorylated or replaced with a mimetic of phosphothreonine, and optionally wherein the serine at the fourth position is phosphorylated or replaced with a mimetic of phosphoserine, the methionine at the third position is oxidized, or both;
SEQ ID NO: 1359, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1368, wherein the tryptophan at the ninth position is oxidized, and optionally wherein the serine at the fourth position is phosphorylated or replaced with a mimetic of phosphoserine;
SEQ ID NO: 1369 or 1370, wherein the serine at the ninth position is phosphorylated or replaced with a mimetic of phosphoserine, and optionally wherein the serine at the fourth position is also phosphorylated or replaced with a mimetic of phosphoserine;
SEQ ID NO: 1379 or 1380, wherein the serine at the fourth position is phosphorylated or replaced with a mimetic of phosphoserine, and optionally wherein the serine at the fifth position is also phosphorylated or replaced with a mimetic of phosphoserine, and further optionally wherein the methionine at the ninth position is oxidized;
SEQ ID NO: 1392, wherein one, two, or all three of the serines at the seventh, eighth, and ninth positions are phosphorylated or replaced with a mimetic of phosphoserine, provided that if the serine at the seventh position is phosphorylated or replaced with a mimetic of phosphoserine, the serine at the eighth position and/or the serine at the ninth position is also phosphorylated or replaced with a mimetic of phosphoserine;
SEQ ID NO: 1412, wherein the serine at the fifth position is phosphorylated or replaced with a mimetic of phosphoserine, and optionally wherein the serine at the fourth position is also phosphorylated or replaced with a mimetic of phosphoserine;
SEQ ID NO: 1421, 1422, 1423, or 1424, wherein the serine at the eighth position is phosphorylated or replaced with a mimetic of phosphoserine, and optionally wherein the threonine at the seventh position is phosphorylated or replaced with a mimetic of phosphothreonine;
SEQ ID NO: 1429, wherein the serine at the fourth position is phosphorylated or replaced with a mimetic of phosphoserine, and optionally wherein the serine at the third position is also phosphorylated or replaced with a mimetic of phosphoserine;
SEQ ID NO: 1430 or 1431, wherein the serine at the fourth position is phosphorylated or replaced with a mimetic of phosphoserine, and optionally wherein the serine at the third position is also phosphorylated or replaced with a mimetic of phosphoserine;
SEQ ID NO: 1440, wherein the serine at the eighth position is phosphorylated or replaced with a mimetic of phosphoserine, and optionally wherein the serine at the fourth position is also phosphorylated or replaced with a mimetic of phosphoserine;
SEQ ID NO: 1448, wherein the serine at the eighth position is phosphorylated or replaced with a mimetic of phosphoserine, and optionally wherein the serine at the seventh position is also phosphorylated or replaced with a mimetic of phosphoserine; and
SEQ ID NO: 1467, wherein the serine at the fourth position is phosphorylated or replaced with a mimetic of phosphoserine, and optionally wherein the serine at the third position is also phosphorylated or replaced with a mimetic of phosphoserine; and
(b) an adjuvant.

59. The composition of claim 56, wherein the at least one synthetic target peptide comprises a substitution of a serine residue with a homo-serine residue.

60. The composition of claim 56, wherein the at least one synthetic target peptide is a phosphopeptide that comprises a non-hydrolyzable phosphate group.

61. The composition of claim 56, wherein the at least one synthetic target peptide is capable of binding to an MHC class I molecule of the HLA-A*0201 allele.

62. The composition of claim 56, further comprising at least one peptide derived from MelanA (MART-1), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

63. The composition of claim 56, wherein the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, incomplete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.


SEQUENCE LISTING (1521 sequences)

SEQ ID NO: 1 - protein, 9 aa, Homo sapiens
  (1)..(9): One or more of the listed amino acids can be modified as set forth in the Tables
AAASPLHML

SEQ ID NO: 2 - protein, 10 aa, Homo sapiens
  (1)..(10): One or more of the listed amino acids can be modified as set forth in the Tables
AADGTPKHSF

SEQ ID NO: 3 - protein, 9 aa, Homo sapiens
  (1)..(9): One or more of the listed amino acids can be modified as set forth in the Tables
AADSPSQNL

SEQ ID NO: 4 - protein, 10 aa, Homo sapiens
  (1)..(10): One or more of the listed amino acids can be modified as set forth in the Tables
AADSPSQNLT

SEQ ID NO: 5 - protein, 10 aa, Homo sapiens
  (1)..(10): One or more of the listed amino acids can be modified as set forth in the Tables
AADTPPLETL

SEQ ID NO: 6 - protein, 11 aa, Homo sapiens
  (1)..(11): One or more of the listed amino acids can be modified as set forth in the Tables
AASDTERDGLA

SEQ ID NO: 7 - protein, 9 aa, Homo sapiens
  (1)..(9): One or more of the listed amino acids can be modified as set forth in the Tables
AASPGAPQM

SEQ ID NO: 8 - protein, 16 aa, Homo sapiens
  (1)..(16): One or more of the listed amino acids can be modified as set forth in the Tables
ADSGEGDFLAEGGGVR

SEQ ID NO: 9 - protein, 10 aa, Homo sapiens
  (1)..(10): One or more of the listed amino acids can be modified as set forth in the Tables
AEAPLPSPKL

SEQ ID NO: 10 - protein, 9 aa, Homo sapiens
  (1)..(9): One or more of the listed amino acids can be modified as set forth in the Tables
AEAPPSKSP

SEQ ID NO: 11 - protein, 12 aa, Homo sapiens
  (1)..(12): One or more of the listed amino acids can be modified as set forth in the Tables
AEFPSSGSNSVL

SEQ ID NO: 12 - protein, 10 aa, Homo sapiens
  (1)..(10): One or more of the listed amino acids can be modified as set forth in the Tables
AEGSPPPKTY

SEQ ID NO: 13 - protein, 9 aa, Homo sapiens
  (1)..(9): One or more of the listed amino acids can be modified as set forth in the Tables
AEISPGSLP

SEQ ID NO: 14 - protein, 12 aa, Homo sapiens
  (1)..(12): One or more of the listed amino acids can be modified as set forth in the Tables
AEISPGSLPVTA

SEQ ID NO: 15 - protein, 9 aa, Homo sapiens
  (1)..(9): One or more of the listed amino acids can be modified as set forth in the Tables
AEKSYQNSP

SEQ ID NO: 16 - protein, 9 aa, Homo sapiens
  (1)..(9): One or more of the listed amino acids can be modified as set forth in the Tables
AELSPKNLL

SEQ ID NO: 17 - protein, 9 aa, Homo sapiens
  (1)..(9): One or more of the listed amino acids can be modified as set forth in the Tables
AELSPSMAP

SEQ ID NO: 18 - protein, 10 aa, Homo sapiens
  (1)..(10): One or more of the listed amino acids can be modified as set forth in the Tables
AELSPTTLSP

SEQ ID NO: 19 - protein, 10 aa, Homo sapiens
  (1)..(10): One or more of the listed amino acids can be modified as set forth in the Tables
AELSPVEQKL

SEQ ID NO: 20 - protein, 9 aa, Homo sapiens
  (1)..(9): One or more of the listed amino acids can be modified as set forth in the Tables
AEMPTQMSP

SEQ ID NO: 21 - protein, 11 aa, Homo sapiens
  (1)..(11): One or more of the listed amino acids can be modified as set forth in the Tables
AEPTPEKEKRF

SEQ ID NO: 22 - protein, 10 aa, Homo sapiens
  (1)..(10): One or more of the listed amino acids can be modified as set forth in the Tables
AERTPELVEL

SEQ ID NO: 23 - protein, 9 aa, Homo sapiens
  (1)..(9): One or more of the listed amino acids can be modified as set forth in the Tables
AESPERVLL

SEQ ID NO: 24 - protein, 8 aa, Homo sapiens
  (1)..(8): One or more of the listed amino acids can be modified as set forth in the Tables
AFSPVRSV

SEQ ID NO: 25 - protein, 9 aa, Homo sapiens
  (1)..(9): One or more of the listed amino acids can be modified as set forth in the Tables
AIMRSPQMV

SEQ ID NO: 26 - protein, 9 aa, Homo sapiens
  (1)..(9): One or more of the listed amino acids can be modified as set forth in the Tables
AIMRSPQMV

SEQ ID NO: 27 - protein, 9 aa, Homo sapiens
  (1)..(9): One or more of the listed amino acids can be modified as set forth in the Tables
AIMRSPQMV

SEQ ID NO: 28 - protein, 9 aa, Homo sapiens
  (1)..(9): One or more of the listed amino acids can be modified as set forth in the Tables
ALAAPSPPR

SEQ ID NO: 29 - protein, 10 aa, Homo sapiens
  (1)..(10): One or more of the listed amino acids can be modified as set forth in the Tables
ALDSPPPPTL

SEQ ID NO: 30 - protein, 9 aa, Homo sapiens
  (1)..(9): One or more of the listed amino acids can be modified as set forth in the Tables
ALDSQVPKV

SEQ ID NO: 31 - protein, 11 aa, Homo sapiens
  (1)..(11): One or more of the listed amino acids can be modified as set forth in the Tables
ALGSRESLATL

SEQ ID NO: 32 - protein, 9 aa, Homo sapiens
  (1)..(9): One or more of the listed amino acids can be modified as set forth in the Tables
ALLDIIRSL

SEQ ID NO: 33 - protein, 11 aa, Homo sapiens
  (1)..(11): One or more of the listed amino acids can be modified as set forth in the Tables
ALMGSPQLVAA

SEQ ID NO: 34 - protein, 10 aa, Homo sapiens
  (1)..(10): One or more of the listed amino acids can be modified as set forth in the Tables
ALRSSPIMRK

SEQ ID NO: 35 - protein, 9 aa, Homo sapiens
  (1)..(9): One or more of the listed amino acids can be modified as set forth in the Tables
ALSSLIHAL

SEQ ID NO: 36 - protein, 9 aa, Homo sapiens
  (1)..(9): One or more of the listed amino acids can be modified as set forth in the Tables
ALSTPVVEK

SEQ ID NO: 37 - protein, 9 aa, Homo sapiens
  (1)..(9): One or more of the listed amino acids can be modified as set forth in the Tables
ALTSELANA

SEQ ID NO: 38 - protein, 9 aa, Homo sapiens
  (1)..(9): One or more of the listed amino acids can be modified as set forth in the Tables
AMAASPHAV

SEQ ID NO: 39 - protein, 9 aa, Homo sapiens
  (1)..(9): One or more of the listed amino acids can be modified as set forth in the Tables
AMDSPLLKY

SEQ ID NO: 40 - protein, 9 aa, Homo sapiens
  (1)..(9): One or more of the listed amino acids can be modified as set forth in the Tables
AMGAGHFSV

SEQ ID NO: 41 - protein, 13 aa, Homo sapiens
  (1)..(13): One or more of the listed amino acids can be modified as set forth in the Tables
AMLGSKSPDPYRL

SEQ ID NO: 42 - protein, 10 aa, Homo sapiens
  (1)..(10): One or more of the listed amino acids can be modified as set forth in the Tables
APAGGSPRML

SEQ ID NO: 43 - protein, 9 aa, Homo sapiens
  (1)..(9): One or more of the listed amino acids can be modified as set forth in the Tables
APASPFRQL

SEQ ID NO: 44 - protein, 10 aa, Homo sapiens
  (1)..(10): One or more of the listed amino acids can be modified as set forth in the Tables
APASPFRQLL

SEQ ID NO: 45 - protein, 9 aa, Homo sapiens
  (1)..(9): One or more of the listed amino acids can be modified as set forth in the Tables
APASPLRPL

SEQ ID NO: 46 - protein, 11 aa, Homo sapiens
  (1)..(11): One or more of the listed amino acids can be modified as set forth in the Tables
APASPNHAGVL

SEQ ID NO: 47 - protein, 8 aa, Homo sapiens
  (1)..(8): One or more of the listed amino acids can be modified as set forth in the Tables
APASPRLL

SEQ ID NO: 48 - protein, 10 aa, Homo sapiens
  (1)..(10): One or more of the listed amino acids can be modified as set forth in the Tables
APASPTHPGL

SEQ ID NO: 49 - protein, 11 aa, Homo sapiens
  (1)..(11): One or more of the listed amino acids can be modified as set forth in the Tables
APASPTHPGLM

SEQ ID NO: 50 - protein, 9 aa, Homo sapiens
  (1)..(9): One or more of the listed amino acids can be modified as set forth in the Tables
APDSPSKQL

SEQ ID NO: 51 - protein, 11 aa, Homo sapiens
  (1)..(11): One or more of the listed amino acids can be modified as set forth in the Tables
APGPGFSSRSL

SEQ ID NO: 52 - protein, 11 aa, Homo sapiens
  (1)..(11): One or more of the listed amino acids can be modified as set forth in the Tables
APKSPSQDVKA

SEQ ID NO: 53 - protein, 9 aa, Homo sapiens
  (1)..(9): One or more of the listed amino acids can be modified as set forth in the Tables
APLARASSL

SEQ ID NO: 54 - protein, 12 aa, Homo sapiens
  (1)..(12): One or more of the listed amino acids can be modified as set forth in the Tables
APRAPSASPLAL

SEQ ID NO: 55 - protein, 10 aa, Homo sapiens
  (1)..(10): One or more of the listed amino acids can be modified as set forth in the Tables
APRTPPGVTF

SEQ ID NO: 56 - protein, 8 aa, Homo sapiens
  (1)..(8): One or more of the listed amino acids can be modified as set forth in the Tables
APSRQISL

SEQ ID NO: 57 - protein, 9 aa, Homo sapiens
  (1)..(9): One or more of the listed amino acids can be modified as set forth in the Tables
APSVRSLSL

SEQ ID NO: 58 - protein, 9 aa, Homo sapiens
  (1)..(9): One or more of the listed amino acids can be modified as set forth in the Tables
APSVRSLSL

SEQ ID NO: 59 - protein, 8 aa, Homo sapiens
  (1)..(8): One or more of the listed amino acids can be modified as set forth in the Tables
APVSPLKF

SEQ ID NO: 60 - protein, 9 aa, Homo sapiens
  (1)..(9): One or more of the listed amino acids can be modified as set forth in the Tables
APVSPRPGL

SEQ ID NO: 61 - protein, 10 aa, Homo sapiens
  (1)..(10): One or more of the listed amino acids can be modified as set forth in the Tables
APVSPSSQKL

SEQ ID NO: 62 - protein, 13 aa, Homo sapiens
  (1)..(13): One or more of the listed amino acids can be modified as set forth in the Tables
APYRGQLASPSSQ

SEQ ID NO: 63 - protein, 8 aa, Homo sapiens
  (1)..(8): One or more of the listed amino acids can be modified as set forth in the Tables
AQDSPTHL

SEQ ID NO: 64 - protein, 10 aa, Homo sapiens
  (1)..(10): One or more of the listed amino acids can be modified as set forth in the Tables
ARASPRLHFL

SEQ ID NO: 65 - protein, 9 aa, Homo sapiens
  (1)..(9): One or more of the listed amino acids can be modified as set forth in the Tables
ARFSGFYSM

SEQ ID NO: 66 - protein, 9 aa, Homo sapiens
  (1)..(9): One or more of the listed amino acids can be modified as set forth in the Tables
ARFSGFYSM

SEQ ID NO: 67 - protein, 9 aa, Homo sapiens
  (1)..(9): One or more of the listed amino acids can be modified as set forth in the Tables
ARFSPKVSL

SEQ ID NO: 68 - protein, 9 aa, Homo sapiens
  (1)..(9): One or more of the listed amino acids can be modified as set forth in the Tables
ARGSLRRLL

SEQ ID NO: 69 - protein, 9 aa, Homo sapiens
  (1)..(9): One or more of the listed amino acids can be modified as set forth in the Tables
ARISRSISL

SEQ ID NO: 70 - protein, 9 aa, Homo sapiens
  (1)..(9): One or more of the listed amino acids can be modified as set forth in the Tables
ARISRSISL

SEQ ID NO: 71 - protein, 10 aa, Homo sapiens
  (1)..(10): One or more of the listed amino acids can be modified as set forth in the Tables
ARTSPINLGL

SEQ ID NO: 72 - protein, 9 aa, Homo sapiens
  (1)..(9): One or more of the listed amino acids can be modified as set forth in the Tables
ARVSPSTSY

SEQ ID NO: 73 - protein, 11 aa, Homo sapiens
  (1)..(11): One or more of the listed amino acids can be modified as set forth in the Tables
ASESPSSLIFY

SEQ ID NO: 74 - protein, 12 aa, Homo sapiens
  (1)..(12): One or more of the listed amino acids can be modified as set forth in the Tables
ASGVAVSDGVIK

SEQ ID NO: 75 - protein, 9 aa, Homo sapiens
  (1)..(9): One or more of the listed amino acids can be modified as set forth in the Tables
ASLSPSVSK

SEQ ID NO: 76 - protein, 9 aa, Homo sapiens
  (1)..(9): One or more of the listed amino acids can be modified as set forth in the Tables
ASLSRPLNY

SEQ ID NO: 77 - protein, 11 aa, Homo sapiens
  (1)..(11): One or more of the listed amino acids can be modified as set forth in the Tables
ASMSPGHPTHL

SEQ ID NO: 78 - protein, 11 aa, Homo sapiens
  (1)..(11): One or more of the listed amino acids can be modified as set forth in the Tables
ASSPPDRIDIF

SEQ ID NO: 79 - protein, 9 aa, Homo sapiens
  (1)..(9): One or more of the listed amino acids can be modified as set forth in the Tables
ASSSPVTLR

SEQ ID NO: 80 - protein, 9 aa, Homo sapiens
  (1)..(9): One or more of the listed amino acids can be modified as set forth in the Tables
ASSSQIIHI

SEQ ID NO: 81 - protein, 9 aa, Homo sapiens
  (1)..(9): One or more of the listed amino acids can be modified as set forth in the Tables
ATIPRPFSV

SEQ ID NO: 82 - protein, 9 aa, Homo sapiens
  (1)..(9): One or more of the listed amino acids can be modified as set forth in the Tables
ATMKRMLSL

SEQ ID NO: 83 - protein, 12 aa, Homo sapiens
  (1)..(12): One or more of the listed amino acids can be modified as set forth in the Tables
AVILPPLSPYFK

SEQ ID NO: 84 - protein, 16 aa, Homo sapiens
  (1)..(16): One or more of the listed amino acids can be modified as set forth in the Tables
AYAQPQTTTPLPAVSG

SEQ ID NO: 85 - protein, 12 aa, Homo sapiens
  (1)..(12): One or more of the listed amino acids can be modified as set forth in the Tables
AYGGLTSPGLSY

SEQ ID NO: 86 - protein, 17 aa, Homo sapiens
  (1)..(17): One or more of the listed amino acids can be modified as set forth in the Tables
DEGPGHHHKPGLGEGTP

SEQ ID NO: 87 - protein, 8 aa, Homo sapiens
  (1)..(8): One or more of the listed amino acids can be modified as set forth in the Tables
DERLRINS

SEQ ID NO: 88 - protein, 9 aa, Homo sapiens
  (1)..(9): One or more of the listed amino acids can be modified as set forth in the Tables
DETERAYSF

SEQ ID NO: 89 - protein, 9 aa, Homo sapiens
  (1)..(9): One or more of the listed amino acids can be modified as set forth in the Tables
DGRRTFPRI

SEQ ID NO: 90 - protein, 8 aa, Homo sapiens
  (1)..(8): One or more of the listed amino acids can be modified as set forth in the Tables
DLRQAHSL

SEQ ID NO: 91 - protein, 10 aa, Homo sapiens
  (1)..(10): One or more of the listed amino acids can be modified as set forth in the Tables
DPLSVSPARW

SEQ ID NO: 92 - protein, 8 aa, Homo sapiens
  (1)..(8): One or more of the listed amino acids can be modified as set forth in the Tables
DRKSPRVL

SEQ ID NO: 93 - protein, 9 aa, Homo sapiens
  (1)..(9): One or more of the listed amino acids can be modified as set forth in the Tables
DRKSPSVSL

SEQ ID NO: 94 - protein, 9 aa, Homo sapiens
  (1)..(9): One or more of the listed amino acids can be modified as set forth in the Tables
DRLGSRPSL

SEQ ID NO: 95 - protein, 9 aa, Homo sapiens
  (1)..(9): One or more of the listed amino acids can be modified as set forth in the Tables
DRQRSPIAL

SEQ ID NO: 96 - protein, 10 aa, Homo sapiens
  (1)..(10): One or more of the listed amino acids can be modified as set forth in the Tables
DRSSPPTTPL

SEQ ID NO: 97 - protein, 9 aa, Homo sapiens
  (1)..(9): One or more of the listed amino acids can be modified as set forth in the Tables
DSFESIESY

SEQ ID NO: 98 - protein, 9 aa, Homo sapiens
  (1)..(9): One or more of the listed amino acids can be modified as set forth in the Tables
DSLARILSF

SEQ ID NO: 99 - protein, 6 aa, Homo sapiens
  (1)..(6): One or more of the listed amino acids can be modified as set forth in the Tables
DSSEEK

SEQ ID NO: 100 - protein, 7 aa, Homo sapiens
  (1)..(7): One or more of the listed amino acids can be modified as set forth in the Tables
DSSEEKF

SEQ ID NO: 101 - protein, 7 aa, Homo sapiens
  (1)..(7): One or more of the listed amino acids can be modified as set forth in the Tables
DSSEEKF

SEQ ID NO: 102 - protein, 8 aa, Homo sapiens
  (1)..(8): One or more of the listed amino acids can be modified as set forth in the Tables
DSSEEKFL

SEQ ID NO: 103 - protein, 9 aa, Homo sapiens
  (1)..(9): One or more of the listed amino acids can be modified as set forth in the Tables
DSSEEKFLR

SEQ ID NO: 104 - protein, 9 aa, Homo sapiens
  (1)..(9): One or more of the listed amino acids can be modified as set forth in the Tables
DSSEEKFLR

SEQ ID NO: 105 - protein, 9 aa, Homo sapiens
  (1)..(9): One or more of the listed amino acids can be modified as set forth in the Tables
DSVSPSESL

SEQ ID NO: 106 - protein, 11 aa, Homo sapiens
  (1)..(11): One or more of the listed amino acids can be modified as set forth in the Tables
DTDSAIGSFRY

SEQ ID NO: 107 - protein, 9 aa, Homo sapiens
  (1)..(9): One or more of the listed amino acids can be modified as set forth in the Tables
DTISPTLGF

SEQ ID NO: 108 - protein, 10 aa, Homo sapiens
  (1)..(10): One or more of the listed amino acids can be modified as set forth in the Tables
DVYSGTPTKV

SEQ ID NO: 109 - protein, 9 aa, Homo sapiens
  (1)..(9): One or more of the listed amino acids can be modified as set forth in the Tables
DYSPYFKTI

SEQ ID NO: 110 - protein, 9 aa, Homo sapiens
  (1)..(9): One or more of the listed amino acids can be modified as set forth in the Tables
EASSVTREL

SEQ ID NO: 111 - protein, 10 aa, Homo sapiens
  (1)..(10): One or more of the listed amino acids can be modified as set forth in the Tables
EEAPQTPVAF

SEQ ID NO: 112 - protein, 11 aa, Homo sapiens
  (1)..(11): One or more of the listed amino acids can be modified as set forth in the Tables
EEFSPRQAQMF

SEQ ID NO: 113 - protein, 9 aa, Homo sapiens
  (1)..(9): One or more of the listed amino acids can be modified as set forth in the Tables
EEIGTPRKF

SEQ ID NO: 114 - protein, 11 aa, Homo sapiens
  (1)..(11): One or more of the listed amino acids can be modified as set forth in the Tables
EELSPLALGRF

SEQ ID NO: 115 - protein, 9 aa, Homo sapiens
  (1)..(9): One or more of the listed amino acids can be modified as set forth in the Tables
EEQSFLQKF

SEQ ID NO: 116 - protein, 10 aa, Homo sapiens
  (1)..(10): One or more of the listed amino acids can be modified as set forth in the Tables
EERSPSWISA

SEQ ID NO: 117 - protein, 10 aa, Homo sapiens
  (1)..(10): One or more of the listed amino acids can be modified as set forth in the Tables
EERSPSWISA

SEQ ID NO: 118 - protein, 9 aa, Homo sapiens
  (1)..(9): One or more of the listed amino acids can be modified as set forth in the Tables
EHVPSSSSI

SEQ ID NO: 119 - protein, 10 aa, Homo sapiens
  (1)..(10): One or more of the listed amino acids can be modified as set forth in the Tables
EILNRSPRNR

SEQ ID NO: 120 - protein, 9 aa, Homo sapiens
  (1)..(9): One or more of the listed amino acids can be modified as set forth in the Tables
ELLPRRNSL

SEQ ID NO: 121 - protein, 14 aa, Homo sapiens
  (1)..(14): One or more of the listed amino acids can be modified as set forth in the Tables
EPRNSLPASPAHQL

SEQ ID NO: 122 - protein, 9 aa, Homo sapiens
  (1)..(9): One or more of the listed amino acids can be modified as set forth in the Tables
ERLKIRGSL

SEQ ID NO: 123 - protein, 9 aa, Homo sapiens
  (1)..(9): One or more of the listed amino acids can be modified as set forth in the Tables
ERVDSLVSL

SEQ ID NO: 124 - protein, 10 aa, Homo sapiens
  (1)..(10): One or more of the listed amino acids can be modified as set forth in the Tables
FAFPGSTNSL

SEQ ID NO: 125 - protein, 10 aa, Homo sapiens
  (1)..(10): One or more of the listed amino acids can be modified as set forth in the Tables
FAFPGSTNSL

SEQ ID NO: 126 - protein, 10 aa, Homo sapiens
  (1)..(10): One or more of the listed amino acids can be modified as set forth in the Tables
FASPTSPPVL

SEQ ID NO: 127 - protein, 9 aa, Homo sapiens
  (1)..(9): One or more of the listed amino acids can be modified as set forth in the Tables
FATIRTASL

SEQ ID NO: 128 - protein, 13 aa, Homo sapiens
  (1)..(13): One or more of the listed amino acids can be modified as set forth in the Tables
FAVSPIPGRGGVL

SEQ ID NO: 129 - protein, 9 aa, Homo sapiens
  (1)..(9): One or more of the listed amino acids can be modified as set forth in the Tables
FAYGSGNSL

SEQ ID NO: 130 - protein, 11 aa, Homo sapiens
  (1)..(11): One or more of the listed amino acids can be modified as set forth in the Tables
FAYSPGGAHGM

SEQ ID NO: 131 - protein, 12 aa, Homo sapiens
  (1)..(12): One or more of the listed amino acids can be modified as set forth in the Tables
FAYSPGGAHGML

SEQ ID NO: 132 - protein, 12 aa, Homo sapiens
  (1)..(12): One or more of the listed amino acids can be modified as set forth in the Tables
FAYSPGGAHGML

SEQ ID NO: 133 - protein, 9 aa, Homo sapiens
  (1)..(9): One or more of the listed amino acids can be modified as set forth in the Tables
FGINSPQAL

SEQ ID NO: 134 - protein, 9 aa, Homo sapiens
  (1)..(9): One or more of the listed amino acids can be modified as set forth in the Tables
FGLARAFSL

SEQ ID NO: 135 - protein, 7 aa, Homo sapiens
  (1)..(7): One or more of the listed amino acids can be modified as set forth in the Tables
FGRKPSL

SEQ ID NO: 136 - protein, 9 aa, Homo sapiens
  (1)..(9): One or more of the listed amino acids can be modified as set forth in the Tables
FGYDSPHDL

SEQ ID NO: 137 - protein, 8 aa, Homo sapiens
  (1)..(8): One or more of the listed amino acids can be modified as set forth in the Tables
FKLSGLSF

SEQ ID NO: 138 - protein, 9 aa, Homo sapiens
  (1)..(9): One or more of the listed amino acids can be modified as set forth in the Tables
FLDSAYFRL

SEQ ID NO: 139 - protein, 10 aa, Homo sapiens
  (1)..(10): One or more of the listed amino acids can be modified as set forth in the Tables
FLLSPSDQEM

SEQ ID NO: 140 - protein, 10 aa, Homo sapiens
  (1)..(10): One or more of the listed amino acids can be modified as set forth in the Tables
FLLSPSDQEM

SEQ ID NO: 141 - protein, 10 aa, Homo sapiens
  (1)..(10): One or more of the listed amino acids can be modified as set forth in the Tables
FLMSDRSLHL

SEQ ID NO: 142 - protein, 9 aa, Homo sapiens
  (1)..(9): One or more of the listed amino acids can be modified as set forth in the Tables
FLSRSIPSL

SEQ ID NO: 143 - protein, 9 aa, Homo sapiens
  (1)..(9): One or more of the listed amino acids can be modified as set forth in the Tables
FPASPSVSL

SEQ ID NO: 144 - protein, 9 aa, Homo sapiens
  (1)..(9): One or more of the listed amino acids can be modified as set forth in the Tables
FPLARQFSL

SEQ ID NO: 145 - protein, 9 aa, Homo sapiens
  (1)..(9): One or more of the listed amino acids can be modified as set forth in the Tables
FPLSPLRKY

SEQ ID NO: 146 - protein, 11 aa, Homo sapiens
  (1)..(11): One or more of the listed amino acids can be modified as set forth in the Tables
FPLSPTKLSQY

SEQ ID NO: 147 - protein, 9 aa, Homo sapiens
  (1)..(9): One or more of the listed amino acids can be modified as set forth in the Tables
FPRASPRAL

SEQ ID NO: 148 - protein, 9 aa, Homo sapiens
  (1)..(9): One or more of the listed amino acids can be modified as set forth in the Tables
FQYSKSPSL

SEQ ID NO: 149 - protein, 9 aa, Homo sapiens
  (1)..(9): One or more of the listed amino acids can be modified as set forth in the Tables
FRFPSGAEL

SEQ ID NO: 150 - protein, 9 aa, Homo sapiens
  (1)..(9): One or more of the listed amino acids can be modified as set forth in the Tables
FRRSPTEDF

SEQ ID NO: 151 - protein, 9 aa, Homo sapiens
  (1)..(9): One or more of the listed amino acids can be modified as set forth in the Tables
FRYSGRTQA

SEQ ID NO: 152 - protein, 9 aa, Homo sapiens
  (1)..(9): One or more of the listed amino acids can be modified as set forth in the Tables
FSFAGFPSA

SEQ ID NO: 153 - protein, 7 aa, Homo sapiens
  (1)..(7): One or more of the listed amino acids can be modified as set forth in the Tables
FSFKKSF

SEQ ID NO: 154 - protein, 9 aa, Homo sapiens
  (1)..(9): One or more of the listed amino acids can be modified as set forth in the Tables
FSFKKSFKL

SEQ ID NO: 155 - protein, 10 aa, Homo sapiens
  (1)..(10): One or more of the listed amino acids can be modified as set forth in the Tables
FSFKKSFKLS

SEQ ID NO: 156 - protein, 10 aa, Homo sapiens
  (1)..(10): One or more of the listed amino acids can be modified as set forth in the Tables
FSSSHEGFSY

SEQ ID NO: 157 - protein, 9 aa, Homo sapiens
  (1)..(9): One or more of the listed amino acids can be modified as set forth in the Tables
FSVASPLTL

SEQ ID NO: 158 - protein, 9 aa, Homo sapiens
  (1)..(9): One or more of the listed amino acids can be modified as set forth in the Tables
FSVSPASTL

SEQ ID NO: 159 - protein, 9 aa, Homo sapiens
  (1)..(9): One or more of the listed amino acids can be modified as set forth in the Tables
FSYSPRLPL

SEQ ID NO: 160 - protein, 10 aa, Homo sapiens
  (1)..(10): One or more of the listed amino acids can be modified as set forth in the Tables
FTDVNSILRY

SEQ ID NO: 161 - protein, 10 aa, Homo sapiens
  (1)..(10): One or more of the listed amino acids can be modified as set forth in the Tables
FVSEGDGGRL

SEQ ID NO: 162 - protein, 9 aa, Homo sapiens
  (1)..(9): One or more of the listed amino acids can be modified as set forth in the Tables
FVTTPTAEL

SEQ ID NO: 163 - protein, 9 aa, Homo sapiens
  (1)..(9): One or more of the listed amino acids can be modified as set forth in the Tables
GARTPSPSL

SEQ ID NO: 164 - protein, 9 aa, Homo sapiens
  (1)..(9): One or more of the listed amino acids can be modified as set forth in the Tables
GATTTAPSL

SEQ ID NO: 165 - protein, 9 aa, Homo sapiens
  (1)..(9): One or more of the listed amino acids can be modified as set forth in the Tables
GAVSPVGEL

SEQ ID NO: 166 - protein, 18 aa, Homo sapiens
  (1)..(18): One or more of the listed amino acids can be modified as set forth in the Tables
GDEGPGHHHKPGLGEGTP

SEQ ID NO: 167 - protein, 9 aa, Homo sapiens
  (1)..(9): One or more of the listed amino acids can be modified as set forth in the Tables
GEASPLSSL

SEQ ID NO: 168 - protein, 9 aa, Homo sapiens
  (1)..(9): One or more of the listed amino acids can be modified as set forth in the Tables
GEFGGFGSV

SEQ ID NO: 169 - protein, 9 aa, Homo sapiens
  (1)..(9): One or more of the listed amino acids can be modified as set forth in the Tables
GEISPTQIL

SEQ ID NO: 170 - protein, 10 aa, Homo sapiens
  (1)..(10): One or more of the listed amino acids can be modified as set forth in the Tables
GEKSPPYGVP

SEQ ID NO: 171 - protein, 9 aa, Homo sapiens
  (1)..(9): One or more of the listed amino acids can be modified as set forth in the Tables
GELPSPGKV

SEQ ID NO: 172 - protein, 11 aa, Homo sapiens
  (1)..(11): One or more of the listed amino acids can be modified as set forth in the Tables
GELPTSPLHLL

SEQ ID NO: 173 - protein, 10 aa, Homo sapiens
  (1)..(10): One or more of the listed amino acids can be modified as set forth in the Tables
GEMSPQRFFF

SEQ ID NO: 174 - protein, 10 aa, Homo sapiens
  (1)..(10): One or more of the listed amino acids can be modified as set forth in the Tables
GEMSPQRFFF

SEQ ID NO: 175 - protein, 10 aa, Homo sapiens
  (1)..(10): One or more of the listed amino acids can be modified as set forth in the Tables
GENSGIGKLF

SEQ ID NO: 176 - protein, 9 aa, Homo sapiens
  (1)..(9): One or more of the listed amino acids can be modified as set forth in the Tables
GEPHSSPEL

SEQ ID NO: 177 - protein, 9 aa, Homo sapiens
  (1)..(9): One or more of the listed amino acids can be modified as set forth in the Tables
GEQSPNVSL

SEQ ID NO: 178 - protein, 9 aa, Homo sapiens
  (1)..(9): One or more of the listed amino acids can be modified as set forth in the Tables
GERSPPRIL

SEQ ID NO: 179 - protein, 9 aa, Homo sapiens
  (1)..(9): One or more of the listed amino acids can be modified as set forth in the Tables
GETSLMRTL

SEQ ID NO: 180 - protein, 10 aa, Homo sapiens
  (1)..(10): One or more of the listed amino acids can be modified as set forth in the Tables
GETSPHTFQL

SEQ ID NO: 181 - protein, 11 aa, Homo sapiens
  (1)..(11): One or more of the listed amino acids can be modified as set forth in the Tables
GETSPRTKITW

SEQ ID NO: 182 - protein, 9 aa, Homo sapiens
  (1)..(9): One or more of the listed amino acids can be modified as set forth in the Tables
GETSYIRVY

SEQ ID NO: 183 - protein, 8 aa, Homo sapiens
  (1)..(8): One or more of the listed amino acids can be modified as set forth in the Tables
GGDSPVRL

SEQ ID NO: 184 - protein, 10 aa, Homo sapiens
  (1)..(10): One or more of the listed amino acids can be modified as set forth in the Tables
GGLTSPGLSY

SEQ ID NO: 185 - protein, 12 aa, Homo sapiens
  (1)..(12): One or more of the listed amino acids can be modified as set forth in the Tables
GGPHFSPEHKEL

SEQ ID NO: 186 - protein, 9 aa, Homo sapiens
GHGSPFPSL

SEQ ID NO: 187 - protein, 13 aa, Homo sapiens
  (1)..(13): One or more of the listed amino acids can be modified as set forth in the Tables
GHHHKPGLGEGTP

SEQ ID NO: 188 - protein, 9 aa, Homo sapiens
  (1)..(9): One or more of the listed amino acids can be modified as set forth in the Tables
GHSKTILCM

SEQ ID NO: 189 - protein, 10 aa, Homo sapiens
  (1)..(10): One or more of the listed amino acids can be modified as set forth in the Tables
GIFPGTPLKK

SEQ ID NO: 190 - protein, 9 aa, Homo sapiens
  (1)..(9): One or more of the listed amino acids can be modified as set forth in the Tables
GIMSPLAKK

SEQ ID NO: 191 - protein, 17 aa, Homo sapiens
  (1)..(17): One or more of the listed amino acids can be modified as set forth in the Tables
GKGGSYSQAASSDSAQG

SEQ ID NO: 192 - protein, 10 aa, Homo sapiens
GLAPNTPGKA

SEQ ID NO: 193 - protein, 9 aa, Homo sapiens
  (1)..(9): One or more of the listed amino acids can be modified as set forth in the Tables
GLAPTPPSM

SEQ ID NO: 194 - protein, 11 aa, Homo sapiens
  (1)..(11): One or more of the listed amino acids can be modified as set forth in the Tables
GLASPTAITPV

SEQ ID NO: 195 - protein, 10 aa, Homo sapiens
  (1)..(10): One or more of the listed amino acids can be modified as set forth in the Tables
GLLARTPPAA

SEQ ID NO: 196 - protein, 10 aa, Homo sapiens
  (1)..(10): One or more of the listed amino acids can be modified as set forth in the Tables
GLLNKTPPTA

SEQ ID NO: 197 - protein, 9 aa, Homo sapiens
  (1)..(9): One or more of the listed amino acids can be modified as set forth in the Tables
GLSSLSIHL

SEQ ID NO: 198 - protein, 9 aa, Homo sapiens
  (1)..(9): One or more of the listed amino acids can be modified as set forth in the Tables
GLTSPGLSY

SEQ ID NO: 199 - protein, 10 aa, Homo sapiens
  (1)..(10): One or more of the listed amino acids can be modified as set forth in the Tables
GLTSPGLSYS

SEQ ID NO: 200 - protein, 11 aa, Homo sapiens
  (1)..(11): One or more of the listed amino acids can be modified as set forth in the Tables
GLTSPGLSYSL

SEQ ID NO: 201 - protein, 11 aa, Homo sapiens
  (1)..(11): One or more of the listed amino acids can be modified as set forth in the Tables
GMLSPGKSIEV

SEQ ID NO: 202 - protein, 15 aa, Homo sapiens
  (1)..(15): One or more of the listed amino acids can be modified as set forth in the Tables
GPGHHHKPGLGEGTP

SEQ ID NO: 203 - protein, 9 aa, Homo sapiens
  (1)..(9): One or more of the listed amino acids can be modified as set forth in the Tables
GPLSRVKSL

SEQ ID NO: 204 - protein, 9 aa, Homo sapiens
  (1)..(9): One or more of the listed amino acids can be modified as set forth in the Tables
GPLVRQISL

SEQ ID NO: 205 - protein, 11 aa, Homo sapiens
  (1)..(11): One or more of the listed amino acids can be modified as set forth in the Tables
GPRAPSPTKPL

SEQ ID NO: 206 - protein, 11 aa, Homo sapiens
  (1)..(11): One or more of the listed amino acids can be modified as set forth in the Tables
GPRPGSPSALL

SEQ ID NO: 207 - protein, 10 aa, Homo sapiens
  (1)..(10): One or more of the listed amino acids can be modified as set forth in the Tables
GPRSASLLSL

SEQ ID NO: 208 - protein, 9 aa, Homo sapiens
  (1)..(9): One or more of the listed amino acids can be modified as set forth in the Tables
GPRSPPVTL

SEQ ID NO: 209 - protein, 9 aa, Homo sapiens
  (1)..(9): One or more of the listed amino acids can be modified as set forth in the Tables
GPSSPWTQL

SEQ ID NO: 210 - protein, 9 aa, Homo sapiens
  (1)..(9): One or more of the listed amino acids can be modified as set forth in the Tables
GSDSPRSSL

SEQ ID NO: 211 - protein, 11 aa, Homo sapiens
  (1)..(11): One or more of the listed amino acids can be modified as set forth in the Tables
GSDVSLTACKV

SEQ ID NO: 212 - protein, 9 aa, Homo sapiens
  (1)..(9): One or more of the listed amino acids can be modified as set forth in the Tables
GSGPEIFTF

SEQ ID NO: 213 - protein, 9 aa, Homo sapiens
  (1)..(9): One or more of the listed amino acids can be modified as set forth in the Tables
GSKSPISQL

SEQ ID NO: 214 - protein, 11 aa, Homo sapiens
  (1)..(11): One or more of the listed amino acids can be modified as set forth in the Tables
GSPHYFSPFRP

SEQ ID NO: 215 - protein, 8 aa, Homo sapiens
  (1)..(8): One or more of the listed amino acids can be modified as set forth in the Tables
GSPIKVTL

SEQ ID NO: 216 - protein, 9 aa, Homo sapiens
  (1)..(9): One or more of the listed amino acids can be modified as set forth in the Tables
GSPIKVTLA

SEQ ID NO: 217 - protein, 9 aa, Homo sapiens
  (1)..(9): One or more of the listed amino acids can be modified as set forth in the Tables
GTFPKALSI

SEQ ID NO: 218 - protein, 9 aa, Homo sapiens
  (1)..(9): One or more of the listed amino acids can be modified as set forth in the Tables
GTISPTSSL

SEQ ID NO: 219 - protein, 12 aa, Homo sapiens
  (1)..(12): One or more of the listed amino acids can be modified as set forth in the Tables
GTPLSQAIIHQY

SEQ ID NO: 220 - protein, 9 aa, Homo sapiens
  (1)..(9): One or more of the listed amino acids can be modified as set forth in the Tables
GTVTPALKL

SEQ ID NO: 221 - protein, 13 aa, Homo sapiens
  (1)..(13): One or more of the listed amino acids can be modified as set forth in the Tables
GTYVPSSPTRLAY

SEQ ID NO: 222 - protein, 10 aa, Homo sapiens
  (1)..(10): One or more of the listed amino acids can be modified as set forth in the Tables
GVISPQELLK

SEQ ID NO: 223 - protein, 11 aa, Homo sapiens
  (1)..(11): One or more of the listed amino acids can be modified as set forth in the Tables
GVISPQELLKK

SEQ ID NO: 224 - protein, 11 aa, Homo sapiens
  (1)..(11): One or more of the listed amino acids can be modified as set forth in the Tables
GVISPRFDVQL

SEQ ID NO: 225 - protein, 12 aa, Homo sapiens
  (1)..(12): One or more of the listed amino acids can be modified as set forth in the Tables
GYVQRNLSLVRG

SEQ ID NO: 226 - protein, 9 aa, Homo sapiens
  (1)..(9): One or more of the listed amino acids can be modified as set forth in the Tables
HAVSPIAKY

SEQ ID NO: 227 - protein, 9 aa, Homo sapiens
  (1)..(9): One or more of the listed amino acids can be modified as set forth in the Tables
HEFMSDTNL

SEQ ID NO: 228 - protein, 9 aa, Homo sapiens
  (1)..(9): One or more of the listed amino acids can be modified as set forth in the Tables
HERGSLASL

SEQ ID NO: 229 - protein, 12 aa, Homo sapiens
  (1)..(12): One or more of the listed amino acids can be modified as set forth in the Tables
HHHKPGLGEGTP

SEQ ID NO: 230 - protein, 11 aa, Homo sapiens
  (1)..(11): One or more of the listed amino acids can be modified as set forth in the Tables
HHKPGLGEGTP

SEQ ID NO: 231 - protein, 9 aa, Homo sapiens
  (1)..(9): One or more of the listed amino acids can be modified as set forth in the Tables
HIPSPAKKV

SEQ ID NO: 232 - protein, 10 aa, Homo sapiens
  (1)..(10): One or more of the listed amino acids can be modified as set forth in the Tables
HKPGLGEGTP

SEQ ID NO: 233 - protein, 9 aa, Homo sapiens
  (1)..(9): One or more of the listed amino acids can be modified as set forth in the Tables
HLYTSLPSL

SEQ ID NO: 234 - protein, 10 aa, Homo sapiens
  (1)..(10): One or more of the listed amino acids can be modified as set forth in the Tables
HPASPAHPLL

SEQ ID NO: 235 - protein, 10 aa, Homo sapiens
  (1)..(10): One or more of the listed amino acids can be modified as set forth in the Tables
HPFHATPNTY

SEQ ID NO: 236 - protein, 9 aa, Homo sapiens
  (1)..(9): One or more of the listed amino acids can be modified as set forth in the Tables
HPLTPLITY

SEQ ID NO: 237 - protein, 9 aa, Homo sapiens
  (1)..(9): One or more of the listed amino acids can be modified as set forth in the Tables
HPRPTSQDL

SEQ ID NO: 238 - protein, 8 aa, Homo sapiens
  (1)..(8): One or more of the listed amino acids can be modified as set forth in the Tables
HPRSPNVL

SEQ ID NO: 239 - protein, 9 aa, Homo sapiens
  (1)..(9): One or more of the listed amino acids can be modified as set forth in the Tables
HPYSPLPGL

SEQ ID NO: 240 - protein, 9 aa, Homo sapiens
  (1)..(9): One or more of the listed amino acids can be modified as set forth in the Tables
HQGKFLQTF

SEQ ID NO: 241 - protein, 10 aa, Homo sapiens
  (1)..(10): One or more of the listed amino acids can be modified as set forth in the Tables
HRFSINGHFY

SEQ ID NO: 242 - protein, 9 aa, Homo sapiens
  (1)..(9): One or more of the listed amino acids can be modified as set forth in the Tables
HRNSMKVFL

SEQ ID NO: 243 - protein, 9 aa, Homo sapiens
  (1)..(9): One or more of the listed amino acids can be modified as set forth in the Tables
HRVSVILKL

SEQ ID NO: 244 - protein, 12 aa, Homo sapiens
  (1)..(12): One or more of the listed amino acids can be modified as set forth in the Tables
HRYPTSIASLAF

SEQ ID NO: 245 - protein, 9 aa, Homo sapiens
  (1)..(9): One or more of the listed amino acids can be modified as set forth in the Tables
HRYSTPHAF

SEQ ID NO: 246 - protein, 10 aa, Homo sapiens
  (1)..(10): One or more of the listed amino acids can be modified as set forth in the Tables
HTASPTGMMK

SEQ ID NO: 247 - protein, 10 aa, Homo sapiens
  (1)..(10): One or more of the listed amino acids can be modified as set forth in the Tables
HTASPTGMMK

SEQ ID NO: 248 - protein, 9 aa, Homo sapiens
  (1)..(9): One or more of the listed amino acids can be modified as set forth in the Tables
HTFSPSPKL

SEQ ID NO: 249 - protein, 8 aa, Homo sapiens
  (1)..(8): One or more of the listed amino acids can be modified as set forth in the Tables
HTISPLDL

SEQ ID NO: 250 - protein, 9 aa, Homo sapiens
  (1)..(9): One or more of the listed amino acids can be modified as set forth in the Tables
HTISPSFQL

SEQ ID NO: 251 - protein, 10 aa, Homo sapiens
  (1)..(10): One or more of the listed amino acids can be modified as set forth in the Tables
HVSLITPTKR

SEQ ID NO: 252 - protein, 9 aa, Homo sapiens
  (1)..(9): One or more of the listed amino acids can be modified as set forth in the Tables
HYSSLVRVL

SEQ ID NO: 253 - protein, 11 aa, Homo sapiens
  (1)..(11): One or more of the listed amino acids can be modified as set forth in the Tables
HYSSRLGSAIF

SEQ ID NO: 254 - protein, 8 aa, Homo sapiens
  (1)..(8): One or more of the listed amino acids can be modified as set forth in the Tables
IADDRQSL

SEQ ID NO: 255 - protein, 9 aa, Homo sapiens
  (1)..(9): One or more of the listed amino acids can be modified as set forth in the Tables
IAKSPHSTV

SEQ ID NO: 256 - protein, 9 aa, Homo sapiens
  (1)..(9): One or more of the listed amino acids can be modified as set forth in the Tables
IAQDHRSSL

SEQ ID NO: 257 - protein, 9 aa, Homo sapiens
  (1)..(9): One or more of the listed amino acids can be modified as set forth in the Tables
IARLPSSTL

SEQ ID NO: 258 - protein, 9 aa, Homo sapiens
  (1)..(9): One or more of the listed amino acids can be modified as set forth in the Tables
IGKMRYVSV

SEQ ID NO: 259 - protein, 9 aa, Homo sapiens
  (1)..(9): One or more of the listed amino acids can be modified as set forth in the Tables
IIHSLETKL

SEQ ID NO: 260 - protein, 12 aa, Homo sapiens
  (1)..(12): One or more of the listed amino acids can be modified as set forth in the Tables
IIQSPSSTGLLK

SEQ ID NO: 261 - protein, 9 aa, Homo sapiens
  (1)..(9): One or more of the listed amino acids can be modified as set forth in the Tables
ILDISEHTL

SEQ ID NO: 262 - protein, 11 aa, Homo sapiens
  (1)..(11): One or more of the listed amino acids can be modified as set forth in the Tables
ILGPPPPSFHL

SEQ ID NO: 263 - protein, 9 aa, Homo sapiens
  (1)..(9): One or more of the listed amino acids can be modified as set forth in the Tables
ILYPRPKSL

SEQ ID NO: 264 - protein, 9 aa, Homo sapiens
  (1)..(9): One or more of the listed amino acids can be modified as set forth in the Tables
IPAPPSSPL

SEQ ID NO: 265 - protein, 8 aa, Homo sapiens
  (1)..(8): One or more of the listed amino acids can be modified as set forth in the Tables
IPHQRSSL

SEQ ID NO: 266 - protein, 9 aa, Homo sapiens
  (1)..(9): One or more of the listed amino acids can be modified as set forth in the Tables
IPKSKFLAL

SEQ ID NO: 267 - protein, 9 aa, Homo sapiens
  (1)..(9): One or more of the listed amino acids can be modified as set forth in the Tables
IPLSKIKTL

SEQ ID NO: 268 - protein, 12 aa, Homo sapiens
  (1)..(12): One or more of the listed amino acids can be modified as set forth in the Tables
IPRPLSLIGSTL

SEQ ID NO: 269 - protein, 11 aa, Homo sapiens
  (1)..(11): One or more of the listed amino acids can be modified as set forth in the Tables
IPRSPFKVKVL

SEQ ID NO: 270 - protein, 11 aa, Homo sapiens
  (1)..(11): One or more of the listed amino acids can be modified as set forth in the Tables
IPRTPLSPSPM

SEQ ID NO: 271 - protein, 10 aa, Homo sapiens
  (1)..(10): One or more of the listed amino acids can be modified as set forth in the Tables
IPSSPQKVAL

SEQ ID NO: 272 - protein, 9 aa, Homo sapiens
  (1)..(9): One or more of the listed amino acids can be modified as set forth in the Tables
IPTSPTSKY

SEQ ID NO: 273 - protein, 9 aa, Homo sapiens
  (1)..(9): One or more of the listed amino acids can be modified as set forth in the Tables
IPVSKPLSL

SEQ ID NO: 274 - protein, 8 aa, Homo sapiens
  (1)..(8): One or more of the listed amino acids can be modified as set forth in the Tables
IPVSPHIY

SEQ ID NO: 275 - protein, 11 aa, Homo sapiens
  (1)..(11): One or more of the listed amino acids can be modified as set forth in the Tables
IPYAPSGEIPK

SEQ ID NO: 276 - protein, 9 aa, Homo sapiens
  (1)..(9): One or more of the listed amino acids can be modified as set forth in the Tables
IRASLTKHF

SEQ ID NO: 277 - protein, 8 aa, Homo sapiens
  (1)..(8): One or more of the listed amino acids can be modified as set forth in the Tables
IRFGRKPS

SEQ ID NO: 278 - protein, 9 aa, Homo sapiens
  (1)..(9): One or more of the listed amino acids can be modified as set forth in the Tables
IRFGRKPSL

SEQ ID NO: 279 - protein, 9 aa, Homo sapiens
  (1)..(9): One or more of the listed amino acids can be modified as set forth in the Tables
IRGSKIRFL

SEQ ID NO: 280 - protein, 9 aa, Homo sapiens
  (1)..(9): One or more of the listed amino acids can be modified as set forth in the Tables
IRKERPISL

SEQ ID NO: 281 - protein, 9 aa, Homo sapiens
  (1)..(9): One or more of the listed amino acids can be modified as set forth in the Tables
IRNSQTRKI

SEQ ID NO: 282 - protein, 9 aa, Homo sapiens
  (1)..(9): One or more of the listed amino acids can be modified as set forth in the Tables
IRSSYIRVL

SEQ ID NO: 283 - protein, 8 aa, Homo sapiens
  (1)..(8): One or more of the listed amino acids can be modified as set forth in the Tables
IRYSGHSL

SEQ ID NO: 284 - protein, 9 aa, Homo sapiens
  (1)..(9): One or more of the listed amino acids can be modified as set forth in the Tables
ISIDSPQKL

SEQ ID NO: 285 - protein, 9 aa, Homo sapiens
  (1)..(9): One or more of the listed amino acids can be modified as set forth in the Tables
ISNSHPLSL

SEQ ID NO: 286 - protein, 9 aa, Homo sapiens
  (1)..(9): One or more of the listed amino acids can be modified as set forth in the Tables
ISSSMHSLY

SEQ ID NO: 287 - protein, 9 aa, Homo sapiens
  (1)..(9): One or more of the listed amino acids can be modified as set forth in the Tables
ISTSPSVAL

SEQ ID NO: 288 - protein, 11 aa, Homo sapiens
  (1)..(11): One or more of the listed amino acids can be modified as set forth in the Tables
ISVSPLATSAL

SEQ ID NO: 289 - protein, 9 aa, Homo sapiens
  (1)..(9): One or more of the listed amino acids can be modified as set forth in the Tables
ISVSRSTSF

SEQ ID NO: 290 - protein, 11 aa, Homo sapiens
  (1)..(11): One or more of the listed amino acids can be modified as set forth in the Tables
ITDLPDHLLSY

SEQ ID NO: 291 - protein, 9 aa, Homo sapiens
  (1)..(9): One or more of the listed amino acids can be modified as set forth in the Tables
ITITPPDRY

SEQ ID NO: 292 - protein, 10 aa, Homo sapiens
  (1)..(10): One or more of the listed amino acids can be modified as set forth in the Tables
ITTSPITVRK

SEQ ID NO: 293 - protein, 9 aa, Homo sapiens
  (1)..(9): One or more of the listed amino acids can be modified as set forth in the Tables
ITTTINPRF

SEQ ID NO: 294 - protein, 9 aa, Homo sapiens
  (1)..(9): One or more of the listed amino acids can be modified as set forth in the Tables
ITYMSPAKL

SEQ ID NO: 295 - protein, 8 aa, Homo sapiens
  (1)..(8): One or more of the listed amino acids can be modified as set forth in the Tables
IVDSPEKL

SEQ ID NO: 296 - protein, 9 aa, Homo sapiens
  (1)..(9): One or more of the listed amino acids can be modified as set forth in the Tables
IVSSLRLAY

SEQ ID NO: 297 - protein, 10 aa, Homo sapiens
  (1)..(10): One or more of the listed amino acids can be modified as set forth in the Tables
IYRSQSPHYF

SEQ ID NO: 298 - protein, 9 aa, Homo sapiens
  (1)..(9): One or more of the listed amino acids can be modified as set forth in the Tables
IYYKSMPNL

SEQ ID NO: 299 - protein, 9 aa, Homo sapiens
  (1)..(9): One or more of the listed amino acids can be modified as set forth in the Tables
IYYQSPLSL

SEQ ID NO: 300 - protein, 10 aa, Homo sapiens
  (1)..(10): One or more of the listed amino acids can be modified as set forth in the Tables
KADSLEVQQM

SEQ ID NO: 301 - protein, 10 aa, Homo sapiens
  (1)..(10): One or more of the listed amino acids can be modified as set forth in the Tables
KADSLEVQQM

SEQ ID NO: 302 - protein, 10 aa, Homo sapiens
  (1)..(10): One or more of the listed amino acids can be modified as set forth in the Tables
KADTVSKTEL

SEQ ID NO: 303 - protein, 8 aa, Homo sapiens
  (1)..(8): One or more of the listed amino acids can be modified as set forth in the Tables
KAFSPVRS

SEQ ID NO: 304 - protein, 9 aa, Homo sapiens
  (1)..(9): One or more of the listed amino acids can be modified as set forth in the Tables
KAFSPVRSV

SEQ ID NO: 305 - protein, 9 aa, Homo sapiens
  (1)..(9): One or more of the listed amino acids can be modified as set forth in the Tables
KAFSPVRSV

SEQ ID NO: 306 - protein, 9 aa, Homo sapiens
  (1)..(9): One or more of the listed amino acids can be modified as set forth in the Tables
KAFSPVRSV

SEQ ID NO: 307 - protein, 9 aa, Homo sapiens
  (1)..(9): One or more of the listed amino acids can be modified as set forth in the Tables
KAFSPVRSV

SEQ ID NO: 308 - protein, 10 aa, Homo sapiens
  (1)..(10): One or more of the listed amino acids can be modified as set forth in the Tables
KAFSPVRSVR

SEQ ID NO: 309 - protein, 10 aa, Homo sapiens
  (1)..(10): One or more of the listed amino acids can be modified as set forth in the Tables
KAFSPVRSVR

SEQ ID NO: 310 - protein, 11 aa, Homo sapiens
  (1)..(11): One or more of the listed amino acids can be modified as set forth in the Tables
KAFSPVRSVRK

SEQ ID NO: 311 - protein, 9 aa, Homo sapiens
  (1)..(9): One or more of the listed amino acids can be modified as set forth in the Tables
KAPSPPPLL

SEQ ID NO: 312 - protein, 9 aa, Homo sapiens
  (1)..(9): One or more of the listed amino acids can be modified as set forth in the Tables
KAPSRQISL

SEQ ID NO: 313 - protein, 9 aa, Homo sapiens
  (1)..(9): One or more of the listed amino acids can be modified as set forth in the Tables
KAPSRQISL

SEQ ID NO: 314 - protein, 9 aa, Homo sapiens
  (1)..(9): One or more of the listed amino acids can be modified as set forth in the Tables
KAPSRQISL

SEQ ID NO: 315 - protein, 9 aa, Homo sapiens
  (1)..(9): One or more of the listed amino acids can be modified as set forth in the Tables
KAVSLFLCY

SEQ ID NO: 316 - protein, 9 aa, Homo sapiens
  (1)..(9): One or more of the listed amino acids can be modified as set forth in the Tables
KAVSLFLCY

SEQ ID NO: 317 - protein, 9 aa, Homo sapiens
  (1)..(9): One or more of the listed amino acids can be modified as set forth in the Tables
KEDSDEVHL

SEQ ID NO: 318 - protein, 9 aa, Homo sapiens
  (1)..(9): One or more of the listed amino acids can be modified as set forth in the Tables
KEKTIHLTL

SEQ ID NO: 319 - protein, 9 aa, Homo sapiens
  (1)..(9): One or more of the listed amino acids can be modified as set forth in the Tables
KELSPAGSI

SEQ ID NO: 320 - protein, 9 aa, Homo sapiens
  (1)..(9): One or more of the listed amino acids can be modified as set forth in the Tables
KEQSPEPHL

SEQ ID NO: 321 - protein, 9 aa, Homo sapiens
  (1)..(9): One or more of the listed amino acids can be modified as set forth in the Tables
KESTLHLVL

SEQ ID NO: 322 - protein, 9 aa, Homo sapiens
  (1)..(9): One or more of the listed amino acids can be modified as set forth in the Tables
KETPDKVEL

SEQ ID NO: 323 - protein, 13 aa, Homo sapiens
  (1)..(13): One or more of the listed amino acids can be modified as set forth in the Tables
KEVDPSTGELQSL

SEQ ID NO: 324 - protein, 13 aa, Homo sapiens
  (1)..(13): One or more of the listed amino acids can be modified as set forth in the Tables
KEVDPSTGELQSL

SEQ ID NO: 325 - protein, 10 aa, Homo sapiens
  (1)..(10): One or more of the listed amino acids can be modified as set forth in the Tables
KFLSPAQYLY

SEQ ID NO: 326 - protein, 8 aa, Homo sapiens
  (1)..(8): One or more of the listed amino acids can be modified as set forth in the Tables
KFSPVRSV

SEQ ID NO: 327 - protein, 9 aa, Homo sapiens
  (1)..(9): One or more of the listed amino acids can be modified as set forth in the Tables
KGFSGTFQL

SEQ ID NO: 328 - protein, 8 aa, Homo sapiens
  (1)..(8): One or more of the listed amino acids can be modified as set forth in the Tables
KIDSPTKV

SEQ ID NO: 329 - protein, 9 aa, Homo sapiens
  (1)..(9): One or more of the listed amino acids can be modified as set forth in the Tables
KIDSPTKVK

SEQ ID NO: 330 - protein, 9 aa, Homo sapiens
  (1)..(9): One or more of the listed amino acids can be modified as set forth in the Tables
KIHTLELKL

SEQ ID NO: 331 - protein, 10 aa, Homo sapiens
  (1)..(10): One or more of the listed amino acids can be modified as set forth in the Tables
KIISAELQKA

SEQ ID NO: 332 - protein, 8 aa, Homo sapiens
  (1)..(8): One or more of the listed amino acids can be modified as set forth in the Tables
KIISIFSG

SEQ ID NO: 333 - protein, 11 aa, Homo sapiens
  (1)..(11): One or more of the listed amino acids can be modified as set forth in the Tables
KIISIFSGTEK

SEQ ID NO: 334 - protein, 9 aa, Homo sapiens
  (1)..(9): One or more of the listed amino acids can be modified as set forth in the Tables
KIKSFVKVY

SEQ ID NO: 335 - protein, 10 aa, Homo sapiens
  (1)..(10): One or more of the listed amino acids can be modified as set forth in the Tables
KIKSLEEIYL

SEQ ID NO: 336 - protein, 9 aa, Homo sapiens
  (1)..(9): One or more of the listed amino acids can be modified as set forth in the Tables
KIMSPRKAL

SEQ ID NO: 337 - protein, 9 aa, Homo sapiens
  (1)..(9): One or more of the listed amino acids can be modified as set forth in the Tables
KIMSPRKAL

SEQ ID NO: 338 - protein, 9 aa, Homo sapiens
  (1)..(9): One or more of the listed amino acids can be modified as set forth in the Tables
KIMSSPLSK

SEQ ID NO: 339 - protein, 9 aa, Homo sapiens
  (1)..(9): One or more of the listed amino acids can be modified as set forth in the Tables
KIMSSPLSK

SEQ ID NO: 340 - protein, 9 aa, Homo sapiens
  (1)..(9): One or more of the listed amino acids can be modified as set forth in the Tables
KIRPHIATL

SEQ ID NO: 341 - protein, 9 aa, Homo sapiens
  (1)..(9): One or more of the listed amino acids can be modified as set forth in the Tables
KISSLEIKL

SEQ ID NO: 342 - protein, 12 aa, Homo sapiens
  (1)..(12): One or more of the listed amino acids can be modified as set forth in the Tables
KLASPSEVVQQV

SEQ ID NO: 343 - protein, 10 aa, Homo sapiens
  (1)..(10): One or more of the listed amino acids can be modified as set forth in the Tables
KLFHGSLEEL

SEQ ID NO: 344 - protein, 10 aa, Homo sapiens
  (1)..(10): One or more of the listed amino acids can be modified as set forth in the Tables
KLHSLIGLGI

SEQ ID NO: 345 - protein, 11 aa, Homo sapiens
  (1)..(11): One or more of the listed amino acids can be modified as set forth in the Tables
KLLDFGSLSNL

SEQ ID NO: 346 - protein, 13 aa, Homo sapiens
  (1)..(13): One or more of the listed amino acids can be modified as set forth in the Tables
KLLDFGSLSNLQV

SEQ ID NO: 347 - protein, 9 aa, Homo sapiens
  (1)..(9): One or more of the listed amino acids can be modified as set forth in the Tables
KLLSYIQRL

SEQ ID NO: 348 - protein, 11 aa, Homo sapiens
  (1)..(11): One or more of the listed amino acids can be modified as set forth in the Tables
KLMSPKADVKL

SEQ ID NO: 349 - protein, 9 aa, Homo sapiens
  (1)..(9): One or more of the listed amino acids can be modified as set forth in the Tables
KLPSGSKKV

SEQ ID NO: 350 - protein, 9 aa, Homo sapiens
  (1)..(9): One or more of the listed amino acids can be modified as set forth in the Tables
KLRSPKSEL

SEQ ID NO: 351 - protein, 12 aa, Homo sapiens
  (1)..(12): One or more of the listed amino acids can be modified as set forth in the Tables
KLWTLVSEQTRV

SEQ ID NO: 352 - protein, 9 aa, Homo sapiens
  (1)..(9): One or more of the listed amino acids can be modified as set forth in the Tables
KLYSISSQV

SEQ ID NO: 353 - protein, 9 aa, Homo sapiens
  (1)..(9): One or more of the listed amino acids can be modified as set forth in the Tables
KLYTYIQSR

SEQ ID NO: 354 - protein, 10 aa, Homo sapiens
  (1)..(10): One or more of the listed amino acids can be modified as set forth in the Tables
KLYTYIQSRF

SEQ ID NO: 355 - protein, 9 aa, Homo sapiens
  (1)..(9): One or more of the listed amino acids can be modified as set forth in the Tables
KMASLARKV

SEQ ID NO: 356 - protein, 10 aa, Homo sapiens
  (1)..(10): One or more of the listed amino acids can be modified as set forth in the Tables
KMDSFLDMQL

SEQ ID NO: 357 - protein, 10 aa, Homo sapiens
  (1)..(10): One or more of the listed amino acids can be modified as set forth in the Tables
KMDSFLDMQL

SEQ ID NO: 358 - protein, 10 aa, Homo sapiens
  (1)..(10): One or more of the listed amino acids can be modified as set forth in the Tables
KMDSFLDMQL

SEQ ID NO: 359 - protein, 11 aa, Homo sapiens
  (1)..(11): One or more of the listed amino acids can be modified as set forth in the Tables
KMLSCAGADRL

SEQ ID NO: 360 - protein, 11 aa, Homo sapiens
  (1)..(11): One or more of the listed amino acids can be modified as set forth in the Tables
KMLSCAGADRL

SEQ ID NO: 361 - protein, 11 aa, Homo sapiens
  (1)..(11): One or more of the listed amino acids can be modified as set forth in the Tables
KMLSCAGADRL

SEQ ID NO: 362 - protein, 9 aa, Homo sapiens
KMLTPRIEL

SEQ ID NO: 363 - protein, 9 aa, Homo sapiens
  (1)..(9): One or more of the listed amino acids can be modified as set forth in the Tables
KMLTPRIEL

SEQ ID NO: 364 - protein, 9 aa, Homo sapiens
  (1)..(9): One or more of the listed amino acids can be modified as set forth in the Tables
KMSSYAFFV

SEQ ID NO: 365 - protein, 10 aa, Homo sapiens
  (1)..(10): One or more of the listed amino acids can be modified as set forth in the Tables
KMVSMKPPGF

SEQ ID NO: 366 - protein, 10 aa, Homo sapiens
  (1)..(10): One or more of the listed amino acids can be modified as set forth in the Tables
KMVSMKPPGF

SEQ ID NO: 367 - protein, 10 aa, Homo sapiens
  (1)..(10): One or more of the listed amino acids can be modified as set forth in the Tables
KMVSMKPPGF

SEQ ID NO: 368 - protein, 11 aa, Homo sapiens
  (1)..(11): One or more of the listed amino acids can be modified as set forth in the Tables
KPASPARRLDL

SEQ ID NO: 369 - protein, 9 aa, Homo sapiens
  (1)..(9): One or more of the listed amino acids can be modified as set forth in the Tables
KPASPTPVI

SEQ ID NO: 370 - protein, 12 aa, Homo sapiens
  (1)..(12): One or more of the listed amino acids can be modified as set forth in the Tables
KPAVSRSRSSSL

SEQ ID NO: 371 - protein, 10 aa, Homo sapiens
  (1)..(10): One or more of the listed amino acids can be modified as set forth in the Tables
KPFKLSGLSF

SEQ ID NO: 372 - protein, 9 aa, Homo sapiens
  (1)..(9): One or more of the listed amino acids can be modified as set forth in the Tables
KPGLGEGTP

SEQ ID NO: 373 - protein, 9 aa, Homo sapiens
  (1)..(9): One or more of the listed amino acids can be modified as set forth in the Tables
KPLIRSQSL

SEQ ID NO: 374 - protein, 10 aa, Homo sapiens
  (1)..(10): One or more of the listed amino acids can be modified as set forth in the Tables
KPMTPKVVTL

SEQ ID NO: 375 - protein, 10 aa, Homo sapiens
  (1)..(10): One or more of the listed amino acids can be modified as set forth in the Tables
KPMTPKVVTL

SEQ ID NO: 376 - protein, 11 aa, Homo sapiens
  (1)..(11): One or more of the listed amino acids can be modified as set forth in the Tables
KPPGTPPPSAL

SEQ ID NO: 377 - protein, 9 aa, Homo sapiens
  (1)..(9): One or more of the listed amino acids can be modified as set forth in the Tables
KPPSPGTVL

SEQ ID NO: 378 - protein, 11 aa, Homo sapiens
  (1)..(11): One or more of the listed amino acids can be modified as set forth in the Tables
KPPSPGTVLAL

SEQ ID NO: 379 - protein, 9 aa, Homo sapiens
  (1)..(9): One or more of the listed amino acids can be modified as set forth in the Tables
KPRRFSRSL

SEQ ID NO: 380 - protein, 10 aa, Homo sapiens
  (1)..(10): One or more of the listed amino acids can be modified as set forth in the Tables
KPSSLRRVTI

SEQ ID NO: 381 - protein, 9 aa, Homo sapiens
  (1)..(9): One or more of the listed amino acids can be modified as set forth in the Tables
KPVGVAASL

SEQ ID NO: 382 - protein, 8 aa, Homo sapiens
  (1)..(8): One or more of the listed amino acids can be modified as set forth in the Tables
KPVSPLLL

SEQ ID NO: 383 - protein, 10 aa, Homo sapiens
  (1)..(10): One or more of the listed amino acids can be modified as set forth in the Tables
KQKSLTNLSF

SEQ ID NO: 384 - protein, 10 aa, Homo sapiens
  (1)..(10): One or more of the listed amino acids can be modified as set forth in the Tables
KQPSFSAKKM

SEQ ID NO: 385 - protein, 8 aa, Homo sapiens
  (1)..(8): One or more of the listed amino acids can be modified as set forth in the Tables
KQYSGKFF

SEQ ID NO: 386 - protein, 9 aa, Homo sapiens
  (1)..(9): One or more of the listed amino acids can be modified as set forth in the Tables
KRASALLNL

SEQ ID NO: 387 - protein, 9 aa, Homo sapiens
  (1)..(9): One or more of the listed amino acids can be modified as set forth in the Tables
KRASRIYNT

SEQ ID NO: 388 - protein, 11 aa, Homo sapiens
  (1)..(11): One or more of the listed amino acids can be modified as set forth in the Tables
KRFSFKKSFKL

SEQ ID NO: 389 - protein, 9 aa, Homo sapiens
  (1)..(9): One or more of the listed amino acids can be modified as set forth in the Tables
KRFSLDFNL

SEQ ID NO: 390 - protein, 9 aa, Homo sapiens
  (1)..(9): One or more of the listed amino acids can be modified as set forth in the Tables
KRISIFLSM

SEQ ID NO: 391 - protein, 9 aa, Homo sapiens
  (1)..(9): One or more of the listed amino acids can be modified as set forth in the Tables
KRISIFLSM

SEQ ID NO: 392 - protein, 12 aa, Homo sapiens
  (1)..(12): One or more of the listed amino acids can be modified as set forth in the Tables
KRISISTSGGSF

SEQ ID NO: 393 - protein, 9 aa, Homo sapiens
KRISRMRLV

SEQ ID NO: 394 - protein, 11 aa, Homo sapiens
  (1)..(11): One or more of the listed amino acids can be modified as set forth in the Tables
KRLSVELTSSL

SEQ ID NO: 395 - protein, 12 aa, Homo sapiens
  (1)..(12): One or more of the listed amino acids can be modified as set forth in the Tables
KRLSVELTSSLF

SEQ ID NO: 396 - protein, 11 aa, Homo sapiens
  (1)..(11): One or more of the listed amino acids can be modified as set forth in the Tables
KRLSVERIYQK

SEQ ID NO: 397 - protein, 9 aa, Homo sapiens
  (1)..(9): One or more of the listed amino acids can be modified as set forth in the Tables
KRLTHVYDL

SEQ ID NO: 398 - protein, 10 aa, Homo sapiens
  (1)..(10): One or more of the listed amino acids can be modified as set forth in the Tables
KRMSNELENY

SEQ ID NO: 399 - protein, 12 aa, Homo sapiens
  (1)..(12): One or more of the listed amino acids can be modified as set forth in the Tables
KRMSVTEGGIKY

SEQ ID NO: 400 - protein, 9 aa, Homo sapiens
  (1)..(9): One or more of the listed amino acids can be modified as set forth in the Tables
KRNSIKKIV

SEQ ID NO: 401 - protein, 9 aa, Homo sapiens
  (1)..(9): One or more of the listed amino acids can be modified as set forth in the Tables
KRNSRLGFL

SEQ ID NO: 402 - protein, 10 aa, Homo sapiens
  (1)..(10): One or more of the listed amino acids can be modified as set forth in the Tables
KRNTFVGTPF

SEQ ID NO: 403 - protein, 9 aa, Homo sapiens
  (1)..(9): One or more of the listed amino acids can be modified as set forth in the Tables
KRRTGALVL

SEQ ID NO: 404 - protein, 7 aa, Homo sapiens
  (1)..(7): One or more of the listed amino acids can be modified as set forth in the Tables
KRSPIFF

SEQ ID NO: 405 - protein, 9 aa, Homo sapiens
  (1)..(9): One or more of the listed amino acids can be modified as set forth in the Tables
KRSSISQLL

SEQ ID NO: 406 - protein, 10 aa, Homo sapiens
KRSSVHGVSF

SEQ ID NO: 407 - protein, 9 aa, Homo sapiens
  (1)..(9): One or more of the listed amino acids can be modified as set forth in the Tables
KRTSKYFSL

SEQ ID NO: 408 - protein, 14 aa, Homo sapiens
  (1)..(14): One or more of the listed amino acids can be modified as set forth in the Tables
KRVSISEGDDKIEY

SEQ ID NO: 409 - protein, 9 aa, Homo sapiens
  (1)..(9): One or more of the listed amino acids can be modified as set forth in the Tables
KRYSAEVRL

SEQ ID NO: 410 - protein, 9 aa, Homo sapiens
  (1)..(9): One or more of the listed amino acids can be modified as set forth in the Tables
KRYSRSLTI

SEQ ID NO: 411 - protein, 9 aa, Homo sapiens
  (1)..(9): One or more of the listed amino acids can be modified as set forth in the Tables
KSDGSFIGY

SEQ ID NO: 412 - protein, 9 aa, Homo sapiens
  (1)..(9): One or more of the listed amino acids can be modified as set forth in the Tables
KSDSPAIQL

SEQ ID NO: 413 - protein, 10 aa, Homo sapiens
  (1)..(10): One or more of the listed amino acids can be modified as set forth in the Tables
KSDSPSTSSI

SEQ ID NO: 414 - protein, 9 aa, Homo sapiens
  (1)..(9): One or more of the listed amino acids can be modified as set forth in the Tables
KSKSMDLGI

SEQ ID NO: 415 - protein, 10 aa, Homo sapiens
  (1)..(10): One or more of the listed amino acids can be modified as set forth in the Tables
KSLSPSGLKI

SEQ ID NO: 416 - protein, 10 aa, Homo sapiens
  (1)..(10): One or more of the listed amino acids can be modified as set forth in the Tables
KSLSPSLLGY

SEQ ID NO: 417 - protein, 9 aa, Homo sapiens
  (1)..(9): One or more of the listed amino acids can be modified as set forth in the Tables
KSPTSPLNM

SEQ ID NO: 418 - protein, 8 aa, Homo sapiens
  (1)..(8): One or more of the listed amino acids can be modified as set forth in the Tables
KSSIIIRM

SEQ ID NO: 419 - protein, 9 aa, Homo sapiens
  (1)..(9): One or more of the listed amino acids can be modified as set forth in the Tables
KSSSLDKQL

SEQ ID NO: 420 - protein, 9 aa, Homo sapiens
  (1)..(9): One or more of the listed amino acids can be modified as set forth in the Tables
KSSSLDKQL

SEQ ID NO: 421 - protein, 11 aa, Homo sapiens
  (1)..(11): One or more of the listed amino acids can be modified as set forth in the Tables
KSYSFIARMKA

SEQ ID NO: 422 - protein, 11 aa, Homo sapiens
  (1)..(11): One or more of the listed amino acids can be modified as set forth in the Tables
KSYSFIARMKA

SEQ ID NO: 423 - protein, 9 aa, Homo sapiens
  (1)..(9): One or more of the listed amino acids can be modified as set forth in the Tables
KSYSRSRSR

SEQ ID NO: 424 - protein, 9 aa, Homo sapiens
  (1)..(9): One or more of the listed amino acids can be modified as set forth in the Tables
KTDGSFIGY

SEQ ID NO: 425 - protein, 11 aa, Homo sapiens
  (1)..(11): One or more of the listed amino acids can be modified as set forth in the Tables
KTESPRTSGVL

SEQ ID NO: 426 - protein, 10 aa, Homo sapiens
  (1)..(10): One or more of the listed amino acids can be modified as set forth in the Tables
KTFSIGKIAK

SEQ ID NO: 427 - protein, 9 aa, Homo sapiens
  (1)..(9): One or more of the listed amino acids can be modified as set forth in the Tables
KTISLTDFL

SEQ ID NO: 428 - protein, 9 aa, Homo sapiens
  (1)..(9): One or more of the listed amino acids can be modified as set forth in the Tables
KTKSIAEEL

SEQ ID NO: 429 - protein, 9 aa, Homo sapiens
  (1)..(9): One or more of the listed amino acids can be modified as set forth in the Tables
KTKSMFFFL

SEQ ID NO: 430 - protein, 9 aa, Homo sapiens
  (1)..(9): One or more of the listed amino acids can be modified as set forth in the Tables
KTLSLVKEL

SEQ ID NO: 431 - protein, 9 aa, Homo sapiens
  (1)..(9): One or more of the listed amino acids can be modified as set forth in the Tables
KTMSGTFLL

SEQ ID NO: 432 - protein, 9 aa, Homo sapiens
  (1)..(9): One or more of the listed amino acids can be modified as set forth in the Tables
KTMSPSQMI

SEQ ID NO: 433 - protein, 9 aa, Homo sapiens
  (1)..(9): One or more of the listed amino acids can be modified as set forth in the Tables
KTPSHTRML

SEQ ID NO: 434 - protein, 9 aa, Homo sapiens
  (1)..(9): One or more of the listed amino acids can be modified as set forth in the Tables
KTPSLTRRI

SEQ ID NO: 435 - protein, 9 aa, Homo sapiens
  (1)..(9): One or more of the listed amino acids can be modified as set forth in the Tables
KTPTSPLKM

SEQ ID NO: 436 - protein, 9 aa, Homo sapiens
  (1)..(9): One or more of the listed amino acids can be modified as set forth in the Tables
KTPTSPLKM

SEQ ID NO: 437 - protein, 10 aa, Homo sapiens
  (1)..(10): One or more of the listed amino acids can be modified as set forth in the Tables
KTQSLPVTEK

SEQ ID NO: 438 - protein, 9 aa, Homo sapiens
  (1)..(9): One or more of the listed amino acids can be modified as set forth in the Tables
KTRSLSVEI

SEQ ID NO: 439 - protein, 11 aa, Homo sapiens
  (1)..(11): One or more of the listed amino acids can be modified as set forth in the Tables
KTRSLSVEIVY

SEQ ID NO: 440 - protein, 11 aa, Homo sapiens
  (1)..(11): One or more of the listed amino acids can be modified as set forth in the Tables
KTRSLSVEIVY

SEQ ID NO: 441 - protein, 10 aa, Homo sapiens
  (1)..(10): One or more of the listed amino acids can be modified as set forth in the Tables
KTVSEPNLKL

SEQ ID NO: 442 - protein, 9 aa, Homo sapiens
  (1)..(9): One or more of the listed amino acids can be modified as set forth in the Tables
KTVSPSPAF

SEQ ID NO: 443 - protein, 11 aa, Homo sapiens
  (1)..(11): One or more of the listed amino acids can be modified as set forth in the Tables
KVDSPTVTTTL

SEQ ID NO: 444 - protein, 9 aa, Homo sapiens
  (1)..(9): One or more of the listed amino acids can be modified as set forth in the Tables
KVIPVTRSL

SEQ ID NO: 445 - protein, 9 aa, Homo sapiens
  (1)..(9): One or more of the listed amino acids can be modified as set forth in the Tables
KVLSSLVTL

SEQ ID NO: 446 - protein, 9 aa, Homo sapiens
  (1)..(9): One or more of the listed amino acids can be modified as set forth in the Tables
KVYSSSEFL

SEQ ID NO: 447 - protein, 9 aa, Homo sapiens
  (1)..(9): One or more of the listed amino acids can be modified as set forth in the Tables
KVYSSSEFL

SEQ ID NO: 448 - protein, 9 aa, Homo sapiens
  (1)..(9): One or more of the listed amino acids can be modified as set forth in the Tables
KVYTPSISK

SEQ ID NO: 449 - protein, 8 aa, Homo sapiens
  (1)..(8): One or more of the listed amino acids can be modified as set forth in the Tables
KYELSVIM

SEQ ID NO: 450 - protein, 10 aa, Homo sapiens
  (1)..(10): One or more of the listed amino acids can be modified as set forth in the Tables
KYPDVASPTL

SEQ ID NO: 451 - protein, 8 aa, Homo sapiens
  (1)..(8): One or more of the listed amino acids can be modified as set forth in the Tables
LADSPLKL

SEQ ID NO: 452 - protein, 9 aa, Homo sapiens
  (1)..(9): One or more of the listed amino acids can be modified as set forth in the Tables
LALTRSSSL

SEQ ID NO: 453 - protein, 10 aa, Homo sapiens
  (1)..(10): One or more of the listed amino acids can be modified as set forth in the Tables
LDEAGQRSTM

SEQ ID NO: 454 - protein, 10 aa, Homo sapiens
  (1)..(10): One or more of the listed amino acids can be modified as set forth in the Tables
LDEAGQRSTM

SEQ ID NO: 455 - protein, 9 aa, Homo sapiens
  (1)..(9): One or more of the listed amino acids can be modified as set forth in the Tables
LEAPPSPSL

SEQ ID NO: 456 - protein, 11 aa, Homo sapiens
  (1)..(11): One or more of the listed amino acids can be modified as set forth in the Tables
LEITPPSSEKL

SEQ ID NO: 457 - protein, 9 aa, Homo sapiens
  (1)..(9): One or more of the listed amino acids can be modified as set forth in the Tables
LESPTTPLL

SEQ ID NO: 458 - protein, 9 aa, Homo sapiens
  (1)..(9): One or more of the listed amino acids can be modified as set forth in the Tables
LESPTTPLL

SEQ ID NO: 459 - protein, 9 aa, Homo sapiens
  (1)..(9): One or more of the listed amino acids can be modified as set forth in the Tables
LESPTTPLL

SEQ ID NO: 460 - protein, 9 aa, Homo sapiens
  (1)..(9): One or more of the listed amino acids can be modified as set forth in the Tables
LIDNSFNRY

SEQ ID NO: 461 - protein, 9 aa, Homo sapiens
  (1)..(9): One or more of the listed amino acids can be modified as set forth in the Tables
LIMPRPNSV

SEQ ID NO: 462 - protein, 9 aa, Homo sapiens
  (1)..(9): One or more of the listed amino acids can be modified as set forth in the Tables
LLARTPPAA

SEQ ID NO: 463 - protein, 9 aa, Homo sapiens
  (1)..(9): One or more of the listed amino acids can be modified as set forth in the Tables
LLNKTPPTA

SEQ ID NO: 464 - protein, 9 aa, Homo sapiens
  (1)..(9): One or more of the listed amino acids can be modified as set forth in the Tables
LMHSFILKA

SEQ ID NO: 465 - protein, 9 aa, Homo sapiens
  (1)..(9): One or more of the listed amino acids can be modified as set forth in the Tables
LPAFKRKTL

SEQ ID NO: 466 - protein, 9 aa, Homo sapiens
  (1)..(9): One or more of the listed amino acids can be modified as set forth in the Tables
LPASPAGRL

SEQ ID NO: 467 - protein, 9 aa, Homo sapiens
  (1)..(9): One or more of the listed amino acids can be modified as set forth in the Tables
LPASPAHQL

SEQ ID NO: 468 - protein, 11 aa, Homo sapiens
  (1)..(11): One or more of the listed amino acids can be modified as set forth in the Tables
LPASPRARLSA

SEQ ID NO: 469 - protein, 9 aa, Homo sapiens
  (1)..(9): One or more of the listed amino acids can be modified as set forth in the Tables
LPASPSVSL

SEQ ID NO: 470 - protein, 9 aa, Homo sapiens
  (1)..(9): One or more of the listed amino acids can be modified as set forth in the Tables
LPASPVARR

SEQ ID NO: 471 - protein, 9 aa, Homo sapiens
  (1)..(9): One or more of the listed amino acids can be modified as set forth in the Tables
LPDPGSPRL

SEQ ID NO: 472 - protein, 9 aa, Homo sapiens
  (1)..(9): One or more of the listed amino acids can be modified as set forth in the Tables
LPESPRLTL

SEQ ID NO: 473 - protein, 9 aa, Homo sapiens
  (1)..(9): One or more of the listed amino acids can be modified as set forth in the Tables
LPKARPMSL

SEQ ID NO: 474 - protein, 9 aa, Homo sapiens
  (1)..(9): One or more of the listed amino acids can be modified as set forth in the Tables
LPKARPMSL

SEQ ID NO: 475 - protein, 9 aa, Homo sapiens
  (1)..(9): One or more of the listed amino acids can be modified as set forth in the Tables
LPLSPKETV

SEQ ID NO: 476 - protein, 11 aa, Homo sapiens
  (1)..(11): One or more of the listed amino acids can be modified as set forth in the Tables
LPLSSSHLNVY

SEQ ID NO: 477 - protein, 9 aa, Homo sapiens
  (1)..(9): One or more of the listed amino acids can be modified as set forth in the Tables
LPNSIASRF

SEQ ID NO: 478 - protein, 10 aa, Homo sapiens
  (1)..(10): One or more of the listed amino acids can be modified as set forth in the Tables
LPRMISHSEL

SEQ ID NO: 479 - protein, 10 aa, Homo sapiens
  (1)..(10): One or more of the listed amino acids can be modified as set forth in the Tables
LPRMISHSEL

SEQ ID NO: 480 - protein, 10 aa, Homo sapiens
  (1)..(10): One or more of the listed amino acids can be modified as set forth in the Tables
LPRPLSPTKL

SEQ ID NO: 481 - protein, 10 aa, Homo sapiens
  (1)..(10): One or more of the listed amino acids can be modified as set forth in the Tables
LPRSPPLKVL

SEQ ID NO: 482 - protein, 9 aa, Homo sapiens
  (1)..(9): One or more of the listed amino acids can be modified as set forth in the Tables
LPRSPRLGH

SEQ ID NO: 483 - protein, 11 aa, Homo sapiens
  (1)..(11): One or more of the listed amino acids can be modified as set forth in the Tables
LPRSSSMAAGL

SEQ ID NO: 484 - protein, 10 aa, Homo sapiens
  (1)..(10): One or more of the listed amino acids can be modified as set forth in the Tables
LPRTPSASSL

SEQ ID NO: 485 - protein, 9 aa, Homo sapiens
  (1)..(9): One or more of the listed amino acids can be modified as set forth in the Tables
LPRTPSYSI

SEQ ID NO: 486 - protein, 9 aa, Homo sapiens
  (1)..(9): One or more of the listed amino acids can be modified as set forth in the Tables
LPSESVSSL

SEQ ID NO: 487 - protein, 10 aa, Homo sapiens
  (1)..(10): One or more of the listed amino acids can be modified as set forth in the Tables
LPSPRGQRVI

SEQ ID NO: 488 - protein, 10 aa, Homo sapiens
  (1)..(10): One or more of the listed amino acids can be modified as set forth in the Tables
LPSPTATSQL

SEQ ID NO: 489 - protein, 9 aa, Homo sapiens
  (1)..(9): One or more of the listed amino acids can be modified as set forth in the Tables
LPSSGRSSL

SEQ ID NO: 490 - protein, 10 aa, Homo sapiens
  (1)..(10): One or more of the listed amino acids can be modified as set forth in the Tables
LPTSPLAMEY

SEQ ID NO: 491 - protein, 10 aa, Homo sapiens
  (1)..(10): One or more of the listed amino acids can be modified as set forth in the Tables
LPTSPLAMEY

SEQ ID NO: 492 - protein, 10 aa, Homo sapiens
  (1)..(10): One or more of the listed amino acids can be modified as set forth in the Tables
LPVSPGHRKT

SEQ ID NO: 493 - protein, 11 aa, Homo sapiens
  (1)..(11): One or more of the listed amino acids can be modified as set forth in the Tables
LPYPVSPKQKY

SEQ ID NO: 494 - protein, 10 aa, Homo sapiens
  (1)..(10): One or more of the listed amino acids can be modified as set forth in the Tables
LQHSFSFAGF

SEQ ID NO: 495 - protein, 11 aa, Homo sapiens
  (1)..(11): One or more of the listed amino acids can be modified as set forth in the Tables
LQISPPLHQHL

SEQ ID NO: 496 - protein, 9 aa, Homo sapiens
  (1)..(9): One or more of the listed amino acids can be modified as set forth in the Tables
LQISPVSSY

SEQ ID NO: 497 - protein, 10 aa, Homo sapiens
  (1)..(10): One or more of the listed amino acids can be modified as set forth in the Tables
LQISPVSSYA

SEQ ID NO: 498 - protein, 9 aa, Homo sapiens
  (1)..(9): One or more of the listed amino acids can be modified as set forth in the Tables
LQLPSPTAT

SEQ ID NO: 499 - protein, 9 aa, Homo sapiens
  (1)..(9): One or more of the listed amino acids can be modified as set forth in the Tables
LSASFRSLY

SEQ ID NO: 500 - protein, 9 aa, Homo sapiens
  (1)..(9): One or more of the listed amino acids can be modified as set forth in the Tables
LSASPLTSL

SEQ ID NO: 501 - protein, 9 aa, Homo sapiens
  (1)..(9): One or more of the listed amino acids can be modified as set forth in the Tables
LSDDGKASL

SEQ ID NO: 502 - protein, 10 aa, Homo sapiens
  (1)..(10): One or more of the listed amino acids can be modified as set forth in the Tables
LSDSPSMGRY

SEQ ID NO: 503 - protein, 9 aa, Homo sapiens
  (1)..(9): One or more of the listed amino acids can be modified as set forth in the Tables
LSEIKFNSY

SEQ ID NO: 504 - protein, 9 aa, Homo sapiens
  (1)..(9): One or more of the listed amino acids can be modified as set forth in the Tables
LSKSEHSLF

SEQ ID NO: 505 - protein, 10 aa, Homo sapiens
  (1)..(10): One or more of the listed amino acids can be modified as set forth in the Tables
LSSSPPATHF

SEQ ID NO: 506 - protein, 10 aa, Homo sapiens
  (1)..(10): One or more of the listed amino acids can be modified as set forth in the Tables
LTDPSSPTIS

SEQ ID NO: 507 - protein, 9 aa, Homo sapiens
  (1)..(9): One or more of the listed amino acids can be modified as set forth in the Tables
LTHSLVLHY

SEQ ID NO: 508 - protein, 9 aa, Homo sapiens
  (1)..(9): One or more of the listed amino acids can be modified as set forth in the Tables
LTKSPLAQM

SEQ ID NO: 509 - protein, 9 aa, Homo sapiens
  (1)..(9): One or more of the listed amino acids can be modified as set forth in the Tables
LTLSPKLQL

SEQ ID NO: 510 - protein, 9 aa, Homo sapiens
  (1)..(9): One or more of the listed amino acids can be modified as set forth in the Tables
LTSSRLLKL

SEQ ID NO: 511 - protein, 9 aa, Homo sapiens
  (1)..(9): One or more of the listed amino acids can be modified as set forth in the Tables
LTYRRRLSY

SEQ ID NO: 512 - protein, 9 aa, Homo sapiens
  (1)..(9): One or more of the listed amino acids can be modified as set forth in the Tables
LVASPRLEK

SEQ ID NO: 513 - protein, 9 aa, Homo sapiens
  (1)..(9): One or more of the listed amino acids can be modified as set forth in the Tables
LVDSVAKTM

SEQ ID NO: 514 - protein, 9 aa, Homo sapiens
  (1)..(9): One or more of the listed amino acids can be modified as set forth in the Tables
LVDSVAKTM

SEQ ID NO: 515 - protein, 10 aa, Homo sapiens
  (1)..(10): One or more of the listed amino acids can be modified as set forth in the Tables
LVVSPGQQTL

SEQ ID NO: 516 - protein, 9 aa, Homo sapiens
  (1)..(9): One or more of the listed amino acids can be modified as set forth in the Tables
LYTYIQSRF

SEQ ID NO: 517 - protein, 11 aa, Homo sapiens
  (1)..(11): One or more of the listed amino acids can be modified as set forth in the Tables
MLAESPSVPRL

SEQ ID NO: 518 - protein, 9 aa, Homo sapiens
  (1)..(9): One or more of the listed amino acids can be modified as set forth in the Tables
MLPSILNQL

SEQ ID NO: 519 - protein, 10 aa, Homo sapiens
  (1)..(10): One or more of the listed amino acids can be modified as set forth in the Tables
MPGSPTKTVY

SEQ ID NO: 520 - protein, 10 aa, Homo sapiens
  (1)..(10): One or more of the listed amino acids can be modified as set forth in the Tables
MPGSPTKTVY

SEQ ID NO: 521 - protein, 9 aa, Homo sapiens
  (1)..(9): One or more of the listed amino acids can be modified as set forth in the Tables
MPHSPTLRV

SEQ ID NO: 522 - protein, 9 aa, Homo sapiens
  (1)..(9): One or more of the listed amino acids can be modified as set forth in the Tables
MPHSPTLRV

SEQ ID NO: 523 - protein, 9 aa, Homo sapiens
  (1)..(9): One or more of the listed amino acids can be modified as set forth in the Tables
MPKFRMPSL

SEQ ID NO: 524 - protein, 12 aa, Homo sapiens
  (1)..(12): One or more of the listed amino acids can be modified as set forth in the Tables
MPLSPDPSHTTL

SEQ ID NO: 525 - protein, 9 aa, Homo sapiens
  (1)..(9): One or more of the listed amino acids can be modified as set forth in the Tables
MPMRSPSKL

SEQ ID NO: 526 - protein, 9 aa, Homo sapiens
  (1)..(9): One or more of the listed amino acids can be modified as set forth in the Tables
MPNSPAPHF

SEQ ID NO: 527 - protein, 9 aa, Homo sapiens
  (1)..(9): One or more of the listed amino acids can be modified as set forth in the Tables
MPNSPAPHF

SEQ ID NO: 528 - protein, 10 aa, Homo sapiens
  (1)..(10): One or more of the listed amino acids can be modified as set forth in the Tables
MPREPSATRL

SEQ ID NO: 529 - protein, 10 aa, Homo sapiens
  (1)..(10): One or more of the listed amino acids can be modified as set forth in the Tables
MPREPSATRL

SEQ ID NO: 530 - protein, 10 aa, Homo sapiens
  (1)..(10): One or more of the listed amino acids can be modified as set forth in the Tables
MPRQPSATRL

SEQ ID NO: 531 - protein, 11 aa, Homo sapiens
  (1)..(11): One or more of the listed amino acids can be modified as set forth in the Tables
MPSPATLSHSL

SEQ ID NO: 532 - protein, 11 aa, Homo sapiens
  (1)..(11): One or more of the listed amino acids can be modified as set forth in the Tables
MPSPATLSHSL

SEQ ID NO: 533 - protein, 10 aa, Homo sapiens
  (1)..(10): One or more of the listed amino acids can be modified as set forth in the Tables
MPSPGGRITL

SEQ ID NO: 534 - protein, 10 aa, Homo sapiens
  (1)..(10): One or more of the listed amino acids can be modified as set forth in the Tables
MPSPGGRITL

SEQ ID NO: 535 - protein, 9 aa, Homo sapiens
  (1)..(9): One or more of the listed amino acids can be modified as set forth in the Tables
MPSPVSPKL

SEQ ID NO: 536 - protein, 9 aa, Homo sapiens
  (1)..(9): One or more of the listed amino acids can be modified as set forth in the Tables
MPSPVSPKL

SEQ ID NO: 537 - protein, 9 aa, Homo sapiens
  (1)..(9): One or more of the listed amino acids can be modified as set forth in the Tables
MPVPTTPEF

SEQ ID NO: 538 - protein, 9 aa, Homo sapiens
  (1)..(9): One or more of the listed amino acids can be modified as set forth in the Tables
MPVPTTPEF

SEQ ID NO: 539 - protein, 9 aa, Homo sapiens
  (1)..(9): One or more of the listed amino acids can be modified as set forth in the Tables
MRLSRELQL

SEQ ID NO: 540 - protein, 9 aa, Homo sapiens
  (1)..(9): One or more of the listed amino acids can be modified as set forth in the Tables
MTDTYRLKY

SEQ ID NO: 541 - protein, 9 aa, Homo sapiens
  (1)..(9): One or more of the listed amino acids can be modified as set forth in the Tables
MTKSSPLKI

SEQ ID NO: 542 - protein, 9 aa, Homo sapiens
  (1)..(9): One or more of the listed amino acids can be modified as set forth in the Tables
MTKSSPLKI

SEQ ID NO: 543 - protein, 9 aa, Homo sapiens
  (1)..(9): One or more of the listed amino acids can be modified as set forth in the Tables
MTKSSPLKI

SEQ ID NO: 544 - protein, 10 aa, Homo sapiens
  (1)..(10): One or more of the listed amino acids can be modified as set forth in the Tables
NAESGRGQVM

SEQ ID NO: 545 - protein, 10 aa, Homo sapiens
  (1)..(10): One or more of the listed amino acids can be modified as set forth in the Tables
NAESGRGQVM

SEQ ID NO: 546 - protein, 8 aa, Homo sapiens
  (1)..(8): One or more of the listed amino acids can be modified as set forth in the Tables
NAISLPTI

SEQ ID NO: 547 - protein, 9 aa, Homo sapiens
  (1)..(9): One or more of the listed amino acids can be modified as set forth in the Tables
NEFHSPIGL

SEQ ID NO: 548 - protein, 9 aa, Homo sapiens
  (1)..(9): One or more of the listed amino acids can be modified as set forth in the Tables
NGIIRSQSF

SEQ ID NO: 549 - protein, 11 aa, Homo sapiens
  (1)..(11): One or more of the listed amino acids can be modified as set forth in the Tables
NIASPGTVHKR

SEQ ID NO: 550 - protein, 9 aa, Homo sapiens
  (1)..(9): One or more of the listed amino acids can be modified as set forth in the Tables
NIPSFIVRL

SEQ ID NO: 551 - protein, 11 aa, Homo sapiens
  (1)..(11): One or more of the listed amino acids can be modified as set forth in the Tables
NLISPVRNGAV

SEQ ID NO: 552 - protein, 9 aa, Homo sapiens
  (1)..(9): One or more of the listed amino acids can be modified as set forth in the Tables
NMDSPGPML

SEQ ID NO: 553 - protein, 9 aa, Homo sapiens
  (1)..(9): One or more of the listed amino acids can be modified as set forth in the Tables
NMDSPGPML

SEQ ID NO: 554 - protein, 9 aa, Homo sapiens
  (1)..(9): One or more of the listed amino acids can be modified as set forth in the Tables
NMDSPGPML

SEQ ID NO: 555 - protein, 9 aa, Homo sapiens
  (1)..(9): One or more of the listed amino acids can be modified as set forth in the Tables
NPSSPEFFM

SEQ ID NO: 556 - protein, 9 aa, Homo sapiens
  (1)..(9): One or more of the listed amino acids can be modified as set forth in the Tables
NRFSPKASL

SEQ ID NO: 557 - protein, 9 aa, Homo sapiens
  (1)..(9): One or more of the listed amino acids can be modified as set forth in the Tables
NRLSKGLQI

SEQ ID NO: 558 - protein, 9 aa, Homo sapiens
  (1)..(9): One or more of the listed amino acids can be modified as set forth in the Tables
NRMSRRIVL

SEQ ID NO: 559 - protein, 9 aa, Homo sapiens
  (1)..(9): One or more of the listed amino acids can be modified as set forth in the Tables
NRMSRRIVL

SEQ ID NO: 560 - protein, 9 aa, Homo sapiens
  (1)..(9): One or more of the listed amino acids can be modified as set forth in the Tables
NRRKSALAL

SEQ ID NO: 561 - protein, 9 aa, Homo sapiens
  (1)..(9): One or more of the listed amino acids can be modified as set forth in the Tables
NRRSPPPSL

SEQ ID NO: 562 - protein, 9 aa, Homo sapiens
  (1)..(9): One or more of the listed amino acids can be modified as set forth in the Tables
NSDLPTSPL

SEQ ID NO: 563 - protein, 9 aa, Homo sapiens
  (1)..(9): One or more of the listed amino acids can be modified as set forth in the Tables
NSLSPRSSL

SEQ ID NO: 564 - protein, 9 aa, Homo sapiens
  (1)..(9): One or more of the listed amino acids can be modified as set forth in the Tables
NSVSPSESL

SEQ ID NO: 565 - protein, 9 aa, Homo sapiens
  (1)..(9): One or more of the listed amino acids can be modified as set forth in the Tables
NYQLSPTKL

SEQ ID NO: 566 - protein, 9 aa, Homo sapiens
  (1)..(9): One or more of the listed amino acids can be modified as set forth in the Tables
PEVSPRPAL

SEQ ID NO: 567 - protein, 9 aa, Homo sapiens
  (1)..(9): One or more of the listed amino acids can be modified as set forth in the Tables
PFKVSPLTF

SEQ ID NO: 568 - protein, 8 aa, Homo sapiens
  (1)..(8): One or more of the listed amino acids can be modified as set forth in the Tables
PIFNRISV

SEQ ID NO: 569 - protein, 9 aa, Homo sapiens
  (1)..(9): One or more of the listed amino acids can be modified as set forth in the Tables
PIFPMARSI

SEQ ID NO: 570 - protein, 16 aa, Homo sapiens
  (1)..(16): One or more of the listed amino acids can be modified as set forth in the Tables
PLVSSSDSPPRPQPAF

SEQ ID NO: 571 - protein, 8 aa, Homo sapiens
  (1)..(8): One or more of the listed amino acids can be modified as set forth in the Tables
PRSPPRAL

SEQ ID NO: 572 - protein, 12 aa, Homo sapiens
  (1)..(12): One or more of the listed amino acids can be modified as set forth in the Tables
PSPPSPLEKTPL

SEQ ID NO: 573 - protein, 9 aa, Homo sapiens
  (1)..(9): One or more of the listed amino acids can be modified as set forth in the Tables
PTSPLAMEY

SEQ ID NO: 574 - protein, 11 aa, Homo sapiens
  (1)..(11): One or more of the listed amino acids can be modified as set forth in the Tables
PWIPPSSPTTF

SEQ ID NO: 575 - protein, 13 aa, Homo sapiens
  (1)..(13): One or more of the listed amino acids can be modified as set forth in the Tables
PYDPALGSPSRLF

SEQ ID NO: 576 - protein, 9 aa, Homo sapiens
  (1)..(9): One or more of the listed amino acids can be modified as set forth in the Tables
QAFLRSVSM

SEQ ID NO: 577 - protein, 9 aa, Homo sapiens
  (1)..(9): One or more of the listed amino acids can be modified as set forth in the Tables
QEKSPKQAL

SEQ ID NO: 578 - protein, 8 aa, Homo sapiens
  (1)..(8): One or more of the listed amino acids can be modified as set forth in the Tables
QKKISTNL

SEQ ID NO: 579 - protein, 9 aa, Homo sapiens
  (1)..(9): One or more of the listed amino acids can be modified as set forth in the Tables
QLDRISVYY

SEQ ID NO: 580 - protein, 9 aa, Homo sapiens
  (1)..(9): One or more of the listed amino acids can be modified as set forth in the Tables
QLSLRTVSL

SEQ ID NO: 581 - protein, 14 aa, Homo sapiens
  (1)..(14): One or more of the listed amino acids can be modified as set forth in the Tables
QPRNSLPASPAHQL

SEQ ID NO: 582 - protein, 10 aa, Homo sapiens
  (1)..(10): One or more of the listed amino acids can be modified as set forth in the Tables
QPRSPVPSAF

SEQ ID NO: 583 - protein, 10 aa, Homo sapiens
  (1)..(10): One or more of the listed amino acids can be modified as set forth in the Tables
QPRTPHSPPL

SEQ ID NO: 584 - protein, 10 aa, Homo sapiens
  (1)..(10): One or more of the listed amino acids can be modified as set forth in the Tables
QPRTPHSPPL

SEQ ID NO: 585 - protein, 10 aa, Homo sapiens
  (1)..(10): One or more of the listed amino acids can be modified as set forth in the Tables
QPRTPSPLVL

SEQ ID NO: 586 - protein, 10 aa, Homo sapiens
  (1)..(10): One or more of the listed amino acids can be modified as set forth in the Tables
QPSSPRVNGL

SEQ ID NO: 587 - protein, 9 aa, Homo sapiens
  (1)..(9): One or more of the listed amino acids can be modified as set forth in the Tables
QPSTPDPFL

SEQ ID NO: 588 - protein, 9 aa, Homo sapiens
  (1)..(9): One or more of the listed amino acids can be modified as set forth in the Tables
QSLLSPLVL

SEQ ID NO: 589 - protein, 9 aa, Homo sapiens
  (1)..(9): One or more of the listed amino acids can be modified as set forth in the Tables
QTISPLSTY

SEQ ID NO: 590 - protein, 9 aa, Homo sapiens
  (1)..(9): One or more of the listed amino acids can be modified as set forth in the Tables
QTPSPRLAL

SEQ ID NO: 591 - protein, 10 aa, Homo sapiens
  (1)..(10): One or more of the listed amino acids can be modified as set forth in the Tables
QTSIQSPSSY

SEQ ID NO: 592 - protein, 10 aa, Homo sapiens
  (1)..(10): One or more of the listed amino acids can be modified as set forth in the Tables
QVDPKKRISM

SEQ ID NO: 593 - protein, 9 aa, Homo sapiens
  (1)..(9): One or more of the listed amino acids can be modified as set forth in the Tables
RAASTARHL

SEQ ID NO: 594 - protein, 9 aa, Homo sapiens
  (1)..(9): One or more of the listed amino acids can be modified as set forth in the Tables
RAATPLPSL

SEQ ID NO: 595 - protein, 9 aa, Homo sapiens
  (1)..(9): One or more of the listed amino acids can be modified as set forth in the Tables
RADSPGRLV

SEQ ID NO: 596 - protein, 8 aa, Homo sapiens
  (1)..(8): One or more of the listed amino acids can be modified as set forth in the Tables
RADSPVHM

SEQ ID NO: 597 - protein, 9 aa, Homo sapiens
  (1)..(9): One or more of the listed amino acids can be modified as set forth in the Tables
RADSPVHME

SEQ ID NO: 598 - protein, 10 aa, Homo sapiens
  (1)..(10): One or more of the listed amino acids can be modified as set forth in the Tables
RADSPVHMEQ

SEQ ID NO: 599 - protein, 11 aa, Homo sapiens
  (1)..(11): One or more of the listed amino acids can be modified as set forth in the Tables
RADSPVHMEQQ

SEQ ID NO: 600 - protein, 9 aa, Homo sapiens
  (1)..(9): One or more of the listed amino acids can be modified as set forth in the Tables
RAESDFVKF

SEQ ID NO: 601 - protein, 10 aa, Homo sapiens
  (1)..(10): One or more of the listed amino acids can be modified as set forth in the Tables
RAESGPDLRY

SEQ ID NO: 602 - protein, 11 aa, Homo sapiens
  (1)..(11): One or more of the listed amino acids can be modified as set forth in the Tables
RAESPGPGSRL

SEQ ID NO: 603 - protein, 9 aa, Homo sapiens
  (1)..(9): One or more of the listed amino acids can be modified as set forth in the Tables
RAESPTPGM

SEQ ID NO: 604 - protein, 9 aa, Homo sapiens
  (1)..(9): One or more of the listed amino acids can be modified as set forth in the Tables
RAESPTPGM

SEQ ID NO: 605 - protein, 9 aa, Homo sapiens
  (1)..(9): One or more of the listed amino acids can be modified as set forth in the Tables
RAGSFSRFY

SEQ ID NO: 606 - protein, 9 aa, Homo sapiens
  (1)..(9): One or more of the listed amino acids can be modified as set forth in the Tables
RAHSLARQM

SEQ ID NO: 607 - protein, 11 aa, Homo sapiens
  (1)..(11): One or more of the listed amino acids can be modified as set forth in the Tables
RAHTPTPGIYM

SEQ ID NO: 608 - protein, 11 aa, Homo sapiens
  (1)..(11): One or more of the listed amino acids can be modified as set forth in the Tables
RAHTPTPGIYM

SEQ ID NO: 609 - protein, 9 aa, Homo sapiens
  (1)..(9): One or more of the listed amino acids can be modified as set forth in the Tables
RAISPREKI

SEQ ID NO: 610 - protein, 9 aa, Homo sapiens
  (1)..(9): One or more of the listed amino acids can be modified as set forth in the Tables
RAKRISQLF

SEQ ID NO: 611 - protein, 9 aa, Homo sapiens
  (1)..(9): One or more of the listed amino acids can be modified as set forth in the Tables
RALSPRVAA

SEQ ID NO: 612 - protein, 11 aa, Homo sapiens
  (1)..(11): One or more of the listed amino acids can be modified as set forth in the Tables
RALSSSVIREL

SEQ ID NO: 613 - protein, 9 aa, Homo sapiens
  (1)..(9): One or more of the listed amino acids can be modified as set forth in the Tables
RALTPSPVM

SEQ ID NO: 614 - protein, 9 aa, Homo sapiens
  (1)..(9): One or more of the listed amino acids can be modified as set forth in the Tables
RASSDIVSL

SEQ ID NO: 615 - protein, 9 aa, Homo sapiens
  (1)..(9): One or more of the listed amino acids can be modified as set forth in the Tables
RASSPFRRV

SEQ ID NO: 616 - protein, 8 aa, Homo sapiens
  (1)..(8): One or more of the listed amino acids can be modified as set forth in the Tables
RASVFVKL

SEQ ID NO: 617 - protein, 8 aa, Homo sapiens
  (1)..(8): One or more of the listed amino acids can be modified as set forth in the Tables
RATSLPSL

SEQ ID NO: 618 - protein, 9 aa, Homo sapiens
  (1)..(9): One or more of the listed amino acids can be modified as set forth in the Tables
RATSNVFAM

SEQ ID NO: 619 - protein, 9 aa, Homo sapiens
  (1)..(9): One or more of the listed amino acids can be modified as set forth in the Tables
RATSNVFAM

SEQ ID NO: 620 - protein, 9 aa, Homo sapiens
  (1)..(9): One or more of the listed amino acids can be modified as set forth in the Tables
RATSPLVSL

SEQ ID NO: 621 - protein, 9 aa, Homo sapiens
  (1)..(9): One or more of the listed amino acids can be modified as set forth in the Tables
RATSRCLQL

SEQ ID NO: 622 - protein, 9 aa, Homo sapiens
  (1)..(9): One or more of the listed amino acids can be modified as set forth in the Tables
RAVSPFAKI

SEQ ID NO: 623 - protein, 9 aa, Homo sapiens
  (1)..(9): One or more of the listed amino acids can be modified as set forth in the Tables
REAPSPLMI

SEQ ID NO: 624 - protein, 10 aa, Homo sapiens
  (1)..(10): One or more of the listed amino acids can be modified as set forth in the Tables
REASIELPSM

SEQ ID NO: 625 - protein, 13 aa, Homo sapiens
  (1)..(13): One or more of the listed amino acids can be modified as set forth in the Tables
REASPLSSNKLIL

SEQ ID NO: 626 - protein, 10 aa, Homo sapiens
  (1)..(10): One or more of the listed amino acids can be modified as set forth in the Tables
REESPLRIKM

SEQ ID NO: 627 - protein, 10 aa, Homo sapiens
  (1)..(10): One or more of the listed amino acids can be modified as set forth in the Tables
REGSFRVTTA

SEQ ID NO: 628 - protein, 10 aa, Homo sapiens
  (1)..(10): One or more of the listed amino acids can be modified as set forth in the Tables
REGSGRFSLP

SEQ ID NO: 629 - protein, 9 aa, Homo sapiens
  (1)..(9): One or more of the listed amino acids can be modified as set forth in the Tables
REISSSPTS

SEQ ID NO: 630 - protein, 9 aa, Homo sapiens
  (1)..(9): One or more of the listed amino acids can be modified as set forth in the Tables
REKSPGRML

SEQ ID NO: 631 - protein, 11 aa, Homo sapiens
  (1)..(11): One or more of the listed amino acids can be modified as set forth in the Tables
RELSGTIKEIL

SEQ ID NO: 632 - protein, 9 aa, Homo sapiens
  (1)..(9): One or more of the listed amino acids can be modified as set forth in the Tables
RENSFGSPL

SEQ ID NO: 633 - protein, 11 aa, Homo sapiens
  (1)..(11): One or more of the listed amino acids can be modified as set forth in the Tables
RENSFGSPLEF

SEQ ID NO: 634 - protein, 11 aa, Homo sapiens
  (1)..(11): One or more of the listed amino acids can be modified as set forth in the Tables
REPSPALGPNL

SEQ ID NO: 635 - protein, 11 aa, Homo sapiens
  (1)..(11): One or more of the listed amino acids can be modified as set forth in the Tables
REPSPLPELAL

SEQ ID NO: 636 - protein, 11 aa, Homo sapiens
  (1)..(11): One or more of the listed amino acids can be modified as set forth in the Tables
REPSPVRYDNL

SEQ ID NO: 637 - protein, 9 aa, Homo sapiens
  (1)..(9): One or more of the listed amino acids can be modified as set forth in the Tables
RERSPGRLF

SEQ ID NO: 638 - protein, 9 aa, Homo sapiens
  (1)..(9): One or more of the listed amino acids can be modified as set forth in the Tables
RERWSFIRA

SEQ ID NO: 639 - protein, 9 aa, Homo sapiens
  (1)..(9): One or more of the listed amino acids can be modified as set forth in the Tables
RESPIPIEI

SEQ ID NO: 640 - protein, 9 aa, Homo sapiens
  (1)..(9): One or more of the listed amino acids can be modified as set forth in the Tables
RESPRPLQL

SEQ ID NO: 641 - protein, 9 aa, Homo sapiens
  (1)..(9): One or more of the listed amino acids can be modified as set forth in the Tables
RESSLGFQL

SEQ ID NO: 642 - protein, 10 aa, Homo sapiens
  (1)..(10): One or more of the listed amino acids can be modified as set forth in the Tables
RETSPNRIGL

SEQ ID NO: 643 - protein, 10 aa, Homo sapiens
  (1)..(10): One or more of the listed amino acids can be modified as set forth in the Tables
RETSPNRIGL

SEQ ID NO: 644 - protein, 9 aa, Homo sapiens
  (1)..(9): One or more of the listed amino acids can be modified as set forth in the Tables
REVESLPAV

SEQ ID NO: 645 - protein, 9 aa, Homo sapiens
  (1)..(9): One or more of the listed amino acids can be modified as set forth in the Tables
REVSPEPIV

SEQ ID NO: 646 - protein, 9 aa, Homo sapiens
  (1)..(9): One or more of the listed amino acids can be modified as set forth in the Tables
REWSPTPSL

SEQ ID NO: 647 - protein, 10 aa, Homo sapiens
  (1)..(10): One or more of the listed amino acids can be modified as set forth in the Tables
REWSPTPSSL

SEQ ID NO: 648 - protein, 9 aa, Homo sapiens
  (1)..(9): One or more of the listed amino acids can be modified as set forth in the Tables
REYGSPLKA

SEQ ID NO: 649 - protein, 8 aa, Homo sapiens
  (1)..(8): One or more of the listed amino acids can be modified as set forth in the Tables
RFSFKKSF

SEQ ID NO: 650 - protein, 9 aa, Homo sapiens
  (1)..(9): One or more of the listed amino acids can be modified as set forth in the Tables
RGDSRPRLV

SEQ ID NO: 651 - protein, 8 aa, Homo sapiens
  (1)..(8): One or more of the listed amino acids can be modified as set forth in the Tables
RGISPIVF

SEQ ID NO: 652 - protein, 8 aa, Homo sapiens
  (1)..(8): One or more of the listed amino acids can be modified as set forth in the Tables
RGSFEVTL

SEQ ID NO: 653 - protein, 9 aa, Homo sapiens
  (1)..(9): One or more of the listed amino acids can be modified as set forth in the Tables
RHPKRSVSL

SEQ ID NO: 654 - protein, 9 aa, Homo sapiens
  (1)..(9): One or more of the listed amino acids can be modified as set forth in the Tables
RIDISPSTL

SEQ ID NO: 655 - protein, 9 aa, Homo sapiens
  (1)..(9): One or more of the listed amino acids can be modified as set forth in the Tables
RIHGSPLQK

SEQ ID NO: 656 - protein, 12 aa, Homo sapiens
  (1)..(12): One or more of the listed amino acids can be modified as set forth in the Tables
RILSATTSGIFL

SEQ ID NO: 657 - protein, 10 aa, Homo sapiens
  (1)..(10): One or more of the listed amino acids can be modified as set forth in the Tables
RILSGVVTKM

SEQ ID NO: 658 - protein, 10 aa, Homo sapiens
  (1)..(10): One or more of the listed amino acids can be modified as set forth in the Tables
RILSGVVTKM

SEQ ID NO: 659 - protein, 12 aa, Homo sapiens
  (1)..(12): One or more of the listed amino acids can be modified as set forth in the Tables
RILSGVVTKMKM

SEQ ID NO: 660 - protein, 12 aa, Homo sapiens
  (1)..(12): One or more of the listed amino acids can be modified as set forth in the Tables
RILSGVVTKMKM

SEQ ID NO: 661 - protein, 9 aa, Homo sapiens
  (1)..(9): One or more of the listed amino acids can be modified as set forth in the Tables
RILSKEYNM

SEQ ID NO: 662 - protein, 9 aa, Homo sapiens
  (1)..(9): One or more of the listed amino acids can be modified as set forth in the Tables
RIPSVQINF

SEQ ID NO: 663 - protein, 10 aa, Homo sapiens
  (1)..(10): One or more of the listed amino acids can be modified as set forth in the Tables
RIRPSTPSQL

SEQ ID NO: 664 - protein, 9 aa, Homo sapiens
  (1)..(9): One or more of the listed amino acids can be modified as set forth in the Tables
RISTPLTGV

SEQ ID NO: 665 - protein, 11 aa, Homo sapiens
  (1)..(11): One or more of the listed amino acids can be modified as set forth in the Tables
RIVSPKNSDLK

SEQ ID NO: 666 - protein, 9 aa, Homo sapiens
  (1)..(9): One or more of the listed amino acids can be modified as set forth in the Tables
RIYSMSLRL

SEQ ID NO: 667 - protein, 9 aa, Homo sapiens
  (1)..(9): One or more of the listed amino acids can be modified as set forth in the Tables
RKASLRQFL

SEQ ID NO: 668 - protein, 9 aa, Homo sapiens
  (1)..(9): One or more of the listed amino acids can be modified as set forth in the Tables
RKNSFVMEY

SEQ ID NO: 669 - protein, 10 aa, Homo sapiens
  (1)..(10): One or more of the listed amino acids can be modified as set forth in the Tables
RKPSAEMNRI

SEQ ID NO: 670 - protein, 9 aa, Homo sapiens
  (1)..(9): One or more of the listed amino acids can be modified as set forth in the Tables
RKPSLAKAL

SEQ ID NO: 671 - protein, 9 aa, Homo sapiens
  (1)..(9): One or more of the listed amino acids can be modified as set forth in the Tables
RKSSIIIRM

SEQ ID NO: 672 - protein, 10 aa, Homo sapiens
  (1)..(10): One or more of the listed amino acids can be modified as set forth in the Tables
RLASLMNLGM

SEQ ID NO: 673 - protein, 9 aa, Homo sapiens
  (1)..(9): One or more of the listed amino acids can be modified as set forth in the Tables
RLASSVLRC

SEQ ID NO: 674 - protein, 9 aa, Homo sapiens
  (1)..(9): One or more of the listed amino acids can be modified as set forth in the Tables
RLASSVLRC

SEQ ID NO: 675 - protein, 9 aa, Homo sapiens
  (1)..(9): One or more of the listed amino acids can be modified as set forth in the Tables
RLASSVLRC

SEQ ID NO: 676 - protein, 9 aa, Homo sapiens
  (1)..(9): One or more of the listed amino acids can be modified as set forth in the Tables
RLASSVLRC

SEQ ID NO: 677 - protein, 9 aa, Homo sapiens
  (1)..(9): One or more of the listed amino acids can be modified as set forth in the Tables
RLASSVLRC

SEQ ID NO: 678 - protein, 9 aa, Homo sapiens
  (1)..(9): One or more of the listed amino acids can be modified as set forth in the Tables
RLASSVLRC

SEQ ID NO: 679 - protein, 10 aa, Homo sapiens
  (1)..(10): One or more of the listed amino acids can be modified as set forth in the Tables
RLASSVLRCG

SEQ ID NO: 680 - protein, 9 aa, Homo sapiens
  (1)..(9): One or more of the listed amino acids can be modified as set forth in the Tables
RLASYLSGC

SEQ ID NO: 681 - protein, 9 aa, Homo sapiens
  (1)..(9): One or more of the listed amino acids can be modified as set forth in the Tables
RLASYLSGC

SEQ ID NO: 682 - protein, 10 aa, Homo sapiens
  (1)..(10): One or more of the listed amino acids can be modified as set forth in the Tables
RLDSIVGPQL

SEQ ID NO: 683 - protein, 10 aa, Homo sapiens
  (1)..(10): One or more of the listed amino acids can be modified as set forth in the Tables
RLDSPLSNRY

SEQ ID NO: 684 - protein, 8 aa, Homo sapiens
  (1)..(8): One or more of the listed amino acids can be modified as set forth in the Tables
RLDSYVRS

SEQ ID NO: 685 - protein, 9 aa, Homo sapiens
  (1)..(9): One or more of the listed amino acids can be modified as set forth in the Tables
RLDSYVRSL

SEQ ID NO: 686 - protein, 9 aa, Homo sapiens
  (1)..(9): One or more of the listed amino acids can be modified as set forth in the Tables
RLESLSYQL

SEQ ID NO: 687 - protein, 11 aa, Homo sapiens
  (1)..(11): One or more of the listed amino acids can be modified as set forth in the Tables
RLFSHPREPAL

SEQ ID NO: 688 - protein, 9 aa, Homo sapiens
  (1)..(9): One or more of the listed amino acids can be modified as set forth in the Tables
RLFSKELRC

SEQ ID NO: 689 - protein, 9 aa, Homo sapiens
  (1)..(9): One or more of the listed amino acids can be modified as set forth in the Tables
RLFSKELRC

SEQ ID NO: 690 - protein, 9 aa, Homo sapiens
  (1)..(9): One or more of the listed amino acids can be modified as set forth in the Tables
RLFSKELRC

SEQ ID NO: 691 - protein, 9 aa, Homo sapiens
  (1)..(9): One or more of the listed amino acids can be modified as set forth in the Tables
RLGSFHELL

SEQ ID NO: 692 - protein, 9 aa, Homo sapiens
  (1)..(9): One or more of the listed amino acids can be modified as set forth in the Tables
RLISQIVSS

SEQ ID NO: 693 - protein, 10 aa, Homo sapiens
  (1)..(10): One or more of the listed amino acids can be modified as set forth in the Tables
RLISQIVSSI

SEQ ID NO: 694 - protein, 12 aa, Homo sapiens
  (1)..(12): One or more of the listed amino acids can be modified as set forth in the Tables
RLISQIVSSITA

SEQ ID NO: 695 - protein, 12 aa, Homo sapiens
  (1)..(12): One or more of the listed amino acids can be modified as set forth in the Tables
RLKSIEERQLLK

SEQ ID NO: 696 - protein, 9 aa, Homo sapiens
  (1)..(9): One or more of the listed amino acids can be modified as set forth in the Tables
RLKSIIQEV

SEQ ID NO: 697 - protein, 11 aa, Homo sapiens
  (1)..(11): One or more of the listed amino acids can be modified as set forth in the Tables
RLLDPSSPLAL

SEQ ID NO: 698 - protein, 8 aa, Homo sapiens
  (1)..(8): One or more of the listed amino acids can be modified as set forth in the Tables
RLLSAAEN

SEQ ID NO: 699 - protein, 10 aa, Homo sapiens
  (1)..(10): One or more of the listed amino acids can be modified as set forth in the Tables
RLLSAAENFL

SEQ ID NO: 700 - protein, 9 aa, Homo sapiens
  (1)..(9): One or more of the listed amino acids can be modified as set forth in the Tables
RLLSPLSSA

SEQ ID NO: 701 - protein, 10 aa, Homo sapiens
  (1)..(10): One or more of the listed amino acids can be modified as set forth in the Tables
RLLSPPLRPR

SEQ ID NO: 702 - protein, 10 aa, Homo sapiens
  (1)..(10): One or more of the listed amino acids can be modified as set forth in the Tables
RLLSVEGSTL

SEQ ID NO: 703 - protein, 9 aa, Homo sapiens
  (1)..(9): One or more of the listed amino acids can be modified as set forth in the Tables
RLLSVNIRV

SEQ ID NO: 704 - protein, 9 aa, Homo sapiens
  (1)..(9): One or more of the listed amino acids can be modified as set forth in the Tables
RLLSWSDNW

SEQ ID NO: 705 - protein, 9 aa, Homo sapiens
  (1)..(9): One or more of the listed amino acids can be modified as set forth in the Tables
RLMSGKVKV

SEQ ID NO: 706 - protein, 9 aa, Homo sapiens
  (1)..(9): One or more of the listed amino acids can be modified as set forth in the Tables
RLMSGKVKV

SEQ ID NO: 707 - protein, 10 aa, Homo sapiens
  (1)..(10): One or more of the listed amino acids can be modified as set forth in the Tables
RLMTPKPVSI

SEQ ID NO: 708 - protein, 10 aa, Homo sapiens
  (1)..(10): One or more of the listed amino acids can be modified as set forth in the Tables
RLMTPTLSFL

SEQ ID NO: 709 - protein, 9 aa, Homo sapiens
  (1)..(9): One or more of the listed amino acids can be modified as set forth in the Tables
RLPSPTSPF

SEQ ID NO: 710 - protein, 9 aa, Homo sapiens
  (1)..(9): One or more of the listed amino acids can be modified as set forth in the Tables
RLPTRLPEI

SEQ ID NO: 711 - protein, 9 aa, Homo sapiens
  (1)..(9): One or more of the listed amino acids can be modified as set forth in the Tables
RLQTQVFKL

SEQ ID NO: 712 - protein, 9 aa, Homo sapiens
  (1)..(9): One or more of the listed amino acids can be modified as set forth in the Tables
RLRSSLVFK

SEQ ID NO: 713 - protein, 9 aa, Homo sapiens
  (1)..(9): One or more of the listed amino acids can be modified as set forth in the Tables
RLRSSVPGV

SEQ ID NO: 714 - protein, 9 aa, Homo sapiens
  (1)..(9): One or more of the listed amino acids can be modified as set forth in the Tables
RLRSSVPGV

SEQ ID NO: 715 - protein, 9 aa, Homo sapiens
  (1)..(9): One or more of the listed amino acids can be modified as set forth in the Tables
RLRSSVPGV

SEQ ID NO: 716 - protein, 8 aa, Homo sapiens
  (1)..(8): One or more of the listed amino acids can be modified as set forth in the Tables
RLSFLVSY

SEQ ID NO: 717 - protein, 9 aa, Homo sapiens
  (1)..(9): One or more of the listed amino acids can be modified as set forth in the Tables
RLSPVPVPR

SEQ ID NO: 718 - protein, 9 aa, Homo sapiens
  (1)..(9): One or more of the listed amino acids can be modified as set forth in the Tables
RLSSVSVTY

SEQ ID NO: 719 - protein, 9 aa, Homo sapiens
  (1)..(9): One or more of the listed amino acids can be modified as set forth in the Tables
RLYKSEPEL

SEQ ID NO: 720 - protein, 10 aa, Homo sapiens
  (1)..(10): One or more of the listed amino acids can be modified as set forth in the Tables
RMISKLEAQV

SEQ ID NO: 721 - protein, 10 aa, Homo sapiens
  (1)..(10): One or more of the listed amino acids can be modified as set forth in the Tables
RMISKLEAQV

SEQ ID NO: 722 - protein, 10 aa, Homo sapiens
  (1)..(10): One or more of the listed amino acids can be modified as set forth in the Tables
RMLSLRDQRL

SEQ ID NO: 723 - protein, 8 aa, Homo sapiens
  (1)..(8): One or more of the listed amino acids can be modified as set forth in the Tables
RMSLLSVV

SEQ ID NO: 724 - protein, 10 aa, Homo sapiens
  (1)..(10): One or more of the listed amino acids can be modified as set forth in the Tables
RMYSPIIYQA

SEQ ID NO: 725 - protein, 9 aa, Homo sapiens
  (1)..(9): One or more of the listed amino acids can be modified as set forth in the Tables
RNKSYSFIA

SEQ ID NO: 726 - protein, 9 aa, Homo sapiens
  (1)..(9): One or more of the listed amino acids can be modified as set forth in the Tables
RPAKSLMSI

SEQ ID NO: 727 - protein, 9 aa, Homo sapiens
  (1)..(9): One or more of the listed amino acids can be modified as set forth in the Tables
RPAKSMDSL

SEQ ID NO: 728 - protein, 10 aa, Homo sapiens
  (1)..(10): One or more of the listed amino acids can be modified as set forth in the Tables
RPARPSRKGL

SEQ ID NO: 729 - protein, 9 aa, Homo sapiens
  (1)..(9): One or more of the listed amino acids can be modified as set forth in the Tables
RPARSVPSI

SEQ ID NO: 730 - protein, 9 aa, Homo sapiens
  (1)..(9): One or more of the listed amino acids can be modified as set forth in the Tables
RPASPALLL

SEQ ID NO: 731 - protein, 9 aa, Homo sapiens
  (1)..(9): One or more of the listed amino acids can be modified as set forth in the Tables
RPASPLMHI

SEQ ID NO: 732 - protein, 9 aa, Homo sapiens
  (1)..(9): One or more of the listed amino acids can be modified as set forth in the Tables
RPASRFEVL

SEQ ID NO: 733 - protein, 10 aa, Homo sapiens
  (1)..(10): One or more of the listed amino acids can be modified as set forth in the Tables
RPASTGGLSL

SEQ ID NO: 734 - protein, 8 aa, Homo sapiens
  (1)..(8): One or more of the listed amino acids can be modified as set forth in the Tables
RPATPHLL

SEQ ID NO: 735 - protein, 9 aa, Homo sapiens
  (1)..(9): One or more of the listed amino acids can be modified as set forth in the Tables
RPDSRLLEL

SEQ ID NO: 736 - protein, 9 aa, Homo sapiens
  (1)..(9): One or more of the listed amino acids can be modified as set forth in the Tables
RPESPAGPF

SEQ ID NO: 737 - protein, 11 aa, Homo sapiens
  (1)..(11): One or more of the listed amino acids can be modified as set forth in the Tables
RPHLSGRKLSL

SEQ ID NO: 738 - protein, 9 aa, Homo sapiens
  (1)..(9): One or more of the listed amino acids can be modified as set forth in the Tables
RPHTPTPGI

SEQ ID NO: 739 - protein, 11 aa, Homo sapiens
  (1)..(11): One or more of the listed amino acids can be modified as set forth in the Tables
RPHTPTPGIYM

SEQ ID NO: 740 - protein, 11 aa, Homo sapiens
  (1)..(11): One or more of the listed amino acids can be modified as set forth in the Tables
RPHTPTPGIYM

SEQ ID NO: 741 - protein, 11 aa, Homo sapiens
  (1)..(11): One or more of the listed amino acids can be modified as set forth in the Tables
RPISVIGGVSL

SEQ ID NO: 742 - protein, 12 aa, Homo sapiens
  (1)..(12): One or more of the listed amino acids can be modified as set forth in the Tables
RPISVIGGVSLY

SEQ ID NO: 743 - protein, 10 aa, Homo sapiens
  (1)..(10): One or more of the listed amino acids can be modified as set forth in the Tables
RPKLHHSLSF

SEQ ID NO: 744 - protein, 10 aa, Homo sapiens
  (1)..(10): One or more of the listed amino acids can be modified as set forth in the Tables
RPKPSSSPVI

SEQ ID NO: 745 - protein, 11 aa, Homo sapiens
  (1)..(11): One or more of the listed amino acids can be modified as set forth in the Tables
RPKPSSSPVIF

SEQ ID NO: 746 - protein, 9 aa, Homo sapiens
  (1)..(9): One or more of the listed amino acids can be modified as set forth in the Tables
RPKSDIVLL

SEQ ID NO: 747 - protein, 10 aa, Homo sapiens
  (1)..(10): One or more of the listed amino acids can be modified as set forth in the Tables
RPKSPGGIQP

SEQ ID NO: 748 - protein, 10 aa, Homo sapiens
  (1)..(10): One or more of the listed amino acids can be modified as set forth in the Tables
RPKSSSPIRL

SEQ ID NO: 749 - protein, 10 aa, Homo sapiens
  (1)..(10): One or more of the listed amino acids can be modified as set forth in the Tables
RPKTPNRASP

SEQ ID NO: 750 - protein, 9 aa, Homo sapiens
  (1)..(9): One or more of the listed amino acids can be modified as set forth in the Tables
RPKTPPPAP

SEQ ID NO: 751 - protein, 9 aa, Homo sapiens
  (1)..(9): One or more of the listed amino acids can be modified as set forth in the Tables
RPLKPLSPL

SEQ ID NO: 752 - protein, 9 aa, Homo sapiens
  (1)..(9): One or more of the listed amino acids can be modified as set forth in the Tables
RPLPPPSSL

SEQ ID NO: 753 - protein, 10 aa, Homo sapiens
  (1)..(10): One or more of the listed amino acids can be modified as set forth in the Tables
RPLSATRKTL

SEQ ID NO: 754 - protein, 11 aa, Homo sapiens
  (1)..(11): One or more of the listed amino acids can be modified as set forth in the Tables
RPLSGSGISAF

SEQ ID NO: 755 - protein, 9 aa, Homo sapiens
  (1)..(9): One or more of the listed amino acids can be modified as set forth in the Tables
RPLSHYSSF

SEQ ID NO: 756 - protein, 9 aa, Homo sapiens
  (1)..(9): One or more of the listed amino acids can be modified as set forth in the Tables
RPLSKQLSA

SEQ ID NO: 757 - protein, 10 aa, Homo sapiens
  (1)..(10): One or more of the listed amino acids can be modified as set forth in the Tables
RPLSLIGSTL

SEQ ID NO: 758 - protein, 10 aa, Homo sapiens
  (1)..(10): One or more of the listed amino acids can be modified as set forth in the Tables
RPLSPGALEL

SEQ ID NO: 759 - protein, 10 aa, Homo sapiens
  (1)..(10): One or more of the listed amino acids can be modified as set forth in the Tables
RPLSPGALQL

SEQ ID NO: 760 - protein, 9 aa, Homo sapiens
  (1)..(9): One or more of the listed amino acids can be modified as set forth in the Tables
RPLSPILHI

SEQ ID NO: 761 - protein, 10 aa, Homo sapiens
  (1)..(10): One or more of the listed amino acids can be modified as set forth in the Tables
RPLSPTAFSL

SEQ ID NO: 762 - protein, 9 aa, Homo sapiens
  (1)..(9): One or more of the listed amino acids can be modified as set forth in the Tables
RPLSSSHEA

SEQ ID NO: 763 - protein, 9 aa, Homo sapiens
  (1)..(9): One or more of the listed amino acids can be modified as set forth in the Tables
RPLTPRTPA

SEQ ID NO: 764 - protein, 9 aa, Homo sapiens
  (1)..(9): One or more of the listed amino acids can be modified as set forth in the Tables
RPLTSPESL

SEQ ID NO: 765 - protein, 9 aa, Homo sapiens
  (1)..(9): One or more of the listed amino acids can be modified as set forth in the Tables
RPMSESPHM

SEQ ID NO: 766 - protein, 9 aa, Homo sapiens
  (1)..(9): One or more of the listed amino acids can be modified as set forth in the Tables
RPPTPTLSL

SEQ ID NO: 767 - protein, 12 aa, Homo sapiens
  (1)..(12): One or more of the listed amino acids can be modified as set forth in the Tables
RPPTSPGVFGAL

SEQ ID NO: 768 - protein, 10 aa, Homo sapiens
  (1)..(10): One or more of the listed amino acids can be modified as set forth in the Tables
RPQRATSNVF

SEQ ID NO: 769 - protein, 9 aa, Homo sapiens
  (1)..(9): One or more of the listed amino acids can be modified as set forth in the Tables
RPQTPKEEA

SEQ ID NO: 770 - protein, 10 aa, Homo sapiens
  (1)..(10): One or more of the listed amino acids can be modified as set forth in the Tables
RPRDTRRISL

SEQ ID NO: 771 - protein, 11 aa, Homo sapiens
  (1)..(11): One or more of the listed amino acids can be modified as set forth in the Tables
RPRGPSPLVTM

SEQ ID NO: 772 - protein, 9 aa, Homo sapiens
  (1)..(9): One or more of the listed amino acids can be modified as set forth in the Tables
RPRHSLNSL

SEQ ID NO: 773 - protein, 12 aa, Homo sapiens
  (1)..(12): One or more of the listed amino acids can be modified as set forth in the Tables
RPRPGTGLGRVM

SEQ ID NO: 774 - protein, 8 aa, Homo sapiens
  (1)..(8): One or more of the listed amino acids can be modified as set forth in the Tables
RPRPSSVL

SEQ ID NO: 775 - protein, 11 aa, Homo sapiens
  (1)..(11): One or more of the listed amino acids can be modified as set forth in the Tables
RPRSGSTGSSL

SEQ ID NO: 776 - protein, 10 aa, Homo sapiens
  (1)..(10): One or more of the listed amino acids can be modified as set forth in the Tables
RPRSISVEEF

SEQ ID NO: 777 - protein, 10 aa, Homo sapiens
  (1)..(10): One or more of the listed amino acids can be modified as set forth in the Tables
RPRSLSSPTV

SEQ ID NO: 778 - protein, 10 aa, Homo sapiens
  (1)..(10): One or more of the listed amino acids can be modified as set forth in the Tables
RPRSLSSPTV

SEQ ID NO: 779 - protein, 12 aa, Homo sapiens
  (1)..(12): One or more of the listed amino acids can be modified as set forth in the Tables
RPRSLSSPTVTL

SEQ ID NO: 780 - protein, 11 aa, Homo sapiens
  (1)..(11): One or more of the listed amino acids can be modified as set forth in the Tables
RPRSPAGQVAA

SEQ ID NO: 781 - protein, 12 aa, Homo sapiens
  (1)..(12): One or more of the listed amino acids can be modified as set forth in the Tables
RPRSPGSNSKVP

SEQ ID NO: 782 - protein, 9 aa, Homo sapiens
  (1)..(9): One or more of the listed amino acids can be modified as set forth in the Tables
RPRSPSPIS

SEQ ID NO: 783 - protein, 10 aa, Homo sapiens
  (1)..(10): One or more of the listed amino acids can be modified as set forth in the Tables
RPRSPSSYDL

SEQ ID NO: 784 - protein, 12 aa, Homo sapiens
  (1)..(12): One or more of the listed amino acids can be modified as set forth in the Tables
RPRSTSQSIVSL

SEQ ID NO: 785 - protein, 10 aa, Homo sapiens
  (1)..(10): One or more of the listed amino acids can be modified as set forth in the Tables
RPRTNTPKQL

SEQ ID NO: 786 - protein, 10 aa, Homo sapiens
  (1)..(10): One or more of the listed amino acids can be modified as set forth in the Tables
RPSLGGRTPL

SEQ ID NO: 787 - protein, 9 aa, Homo sapiens
  (1)..(9): One or more of the listed amino acids can be modified as set forth in the Tables
RPSSAPDLM

SEQ ID NO: 788 - protein, 9 aa, Homo sapiens
  (1)..(9): One or more of the listed amino acids can be modified as set forth in the Tables
RPSSGFYEL

SEQ ID NO: 789 - protein, 8 aa, Homo sapiens
  (1)..(8): One or more of the listed amino acids can be modified as set forth in the Tables
RPSSLPDL

SEQ ID NO: 790 - protein, 9 aa, Homo sapiens
  (1)..(9): One or more of the listed amino acids can be modified as set forth in the Tables
RPSSPALYF

SEQ ID NO: 791 - protein, 9 aa, Homo sapiens
  (1)..(9): One or more of the listed amino acids can be modified as set forth in the Tables
RPSSPIPLL

SEQ ID NO: 792 - protein, 9 aa, Homo sapiens
  (1)..(9): One or more of the listed amino acids can be modified as set forth in the Tables
RPSSPPPFL

SEQ ID NO: 793 - protein, 13 aa, Homo sapiens
  (1)..(13): One or more of the listed amino acids can be modified as set forth in the Tables
RPSSPRAGAPHAL

SEQ ID NO: 794 - protein, 10 aa, Homo sapiens
  (1)..(10): One or more of the listed amino acids can be modified as set forth in the Tables
RPSSPRVEDL

SEQ ID NO: 795 - protein, 9 aa, Homo sapiens
  (1)..(9): One or more of the listed amino acids can be modified as set forth in the Tables
RPSSPSTSW

SEQ ID NO: 796 - protein, 9 aa, Homo sapiens
  (1)..(9): One or more of the listed amino acids can be modified as set forth in the Tables
RPSSRAVLY

SEQ ID NO: 797 - protein, 11 aa, Homo sapiens
  (1)..(11): One or more of the listed amino acids can be modified as set forth in the Tables
RPSSRVALMVL

SEQ ID NO: 798 - protein, 10 aa, Homo sapiens
  (1)..(10): One or more of the listed amino acids can be modified as set forth in the Tables
RPSSVLIEQL

SEQ ID NO: 799 - protein, 9 aa, Homo sapiens
  (1)..(9): One or more of the listed amino acids can be modified as set forth in the Tables
RPSTPGLSV

SEQ ID NO: 800 - protein, 10 aa, Homo sapiens
  (1)..(10): One or more of the listed amino acids can be modified as set forth in the Tables
RPSTPHTITL

SEQ ID NO: 801 - protein, 10 aa, Homo sapiens
  (1)..(10): One or more of the listed amino acids can be modified as set forth in the Tables
RPSTPSRLAL

SEQ ID NO: 802 - protein, 10 aa, Homo sapiens
  (1)..(10): One or more of the listed amino acids can be modified as set forth in the Tables
RPSTPTIDVL

SEQ ID NO: 803 - protein, 9 aa, Homo sapiens
  (1)..(9): One or more of the listed amino acids can be modified as set forth in the Tables
RPSTPTINV

SEQ ID NO: 804 - protein, 10 aa, Homo sapiens
  (1)..(10): One or more of the listed amino acids can be modified as set forth in the Tables
RPSTPTINVL

SEQ ID NO: 805 - protein, 10 aa, Homo sapiens
  (1)..(10): One or more of the listed amino acids can be modified as set forth in the Tables
RPTSISWDGL

SEQ ID NO: 806 - protein, 9 aa, Homo sapiens
  (1)..(9): One or more of the listed amino acids can be modified as set forth in the Tables
RPTSPIQIM

SEQ ID NO: 807 - protein, 10 aa, Homo sapiens
  (1)..(10): One or more of the listed amino acids can be modified as set forth in the Tables
RPTSRLNRLP

SEQ ID NO: 808 - protein, 10 aa, Homo sapiens
  (1)..(10): One or more of the listed amino acids can be modified as set forth in the Tables
RPVDPRRRSL

SEQ ID NO: 809 - protein, 9 aa, Homo sapiens
  (1)..(9): One or more of the listed amino acids can be modified as set forth in the Tables
RPVSPAGPP

SEQ ID NO: 810 - protein, 9 aa, Homo sapiens
  (1)..(9): One or more of the listed amino acids can be modified as set forth in the Tables
RPVSPAPGA

SEQ ID NO: 811 - protein, 11 aa, Homo sapiens
  (1)..(11): One or more of the listed amino acids can be modified as set forth in the Tables
RPVSPGKDITA

SEQ ID NO: 812 - protein, 9 aa, Homo sapiens
  (1)..(9): One or more of the listed amino acids can be modified as set forth in the Tables
RPVSPHSDF

SEQ ID NO: 813 - protein, 9 aa, Homo sapiens
  (1)..(9): One or more of the listed amino acids can be modified as set forth in the Tables
RPVSPPQKA

SEQ ID NO: 814 - protein, 9 aa, Homo sapiens
  (1)..(9): One or more of the listed amino acids can be modified as set forth in the Tables
RPVSPSAYM

SEQ ID NO: 815 - protein, 9 aa, Homo sapiens
  (1)..(9): One or more of the listed amino acids can be modified as set forth in the Tables
RPVSPSSLL

SEQ ID NO: 816 - protein, 9 aa, Homo sapiens
  (1)..(9): One or more of the listed amino acids can be modified as set forth in the Tables
RPVTPITNF

SEQ ID NO: 817 - protein, 9 aa, Homo sapiens
  (1)..(9): One or more of the listed amino acids can be modified as set forth in the Tables
RPVTPPRTA

SEQ ID NO: 818 - protein, 11 aa, Homo sapiens
  (1)..(11): One or more of the listed amino acids can be modified as set forth in the Tables
RPWSPPPTGSL

SEQ ID NO: 819 - protein, 10 aa, Homo sapiens
  (1)..(10): One or more of the listed amino acids can be modified as set forth in the Tables
RPYPSPGAVL

SEQ ID NO: 820 - protein, 8 aa, Homo sapiens
  (1)..(8): One or more of the listed amino acids can be modified as set forth in the Tables
RPYSPPFF

SEQ ID NO: 821 - protein, 10 aa, Homo sapiens
  (1)..(10): One or more of the listed amino acids can be modified as set forth in the Tables
RPYSPSEYAL

SEQ ID NO: 822 - protein, 10 aa, Homo sapiens
  (1)..(10): One or more of the listed amino acids can be modified as set forth in the Tables
RPYSPSQYAL

SEQ ID NO: 823 - protein, 10 aa, Homo sapiens
  (1)..(10): One or more of the listed amino acids can be modified as set forth in the Tables
RPYTNKVITL

SEQ ID NO: 824 - protein, 10 aa, Homo sapiens
  (1)..(10): One or more of the listed amino acids can be modified as set forth in the Tables
RQASIELPSM

SEQ ID NO: 825 - protein, 12 aa, Homo sapiens
  (1)..(12): One or more of the listed amino acids can be modified as set forth in the Tables
RQASIELPSMAV

SEQ ID NO: 826 - protein, 13 aa, Homo sapiens
  (1)..(13): One or more of the listed amino acids can be modified as set forth in the Tables
RQASIELPSMAVA

SEQ ID NO: 827 - protein, 9 aa, Homo sapiens
  (1)..(9): One or more of the listed amino acids can be modified as set forth in the Tables
RQASPPRRL

SEQ ID NO: 828 - protein, 9 aa, Homo sapiens
  (1)..(9): One or more of the listed amino acids can be modified as set forth in the Tables
RQFMRRTSL

SEQ ID NO: 829 - protein, 9 aa, Homo sapiens
  (1)..(9): One or more of the listed amino acids can be modified as set forth in the Tables
RQFMRRTSL

SEQ ID NO: 830 - protein, 9 aa, Homo sapiens
  (1)..(9): One or more of the listed amino acids can be modified as set forth in the Tables
RQISTSGEL

SEQ ID NO: 831 - protein, 10 aa, Homo sapiens
  (1)..(10): One or more of the listed amino acids can be modified as set forth in the Tables
RQMSGAQIKI

SEQ ID NO: 832 - protein, 9 aa, Homo sapiens
  (1)..(9): One or more of the listed amino acids can be modified as set forth in the Tables
RQMSRFKEA

SEQ ID NO: 833 - protein, 9 aa, Homo sapiens
  (1)..(9): One or more of the listed amino acids can be modified as set forth in the Tables
RQPSEEEII

SEQ ID NO: 834 - protein, 11 aa, Homo sapiens
  (1)..(11): One or more of the listed amino acids can be modified as set forth in the Tables
RQPSEEEIIKL

SEQ ID NO: 835 - protein, 10 aa, Homo sapiens
  (1)..(10): One or more of the listed amino acids can be modified as set forth in the Tables
RQPSIELPSM

SEQ ID NO: 836 - protein, 10 aa, Homo sapiens
  (1)..(10): One or more of the listed amino acids can be modified as set forth in the Tables
RQPSIELPSM

SEQ ID NO: 837 - protein, 9 aa, Homo sapiens
  (1)..(9): One or more of the listed amino acids can be modified as set forth in the Tables
RQPSLAKRV

SEQ ID NO: 838 - protein, 9 aa, Homo sapiens
  (1)..(9): One or more of the listed amino acids can be modified as set forth in the Tables
RQPSLKRSL

SEQ ID NO: 839 - protein, 9 aa, Homo sapiens
  (1)..(9): One or more of the listed amino acids can be modified as set forth in the Tables
RQSSFEPEF

SEQ ID NO: 840 - protein, 9 aa, Homo sapiens
  (1)..(9): One or more of the listed amino acids can be modified as set forth in the Tables
RQYSVTDAL

SEQ ID NO: 841 - protein, 9 aa, Homo sapiens
  (1)..(9): One or more of the listed amino acids can be modified as set forth in the Tables
RRASLSYSF

SEQ ID NO: 842 - protein, 9 aa, Homo sapiens
  (1)..(9): One or more of the listed amino acids can be modified as set forth in the Tables
RRFSDFLGL

SEQ ID NO: 843 - protein, 10 aa, Homo sapiens
  (1)..(10): One or more of the listed amino acids can be modified as set forth in the Tables
RRFSFSGNTL

SEQ ID NO: 844 - protein, 8 aa, Homo sapiens
  (1)..(8): One or more of the listed amino acids can be modified as set forth in the Tables
RRFSGLLN

SEQ ID NO: 845 - protein, 9 aa, Homo sapiens
  (1)..(9): One or more of the listed amino acids can be modified as set forth in the Tables
RRFSGLLNC

SEQ ID NO: 846 - protein, 9 aa, Homo sapiens
  (1)..(9): One or more of the listed amino acids can be modified as set forth in the Tables
RRFSGLLNC

SEQ ID NO: 847 - protein, 9 aa, Homo sapiens
  (1)..(9): One or more of the listed amino acids can be modified as set forth in the Tables
RRFSGLLNC

SEQ ID NO: 848 - protein, 9 aa, Homo sapiens
  (1)..(9): One or more of the listed amino acids can be modified as set forth in the Tables
RRFSLSPSL

SEQ ID NO: 849 - protein, 11 aa, Homo sapiens
  (1)..(11): One or more of the listed amino acids can be modified as set forth in the Tables
RRFSLTTLRNF

SEQ ID NO: 850 - protein, 11 aa, Homo sapiens
  (1)..(11): One or more of the listed amino acids can be modified as set forth in the Tables
RRFSLTTLRNY

SEQ ID NO: 851 - protein, 9 aa, Homo sapiens
  (1)..(9): One or more of the listed amino acids can be modified as set forth in the Tables
RRFSPPRRM

SEQ ID NO: 852 - protein, 10 aa, Homo sapiens
  (1)..(10): One or more of the listed amino acids can be modified as set forth in the Tables
RRFSPPRRML

SEQ ID NO: 853 - protein, 11 aa, Homo sapiens
  (1)..(11): One or more of the listed amino acids can be modified as set forth in the Tables
RRFSSSDFSDL

SEQ ID NO: 854 - protein, 9 aa, Homo sapiens
  (1)..(9): One or more of the listed amino acids can be modified as set forth in the Tables
RRFSSYSQM

SEQ ID NO: 855 - protein, 9 aa, Homo sapiens
  (1)..(9): One or more of the listed amino acids can be modified as set forth in the Tables
RRFSTEYEL

SEQ ID NO: 856 - protein, 11 aa, Homo sapiens
  (1)..(11): One or more of the listed amino acids can be modified as set forth in the Tables
RRFSVSTLRNL

SEQ ID NO: 857 - protein, 13 aa, Homo sapiens
  (1)..(13): One or more of the listed amino acids can be modified as set forth in the Tables
RRFSVSTLRNLGL

SEQ ID NO: 858 - protein, 14 aa, Homo sapiens
  (1)..(14): One or more of the listed amino acids can be modified as set forth in the Tables
RRFSVSTLRNLGLG

SEQ ID NO: 859 - protein, 15 aa, Homo sapiens
  (1)..(15): One or more of the listed amino acids can be modified as set forth in the Tables
RRFSVSTLRNLGLGK

SEQ ID NO: 860 - protein, 9 aa, Homo sapiens
  (1)..(9): One or more of the listed amino acids can be modified as set forth in the Tables
RRFSVTLRL

SEQ ID NO: 861 - protein, 9 aa, Homo sapiens
  (1)..(9): One or more of the listed amino acids can be modified as set forth in the Tables
RRFTEIYEF

SEQ ID NO: 862 - protein, 10 aa, Homo sapiens
  (1)..(10): One or more of the listed amino acids can be modified as set forth in the Tables
RRGSFEVTLL

SEQ ID NO: 863 - protein, 9 aa, Homo sapiens
  (1)..(9): One or more of the listed amino acids can be modified as set forth in the Tables
RRGSFPLAA

SEQ ID NO: 864 - protein, 9 aa, Homo sapiens
  (1)..(9): One or more of the listed amino acids can be modified as set forth in the Tables
RRGSGPEIF

SEQ ID NO: 865 - protein, 10 aa, Homo sapiens
  (1)..(10): One or more of the listed amino acids can be modified as set forth in the Tables
RRGSGPEIFT

SEQ ID NO: 866 - protein, 11 aa, Homo sapiens
  (1)..(11): One or more of the listed amino acids can be modified as set forth in the Tables
RRGSGPEIFTF

SEQ ID NO: 867 - protein, 9 aa, Homo sapiens
  (1)..(9): One or more of the listed amino acids can be modified as set forth in the Tables
RRGSLLGSM

SEQ ID NO: 868 - protein, 9 aa, Homo sapiens
  (1)..(9): One or more of the listed amino acids can be modified as set forth in the Tables
RRGSLTLTI

SEQ ID NO: 869 - protein, 9 aa, Homo sapiens
  (1)..(9): One or more of the listed amino acids can be modified as set forth in the Tables
RRGSNVALM

SEQ ID NO: 870 - protein, 9 aa, Homo sapiens
  (1)..(9): One or more of the listed amino acids can be modified as set forth in the Tables
RRGSPVRQL

SEQ ID NO: 871 - protein, 10 aa, Homo sapiens
  (1)..(10): One or more of the listed amino acids can be modified as set forth in the Tables
RRGSYPFIDF

SEQ ID NO: 872 - protein, 9 aa, Homo sapiens
  (1)..(9): One or more of the listed amino acids can be modified as set forth in the Tables
RRHSLENKV

SEQ ID NO: 873 - protein, 12 aa, Homo sapiens
  (1)..(12): One or more of the listed amino acids can be modified as set forth in the Tables
RRIDISPSTFRK

SEQ ID NO: 874 - protein, 10 aa, Homo sapiens
  (1)..(10): One or more of the listed amino acids can be modified as set forth in the Tables
RRIDISPSTL

SEQ ID NO: 875 - protein, 9 aa, Homo sapiens
  (1)..(9): One or more of the listed amino acids can be modified as set forth in the Tables
RRISIGSLF

SEQ ID NO: 876 - protein, 9 aa, Homo sapiens
  (1)..(9): One or more of the listed amino acids can be modified as set forth in the Tables
RRISLTKRL

SEQ ID NO: 877 - protein, 9 aa, Homo sapiens
  (1)..(9): One or more of the listed amino acids can be modified as set forth in the Tables
RRISVFKYV

SEQ ID NO: 878 - protein, 9 aa, Homo sapiens
  (1)..(9): One or more of the listed amino acids can be modified as set forth in the Tables
RRISVTSKV

SEQ ID NO: 879 - protein, 9 aa, Homo sapiens
  (1)..(9): One or more of the listed amino acids can be modified as set forth in the Tables
RRKSDDVHL

SEQ ID NO: 880 - protein, 9 aa, Homo sapiens
  (1)..(9): One or more of the listed amino acids can be modified as set forth in the Tables
RRKSLVLKF

SEQ ID NO: 881 - protein, 9 aa, Homo sapiens
  (1)..(9): One or more of the listed amino acids can be modified as set forth in the Tables
RRLSAARLL

SEQ ID NO: 882 - protein, 9 aa, Homo sapiens
  (1)..(9): One or more of the listed amino acids can be modified as set forth in the Tables
RRLSFQAEY

SEQ ID NO: 883 - protein, 9 aa, Homo sapiens
  (1)..(9): One or more of the listed amino acids can be modified as set forth in the Tables
RRLSFSTRL

SEQ ID NO: 884 - protein, 10 aa, Homo sapiens
  (1)..(10): One or more of the listed amino acids can be modified as set forth in the Tables
RRLSGELISM

SEQ ID NO: 885 - protein, 9 aa, Homo sapiens
  (1)..(9): One or more of the listed amino acids can be modified as set forth in the Tables
RRLSLFLVL

SEQ ID NO: 886 - protein, 9 aa, Homo sapiens
  (1)..(9): One or more of the listed amino acids can be modified as set forth in the Tables
RRLSLPGLL

SEQ ID NO: 887 - protein, 9 aa, Homo sapiens
  (1)..(9): One or more of the listed amino acids can be modified as set forth in the Tables
RRLSLSRSL

SEQ ID NO: 888 - protein, 7 aa, Homo sapiens
  (1)..(7): One or more of the listed amino acids can be modified as set forth in the Tables
RRLSRKL

SEQ ID NO: 889 - protein, 9 aa, Homo sapiens
  (1)..(9): One or more of the listed amino acids can be modified as set forth in the Tables
RRLSSQFEN

SEQ ID NO: 890 - protein, 11 aa, Homo sapiens
  (1)..(11): One or more of the listed amino acids can be modified as set forth in the Tables
RRLSVEIYDKF

SEQ ID NO: 891 - protein, 9 aa, Homo sapiens
  (1)..(9): One or more of the listed amino acids can be modified as set forth in the Tables
RRLTLHSVF

SEQ ID NO: 892 - protein, 10 aa, Homo sapiens
  (1)..(10): One or more of the listed amino acids can be modified as set forth in the Tables
RRMSFSGIFR

SEQ ID NO: 893 - protein, 10 aa, Homo sapiens
  (1)..(10): One or more of the listed amino acids can be modified as set forth in the Tables
RRMSFSGIFR

SEQ ID NO: 894 - protein, 9 aa, Homo sapiens
  (1)..(9): One or more of the listed amino acids can be modified as set forth in the Tables
RRMSLLSVV

SEQ ID NO: 895 - protein, 11 aa, Homo sapiens
  (1)..(11): One or more of the listed amino acids can be modified as set forth in the Tables
RRMSVAEQVDY

SEQ ID NO: 896 - protein, 10 aa, Homo sapiens
  (1)..(10): One or more of the listed amino acids can be modified as set forth in the Tables
RRMSVGDRAG

SEQ ID NO: 897 - protein, 10 aa, Homo sapiens
  (1)..(10): One or more of the listed amino acids can be modified as set forth in the Tables
RRNSFIGTPY

SEQ ID NO: 898 - protein, 10 aa, Homo sapiens
  (1)..(10): One or more of the listed amino acids can be modified as set forth in the Tables
RRNSISLREL

SEQ ID NO: 899 - protein, 10 aa, Homo sapiens
  (1)..(10): One or more of the listed amino acids can be modified as set forth in the Tables
RRNSKIFLDL

SEQ ID NO: 900 - protein, 9 aa, Homo sapiens
  (1)..(9): One or more of the listed amino acids can be modified as set forth in the Tables
RRNSLLHGY

SEQ ID NO: 901 - protein, 8 aa, Homo sapiens
  (1)..(8): One or more of the listed amino acids can be modified as set forth in the Tables
RRPKTLRL

SEQ ID NO: 902 - protein, 9 aa, Homo sapiens
  (1)..(9): One or more of the listed amino acids can be modified as set forth in the Tables
RRPSHEGYL

SEQ ID NO: 903 - protein, 9 aa, Homo sapiens
  (1)..(9): One or more of the listed amino acids can be modified as set forth in the Tables
RRPSKPRLI

SEQ ID NO: 904 - protein, 10 aa, Homo sapiens
  (1)..(10): One or more of the listed amino acids can be modified as set forth in the Tables
RRPSLQGNTL

SEQ ID NO: 905 - protein, 10 aa, Homo sapiens
  (1)..(10): One or more of the listed amino acids can be modified as set forth in the Tables
RRPSQNAISF

SEQ ID NO: 906 - protein, 11 aa, Homo sapiens
  (1)..(11): One or more of the listed amino acids can be modified as set forth in the Tables
RRPSQNAISFF

SEQ ID NO: 907 - protein, 9 aa, Homo sapiens
  (1)..(9): One or more of the listed amino acids can be modified as set forth in the Tables
RRPSQPYMF

SEQ ID NO: 908 - protein, 9 aa, Homo sapiens
  (1)..(9): One or more of the listed amino acids can be modified as set forth in the Tables
RRPSRPHMF

SEQ ID NO: 909 - protein, 10 aa, Homo sapiens
  (1)..(10): One or more of the listed amino acids can be modified as set forth in the Tables
RRPSRPHMFP

SEQ ID NO: 910 - protein, 9 aa, Homo sapiens
  (1)..(9): One or more of the listed amino acids can be modified as set forth in the Tables
RRPSYTLGM

SEQ ID NO: 911 - protein, 9 aa, Homo sapiens
  (1)..(9): One or more of the listed amino acids can be modified as set forth in the Tables
RRQSFAVLR

SEQ ID NO: 912 - protein, 9 aa, Homo sapiens
  (1)..(9): One or more of the listed amino acids can be modified as set forth in the Tables
RRQSVSRLL

SEQ ID NO: 913 - protein, 9 aa, Homo sapiens
  (1)..(9): One or more of the listed amino acids can be modified as set forth in the Tables
RRREDSYHV

SEQ ID NO: 914 - protein, 9 aa, Homo sapiens
  (1)..(9): One or more of the listed amino acids can be modified as set forth in the Tables
RRRSAPPEL

SEQ ID NO: 915 - protein, 9 aa, Homo sapiens
  (1)..(9): One or more of the listed amino acids can be modified as set forth in the Tables
RRRSAVHML

SEQ ID NO: 916 - protein, 9 aa, Homo sapiens
  (1)..(9): One or more of the listed amino acids can be modified as set forth in the Tables
RRRSRVFDL

SEQ ID NO: 917 - protein, 9 aa, Homo sapiens
  (1)..(9): One or more of the listed amino acids can be modified as set forth in the Tables
RRSSDIISL

SEQ ID NO: 918 - protein, 9 aa, Homo sapiens
  (1)..(9): One or more of the listed amino acids can be modified as set forth in the Tables
RRSSIPITV

SEQ ID NO: 919 - protein, 9 aa, Homo sapiens
  (1)..(9): One or more of the listed amino acids can be modified as set forth in the Tables
RRSSIQSTF

SEQ ID NO: 920 - protein, 9 aa, Homo sapiens
  (1)..(9): One or more of the listed amino acids can be modified as set forth in the Tables
RRSSISSWL

SEQ ID NO: 921 - protein, 9 aa, Homo sapiens
  (1)..(9): One or more of the listed amino acids can be modified as set forth in the Tables
RRSSLLSLM

SEQ ID NO: 922 - protein, 9 aa, Homo sapiens
  (1)..(9): One or more of the listed amino acids can be modified as set forth in the Tables
RRSSLLSLM

SEQ ID NO: 923 - protein, 9 aa, Homo sapiens
  (1)..(9): One or more of the listed amino acids can be modified as set forth in the Tables
RRSSQSWSL

SEQ ID NO: 924 - protein, 9 aa, Homo sapiens
  (1)..(9): One or more of the listed amino acids can be modified as set forth in the Tables
RRSSYLLAI

SEQ ID NO: 925 - protein, 9 aa, Homo sapiens
  (1)..(9): One or more of the listed amino acids can be modified as set forth in the Tables
RRVSIGVQL

SEQ ID NO: 926 - protein, 8 aa, Homo sapiens
  (1)..(8): One or more of the listed amino acids can be modified as set forth in the Tables
RRVSPLNL

SEQ ID NO: 927 - protein, 13 aa, Homo sapiens
  (1)..(13): One or more of the listed amino acids can be modified as set forth in the Tables
RRVSPLNLSSVTP

SEQ ID NO: 928 - protein, 11 aa, Homo sapiens
  (1)..(11): One or more of the listed amino acids can be modified as set forth in the Tables
RRVSSNGIFDL

SEQ ID NO: 929 - protein, 13 aa, Homo sapiens
  (1)..(13): One or more of the listed amino acids can be modified as set forth in the Tables
RRYSASTVDVIEM

SEQ ID NO: 930 - protein, 9 aa, Homo sapiens
  (1)..(9): One or more of the listed amino acids can be modified as set forth in the Tables
RRYSDLTTL

SEQ ID NO: 931 - protein, 9 aa, Homo sapiens
  (1)..(9): One or more of the listed amino acids can be modified as set forth in the Tables
RRYSDPPTY

SEQ ID NO: 932 - protein, 12 aa, Homo sapiens
  (1)..(12): One or more of the listed amino acids can be modified as set forth in the Tables
RRYSLPLKSIYM

SEQ ID NO: 933 - protein, 8 aa, Homo sapiens
  (1)..(8): One or more of the listed amino acids can be modified as set forth in the Tables
RSASLAKL

SEQ ID NO: 934 - protein, 10 aa, Homo sapiens
  (1)..(10): One or more of the listed amino acids can be modified as set forth in the Tables
RSASLAKLGY

SEQ ID NO: 935 - protein, 10 aa, Homo sapiens
  (1)..(10): One or more of the listed amino acids can be modified as set forth in the Tables
RSASPSSQGW

SEQ ID NO: 936 - protein, 9 aa, Homo sapiens
  (1)..(9): One or more of the listed amino acids can be modified as set forth in the Tables
RSASPTVPR

SEQ ID NO: 937 - protein, 9 aa, Homo sapiens
  (1)..(9): One or more of the listed amino acids can be modified as set forth in the Tables
RSASQERSL

SEQ ID NO: 938 - protein, 10 aa, Homo sapiens
  (1)..(10): One or more of the listed amino acids can be modified as set forth in the Tables
RSASVGAEEY

SEQ ID NO: 939 - protein, 9 aa, Homo sapiens
  (1)..(9): One or more of the listed amino acids can be modified as set forth in the Tables
RSDSSQPML

SEQ ID NO: 940 - protein, 9 aa, Homo sapiens
  (1)..(9): One or more of the listed amino acids can be modified as set forth in the Tables
RSDSSQPML

SEQ ID NO: 941 - protein, 10 aa, Homo sapiens
  (1)..(10): One or more of the listed amino acids can be modified as set forth in the Tables
RSESTENQSY

SEQ ID NO: 942 - protein, 9 aa, Homo sapiens
  (1)..(9): One or more of the listed amino acids can be modified as set forth in the Tables
RSFSGLIKR

SEQ ID NO: 943 - protein, 11 aa, Homo sapiens
  (1)..(11): One or more of the listed amino acids can be modified as set forth in the Tables
RSFSPKSPLEL

SEQ ID NO: 944 - protein, 9 aa, Homo sapiens
  (1)..(9): One or more of the listed amino acids can be modified as set forth in the Tables
RSFSPTMKV

SEQ ID NO: 945 - protein, 9 aa, Homo sapiens
  (1)..(9): One or more of the listed amino acids can be modified as set forth in the Tables
RSFSVEREL

SEQ ID NO: 946 - protein, 8 aa, Homo sapiens
  (1)..(8): One or more of the listed amino acids can be modified as set forth in the Tables
RSFTPLSI

SEQ ID NO: 947 - protein, 10 aa, Homo sapiens
  (1)..(10): One or more of the listed amino acids can be modified as set forth in the Tables
RSFTPLSILK

SEQ ID NO: 948 - protein, 9 aa, Homo sapiens
  (1)..(9): One or more of the listed amino acids can be modified as set forth in the Tables
RSHSLHYLF

SEQ ID NO: 949 - protein, 9 aa, Homo sapiens
  (1)..(9): One or more of the listed amino acids can be modified as set forth in the Tables
RSHSPLRSK

SEQ ID NO: 950 - protein, 9 aa, Homo sapiens
  (1)..(9): One or more of the listed amino acids can be modified as set forth in the Tables
RSHSPMSNR

SEQ ID NO: 951 - protein, 9 aa, Homo sapiens
  (1)..(9): One or more of the listed amino acids can be modified as set forth in the Tables
RSHSPPLKL

SEQ ID NO: 952 - protein, 9 aa, Homo sapiens
  (1)..(9): One or more of the listed amino acids can be modified as set forth in the Tables
RSHSSPASL

SEQ ID NO: 953 - protein, 10 aa, Homo sapiens
  (1)..(10): One or more of the listed amino acids can be modified as set forth in the Tables
RSISASDLTF

SEQ ID NO: 954 - protein, 10 aa, Homo sapiens
  (1)..(10): One or more of the listed amino acids can be modified as set forth in the Tables
RSISNEGLTL

SEQ ID NO: 955 - protein, 9 aa, Homo sapiens
  (1)..(9): One or more of the listed amino acids can be modified as set forth in the Tables
RSISSLLRF

SEQ ID NO: 956 - protein, 9 aa, Homo sapiens
  (1)..(9): One or more of the listed amino acids can be modified as set forth in the Tables
RSISTPTCL

SEQ ID NO: 957 - protein, 9 aa, Homo sapiens
  (1)..(9): One or more of the listed amino acids can be modified as set forth in the Tables
RSISTPTCL

SEQ ID NO: 958 - protein, 9 aa, Homo sapiens
  (1)..(9): One or more of the listed amino acids can be modified as set forth in the Tables
RSISTPTCL

SEQ ID NO: 959 - protein, 9 aa, Homo sapiens
  (1)..(9): One or more of the listed amino acids can be modified as set forth in the Tables
RSISTPTCL

SEQ ID NO: 960 - protein, 9 aa, Homo sapiens
  (1)..(9): One or more of the listed amino acids can be modified as set forth in the Tables
RSKSATLLY

SEQ ID NO: 961 - protein, 9 aa, Homo sapiens
  (1)..(9): One or more of the listed amino acids can be modified as set forth in the Tables
RSKSLTNLV

SEQ ID NO: 962 - protein, 9 aa, Homo sapiens
  (1)..(9): One or more of the listed amino acids can be modified as set forth in the Tables
RSKSSIMYF

SEQ ID NO: 963 - protein, 9 aa, Homo sapiens
  (1)..(9): One or more of the listed amino acids can be modified as set forth in the Tables
RSKTPPKSY

SEQ ID NO: 964 - protein, 11 aa, Homo sapiens
  (1)..(11): One or more of the listed amino acids can be modified as set forth in the Tables
RSLGSVQAPSY

SEQ ID NO: 965 - protein, 9 aa, Homo sapiens
  (1)..(9): One or more of the listed amino acids can be modified as set forth in the Tables
RSLSASPAL

SEQ ID NO: 966 - protein, 10 aa, Homo sapiens
  (1)..(10): One or more of the listed amino acids can be modified as set forth in the Tables
RSLSERLLQL

SEQ ID NO: 967 - protein, 9 aa, Homo sapiens
  (1)..(9): One or more of the listed amino acids can be modified as set forth in the Tables
RSLSFSDEM

SEQ ID NO: 968 - protein, 11 aa, Homo sapiens
  (1)..(11): One or more of the listed amino acids can be modified as set forth in the Tables
RSLSPFRRHSW

SEQ ID NO: 969 - protein, 11 aa, Homo sapiens
  (1)..(11): One or more of the listed amino acids can be modified as set forth in the Tables
RSLSPFRRHSW

SEQ ID NO: 970 - protein, 12 aa, Homo sapiens
  (1)..(12): One or more of the listed amino acids can be modified as set forth in the Tables
RSLSPGGAALGY

SEQ ID NO: 971 - protein, 10 aa, Homo sapiens
  (1)..(10): One or more of the listed amino acids can be modified as set forth in the Tables
RSLSPILPGR

SEQ ID NO: 972 - protein, 9 aa, Homo sapiens
  (1)..(9): One or more of the listed amino acids can be modified as set forth in the Tables
RSLSPLIKF

SEQ ID NO: 973 - protein, 9 aa, Homo sapiens
  (1)..(9): One or more of the listed amino acids can be modified as set forth in the Tables
RSLSPMSGL

SEQ ID NO: 974 - protein, 11 aa, Homo sapiens
  (1)..(11): One or more of the listed amino acids can be modified as set forth in the Tables
RSLSPSSNSAF

SEQ ID NO: 975 - protein, 9 aa, Homo sapiens
  (1)..(9): One or more of the listed amino acids can be modified as set forth in the Tables
RSLSRVRVL

SEQ ID NO: 976 - protein, 9 aa, Homo sapiens
  (1)..(9): One or more of the listed amino acids can be modified as set forth in the Tables
RSLSSGESL

SEQ ID NO: 977 - protein, 10 aa, Homo sapiens
  (1)..(10): One or more of the listed amino acids can be modified as set forth in the Tables
RSLSSYRGKY

SEQ ID NO: 978 - protein, 9 aa, Homo sapiens
  (1)..(9): One or more of the listed amino acids can be modified as set forth in the Tables
RSLSTTNVF

SEQ ID NO: 979 - protein, 9 aa, Homo sapiens
  (1)..(9): One or more of the listed amino acids can be modified as set forth in the Tables
RSLSVGSEF

SEQ ID NO: 980 - protein, 9 aa, Homo sapiens
  (1)..(9): One or more of the listed amino acids can be modified as set forth in the Tables
RSLSVPVDL

SEQ ID NO: 981 - protein, 8 aa, Homo sapiens
  (1)..(8): One or more of the listed amino acids can be modified as set forth in the Tables
RSLTHLSL

SEQ ID NO: 982 - protein, 9 aa, Homo sapiens
  (1)..(9): One or more of the listed amino acids can be modified as set forth in the Tables
RSLTHPPTI

SEQ ID NO: 983 - protein, 9 aa, Homo sapiens
  (1)..(9): One or more of the listed amino acids can be modified as set forth in the Tables
RSMSGGHGL

SEQ ID NO: 984 - protein, 9 aa, Homo sapiens
  (1)..(9): One or more of the listed amino acids can be modified as set forth in the Tables
RSNSLVSTF

SEQ ID NO: 985 - protein, 9 aa, Homo sapiens
  (1)..(9): One or more of the listed amino acids can be modified as set forth in the Tables
RSNSPLPSI

SEQ ID NO: 986 - protein, 11 aa, Homo sapiens
  (1)..(11): One or more of the listed amino acids can be modified as set forth in the Tables
RSPEPDPYLSY

SEQ ID NO: 987 - protein, 9 aa, Homo sapiens
  (1)..(9): One or more of the listed amino acids can be modified as set forth in the Tables
RSPSFNMQL

SEQ ID NO: 988 - protein, 10 aa, Homo sapiens
  (1)..(10): One or more of the listed amino acids can be modified as set forth in the Tables
RSPSKPTLAY

SEQ ID NO: 989 - protein, 9 aa, Homo sapiens
  (1)..(9): One or more of the listed amino acids can be modified as set forth in the Tables
RSPSPKTSL

SEQ ID NO: 990 - protein, 11 aa, Homo sapiens
  (1)..(11): One or more of the listed amino acids can be modified as set forth in the Tables
RSPSPSFRWPF

SEQ ID NO: 991 - protein, 10 aa, Homo sapiens
  (1)..(10): One or more of the listed amino acids can be modified as set forth in the Tables
RSPSPTLSYY

SEQ ID NO: 992 - protein, 10 aa, Homo sapiens
  (1)..(10): One or more of the listed amino acids can be modified as set forth in the Tables
RSPTKSSLDY

SEQ ID NO: 993 - protein, 11 aa, Homo sapiens
  (1)..(11): One or more of the listed amino acids can be modified as set forth in the Tables
RSPTKSSLDYR

SEQ ID NO: 994 - protein, 9 aa, Homo sapiens
  (1)..(9): One or more of the listed amino acids can be modified as set forth in the Tables
RSRPALSPL

SEQ ID NO: 995 - protein, 10 aa, Homo sapiens
  (1)..(10): One or more of the listed amino acids can be modified as set forth in the Tables
RSRSDNALHL

SEQ ID NO: 996 - protein, 9 aa, Homo sapiens
  (1)..(9): One or more of the listed amino acids can be modified as set forth in the Tables
RSRSPLGFY

SEQ ID NO: 997 - protein, 9 aa, Homo sapiens
  (1)..(9): One or more of the listed amino acids can be modified as set forth in the Tables
RSRSPPPVS

SEQ ID NO: 998 - protein, 9 aa, Homo sapiens
  (1)..(9): One or more of the listed amino acids can be modified as set forth in the Tables
RSRSRDRMY

SEQ ID NO: 999 - protein, 9 aa, Homo sapiens
  (1)..(9): One or more of the listed amino acids can be modified as set forth in the Tables
RSRSVPVSF

SEQ ID NO: 1000 - protein, 10 aa, Homo sapiens
  (1)..(10): One or more of the listed amino acids can be modified as set forth in the Tables
RSRSYSPRRY

SEQ ID NO: 1001 - protein, 9 aa, Homo sapiens
  (1)..(9): One or more of the listed amino acids can be modified as set forth in the Tables
RSRSYTPEY

SEQ ID NO: 1002 - protein, 8 aa, Homo sapiens
  (1)..(8): One or more of the listed amino acids can be modified as set forth in the Tables
RSSFLQVF

SEQ ID NO: 1003 - protein, 9 aa, Homo sapiens
  (1)..(9): One or more of the listed amino acids can be modified as set forth in the Tables
RSSPRTISF

SEQ ID NO: 1004 - protein, 10 aa, Homo sapiens
  (1)..(10): One or more of the listed amino acids can be modified as set forth in the Tables
RSSQFGSLEF

SEQ ID NO: 1005 - protein, 9 aa, Homo sapiens
  (1)..(9): One or more of the listed amino acids can be modified as set forth in the Tables
RSSSAPLGL

SEQ ID NO: 1006 - protein, 10 aa, Homo sapiens
  (1)..(10): One or more of the listed amino acids can be modified as set forth in the Tables
RSSSFKDFAK

SEQ ID NO: 1007 - protein, 9 aa, Homo sapiens
  (1)..(9): One or more of the listed amino acids can be modified as set forth in the Tables
RSSSFSDTL

SEQ ID NO: 1008 - protein, 10 aa, Homo sapiens
  (1)..(10): One or more of the listed amino acids can be modified as set forth in the Tables
RSSSFVLPKL

SEQ ID NO: 1009 - protein, 10 aa, Homo sapiens
  (1)..(10): One or more of the listed amino acids can be modified as set forth in the Tables
RSSSFVLPKL

SEQ ID NO: 1010 - protein, 9 aa, Homo sapiens
  (1)..(9): One or more of the listed amino acids can be modified as set forth in the Tables
RSSSLQRRV

SEQ ID NO: 1011 - protein, 10 aa, Homo sapiens
  (1)..(10): One or more of the listed amino acids can be modified as set forth in the Tables
RSSSLSDFSW

SEQ ID NO: 1012 - protein, 9 aa, Homo sapiens
  (1)..(9): One or more of the listed amino acids can be modified as set forth in the Tables
RSSSPFLSK

SEQ ID NO: 1013 - protein, 8 aa, Homo sapiens
  (1)..(8): One or more of the listed amino acids can be modified as set forth in the Tables
RSSSPLQL

SEQ ID NO: 1014 - protein, 10 aa, Homo sapiens
  (1)..(10): One or more of the listed amino acids can be modified as set forth in the Tables
RSSSPPILTK

SEQ ID NO: 1015 - protein, 9 aa, Homo sapiens
  (1)..(9): One or more of the listed amino acids can be modified as set forth in the Tables
RSSSPVTEL

SEQ ID NO: 1016 - protein, 9 aa, Homo sapiens
  (1)..(9): One or more of the listed amino acids can be modified as set forth in the Tables
RSSTPLPTI

SEQ ID NO: 1017 - protein, 9 aa, Homo sapiens
  (1)..(9): One or more of the listed amino acids can be modified as set forth in the Tables
RSTSLSLKY

SEQ ID NO: 1018 - protein, 9 aa, Homo sapiens
  (1)..(9): One or more of the listed amino acids can be modified as set forth in the Tables
RSVSGFLHF

SEQ ID NO: 1019 - protein, 9 aa, Homo sapiens
  (1)..(9): One or more of the listed amino acids can be modified as set forth in the Tables
RSVSLDSQM

SEQ ID NO: 1020 - protein, 11 aa, Homo sapiens
  (1)..(11): One or more of the listed amino acids can be modified as set forth in the Tables
RSVSLDSQMGY

SEQ ID NO: 1021 - protein, 8 aa, Homo sapiens
  (1)..(8): One or more of the listed amino acids can be modified as set forth in the Tables
RSVSPTFL

SEQ ID NO: 1022 - protein, 9 aa, Homo sapiens
  (1)..(9): One or more of the listed amino acids can be modified as set forth in the Tables
RSVSPTTEM

SEQ ID NO: 1023 - protein, 9 aa, Homo sapiens
  (1)..(9): One or more of the listed amino acids can be modified as set forth in the Tables
RSVSPVQDL

SEQ ID NO: 1024 - protein, 9 aa, Homo sapiens
  (1)..(9): One or more of the listed amino acids can be modified as set forth in the Tables
RSWSPPPEV

SEQ ID NO: 1025 - protein, 11 aa, Homo sapiens
  (1)..(11): One or more of the listed amino acids can be modified as set forth in the Tables
RSWSPPPEVSR

SEQ ID NO: 1026 - protein, 10 aa, Homo sapiens
  (1)..(10): One or more of the listed amino acids can be modified as set forth in the Tables
RSYSDPPLKF

SEQ ID NO: 1027 - protein, 11 aa, Homo sapiens
  (1)..(11): One or more of the listed amino acids can be modified as set forth in the Tables
RSYSPERSKSY

SEQ ID NO: 1028 - protein, 13 aa, Homo sapiens
  (1)..(13): One or more of the listed amino acids can be modified as set forth in the Tables
RSYSPERSKSYSF

SEQ ID NO: 1029 - protein, 9 aa, Homo sapiens
  (1)..(9): One or more of the listed amino acids can be modified as set forth in the Tables
RSYSRLETL

SEQ ID NO: 1030 - protein, 13 aa, Homo sapiens
  (1)..(13): One or more of the listed amino acids can be modified as set forth in the Tables
RTASLSNQECQLY

SEQ ID NO: 1031 - protein, 9 aa, Homo sapiens
  (1)..(9): One or more of the listed amino acids can be modified as set forth in the Tables
RTASLVSGL

SEQ ID NO: 1032 - protein, 10 aa, Homo sapiens
  (1)..(10): One or more of the listed amino acids can be modified as set forth in the Tables
RTASPPALPK

SEQ ID NO: 1033 - protein, 12 aa, Homo sapiens
  (1)..(12): One or more of the listed amino acids can be modified as set forth in the Tables
RTATADDKKLQF

SEQ ID NO: 1034 - protein, 9 aa, Homo sapiens
  (1)..(9): One or more of the listed amino acids can be modified as set forth in the Tables
RTDSIGEKL

SEQ ID NO: 1035 - protein, 12 aa, Homo sapiens
  (1)..(12): One or more of the listed amino acids can be modified as set forth in the Tables
RTDSIGEKLGRY

SEQ ID NO: 1036 - protein, 9 aa, Homo sapiens
  (1)..(9): One or more of the listed amino acids can be modified as set forth in the Tables
RTDSREQKL

SEQ ID NO: 1037 - protein, 9 aa, Homo sapiens
  (1)..(9): One or more of the listed amino acids can be modified as set forth in the Tables
RTDSRGVNL

SEQ ID NO: 1038 - protein, 10 aa, Homo sapiens
  (1)..(10): One or more of the listed amino acids can be modified as set forth in the Tables
RTFSDESNVL

SEQ ID NO: 1039 - protein, 9 aa, Homo sapiens
  (1)..(9): One or more of the listed amino acids can be modified as set forth in the Tables
RTFSESSVW

SEQ ID NO: 1040 - protein, 7 aa, Homo sapiens
  (1)..(7): One or more of the listed amino acids can be modified as set forth in the Tables
RTFSPTY

SEQ ID NO: 1041 - protein, 11 aa, Homo sapiens
  (1)..(11): One or more of the listed amino acids can be modified as set forth in the Tables
RTFSPTYGLLR

SEQ ID NO: 1042 - protein, 9 aa, Homo sapiens
  (1)..(9): One or more of the listed amino acids can be modified as set forth in the Tables
RTFSYIKNK

SEQ ID NO: 1043 - protein, 9 aa, Homo sapiens
  (1)..(9): One or more of the listed amino acids can be modified as set forth in the Tables
RTGSPALGL

SEQ ID NO: 1044 - protein, 11 aa, Homo sapiens
  (1)..(11): One or more of the listed amino acids can be modified as set forth in the Tables
RTISAQDTLAY

SEQ ID NO: 1045 - protein, 11 aa, Homo sapiens
  (1)..(11): One or more of the listed amino acids can be modified as set forth in the Tables
RTISNPEVVMK

SEQ ID NO: 1046 - protein, 11 aa, Homo sapiens
  (1)..(11): One or more of the listed amino acids can be modified as set forth in the Tables
RTISPPTLGTL

SEQ ID NO: 1047 - protein, 9 aa, Homo sapiens
  (1)..(9): One or more of the listed amino acids can be modified as set forth in the Tables
RTISQSSSL

SEQ ID NO: 1048 - protein, 9 aa, Homo sapiens
  (1)..(9): One or more of the listed amino acids can be modified as set forth in the Tables
RTISVILFL

SEQ ID NO: 1049 - protein, 10 aa, Homo sapiens
  (1)..(10): One or more of the listed amino acids can be modified as set forth in the Tables
RTLHSPPLQL

SEQ ID NO: 1050 - protein, 9 aa, Homo sapiens
  (1)..(9): One or more of the listed amino acids can be modified as set forth in the Tables
RTLSMDKGF

SEQ ID NO: 1051 - protein, 9 aa, Homo sapiens
  (1)..(9): One or more of the listed amino acids can be modified as set forth in the Tables
RTLSPSSGY

SEQ ID NO: 1052 - protein, 9 aa, Homo sapiens
  (1)..(9): One or more of the listed amino acids can be modified as set forth in the Tables
RTLSVESLI

SEQ ID NO: 1053 - protein, 9 aa, Homo sapiens
  (1)..(9): One or more of the listed amino acids can be modified as set forth in the Tables
RTMSPIQVL

SEQ ID NO: 1054 - protein, 9 aa, Homo sapiens
  (1)..(9): One or more of the listed amino acids can be modified as set forth in the Tables
RTMSPIQVL

SEQ ID NO: 1055 - protein, 9 aa, Homo sapiens
  (1)..(9): One or more of the listed amino acids can be modified as set forth in the Tables
RTPSISFHH

SEQ ID NO: 1056 - protein, 10 aa, Homo sapiens
  (1)..(10): One or more of the listed amino acids can be modified as set forth in the Tables
RTPSPARPAL

SEQ ID NO: 1057 - protein, 12 aa, Homo sapiens
  (1)..(12): One or more of the listed amino acids can be modified as set forth in the Tables
RTPSPKSLPSYL

SEQ ID NO: 1058 - protein, 9 aa, Homo sapiens
  (1)..(9): One or more of the listed amino acids can be modified as set forth in the Tables
RTPSQIIRK

SEQ ID NO: 1059 - protein, 11 aa, Homo sapiens
  (1)..(11): One or more of the listed amino acids can be modified as set forth in the Tables
RTPSSSSTLAY

SEQ ID NO: 1060 - protein, 9 aa, Homo sapiens
  (1)..(9): One or more of the listed amino acids can be modified as set forth in the Tables
RTRSLPITI

SEQ ID NO: 1061 - protein, 9 aa, Homo sapiens
  (1)..(9): One or more of the listed amino acids can be modified as set forth in the Tables
RTSSFALNL

SEQ ID NO: 1062 - protein, 11 aa, Homo sapiens
  (1)..(11): One or more of the listed amino acids can be modified as set forth in the Tables
RTSSQRSTLTY

SEQ ID NO: 1063 - protein, 9 aa, Homo sapiens
  (1)..(9): One or more of the listed amino acids can be modified as set forth in the Tables
RTVSPAHVL

SEQ ID NO: 1064 - protein, 9 aa, Homo sapiens
  (1)..(9): One or more of the listed amino acids can be modified as set forth in the Tables
RTVSPELIL

SEQ ID NO: 1065 - protein, 9 aa, Homo sapiens
  (1)..(9): One or more of the listed amino acids can be modified as set forth in the Tables
RTYSLGSAL

SEQ ID NO: 1066 - protein, 9 aa, Homo sapiens
  (1)..(9): One or more of the listed amino acids can be modified as set forth in the Tables
RVASPKLVM

SEQ ID NO: 1067 - protein, 9 aa, Homo sapiens
  (1)..(9): One or more of the listed amino acids can be modified as set forth in the Tables
RVASPSRKV

SEQ ID NO: 1068 - protein, 9 aa, Homo sapiens
  (1)..(9): One or more of the listed amino acids can be modified as set forth in the Tables
RVASWAVSF

SEQ ID NO: 1069 - protein, 9 aa, Homo sapiens
  (1)..(9): One or more of the listed amino acids can be modified as set forth in the Tables
RVDSLEFSL

SEQ ID NO: 1070 - protein, 8 aa, Homo sapiens
RVDSPVTV

SEQ ID NO: 1071 - protein, 9 aa, Homo sapiens
  (1)..(9): One or more of the listed amino acids can be modified as set forth in the Tables
RVDSTTCLF

SEQ ID NO: 1072 - protein, 9 aa, Homo sapiens
  (1)..(9): One or more of the listed amino acids can be modified as set forth in the Tables
RVKSWADNL

SEQ ID NO: 1073 - protein, 12 aa, Homo sapiens
  (1)..(12): One or more of the listed amino acids can be modified as set forth in the Tables
RVKVDGPRSPSY

SEQ ID NO: 1074 - protein, 10 aa, Homo sapiens
  (1)..(10): One or more of the listed amino acids can be modified as set forth in the Tables
RVMSSPSAMK

SEQ ID NO: 1075 - protein, 9 aa, Homo sapiens
  (1)..(9): One or more of the listed amino acids can be modified as set forth in the Tables
RVPSINQKI

SEQ ID NO: 1076 - protein, 9 aa, Homo sapiens
  (1)..(9): One or more of the listed amino acids can be modified as set forth in the Tables
RVPSKSLDL

SEQ ID NO: 1077 - protein, 9 aa, Homo sapiens
  (1)..(9): One or more of the listed amino acids can be modified as set forth in the Tables
RVPSKSLDL

SEQ ID NO: 1078 - protein, 10 aa, Homo sapiens
  (1)..(10): One or more of the listed amino acids can be modified as set forth in the Tables
RVPSPTPAPK

SEQ ID NO: 1079 - protein, 11 aa, Homo sapiens
  (1)..(11): One or more of the listed amino acids can be modified as set forth in the Tables
RVRRSSFLNAK

SEQ ID NO: 1080 - protein, 9 aa, Homo sapiens
  (1)..(9): One or more of the listed amino acids can be modified as set forth in the Tables
RVSSLTLHL

SEQ ID NO: 1081 - protein, 9 aa, Homo sapiens
  (1)..(9): One or more of the listed amino acids can be modified as set forth in the Tables
RVSSPLASF

SEQ ID NO: 1082 - protein, 9 aa, Homo sapiens
  (1)..(9): One or more of the listed amino acids can be modified as set forth in the Tables
RVVPSPLQF

SEQ ID NO: 1083 - protein, 9 aa, Homo sapiens
  (1)..(9): One or more of the listed amino acids can be modified as set forth in the Tables
RVVSPGIDL

SEQ ID NO: 1084 - protein, 10 aa, Homo sapiens
  (1)..(10): One or more of the listed amino acids can be modified as set forth in the Tables
RVWEDRPSSA

SEQ ID NO: 1085 - protein, 10 aa, Homo sapiens
  (1)..(10): One or more of the listed amino acids can be modified as set forth in the Tables
RVYTYIQSRF

SEQ ID NO: 1086 - protein, 12 aa, Homo sapiens
  (1)..(12): One or more of the listed amino acids can be modified as set forth in the Tables
RYLGGSMDLSTF

SEQ ID NO: 1087 - protein, 9 aa, Homo sapiens
  (1)..(9): One or more of the listed amino acids can be modified as set forth in the Tables
RYPTSIASL

SEQ ID NO: 1088 - protein, 13 aa, Homo sapiens
  (1)..(13): One or more of the listed amino acids can be modified as set forth in the Tables
RYRSPEPDPYLSY

SEQ ID NO: 1089 - protein, 10 aa, Homo sapiens
  (1)..(10): One or more of the listed amino acids can be modified as set forth in the Tables
SAASPVVSSM

SEQ ID NO: 1090 - protein, 9 aa, Homo sapiens
  (1)..(9): One or more of the listed amino acids can be modified as set forth in the Tables
SAESKTIEF

SEQ ID NO: 1091 - protein, 14 aa, Homo sapiens
  (1)..(14): One or more of the listed amino acids can be modified as set forth in the Tables
SAGGSAEALLSDLH

SEQ ID NO: 1092 - protein, 16 aa, Homo sapiens
  (1)..(16): One or more of the listed amino acids can be modified as set forth in the Tables
SAGGSAEALLSDLHAF

SEQ ID NO: 1093 - protein, 9 aa, Homo sapiens
  (1)..(9): One or more of the listed amino acids can be modified as set forth in the Tables
SAISPKSSL

SEQ ID NO: 1094 - protein, 9 aa, Homo sapiens
  (1)..(9): One or more of the listed amino acids can be modified as set forth in the Tables
SAISPTPEI

SEQ ID NO: 1095 - protein, 9 aa, Homo sapiens
  (1)..(9): One or more of the listed amino acids can be modified as set forth in the Tables
SAKSPLPSY

SEQ ID NO: 1096 - protein, 9 aa, Homo sapiens
  (1)..(9): One or more of the listed amino acids can be modified as set forth in the Tables
SAMSPTHHL

SEQ ID NO: 1097 - protein, 11 aa, Homo sapiens
  (1)..(11): One or more of the listed amino acids can be modified as set forth in the Tables
SAQGSDVSLTA

SEQ ID NO: 1098 - protein, 12 aa, Homo sapiens
  (1)..(12): One or more of the listed amino acids can be modified as set forth in the Tables
SAYGGLTSPGLS

SEQ ID NO: 1099 - protein, 13 aa, Homo sapiens
  (1)..(13): One or more of the listed amino acids can be modified as set forth in the Tables
SAYGGLTSPGLSY

SEQ ID NO: 1100 - protein, 15 aa, Homo sapiens
  (1)..(15): One or more of the listed amino acids can be modified as set forth in the Tables
SAYGGLTSPGLSYSL

SEQ ID NO: 1101 - protein, 10 aa, Homo sapiens
  (1)..(10): One or more of the listed amino acids can be modified as set forth in the Tables
SDDEKMPDLE

SEQ ID NO: 1102 - protein, 9 aa, Homo sapiens
  (1)..(9): One or more of the listed amino acids can be modified as set forth in the Tables
SDMPRAHSF

SEQ ID NO: 1103 - protein, 9 aa, Homo sapiens
  (1)..(9): One or more of the listed amino acids can be modified as set forth in the Tables
SDMPRAHSF

SEQ ID NO: 1104 - protein, 12 aa, Homo sapiens
  (1)..(12): One or more of the listed amino acids can be modified as set forth in the Tables
SDSAQGSESHSL

SEQ ID NO: 1105 - protein, 11 aa, Homo sapiens
  (1)..(11): One or more of the listed amino acids can be modified as set forth in the Tables
SDSPPRPQPAF

SEQ ID NO: 1106 - protein, 14 aa, Homo sapiens
  (1)..(14): One or more of the listed amino acids can be modified as set forth in the Tables
SDSPPRPQPAFKYQ

SEQ ID NO: 1107 - protein, 15 aa, Homo sapiens
  (1)..(15): One or more of the listed amino acids can be modified as set forth in the Tables
SDYAVHPMSPVGRTS

SEQ ID NO: 1108 - protein, 15 aa, Homo sapiens
  (1)..(15): One or more of the listed amino acids can be modified as set forth in the Tables
SDYAVHPMSPVGRTS

SEQ ID NO: 1109 - protein, 9 aa, Homo sapiens
  (1)..(9): One or more of the listed amino acids can be modified as set forth in the Tables
SEASPSREA

SEQ ID NO: 1110 - protein, 10 aa, Homo sapiens
  (1)..(10): One or more of the listed amino acids can be modified as set forth in the Tables
SEASPSREAI

SEQ ID NO: 1111 - protein, 9 aa, Homo sapiens
  (1)..(9): One or more of the listed amino acids can be modified as set forth in the Tables
SEDSSRGAF

SEQ ID NO: 1112 - protein, 11 aa, Homo sapiens
  (1)..(11): One or more of the listed amino acids can be modified as set forth in the Tables
SEFTGFSGMSF

SEQ ID NO: 1113 - protein, 9 aa, Homo sapiens
  (1)..(9): One or more of the listed amino acids can be modified as set forth in the Tables
SEGSLDRLY

SEQ ID NO: 1114 - protein, 9 aa, Homo sapiens
  (1)..(9): One or more of the listed amino acids can be modified as set forth in the Tables
SELSPGRSV

SEQ ID NO: 1115 - protein, 9 aa, Homo sapiens
  (1)..(9): One or more of the listed amino acids can be modified as set forth in the Tables
SELTPSESL

SEQ ID NO: 1116 - protein, 9 aa, Homo sapiens
  (1)..(9): One or more of the listed amino acids can be modified as set forth in the Tables
SERIMQLSL

SEQ ID NO: 1117 - protein, 9 aa, Homo sapiens
  (1)..(9): One or more of the listed amino acids can be modified as set forth in the Tables
SESKSMPVL

SEQ ID NO: 1118 - protein, 10 aa, Homo sapiens
  (1)..(10): One or more of the listed amino acids can be modified as set forth in the Tables
SEVSPSGVGF

SEQ ID NO: 1119 - protein, 9 aa, Homo sapiens
  (1)..(9): One or more of the listed amino acids can be modified as set forth in the Tables
SEYQWITSP

SEQ ID NO: 1120 - protein, 9 aa, Homo sapiens
  (1)..(9): One or more of the listed amino acids can be modified as set forth in the Tables
SFDSGIAGL

SEQ ID NO: 1121 - protein, 9 aa, Homo sapiens
  (1)..(9): One or more of the listed amino acids can be modified as set forth in the Tables
SFDSGSVRL

SEQ ID NO: 1122 - protein, 9 aa, Homo sapiens
  (1)..(9): One or more of the listed amino acids can be modified as set forth in the Tables
SFLPRTLSL

SEQ ID NO: 1123 - protein, 8 aa, Homo sapiens
  (1)..(8): One or more of the listed amino acids can be modified as set forth in the Tables
SGPEIFTF

SEQ ID NO: 1124 - protein, 8 aa, Homo sapiens
  (1)..(8): One or more of the listed amino acids can be modified as set forth in the Tables
SIDSPEKL

SEQ ID NO: 1125 - protein, 7 aa, Homo sapiens
  (1)..(7): One or more of the listed amino acids can be modified as set forth in the Tables
SIELPSM

SEQ ID NO: 1126 - protein, 10 aa, Homo sapiens
  (1)..(10): One or more of the listed amino acids can be modified as set forth in the Tables
SIGSPVKVGK

SEQ ID NO: 1127 - protein, 9 aa, Homo sapiens
  (1)..(9): One or more of the listed amino acids can be modified as set forth in the Tables
SIISPDFSF

SEQ ID NO: 1128 - protein, 9 aa, Homo sapiens
  (1)..(9): One or more of the listed amino acids can be modified as set forth in the Tables
SIISPNFSF

SEQ ID NO: 1129 - protein, 9 aa, Homo sapiens
  (1)..(9): One or more of the listed amino acids can be modified as set forth in the Tables
SILSRTPSV

SEQ ID NO: 1130 - protein, 9 aa, Homo sapiens
  (1)..(9): One or more of the listed amino acids can be modified as set forth in the Tables
SIMSFHIDL

SEQ ID NO: 1131 - protein, 9 aa, Homo sapiens
  (1)..(9): One or more of the listed amino acids can be modified as set forth in the Tables
SIPSGYLEL

SEQ ID NO: 1132 - protein, 9 aa, Homo sapiens
  (1)..(9): One or more of the listed amino acids can be modified as set forth in the Tables
SIRYSGHSL

SEQ ID NO: 1133 - protein, 9 aa, Homo sapiens
  (1)..(9): One or more of the listed amino acids can be modified as set forth in the Tables
SISSIDREL

SEQ ID NO: 1134 - protein, 9 aa, Homo sapiens
  (1)..(9): One or more of the listed amino acids can be modified as set forth in the Tables
SISSVSNTF

SEQ ID NO: 1135 - protein, 14 aa, Homo sapiens
  (1)..(14): One or more of the listed amino acids can be modified as set forth in the Tables
SISVQVNSIKFDSE

SEQ ID NO: 1136 - protein, 13 aa, Homo sapiens
  (1)..(13): One or more of the listed amino acids can be modified as set forth in the Tables
SITITPPDRYDSL

SEQ ID NO: 1137 - protein, 19 aa, Homo sapiens
  (1)..(19): One or more of the listed amino acids can be modified as set forth in the Tables
SKSPSLSPSPPSPLEKTPL

SEQ ID NO: 1138 - protein, 9 aa, Homo sapiens
  (1)..(9): One or more of the listed amino acids can be modified as set forth in the Tables
SLASLLAKV

SEQ ID NO: 1139 - protein, 9 aa, Homo sapiens
  (1)..(9): One or more of the listed amino acids can be modified as set forth in the Tables
SLDSLDLRV

SEQ ID NO: 1140 - protein, 10 aa, Homo sapiens
  (1)..(10): One or more of the listed amino acids can be modified as set forth in the Tables
SLDSPGPEKM

SEQ ID NO: 1141 - protein, 12 aa, Homo sapiens
  (1)..(12): One or more of the listed amino acids can be modified as set forth in the Tables
SLDSPGPEKMAL

SEQ ID NO: 1142 - protein, 12 aa, Homo sapiens
  (1)..(12): One or more of the listed amino acids can be modified as set forth in the Tables
SLDSPGPEKMAL

SEQ ID NO: 1143 - protein, 11 aa, Homo sapiens
  (1)..(11): One or more of the listed amino acids can be modified as set forth in the Tables
SLDSQQDSMKY

SEQ ID NO: 1144 - protein, 11 aa, Homo sapiens
  (1)..(11): One or more of the listed amino acids can be modified as set forth in the Tables
SLDSQQDSMKY

SEQ ID NO: 1145 - protein, 9 aa, Homo sapiens
  (1)..(9): One or more of the listed amino acids can be modified as set forth in the Tables
SLDSSNSGF

SEQ ID NO: 1146 - protein, 13 aa, Homo sapiens
  (1)..(13): One or more of the listed amino acids can be modified as set forth in the Tables
SLEEPKQANGGAY

SEQ ID NO: 1147 - protein, 8 aa, Homo sapiens
  (1)..(8): One or more of the listed amino acids can be modified as set forth in the Tables
SLGPIRSL

SEQ ID NO: 1148 - protein, 10 aa, Homo sapiens
  (1)..(10): One or more of the listed amino acids can be modified as set forth in the Tables
SLHSLGSVSL

SEQ ID NO: 1149 - protein, 9 aa, Homo sapiens
  (1)..(9): One or more of the listed amino acids can be modified as set forth in the Tables
SLIDGYYRL

SEQ ID NO: 1150 - protein, 9 aa, Homo sapiens
  (1)..(9): One or more of the listed amino acids can be modified as set forth in the Tables
SLLSELQHA

SEQ ID NO: 1151 - protein, 9 aa, Homo sapiens
  (1)..(9): One or more of the listed amino acids can be modified as set forth in the Tables
SLLSLQTEL

SEQ ID NO: 1152 - protein, 9 aa, Homo sapiens
  (1)..(9): One or more of the listed amino acids can be modified as set forth in the Tables
SLLSVSHAL

SEQ ID NO: 1153 - protein, 9 aa, Homo sapiens
  (1)..(9): One or more of the listed amino acids can be modified as set forth in the Tables
SLMSPGRRK

SEQ ID NO: 1154 - protein, 10 aa, Homo sapiens
  (1)..(10): One or more of the listed amino acids can be modified as set forth in the Tables
SLMTISHPGL

SEQ ID NO: 1155 - protein, 9 aa, Homo sapiens
  (1)..(9): One or more of the listed amino acids can be modified as set forth in the Tables
SLNSSPVSK

SEQ ID NO: 1156 - protein, 9 aa, Homo sapiens
  (1)..(9): One or more of the listed amino acids can be modified as set forth in the Tables
SLSSERYYL

SEQ ID NO: 1157 - protein, 9 aa, Homo sapiens
  (1)..(9): One or more of the listed amino acids can be modified as set forth in the Tables
SLSSPTVTL

SEQ ID NO: 1158 - protein, 11 aa, Homo sapiens
  (1)..(11): One or more of the listed amino acids can be modified as set forth in the Tables
SMKSPLYLVSR

SEQ ID NO: 1159 - protein, 11 aa, Homo sapiens
  (1)..(11): One or more of the listed amino acids can be modified as set forth in the Tables
SMKSPLYLVSR

SEQ ID NO: 1160 - protein, 9 aa, Homo sapiens
  (1)..(9): One or more of the listed amino acids can be modified as set forth in the Tables
SMTRSPPRV

SEQ ID NO: 1161 - protein, 9 aa, Homo sapiens
  (1)..(9): One or more of the listed amino acids can be modified as set forth in the Tables
SPASPLKEL

SEQ ID NO: 1162 - protein, 9 aa, Homo sapiens
  (1)..(9): One or more of the listed amino acids can be modified as set forth in the Tables
SPDHSDHTL

SEQ ID NO: 1163 - protein, 9 aa, Homo sapiens
  (1)..(9): One or more of the listed amino acids can be modified as set forth in the Tables
SPDSSQSSL

SEQ ID NO: 1164 - protein, 9 aa, Homo sapiens
  (1)..(9): One or more of the listed amino acids can be modified as set forth in the Tables
SPFLSKRSL

SEQ ID NO: 1165 - protein, 10 aa, Homo sapiens
  (1)..(10): One or more of the listed amino acids can be modified as set forth in the Tables
SPFQSSPLSL

SEQ ID NO: 1166 - protein, 10 aa, Homo sapiens
  (1)..(10): One or more of the listed amino acids can be modified as set forth in the Tables
SPFQSSPLSL

SEQ ID NO: 1167 - protein, 10 aa, Homo sapiens
  (1)..(10): One or more of the listed amino acids can be modified as set forth in the Tables
SPFSSRSPSL

SEQ ID NO: 1168 - protein, 9 aa, Homo sapiens
  (1)..(9): One or more of the listed amino acids can be modified as set forth in the Tables
SPGSPLHSL

SEQ ID NO: 1169 - protein, 9 aa, Homo sapiens
  (1)..(9): One or more of the listed amino acids can be modified as set forth in the Tables
SPGSPLVSM

SEQ ID NO: 1170 - protein, 9 aa, Homo sapiens
  (1)..(9): One or more of the listed amino acids can be modified as set forth in the Tables
SPHSPFYQL

SEQ ID NO: 1171 - protein, 9 aa, Homo sapiens
  (1)..(9): One or more of the listed amino acids can be modified as set forth in the Tables
SPHTPSTHF

SEQ ID NO: 1172 - protein, 11 aa, Homo sapiens
  (1)..(11): One or more of the listed amino acids can be modified as set forth in the Tables
SPHYFSPFRPY

SEQ ID NO: 1173 - protein, 11 aa, Homo sapiens
  (1)..(11): One or more of the listed amino acids can be modified as set forth in the Tables
SPIAPRSPAKL

SEQ ID NO: 1174 - protein, 7 aa, Homo sapiens
  (1)..(7): One or more of the listed amino acids can be modified as set forth in the Tables
SPIKVTL

SEQ ID NO: 1175 - protein, 9 aa, Homo sapiens
  (1)..(9): One or more of the listed amino acids can be modified as set forth in the Tables
SPKPPTRSP

SEQ ID NO: 1176 - protein, 12 aa, Homo sapiens
  (1)..(12): One or more of the listed amino acids can be modified as set forth in the Tables
SPKSGSPKSSSL

SEQ ID NO: 1177 - protein, 10 aa, Homo sapiens
  (1)..(10): One or more of the listed amino acids can be modified as set forth in the Tables
SPKSPGLKAM

SEQ ID NO: 1178 - protein, 10 aa, Homo sapiens
  (1)..(10): One or more of the listed amino acids can be modified as set forth in the Tables
SPLSKIGIEL

SEQ ID NO: 1179 - protein, 9 aa, Homo sapiens
  (1)..(9): One or more of the listed amino acids can be modified as set forth in the Tables
SPLSPTETF

SEQ ID NO: 1180 - protein, 9 aa, Homo sapiens
  (1)..(9): One or more of the listed amino acids can be modified as set forth in the Tables
SPLTKSISL

SEQ ID NO: 1181 - protein, 10 aa, Homo sapiens
  (1)..(10): One or more of the listed amino acids can be modified as set forth in the Tables
SPPDSPGRTL

SEQ ID NO: 1182 - protein, 10 aa, Homo sapiens
  (1)..(10): One or more of the listed amino acids can be modified as set forth in the Tables
SPPNLTPKPL

SEQ ID NO: 1183 - protein, 10 aa, Homo sapiens
  (1)..(10): One or more of the listed amino acids can be modified as set forth in the Tables
SPPSPARWSL

SEQ ID NO: 1184 - protein, 11 aa, Homo sapiens
  (1)..(11): One or more of the listed amino acids can be modified as set forth in the Tables
SPPSPLEKTPL

SEQ ID NO: 1185 - protein, 10 aa, Homo sapiens
  (1)..(10): One or more of the listed amino acids can be modified as set forth in the Tables
SPRDSPAVSL

SEQ ID NO: 1186 - protein, 11 aa, Homo sapiens
  (1)..(11): One or more of the listed amino acids can be modified as set forth in the Tables
SPRGSGSSTSL

SEQ ID NO: 1187 - protein, 10 aa, Homo sapiens
  (1)..(10): One or more of the listed amino acids can be modified as set forth in the Tables
SPRLPRSPRL

SEQ ID NO: 1188 - protein, 10 aa, Homo sapiens
  (1)..(10): One or more of the listed amino acids can be modified as set forth in the Tables
SPRPPNSPSI

SEQ ID NO: 1189 - protein, 12 aa, Homo sapiens
  (1)..(12): One or more of the listed amino acids can be modified as set forth in the Tables
SPRPPNSPSISI

SEQ ID NO: 1190 - protein, 10 aa, Homo sapiens
SPRRSLGLAL

SEQ ID NO: 1191 - protein, 10 aa, Homo sapiens
  (1)..(10): One or more of the listed amino acids can be modified as set forth in the Tables
SPRRSRSISL

SEQ ID NO: 1192 - protein, 9 aa, Homo sapiens
  (1)..(9): One or more of the listed amino acids can be modified as set forth in the Tables
SPRSESGGL

SEQ ID NO: 1193 - protein, 14 aa, Homo sapiens
  (1)..(14): One or more of the listed amino acids can be modified as set forth in the Tables
SPRSPGPLPGARGL

SEQ ID NO: 1194 - protein, 9 aa, Homo sapiens
  (1)..(9): One or more of the listed amino acids can be modified as set forth in the Tables
SPRSPGRSL

SEQ ID NO: 1195 - protein, 10 aa, Homo sapiens
  (1)..(10): One or more of the listed amino acids can be modified as set forth in the Tables
SPRSPISPEL

SEQ ID NO: 1196 - protein, 10 aa, Homo sapiens
  (1)..(10): One or more of the listed amino acids can be modified as set forth in the Tables
SPRSPQLSDF

SEQ ID NO: 1197 - protein, 9 aa, Homo sapiens
  (1)..(9): One or more of the listed amino acids can be modified as set forth in the Tables
SPRSPTPSY

SEQ ID NO: 1198 - protein, 10 aa, Homo sapiens
  (1)..(10): One or more of the listed amino acids can be modified as set forth in the Tables
SPRSPVPTTL

SEQ ID NO: 1199 - protein, 9 aa, Homo sapiens
  (1)..(9): One or more of the listed amino acids can be modified as set forth in the Tables
SPRTPPQRF

SEQ ID NO: 1200 - protein, 9 aa, Homo sapiens
  (1)..(9): One or more of the listed amino acids can be modified as set forth in the Tables
SPRTPSNTP

SEQ ID NO: 1201 - protein, 12 aa, Homo sapiens
  (1)..(12): One or more of the listed amino acids can be modified as set forth in the Tables
SPRTPTPFKHAL

SEQ ID NO: 1202 - protein, 11 aa, Homo sapiens
  (1)..(11): One or more of the listed amino acids can be modified as set forth in the Tables
SPRTPVSPVKF

SEQ ID NO: 1203 - protein, 9 aa, Homo sapiens
  (1)..(9): One or more of the listed amino acids can be modified as set forth in the Tables
SPSFGDPQL

SEQ ID NO: 1204 - protein, 21 aa, Homo sapiens
  (1)..(21): One or more of the listed amino acids can be modified as set forth in the Tables
SPSKSPSLSPSPPSPLEKTPL

SEQ ID NO: 1205 - protein, 17 aa, Homo sapiens
  (1)..(17): One or more of the listed amino acids can be modified as set forth in the Tables
SPSLSPSPPSPLEKTPL

SEQ ID NO: 1206 - protein, 9 aa, Homo sapiens
  (1)..(9): One or more of the listed amino acids can be modified as set forth in the Tables
SPSSPRVRL

SEQ ID NO: 1207 - protein, 9 aa, Homo sapiens
  (1)..(9): One or more of the listed amino acids can be modified as set forth in the Tables
SPTSPFSSL

SEQ ID NO: 1208 - protein, 11 aa, Homo sapiens
  (1)..(11): One or more of the listed amino acids can be modified as set forth in the Tables
SPVNKVRRVSF

SEQ ID NO: 1209 - protein, 9 aa, Homo sapiens
  (1)..(9): One or more of the listed amino acids can be modified as set forth in the Tables
SPVSPMKEL

SEQ ID NO: 1210 - protein, 18 aa, Homo sapiens
  (1)..(18): One or more of the listed amino acids can be modified as set forth in the Tables
SQAASSDSAQGSDVSLTA

SEQ ID NO: 1211 - protein, 8 aa, Homo sapiens
  (1)..(8): One or more of the listed amino acids can be modified as set forth in the Tables
SQDSPRKL

SEQ ID NO: 1212 - protein, 9 aa, Homo sapiens
  (1)..(9): One or more of the listed amino acids can be modified as set forth in the Tables
SQILRTPSL

SEQ ID NO: 1213 - protein, 10 aa, Homo sapiens
  (1)..(10): One or more of the listed amino acids can be modified as set forth in the Tables
SRHSGPFFTF

SEQ ID NO: 1214 - protein, 9 aa, Homo sapiens
  (1)..(9): One or more of the listed amino acids can be modified as set forth in the Tables
SRIPLVRSF

SEQ ID NO: 1215 - protein, 9 aa, Homo sapiens
  (1)..(9): One or more of the listed amino acids can be modified as set forth in the Tables
SRKSFVFEL

SEQ ID NO: 1216 - protein, 9 aa, Homo sapiens
  (1)..(9): One or more of the listed amino acids can be modified as set forth in the Tables
SRLSLRRSL

SEQ ID NO: 1217 - protein, 9 aa, Homo sapiens
  (1)..(9): One or more of the listed amino acids can be modified as set forth in the Tables
SRLSLRRSL

SEQ ID NO: 1218 - protein, 9 aa, Homo sapiens
  (1)..(9): One or more of the listed amino acids can be modified as set forth in the Tables
SRNQSPQRL

SEQ ID NO: 1219 - protein, 10 aa, Homo sapiens
  (1)..(10): One or more of the listed amino acids can be modified as set forth in the Tables
SRPSMSPTPL

SEQ ID NO: 1220 - protein, 9 aa, Homo sapiens
  (1)..(9): One or more of the listed amino acids can be modified as set forth in the Tables
SRPSSSRSY

SEQ ID NO: 1221 - protein, 9 aa, Homo sapiens
  (1)..(9): One or more of the listed amino acids can be modified as set forth in the Tables
SRSSSVLSL

SEQ ID NO: 1222 - protein, 9 aa, Homo sapiens
  (1)..(9): One or more of the listed amino acids can be modified as set forth in the Tables
SRSSSVLSL

SEQ ID NO: 1223 - protein, 10 aa, Homo sapiens
  (1)..(10): One or more of the listed amino acids can be modified as set forth in the Tables
SRYSGVNQSM

SEQ ID NO: 1224 - protein, 10 aa, Homo sapiens
  (1)..(10): One or more of the listed amino acids can be modified as set forth in the Tables
SRYSGVNQSM

SEQ ID NO: 1225 - protein, 9 aa, Homo sapiens
  (1)..(9): One or more of the listed amino acids can be modified as set forth in the Tables
SSAVDTLRS

SEQ ID NO: 1226 - protein, 10 aa, Homo sapiens
  (1)..(10): One or more of the listed amino acids can be modified as set forth in the Tables
SSDPASQLSY

SEQ ID NO: 1227 - protein, 14 aa, Homo sapiens
  (1)..(14): One or more of the listed amino acids can be modified as set forth in the Tables
SSDSAQGSDVSLTA

SEQ ID NO: 1228 - protein, 12 aa, Homo sapiens
  (1)..(12): One or more of the listed amino acids can be modified as set forth in the Tables
SSDSPPRPQPAF

SEQ ID NO: 1229 - protein, 9 aa, Homo sapiens
  (1)..(9): One or more of the listed amino acids can be modified as set forth in the Tables
SSDSPTNHF

SEQ ID NO: 1230 - protein, 13 aa, Homo sapiens
  (1)..(13): One or more of the listed amino acids can be modified as set forth in the Tables
SSGRSPSKAVAAR

SEQ ID NO: 1231 - protein, 9 aa, Homo sapiens
  (1)..(9): One or more of the listed amino acids can be modified as set forth in the Tables
SSIPSTLSL

SEQ ID NO: 1232 - protein, 8 aa, Homo sapiens
  (1)..(8): One or more of the listed amino acids can be modified as set forth in the Tables
SSISPVRL

SEQ ID NO: 1233 - protein, 9 aa, Homo sapiens
  (1)..(9): One or more of the listed amino acids can be modified as set forth in the Tables
SSLPRYLGL

SEQ ID NO: 1234 - protein, 9 aa, Homo sapiens
  (1)..(9): One or more of the listed amino acids can be modified as set forth in the Tables
SSMKSPLYL

SEQ ID NO: 1235 - protein, 9 aa, Homo sapiens
  (1)..(9): One or more of the listed amino acids can be modified as set forth in the Tables
SSMSPLPQM

SEQ ID NO: 1236 - protein, 7 aa, Homo sapiens
  (1)..(7): One or more of the listed amino acids can be modified as set forth in the Tables
SSPEFFM

SEQ ID NO: 1237 - protein, 10 aa, Homo sapiens
  (1)..(10): One or more of the listed amino acids can be modified as set forth in the Tables
SSPRSPTTTL

SEQ ID NO: 1238 - protein, 9 aa, Homo sapiens
  (1)..(9): One or more of the listed amino acids can be modified as set forth in the Tables
SSSGSPHLY

SEQ ID NO: 1239 - protein, 11 aa, Homo sapiens
  (1)..(11): One or more of the listed amino acids can be modified as set forth in the Tables
SSSSSGSPHLY

SEQ ID NO: 1240 - protein, 9 aa, Homo sapiens
  (1)..(9): One or more of the listed amino acids can be modified as set forth in the Tables
SSVPGVRLL

SEQ ID NO: 1241 - protein, 10 aa, Homo sapiens
  (1)..(10): One or more of the listed amino acids can be modified as set forth in the Tables
SSVPGVRLLQ

SEQ ID NO: 1242 - protein, 11 aa, Homo sapiens
  (1)..(11): One or more of the listed amino acids can be modified as set forth in the Tables
SSVPGVRLLQD

SEQ ID NO: 1243 - protein, 14 aa, Homo sapiens
  (1)..(14): One or more of the listed amino acids can be modified as set forth in the Tables
SSVPGVRLLQDSVD

SEQ ID NO: 1244 - protein, 14 aa, Homo sapiens
  (1)..(14): One or more of the listed amino acids can be modified as set forth in the Tables
SSVPGVRLLQDSVD

SEQ ID NO: 1245 - protein, 9 aa, Homo sapiens
  (1)..(9): One or more of the listed amino acids can be modified as set forth in the Tables
SSVSPAVSK

SEQ ID NO: 1246 - protein, 9 aa, Homo sapiens
  (1)..(9): One or more of the listed amino acids can be modified as set forth in the Tables
SSYPRPLTY

SEQ ID NO: 1247 - protein, 11 aa, Homo sapiens
  (1)..(11): One or more of the listed amino acids can be modified as set forth in the Tables
STDSGLGLGCY

SEQ ID NO: 1248 - protein, 11 aa, Homo sapiens
  (1)..(11): One or more of the listed amino acids can be modified as set forth in the Tables
STDSGLGLGCY

SEQ ID NO: 1249 - protein, 8 aa, Homo sapiens
  (1)..(8): One or more of the listed amino acids can be modified as set forth in the Tables
STDSPRLL

SEQ ID NO: 1250 - protein, 10 aa, Homo sapiens
  (1)..(10): One or more of the listed amino acids can be modified as set forth in the Tables
STFSTNYRSL

SEQ ID NO: 1251 - protein, 10 aa, Homo sapiens
  (1)..(10): One or more of the listed amino acids can be modified as set forth in the Tables
STFSTNYRSL

SEQ ID NO: 1252 - protein, 9 aa, Homo sapiens
  (1)..(9): One or more of the listed amino acids can be modified as set forth in the Tables
STIAILNSV

SEQ ID NO: 1253 - protein, 12 aa, Homo sapiens
  (1)..(12): One or more of the listed amino acids can be modified as set forth in the Tables
STISLVTGETER

SEQ ID NO: 1254 - protein, 10 aa, Homo sapiens
  (1)..(10): One or more of the listed amino acids can be modified as set forth in the Tables
STISPSGAFG

SEQ ID NO: 1255 - protein, 12 aa, Homo sapiens
  (1)..(12): One or more of the listed amino acids can be modified as set forth in the Tables
STISPSGAFGLF

SEQ ID NO: 1256 - protein, 9 aa, Homo sapiens
  (1)..(9): One or more of the listed amino acids can be modified as set forth in the Tables
STKSTELLL

SEQ ID NO: 1257 - protein, 9 aa, Homo sapiens
  (1)..(9): One or more of the listed amino acids can be modified as set forth in the Tables
STKSTELLL

SEQ ID NO: 1258 - protein, 10 aa, Homo sapiens
  (1)..(10): One or more of the listed amino acids can be modified as set forth in the Tables
STLLASPMLK

SEQ ID NO: 1259 - protein, 10 aa, Homo sapiens
  (1)..(10): One or more of the listed amino acids can be modified as set forth in the Tables
STLLASPMLK

SEQ ID NO: 1260 - protein, 10 aa, Homo sapiens
  (1)..(10): One or more of the listed amino acids can be modified as set forth in the Tables
STMSLNIITV

SEQ ID NO: 1261 - protein, 9 aa, Homo sapiens
  (1)..(9): One or more of the listed amino acids can be modified as set forth in the Tables
STPSGYLEL

SEQ ID NO: 1262 - protein, 9 aa, Homo sapiens
  (1)..(9): One or more of the listed amino acids can be modified as set forth in the Tables
STSSGRLLY

SEQ ID NO: 1263 - protein, 8 aa, Homo sapiens
  (1)..(8): One or more of the listed amino acids can be modified as set forth in the Tables
SVASPLTL

SEQ ID NO: 1264 - protein, 11 aa, Homo sapiens
  (1)..(11): One or more of the listed amino acids can be modified as set forth in the Tables
SVFRHFGSFQK

SEQ ID NO: 1265 - protein, 12 aa, Homo sapiens
  (1)..(12): One or more of the listed amino acids can be modified as set forth in the Tables
SVGSDDELGPIR

SEQ ID NO: 1266 - protein, 9 aa, Homo sapiens
  (1)..(9): One or more of the listed amino acids can be modified as set forth in the Tables
SVINVFVGR

SEQ ID NO: 1267 - protein, 9 aa, Homo sapiens
  (1)..(9): One or more of the listed amino acids can be modified as set forth in the Tables
SVISDDSVL

SEQ ID NO: 1268 - protein, 10 aa, Homo sapiens
  (1)..(10): One or more of the listed amino acids can be modified as set forth in the Tables
SVISQERLSY

SEQ ID NO: 1269 - protein, 11 aa, Homo sapiens
  (1)..(11): One or more of the listed amino acids can be modified as set forth in the Tables
SVISSESGNTY

SEQ ID NO: 1270 - protein, 10 aa, Homo sapiens
  (1)..(10): One or more of the listed amino acids can be modified as set forth in the Tables
SVKSPEVQLL

SEQ ID NO: 1271 - protein, 9 aa, Homo sapiens
  (1)..(9): One or more of the listed amino acids can be modified as set forth in the Tables
SVLPRALSL

SEQ ID NO: 1272 - protein, 9 aa, Homo sapiens
  (1)..(9): One or more of the listed amino acids can be modified as set forth in the Tables
SVLSYTSVR

SEQ ID NO: 1273 - protein, 9 aa, Homo sapiens
  (1)..(9): One or more of the listed amino acids can be modified as set forth in the Tables
SVLVRQISL

SEQ ID NO: 1274 - protein, 10 aa, Homo sapiens
  (1)..(10): One or more of the listed amino acids can be modified as set forth in the Tables
SVMQSPLVGV

SEQ ID NO: 1275 - protein, 13 aa, Homo sapiens
  (1)..(13): One or more of the listed amino acids can be modified as set forth in the Tables
SVPGVRLLQDSVD

SEQ ID NO: 1276 - protein, 11 aa, Homo sapiens
  (1)..(11): One or more of the listed amino acids can be modified as set forth in the Tables
SVQSDQGYISR

SEQ ID NO: 1277 - protein, 9 aa, Homo sapiens
  (1)..(9): One or more of the listed amino acids can be modified as set forth in the Tables
SVRRSVLMK

SEQ ID NO: 1278 - protein, 9 aa, Homo sapiens
  (1)..(9): One or more of the listed amino acids can be modified as set forth in the Tables
SVRSLSLSL

SEQ ID NO: 1279 - protein, 9 aa, Homo sapiens
  (1)..(9): One or more of the listed amino acids can be modified as set forth in the Tables
SVRSPTPYK

SEQ ID NO: 1280 - protein, 10 aa, Homo sapiens
  (1)..(10): One or more of the listed amino acids can be modified as set forth in the Tables
SVSRSPVPEK

SEQ ID NO: 1281 - protein, 9 aa, Homo sapiens
  (1)..(9): One or more of the listed amino acids can be modified as set forth in the Tables
SVSSLEVHF

SEQ ID NO: 1282 - protein, 9 aa, Homo sapiens
  (1)..(9): One or more of the listed amino acids can be modified as set forth in the Tables
SVSSLEVHF

SEQ ID NO: 1283 - protein, 8 aa, Homo sapiens
  (1)..(8): One or more of the listed amino acids can be modified as set forth in the Tables
SVSSSSYR

SEQ ID NO: 1284 - protein, 9 aa, Homo sapiens
  (1)..(9): One or more of the listed amino acids can be modified as set forth in the Tables
SVTSPIKMK

SEQ ID NO: 1285 - protein, 9 aa, Homo sapiens
  (1)..(9): One or more of the listed amino acids can be modified as set forth in the Tables
SYMGHFDLL

SEQ ID NO: 1286 - protein, 10 aa, Homo sapiens
  (1)..(10): One or more of the listed amino acids can be modified as set forth in the Tables
SYPSPVPTSF

SEQ ID NO: 1287 - protein, 11 aa, Homo sapiens
  (1)..(11): One or more of the listed amino acids can be modified as set forth in the Tables
SYSFSSSSIGH

SEQ ID NO: 1288 - protein, 11 aa, Homo sapiens
  (1)..(11): One or more of the listed amino acids can be modified as set forth in the Tables
SYSFSSSSIGH

SEQ ID NO: 1289 - protein, 13 aa, Homo sapiens
  (1)..(13): One or more of the listed amino acids can be modified as set forth in the Tables
SYSYSFSSSSIGH

SEQ ID NO: 1290 - protein, 13 aa, Homo sapiens
  (1)..(13): One or more of the listed amino acids can be modified as set forth in the Tables
SYSYSFSSSSIGH

SEQ ID NO: 1291 - protein, 9 aa, Homo sapiens
  (1)..(9): One or more of the listed amino acids can be modified as set forth in the Tables
SYYSLPRSF

SEQ ID NO: 1292 - protein, 9 aa, Homo sapiens
  (1)..(9): One or more of the listed amino acids can be modified as set forth in the Tables
SYYSPSIGF

SEQ ID NO: 1293 - protein, 11 aa, Homo sapiens
  (1)..(11): One or more of the listed amino acids can be modified as set forth in the Tables
SYYSPSIGFSY

SEQ ID NO: 1294 - protein, 9 aa, Homo sapiens
  (1)..(9): One or more of the listed amino acids can be modified as set forth in the Tables
TAISPPLSV

SEQ ID NO: 1295 - protein, 12 aa, Homo sapiens
  (1)..(12): One or more of the listed amino acids can be modified as set forth in the Tables
TAPLVPPLSPQY

SEQ ID NO: 1296 - protein, 9 aa, Homo sapiens
  (1)..(9): One or more of the listed amino acids can be modified as set forth in the Tables
TASPVAVSL

SEQ ID NO: 1297 - protein, 9 aa, Homo sapiens
  (1)..(9): One or more of the listed amino acids can be modified as set forth in the Tables
TATSPLTSY

SEQ ID NO: 1298 - protein, 9 aa, Homo sapiens
  (1)..(9): One or more of the listed amino acids can be modified as set forth in the Tables
TEASPESML

SEQ ID NO: 1299 - protein, 9 aa, Homo sapiens
  (1)..(9): One or more of the listed amino acids can be modified as set forth in the Tables
TEDSNLRLF

SEQ ID NO: 1300 - protein, 9 aa, Homo sapiens
  (1)..(9): One or more of the listed amino acids can be modified as set forth in the Tables
TELPKRLSL

SEQ ID NO: 1301 - protein, 12 aa, Homo sapiens
  (1)..(12): One or more of the listed amino acids can be modified as set forth in the Tables
TEPLPEKTQESL

SEQ ID NO: 1302 - protein, 13 aa, Homo sapiens
  (1)..(13): One or more of the listed amino acids can be modified as set forth in the Tables
TESSPGSRQIQLW

SEQ ID NO: 1303 - protein, 8 aa, Homo sapiens
  (1)..(8): One or more of the listed amino acids can be modified as set forth in the Tables
THSLLLLL

SEQ ID NO: 1304 - protein, 8 aa, Homo sapiens
  (1)..(8): One or more of the listed amino acids can be modified as set forth in the Tables
THSLLLLL

SEQ ID NO: 1305 - protein, 9 aa, Homo sapiens
  (1)..(9): One or more of the listed amino acids can be modified as set forth in the Tables
TIRSPTTVL

SEQ ID NO: 1306 - protein, 11 aa, Homo sapiens
  (1)..(11): One or more of the listed amino acids can be modified as set forth in the Tables
TITPPDRYDSL

SEQ ID NO: 1307 - protein, 8 aa, Homo sapiens
  (1)..(8): One or more of the listed amino acids can be modified as set forth in the Tables
TKSSPLKI

SEQ ID NO: 1308 - protein, 9 aa, Homo sapiens
  (1)..(9): One or more of the listed amino acids can be modified as set forth in the Tables
TLASPSVFK

SEQ ID NO: 1309 - protein, 10 aa, Homo sapiens
  (1)..(10): One or more of the listed amino acids can be modified as set forth in the Tables
TLDSLDFARY

SEQ ID NO: 1310 - protein, 11 aa, Homo sapiens
  (1)..(11): One or more of the listed amino acids can be modified as set forth in the Tables
TLESTTVGTSV

SEQ ID NO: 1311 - protein, 9 aa, Homo sapiens
  (1)..(9): One or more of the listed amino acids can be modified as set forth in the Tables
TLLASPMLK

SEQ ID NO: 1312 - protein, 10 aa, Homo sapiens
  (1)..(10): One or more of the listed amino acids can be modified as set forth in the Tables
TLLSPSSIKV

SEQ ID NO: 1313 - protein, 9 aa, Homo sapiens
  (1)..(9): One or more of the listed amino acids can be modified as set forth in the Tables
TLSSIRHMI

SEQ ID NO: 1314 - protein, 9 aa, Homo sapiens
  (1)..(9): One or more of the listed amino acids can be modified as set forth in the Tables
TLSSIRHMI

SEQ ID NO: 1315 - protein, 10 aa, Homo sapiens
  (1)..(10): One or more of the listed amino acids can be modified as set forth in the Tables
TMASPGKDNY

SEQ ID NO: 1316 - protein, 9 aa, Homo sapiens
  (1)..(9): One or more of the listed amino acids can be modified as set forth in the Tables
TMFLRETSL

SEQ ID NO: 1317 - protein, 10 aa, Homo sapiens
  (1)..(10): One or more of the listed amino acids can be modified as set forth in the Tables
TPAPSRTASF

SEQ ID NO: 1318 - protein, 9 aa, Homo sapiens
  (1)..(9): One or more of the listed amino acids can be modified as set forth in the Tables
TPASPNREL

SEQ ID NO: 1319 - protein, 9 aa, Homo sapiens
  (1)..(9): One or more of the listed amino acids can be modified as set forth in the Tables
TPATPTSQF

SEQ ID NO: 1320 - protein, 9 aa, Homo sapiens
  (1)..(9): One or more of the listed amino acids can be modified as set forth in the Tables
TPHTPKSLL

SEQ ID NO: 1321 - protein, 11 aa, Homo sapiens
  (1)..(11): One or more of the listed amino acids can be modified as set forth in the Tables
TPISPGRASGM

SEQ ID NO: 1322 - protein, 14 aa, Homo sapiens
  (1)..(14): One or more of the listed amino acids can be modified as set forth in the Tables
TPISPGRASGMTTL

SEQ ID NO: 1323 - protein, 10 aa, Homo sapiens
  (1)..(10): One or more of the listed amino acids can be modified as set forth in the Tables
TPISPLKTGV

SEQ ID NO: 1324 - protein, 10 aa, Homo sapiens
  (1)..(10): One or more of the listed amino acids can be modified as set forth in the Tables
TPKSPGASNF

SEQ ID NO: 1325 - protein, 12 aa, Homo sapiens
  (1)..(12): One or more of the listed amino acids can be modified as set forth in the Tables
TPPSSEKLVSVM

SEQ ID NO: 1326 - protein, 11 aa, Homo sapiens
  (1)..(11): One or more of the listed amino acids can be modified as set forth in the Tables
TPQPSRPVSPA

SEQ ID NO: 1327 - protein, 12 aa, Homo sapiens
  (1)..(12): One or more of the listed amino acids can be modified as set forth in the Tables
TPQPSRPVSPAG

SEQ ID NO: 1328 - protein, 11 aa, Homo sapiens
  (1)..(11): One or more of the listed amino acids can be modified as set forth in the Tables
TPRPASPGPSL

SEQ ID NO: 1329 - protein, 10 aa, Homo sapiens
  (1)..(10): One or more of the listed amino acids can be modified as set forth in the Tables
TPRTPRTPQL

SEQ ID NO: 1330 - protein, 10 aa, Homo sapiens
  (1)..(10): One or more of the listed amino acids can be modified as set forth in the Tables
TPRTPRTPQL

SEQ ID NO: 1331 - protein, 9 aa, Homo sapiens
  (1)..(9): One or more of the listed amino acids can be modified as set forth in the Tables
TPSPARPAL

SEQ ID NO: 1332 - protein, 9 aa, Homo sapiens
  (1)..(9): One or more of the listed amino acids can be modified as set forth in the Tables
TPSSFDTHF

SEQ ID NO: 1333 - protein, 9 aa, Homo sapiens
  (1)..(9): One or more of the listed amino acids can be modified as set forth in the Tables
TPSSREGTL

SEQ ID NO: 1334 - protein, 9 aa, Homo sapiens
  (1)..(9): One or more of the listed amino acids can be modified as set forth in the Tables
TPVSPGSTF

SEQ ID NO: 1335 - protein, 9 aa, Homo sapiens
  (1)..(9): One or more of the listed amino acids can be modified as set forth in the Tables
TPVSPRLHV

SEQ ID NO: 1336 - protein, 9 aa, Homo sapiens
  (1)..(9): One or more of the listed amino acids can be modified as set forth in the Tables
TPVSPTASM

SEQ ID NO: 1337 - protein, 8 aa, Homo sapiens
  (1)..(8): One or more of the listed amino acids can be modified as set forth in the Tables
TPVSPVKF

SEQ ID NO: 1338 - protein, 9 aa, Homo sapiens
  (1)..(9): One or more of the listed amino acids can be modified as set forth in the Tables
TRDSLLIHL

SEQ ID NO: 1339 - protein, 8 aa, Homo sapiens
  (1)..(8): One or more of the listed amino acids can be modified as set forth in the Tables
TRLSPLEL

SEQ ID NO: 1340 - protein, 9 aa, Homo sapiens
  (1)..(9): One or more of the listed amino acids can be modified as set forth in the Tables
TRMSTVSEL

SEQ ID NO: 1341 - protein, 9 aa, Homo sapiens
  (1)..(9): One or more of the listed amino acids can be modified as set forth in the Tables
TRMSTVSEL

SEQ ID NO: 1342 - protein, 9 aa, Homo sapiens
  (1)..(9): One or more of the listed amino acids can be modified as set forth in the Tables
TRSSAVRLR

SEQ ID NO: 1343 - protein, 9 aa, Homo sapiens
  (1)..(9): One or more of the listed amino acids can be modified as set forth in the Tables
TRSSPVRKL

SEQ ID NO: 1344 - protein, 9 aa, Homo sapiens
  (1)..(9): One or more of the listed amino acids can be modified as set forth in the Tables
TRYPTILQL

SEQ ID NO: 1345 - protein, 10 aa, Homo sapiens
  (1)..(10): One or more of the listed amino acids can be modified as set forth in the Tables
TSDSPPHNDI

SEQ ID NO: 1346 - protein, 15 aa, Homo sapiens
  (1)..(15): One or more of the listed amino acids can be modified as set forth in the Tables
TSFSVGSDDELGPIR

SEQ ID NO: 1347 - protein, 13 aa, Homo sapiens
  (1)..(13): One or more of the listed amino acids can be modified as set forth in the Tables
TSGPGSRISSSSF

SEQ ID NO: 1348 - protein, 9 aa, Homo sapiens
  (1)..(9): One or more of the listed amino acids can be modified as set forth in the Tables
TSISPALAR

SEQ ID NO: 1349 - protein, 11 aa, Homo sapiens
  (1)..(11): One or more of the listed amino acids can be modified as set forth in the Tables
TSISPSRHGAL

SEQ ID NO: 1350 - protein, 9 aa, Homo sapiens
  (1)..(9): One or more of the listed amino acids can be modified as set forth in the Tables
TSPSYIDKL

SEQ ID NO: 1351 - protein, 9 aa, Homo sapiens
  (1)..(9): One or more of the listed amino acids can be modified as set forth in the Tables
TSVSPAPDK

SEQ ID NO: 1352 - protein, 11 aa, Homo sapiens
  (1)..(11): One or more of the listed amino acids can be modified as set forth in the Tables
TTDPLIRWDSY

SEQ ID NO: 1353 - protein, 9 aa, Homo sapiens
  (1)..(9): One or more of the listed amino acids can be modified as set forth in the Tables
TVDSPPWQL

SEQ ID NO: 1354 - protein, 11 aa, Homo sapiens
  (1)..(11): One or more of the listed amino acids can be modified as set forth in the Tables
TVFSPTLPAAR

SEQ ID NO: 1355 - protein, 9 aa, Homo sapiens
  (1)..(9): One or more of the listed amino acids can be modified as set forth in the Tables
TVKQKYLSF

SEQ ID NO: 1356 - protein, 9 aa, Homo sapiens
  (1)..(9): One or more of the listed amino acids can be modified as set forth in the Tables
TVNSPAIYK

SEQ ID NO: 1357 - protein, 10 aa, Homo sapiens
  (1)..(10): One or more of the listed amino acids can be modified as set forth in the Tables
TVNSPAIYKF

SEQ ID NO: 1358 - protein, 9 aa, Homo sapiens
  (1)..(9): One or more of the listed amino acids can be modified as set forth in the Tables
TVTPVPPPQ

SEQ ID NO: 1359 - protein, 12 aa, Homo sapiens
  (1)..(12): One or more of the listed amino acids can be modified as set forth in the Tables
TVYSSEEAELLK

SEQ ID NO: 1360 - protein, 9 aa, Homo sapiens
  (1)..(9): One or more of the listed amino acids can be modified as set forth in the Tables
TYEGIFKTL

SEQ ID NO: 1361 - protein, 10 aa, Homo sapiens
  (1)..(10): One or more of the listed amino acids can be modified as set forth in the Tables
VADSPAEVAL

SEQ ID NO: 1362 - protein, 11 aa, Homo sapiens
  (1)..(11): One or more of the listed amino acids can be modified as set forth in the Tables
VADSPRDTASL

SEQ ID NO: 1363 - protein, 9 aa, Homo sapiens
  (1)..(9): One or more of the listed amino acids can be modified as set forth in the Tables
VADTSIQKL

SEQ ID NO: 1364 - protein, 9 aa, Homo sapiens
  (1)..(9): One or more of the listed amino acids can be modified as set forth in the Tables
VEFPHSPEI

SEQ ID NO: 1365 - protein, 10 aa, Homo sapiens
  (1)..(10): One or more of the listed amino acids can be modified as set forth in the Tables
VEKLPDSPAL

SEQ ID NO: 1366 - protein, 7 aa, Homo sapiens
  (1)..(7): One or more of the listed amino acids can be modified as set forth in the Tables
VELSPAR

SEQ ID NO: 1367 - protein, 9 aa, Homo sapiens
  (1)..(9): One or more of the listed amino acids can be modified as set forth in the Tables
VELSPARSW

SEQ ID NO: 1368 - protein, 9 aa, Homo sapiens
  (1)..(9): One or more of the listed amino acids can be modified as set forth in the Tables
VELSPARSW

SEQ ID NO: 1369 - protein, 10 aa, Homo sapiens
  (1)..(10): One or more of the listed amino acids can be modified as set forth in the Tables
VETSFRKLSF

SEQ ID NO: 1370 - protein, 10 aa, Homo sapiens
  (1)..(10): One or more of the listed amino acids can be modified as set forth in the Tables
VETSFRKLSF

SEQ ID NO: 1371 - protein, 11 aa, Homo sapiens
  (1)..(11): One or more of the listed amino acids can be modified as set forth in the Tables
VGSDDELGPIR

SEQ ID NO: 1372 - protein, 9 aa, Homo sapiens
  (1)..(9): One or more of the listed amino acids can be modified as set forth in the Tables
VIMSIRTKL

SEQ ID NO: 1373 - protein, 9 aa, Homo sapiens
  (1)..(9): One or more of the listed amino acids can be modified as set forth in the Tables
VIMSIRTKL

SEQ ID NO: 1374 - protein, 11 aa, Homo sapiens
  (1)..(11): One or more of the listed amino acids can be modified as set forth in the Tables
VISDGGDSEQF

SEQ ID NO: 1375 - protein, 10 aa, Homo sapiens
  (1)..(10): One or more of the listed amino acids can be modified as set forth in the Tables
VLASPLKTGR

SEQ ID NO: 1376 - protein, 9 aa, Homo sapiens
  (1)..(9): One or more of the listed amino acids can be modified as set forth in the Tables
VLDSPASKK

SEQ ID NO: 1377 - protein, 11 aa, Homo sapiens
  (1)..(11): One or more of the listed amino acids can be modified as set forth in the Tables
VLEKSPGKLLV

SEQ ID NO: 1378 - protein, 9 aa, Homo sapiens
  (1)..(9): One or more of the listed amino acids can be modified as set forth in the Tables
VLFSSPPQM

SEQ ID NO: 1379 - protein, 9 aa, Homo sapiens
  (1)..(9): One or more of the listed amino acids can be modified as set forth in the Tables
VLFSSPPQM

SEQ ID NO: 1380 - protein, 10 aa, Homo sapiens
  (1)..(10): One or more of the listed amino acids can be modified as set forth in the Tables
VLMKSPSPAL

SEQ ID NO: 1381 - protein, 10 aa, Homo sapiens
  (1)..(10): One or more of the listed amino acids can be modified as set forth in the Tables
VLMKSPSPAL

SEQ ID NO: 1382 - protein, 9 aa, Homo sapiens
  (1)..(9): One or more of the listed amino acids can be modified as set forth in the Tables
VLYSPQMAL

SEQ ID NO: 1383 - protein, 8 aa, Homo sapiens
  (1)..(8): One or more of the listed amino acids can be modified as set forth in the Tables
VMDSPVHL

SEQ ID NO: 1384 - protein, 8 aa, Homo sapiens
  (1)..(8): One or more of the listed amino acids can be modified as set forth in the Tables
VMDSPVHL

SEQ ID NO: 1385 - protein, 10 aa, Homo sapiens
  (1)..(10): One or more of the listed amino acids can be modified as set forth in the Tables
VMFPGNSPSY

SEQ ID NO: 1386 - protein, 10 aa, Homo sapiens
  (1)..(10): One or more of the listed amino acids can be modified as set forth in the Tables
VMFRTPLASV

SEQ ID NO: 1387 - protein, 9 aa, Homo sapiens
  (1)..(9): One or more of the listed amino acids can be modified as set forth in the Tables
VMIGSPKKV

SEQ ID NO: 1388 - protein, 9 aa, Homo sapiens
  (1)..(9): One or more of the listed amino acids can be modified as set forth in the Tables
VMQSPLVGV

SEQ ID NO: 1389 - protein, 9 aa, Homo sapiens
  (1)..(9): One or more of the listed amino acids can be modified as set forth in the Tables
VMTSLQQEY

SEQ ID NO: 1390 - protein, 12 aa, Homo sapiens
  (1)..(12): One or more of the listed amino acids can be modified as set forth in the Tables
VPGVRLLQDSVD

SEQ ID NO: 1391 - protein, 9 aa, Homo sapiens
  (1)..(9): One or more of the listed amino acids can be modified as set forth in the Tables
VPKKPPPSP

SEQ ID NO: 1392 - protein, 10 aa, Homo sapiens
  (1)..(10): One or more of the listed amino acids can be modified as set forth in the Tables
VPKSGRSSSL

SEQ ID NO: 1393 - protein, 9 aa, Homo sapiens
  (1)..(9): One or more of the listed amino acids can be modified as set forth in the Tables
VPKSPAFAL

SEQ ID NO: 1394 - protein, 10 aa, Homo sapiens
  (1)..(10): One or more of the listed amino acids can be modified as set forth in the Tables
VPRPSTPSRL

SEQ ID NO: 1395 - protein, 9 aa, Homo sapiens
  (1)..(9): One or more of the listed amino acids can be modified as set forth in the Tables
VPRSPVIKI

SEQ ID NO: 1396 - protein, 13 aa, Homo sapiens
  (1)..(13): One or more of the listed amino acids can be modified as set forth in the Tables
VPRTPSRERSSSA

SEQ ID NO: 1397 - protein, 9 aa, Homo sapiens
  (1)..(9): One or more of the listed amino acids can be modified as set forth in the Tables
VPRTPVGKF

SEQ ID NO: 1398 - protein, 9 aa, Homo sapiens
  (1)..(9): One or more of the listed amino acids can be modified as set forth in the Tables
VPSSPLRKA

SEQ ID NO: 1399 - protein, 10 aa, Homo sapiens
  (1)..(10): One or more of the listed amino acids can be modified as set forth in the Tables
VPTSPKGRLL

SEQ ID NO: 1400 - protein, 9 aa, Homo sapiens
  (1)..(9): One or more of the listed amino acids can be modified as set forth in the Tables
VPVSPGQQL

SEQ ID NO: 1401 - protein, 9 aa, Homo sapiens
  (1)..(9): One or more of the listed amino acids can be modified as set forth in the Tables
VRLLQDSVD

SEQ ID NO: 1402 - protein, 9 aa, Homo sapiens
  (1)..(9): One or more of the listed amino acids can be modified as set forth in the Tables
VRQSPGPAL

SEQ ID NO: 1403 - protein, 9 aa, Homo sapiens
  (1)..(9): One or more of the listed amino acids can be modified as set forth in the Tables
VRTPSVQSL

SEQ ID NO: 1404 - protein, 9 aa, Homo sapiens
  (1)..(9): One or more of the listed amino acids can be modified as set forth in the Tables
VRYSQLLGL

SEQ ID NO: 1405 - protein, 9 aa, Homo sapiens
  (1)..(9): One or more of the listed amino acids can be modified as set forth in the Tables
VSDSPSHIA

SEQ ID NO: 1406 - protein, 10 aa, Homo sapiens
  (1)..(10): One or more of the listed amino acids can be modified as set forth in the Tables
VSDSPSHIAT

SEQ ID NO: 1407 - protein, 22 aa, Homo sapiens
  (1)..(22): One or more of the listed amino acids can be modified as set forth in the Tables
VSPSKSPSLSPSPPSPLEKTPL

SEQ ID NO: 1408 - protein, 22 aa, Homo sapiens
  (1)..(22): One or more of the listed amino acids can be modified as set forth in the Tables
VSPSKSPSLSPSPPSPLEKTPL

SEQ ID NO: 1409 - protein, 10 aa, Homo sapiens
  (1)..(10): One or more of the listed amino acids can be modified as set forth in the Tables
VSSPPPYTAY

SEQ ID NO: 1410 - protein, 14 aa, Homo sapiens
  (1)..(14): One or more of the listed amino acids can be modified as set forth in the Tables
VSSSDSPPRPQPAF

SEQ ID NO: 1411 - protein, 9 aa, Homo sapiens
  (1)..(9): One or more of the listed amino acids can be modified as set forth in the Tables
VSSSPRELL

SEQ ID NO: 1412 - protein, 9 aa, Homo sapiens
  (1)..(9): One or more of the listed amino acids can be modified as set forth in the Tables
VTKSSPRAL

SEQ ID NO: 1413 - protein, 9 aa, Homo sapiens
  (1)..(9): One or more of the listed amino acids can be modified as set forth in the Tables
VTTPNRLIY

SEQ ID NO: 1414 - protein, 9 aa, Homo sapiens
  (1)..(9): One or more of the listed amino acids can be modified as set forth in the Tables
VTTPTGYKY

SEQ ID NO: 1415 - protein, 11 aa, Homo sapiens
  (1)..(11): One or more of the listed amino acids can be modified as set forth in the Tables
VTTSTRTYSLG

SEQ ID NO: 1416 - protein, 11 aa, Homo sapiens
  (1)..(11): One or more of the listed amino acids can be modified as set forth in the Tables
VVSEVDIAKAD

SEQ ID NO: 1417 - protein, 10 aa, Homo sapiens
  (1)..(10): One or more of the listed amino acids can be modified as set forth in the Tables
VVSSPKLAPK

SEQ ID NO: 1418 - protein, 12 aa, Homo sapiens
  (1)..(12): One or more of the listed amino acids can be modified as set forth in the Tables
VYIPMSPGAHHF

SEQ ID NO: 1419 - protein, 12 aa, Homo sapiens
  (1)..(12): One or more of the listed amino acids can be modified as set forth in the Tables
VYIPMSPGAHHF

SEQ ID NO: 1420 - protein, 9 aa, Homo sapiens
  (1)..(9): One or more of the listed amino acids can be modified as set forth in the Tables
VYLPTHTSL

SEQ ID NO: 1421 - protein, 10 aa, Homo sapiens
  (1)..(10): One or more of the listed amino acids can be modified as set forth in the Tables
VYLPTHTSLL

SEQ ID NO: 1422 - protein, 11 aa, Homo sapiens
  (1)..(11): One or more of the listed amino acids can be modified as set forth in the Tables
VYLPTHTSLLN

SEQ ID NO: 1423 - protein, 12 aa, Homo sapiens
  (1)..(12): One or more of the listed amino acids can be modified as set forth in the Tables
VYLPTHTSLLNL

SEQ ID NO: 1424 - protein, 13 aa, Homo sapiens
  (1)..(13): One or more of the listed amino acids can be modified as set forth in the Tables
VYLPTHTSLLNLT

SEQ ID NO: 1425 - protein, 9 aa, Homo sapiens
  (1)..(9): One or more of the listed amino acids can be modified as set forth in the Tables
WEFGKRDSL

SEQ ID NO: 1426 - protein, 9 aa, Homo sapiens
  (1)..(9): One or more of the listed amino acids can be modified as set forth in the Tables
WIGLNSLSF

SEQ ID NO: 1427 - protein, 9 aa, Homo sapiens
  (1)..(9): One or more of the listed amino acids can be modified as set forth in the Tables
YAFEGTGSL

SEQ ID NO: 1428 - protein, 15 aa, Homo sapiens
  (1)..(15): One or more of the listed amino acids can be modified as set forth in the Tables
YAQPQTTTPLPAVSG

SEQ ID NO: 1429 - protein, 9 aa, Homo sapiens
  (1)..(9): One or more of the listed amino acids can be modified as set forth in the Tables
YASSKLLKI

SEQ ID NO: 1430 - protein, 11 aa, Homo sapiens
  (1)..(11): One or more of the listed amino acids can be modified as set forth in the Tables
YCISPSTAAQF

SEQ ID NO: 1431 - protein, 11 aa, Homo sapiens
  (1)..(11): One or more of the listed amino acids can be modified as set forth in the Tables
YCISPSTAAQF

SEQ ID NO: 1432 - protein, 9 aa, Homo sapiens
  (1)..(9): One or more of the listed amino acids can be modified as set forth in the Tables
YEFSPVKML

SEQ ID NO: 1433 - protein, 9 aa, Homo sapiens
  (1)..(9): One or more of the listed amino acids can be modified as set forth in the Tables
YEGSPIKVT

SEQ ID NO: 1434 - protein, 10 aa, Homo sapiens
  (1)..(10): One or more of the listed amino acids can be modified as set forth in the Tables
YEGSPIKVTL

SEQ ID NO: 1435 - protein, 9 aa, Homo sapiens
  (1)..(9): One or more of the listed amino acids can be modified as set forth in the Tables
YESPGKIFL

SEQ ID NO: 1436 - protein, 8 aa, Homo sapiens
  (1)..(8): One or more of the listed amino acids can be modified as set forth in the Tables
YGDRTSTF

SEQ ID NO: 1437 - protein, 9 aa, Homo sapiens
  (1)..(9): One or more of the listed amino acids can be modified as set forth in the Tables
YGITSPISL

SEQ ID NO: 1438 - protein, 11 aa, Homo sapiens
  (1)..(11): One or more of the listed amino acids can be modified as set forth in the Tables
YHLSPRAFLHY

SEQ ID NO: 1439 - protein, 9 aa, Homo sapiens
  (1)..(9): One or more of the listed amino acids can be modified as set forth in the Tables
YIKTELISV

SEQ ID NO: 1440 - protein, 10 aa, Homo sapiens
  (1)..(10): One or more of the listed amino acids can be modified as set forth in the Tables
YLDSGIHSGA

SEQ ID NO: 1441 - protein, 9 aa, Homo sapiens
  (1)..(9): One or more of the listed amino acids can be modified as set forth in the Tables
YLPSFFTKL

SEQ ID NO: 1442 - protein, 9 aa, Homo sapiens
  (1)..(9): One or more of the listed amino acids can be modified as set forth in the Tables
YLPTHTSLL

SEQ ID NO: 1443 - protein, 11 aa, Homo sapiens
  (1)..(11): One or more of the listed amino acids can be modified as set forth in the Tables
YLRSVGDGETV

SEQ ID NO: 1444 - protein, 11 aa, Homo sapiens
  (1)..(11): One or more of the listed amino acids can be modified as set forth in the Tables
YLVSPITGEKI

SEQ ID NO: 1445 - protein, 9 aa, Homo sapiens
  (1)..(9): One or more of the listed amino acids can be modified as set forth in the Tables
YPHSPGSQY

SEQ ID NO: 1446 - protein, 11 aa, Homo sapiens
  (1)..(11): One or more of the listed amino acids can be modified as set forth in the Tables
YPLQISPVSSY

SEQ ID NO: 1447 - protein, 9 aa, Homo sapiens
  (1)..(9): One or more of the listed amino acids can be modified as set forth in the Tables
YPRLSIPNL

SEQ ID NO: 1448 - protein, 9 aa, Homo sapiens
  (1)..(9): One or more of the listed amino acids can be modified as set forth in the Tables
YPSFRRSSL

SEQ ID NO: 1449 - protein, 9 aa, Homo sapiens
  (1)..(9): One or more of the listed amino acids can be modified as set forth in the Tables
YPVSPKQKY

SEQ ID NO: 1450 - protein, 9 aa, Homo sapiens
  (1)..(9): One or more of the listed amino acids can be modified as set forth in the Tables
YRNDSSSSL

SEQ ID NO: 1451 - protein, 9 aa, Homo sapiens
  (1)..(9): One or more of the listed amino acids can be modified as set forth in the Tables
YRRSVPTWL

SEQ ID NO: 1452 - protein, 10 aa, Homo sapiens
  (1)..(10): One or more of the listed amino acids can be modified as set forth in the Tables
YSDRSSGGSY

SEQ ID NO: 1453 - protein, 10 aa, Homo sapiens
  (1)..(10): One or more of the listed amino acids can be modified as set forth in the Tables
YSESRSSLDY

SEQ ID NO: 1454 - protein, 9 aa, Homo sapiens
  (1)..(9): One or more of the listed amino acids can be modified as set forth in the Tables
YSFCGTVEY

SEQ ID NO: 1455 - protein, 9 aa, Homo sapiens
  (1)..(9): One or more of the listed amino acids can be modified as set forth in the Tables
YSFSPSKSY

SEQ ID NO: 1456 - protein, 10 aa, Homo sapiens
  (1)..(10): One or more of the listed amino acids can be modified as set forth in the Tables
YSFSSSSIGH

SEQ ID NO: 1457 - protein, 10 aa, Homo sapiens
  (1)..(10): One or more of the listed amino acids can be modified as set forth in the Tables
YSLDSPGPEK

SEQ ID NO: 1458 - protein, 11 aa, Homo sapiens
  (1)..(11): One or more of the listed amino acids can be modified as set forth in the Tables
YSLDSPGPEKM

SEQ ID NO: 1459 - protein, 13 aa, Homo sapiens
  (1)..(13): One or more of the listed amino acids can be modified as set forth in the Tables
YSLDSPGPEKMAL

SEQ ID NO: 1460 - protein, 13 aa, Homo sapiens
  (1)..(13): One or more of the listed amino acids can be modified as set forth in the Tables
YSLDSPGPEKMAL

SEQ ID NO: 1461 - protein, 9 aa, Homo sapiens
  (1)..(9): One or more of the listed amino acids can be modified as set forth in the Tables
YSLSPRPSY

SEQ ID NO: 1462 - protein, 9 aa, Homo sapiens
  (1)..(9): One or more of the listed amino acids can be modified as set forth in the Tables
YSLSPSKSY

SEQ ID NO: 1463 - protein, 11 aa, Homo sapiens
  (1)..(11): One or more of the listed amino acids can be modified as set forth in the Tables
YSLSPSKSYKY

SEQ ID NO: 1464 - protein, 8 aa, Homo sapiens
  (1)..(8): One or more of the listed amino acids can be modified as set forth in the Tables
YSSLVRVL

SEQ ID NO: 1465 - protein, 10 aa, Homo sapiens
  (1)..(10): One or more of the listed amino acids can be modified as set forth in the Tables
YSTTPGGTLY

SEQ ID NO: 1466 - protein, 10 aa, Homo sapiens
  (1)..(10): One or more of the listed amino acids can be modified as set forth in the Tables
YTDSESSASL

SEQ ID NO: 1467 - protein, 10 aa, Homo sapiens
  (1)..(10): One or more of the listed amino acids can be modified as set forth in the Tables
YTSSRDAFGY

SEQ ID NO: 1468 - protein, 8 aa, Homo sapiens
  (1)..(8): One or more of the listed amino acids can be modified as set forth in the Tables
YVDAETSL

SEQ ID NO: 1469 - protein, 9 aa, Homo sapiens
  (1)..(9): One or more of the listed amino acids can be modified as set forth in the Tables
YVKLTPVSL

SEQ ID NO: 1470 - protein, 9 aa, Homo sapiens
  (1)..(9): One or more of the listed amino acids can be modified as set forth in the Tables
YVPDSPALL

SEQ ID NO: 1471 - protein, 9 aa, Homo sapiens
  (1)..(9): One or more of the listed amino acids can be modified as set forth in the Tables
YVSSPDPQL

SEQ ID NO: 1472 - protein, 15 aa, Clostridium tetani
QYIKANSKFIGITEL

SEQ ID NO: 1473 - protein, 16 aa, Clostridium tetani
AQYIKANSKFIGITEL

SEQ ID NO: 1474 - protein, 9 aa, Homo sapiens
HLFGYSWYK

SEQ ID NO: 1475 - protein, 9 aa, Homo sapiens
YLSGADLNL

SEQ ID NO: 1476 - protein, 9 aa, Homo sapiens
EIWTHSYKV

SEQ ID NO: 1477 - protein, 9 aa, Homo sapiens
ALLAVGATK

SEQ ID NO: 1478 - protein, 16 aa, Homo sapiens
WNRQLYPEWTEAQRLD

SEQ ID NO: 1479 - protein, 9 aa, Homo sapiens
ALNFPGSQK

SEQ ID NO: 1480 - protein, 9 aa, Homo sapiens
SQNFPGSQK

SEQ ID NO: 1481 - protein, 9 aa, Homo sapiens
KTWGQYWQV

SEQ ID NO: 1482 - protein, 9 aa, Homo sapiens
ITDQVPFSV

SEQ ID NO: 1483 - protein, 9 aa, Homo sapiens
IMDQVPFSV

SEQ ID NO: 1484 - protein, 9 aa, Homo sapiens
YLEPGPVTA

SEQ ID NO: 1485 - protein, 10 aa, Homo sapiens
VLYRYGSFSV

SEQ ID NO: 1486 - protein, 9 aa, Homo sapiens
LIYRRRLMK

SEQ ID NO: 1487 - protein, 9 aa, Homo sapiens
KIFGSLAFL

SEQ ID NO: 1488 - protein, 9 aa, Homo sapiens
VLRENTSPK

SEQ ID NO: 1489 - protein, 14 aa, Homo sapiens
LLKYRAREPVTKAE

SEQ ID NO: 1490 - protein, 9 aa, Homo sapiens
SLFRAVITK

SEQ ID NO: 1491 - protein, 9 aa, Homo sapiens
EADPTGHSY

SEQ ID NO: 1492 - protein, 9 aa, Homo sapiens
EVDPIGHLY

SEQ ID NO: 1493 - protein, 15 aa, Homo sapiens
TSYVKVLHHMVKISG

SEQ ID NO: 1494 - protein, 9 aa, Homo sapiens
GLYDGMEHL

SEQ ID NO: 1495 - protein, 9 aa, Homo sapiens
AAGIGILTV

SEQ ID NO: 1496 - protein, 23 aa, Homo sapiens
RNGYRALMDKSLHVGTQCALTRR

SEQ ID NO: 1497 - protein, 20 aa, Homo sapiens
VPNAPPAYEKLSAEQSPPPY

SEQ ID NO: 1498 - protein, 12 aa, Homo sapiens
PNAPPAYEKLSA

SEQ ID NO: 1499 - protein, 12 aa, Homo sapiens
PNAPPAYEKLSA

SEQ ID NO: 1500 - protein, 9 aa, Homo sapiens
APPAYEKLS

SEQ ID NO: 1501 - protein, 12 aa, Homo sapiens
APPAYEKLSAEQ

SEQ ID NO: 1502 - protein, 15 aa, Homo sapiens
APPAYEKLSAEQSPP

SEQ ID NO: 1503 - protein, 16 aa, Homo sapiens
APPAYEKLSAEQSPPP

SEQ ID NO: 1504 - protein, 17 aa, Homo sapiens
APPAYEKLSAEQSPPPY

SEQ ID NO: 1505 - protein, 9 aa, Homo sapiens
PPAYEKLSA

SEQ ID NO: 1506 - protein, 12 aa, Homo sapiens
PPAYEKLSAEQS

SEQ ID NO: 1507 - protein, 9 aa, Homo sapiens
PAYEKLSAE

SEQ ID NO: 1508 - protein, 12 aa, Homo sapiens
PAYEKLSAEQSP

SEQ ID NO: 1509 - protein, 12 aa, Homo sapiens
AYEKLSAEQSPP

SEQ ID NO: 1510 - protein, 12 aa, Homo sapiens
YEKLSAEQSPPP

SEQ ID NO: 1511 - protein, 9 aa, Homo sapiens
AAQERRVPR

SEQ ID NO: 1512 - protein, 9 aa, Homo sapiens
LLGPGRPYR

SEQ ID NO: 1513 - protein, 10 aa, Homo sapiens
ASGPGGGAPR

SEQ ID NO: 1514 - protein, 16 aa, Homo sapiens
AQYIKANSKFIGITEL

SEQ ID NO: 1515 - protein, 9 aa, Homo sapiens
RLSNRLLLR

SEQ ID NO: 1516 - protein, 16 aa, Homo sapiens
AQNILLSNAPLGPQFP

SEQ ID NO: 1517 - protein, 11 aa, Homo sapiens
SSDYVIPIGTY

SEQ ID NO: 1518 - protein, 13 aa, Homo sapiens
SDAEKSDICTDEY

SEQ ID NO: 1519 - protein, 9 aa, Homo sapiens
KCDICTDEY

SEQ ID NO: 1520 - protein, 9 aa, Homo sapiens
YMDGTMSQV

SEQ ID NO: 1521 - protein, 21 aa, Homo sapiens [sequence omitted]